SEQUENCE LISTING (8 sequences)

SEQ ID NO: 1 - protein, 824 aa, Homo Sapien
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAEDDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHPSNVEMLSSMDSASVRIIKPFPAPQTPGRLQPAPVIPSAPAAPKLDHQRMDTIQEDPSTDSHMDEDGFEKDPFPNSSTAAKSFEDLTDHPVTRSEKAASFKLQRQNRVDSKETEC

SEQ ID NO: 2 - protein, 401 aa, Homo Sapien
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 3 - protein, 381 aa, Homo Sapien
MHLLAILFCALWSAVLAENSDDYDLMYVNLDNEIDNGLHPTEDPTPCACGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 4 - protein, 344 aa, Homo Sapien
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 5 - protein, 455 aa, Homo Sapien
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 6 - protein, 328 aa, Homo Sapien
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 7 - protein, 250 aa, Homo Sapien
MRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 8 - protein, 267 aa, Homo Sapien
MSLRVLGSGTWPSAPKMFLLLTALQVLAIAMTQSQEDENKIIGGHTCTRSSQPWQAALLAGPRRRFLCGGALLSGQWVITAAHCGRPILQVALGKHNLRRWEATQQVLRVVRQVTHPNYNSRTHDNDLMLLQLQQPARIGRAVRPIEVTQACASPGTSCRVSGWGTISSPIARYPASLQCVNINISPDEVCQKAYPRTITPGMVCAGVPQGGKDSCQGDSGGPLVCRGQLQGLVSWGMERCALPGYPGVYTNLCKYRSWIEETMRDK

(19) United States
(12) Patent Application Publication
(10) Pub. No.: US 2012/0178111 A1
(43) Pub. Date: Jul. 12, 2012

(54) METHODS AND COMPOSITIONS FOR THE DETECTION OF LUNG CANCERS

(76) Inventors: Eleftherios P. Diamandis, Toronto (CA); Chris Planque, Montpellier (FR)

(21) Appl. No.: 13/497,629

(22) PCT Filed: Sep. 23, 2010
(86) PCT No.: PCT/CA10/01511

§ 371 Date: Mar. 22, 2012

Related U.S. Application Data
(60) Provisional application No. 61/245,156, filed on Sep. 23, 2009.

(51) Int. Cl. G01N 33/574; G01N 33/566

(52) U.S. Cl. 435/7.92; 436/501; 435/7.1

(57) ABSTRACT

A method of screening for, diagnosing or detecting lung cancer in a subject, the method comprising: a) determining a level of a biomarker or a plurality of biomarkers in a sample from the subject, wherein the biomarker(s) is/are selected from the biomarkers listed in Table 8, and b) comparing the level of each biomarker in the sample with a control; wherein an increased level of any one of the biomarkers compared to the control is indicative that the subject has lung cancer Biomarkers were identified by shot-gun proteomics analysis of lung cancer cell-lines H1688, H520, H460 and H23. These lines are of differing histo-types, and were grown on serum-free media.

### RELATED APPLICATIONS

This is a Patent Cooperation Treaty Application which claims the benefit of 35 U.S.C. 119 based on the priority of corresponding U.S. Provisional Patent Application No. 61/245,156 filed Sep. 23, 2009, which is incorporated herein in its entirety.

### FIELD OF THE DISCLOSURE

The disclosure relates to methods and compositions for the detection of lung cancers and specifically to the use of biomarkers and compositions comprising agents that bind the biomarkers for the detection of lung cancers.

### BACKGROUND OF THE DISCLOSURE

Lung cancer is the leading cause of cancer-related mortality worldwide in both men and women. An estimated 213,000 new cases and 160,000 deaths from lung cancer occur in the United States every year (http://www.cancer.gov/cancertopics/types/lung). According to the World Health Organization, lung cancers are largely classified into two histologically distinct types, based on the size and appearance of the malignant cells: small cell (SCLC) and non-small cell lung cancer (NSCLC). NSCLC, which comprises more than 80% of lung cancers, can be further divided into adenocarcinoma (ADC), squamous cell carcinoma (SCC) and large cell carcinoma (LCC).

Despite advances in treatments such as surgery, chemotherapy and radiotherapy, the clinical outcome for patients with lung cancer still remains poor. The overall five-year survival rate is only 10 to 15% [1], mainly because at the time of diagnosis, most lung cancer patients are at advanced stages. In this context, there is a critical need to detect lung cancer earlier, by improving the current diagnostic methods such as computed tomography and chest X-ray and by discovering useful diagnostic and prognostic biomarkers. To date, a number of serum biomarkers for lung cancer have been studied, including carcinoembryonic antigen (CEA), squamous cell carcinoma antigen (SCC-Ag), neuron specific enolase (NSE), tissue polypeptide antigen (TPA), cytokeratin 19 fragment (CYFRA 21-1) and progastrin-releasing peptide (Pro-GRP). They are elevated in serum of patients with lung cancer, but they are not sensitive or specific enough, alone or in combination, to reliably diagnose asymptomatic patients with lung cancer.

Recently, new approaches in clinical proteomics have been developed to identify novel biomarkers of lung pathology (chronic obstructive pulmonary disease [COPD], asthma, pleural effusion, cancer) and to gain insights into disease mechanisms in which proteins play a major role. Some proteomic analyses of various biological fluids associated with the human airway have been reported, including nasal lavage fluid [2-4], bronchoalveolar lavage fluid [5, 6] and saliva [7, 8]. By using a combination of 2-DE analysis and GeLC-MS/MS, Nicholas et al., identified 258 proteins in human sputum and, among them, 191 were of human origin. Proteins included lower and upper airway secretory products, cellular products and inflammatory cell-derived products [9]. In addition, Casado et al., used CapLC-ESI-Q/TOF-MS to investigate the proteome profiles of hypertonic saline-induced sputum samples from healthy smokers and patients with COPD of different severity [10]. A total of 203 unique proteins were identified, of which some may be markers of COPD severity. The proteomic profiling of human pleural effusion from 43 lung adenocarcinoma was also studied using a two-dimensional (2D) nano-HPLC-ESI-MS/MS system [11]. The results revealed 1,415 unique proteins, of which 124 were identified with higher confidence (at least two unique peptides sequences matched). However, there are inherent limitations of using MS for biomarker discovery in complex biological mixtures such as fluids or serum [12, 13], requiring methodologies for depletion of high abundance proteins such as albumin and immunoglobulins. These limitations illustrate the need to find other sources to mine for biomarker discovery.

One approach to overcome this limitation posed by complex mixtures is by using a cell culture model, where cells are grown in serum-free media (SFM), used to perform proteomic analysis. This model offers various advantages over the traditional cultures in serum-supplemented media: it reduces complexity by avoiding interferences from nutritional proteins present in the media, increases the reproducibility and allows detection of low abundance proteins. This strategy has been successfully used for the discovery of novel breast and prostate biomarkers [14, 15]. This technique was also reported in lung-related proteomic approaches. Tachibana et al., reported the regulatory roles of β1-integrin in morphological differentiation in CADO LC6 cells, a SCLC cell line cultured in serum-free media [16]. To explore serum biomarkers of lung cancer at early stage, M-BE, an SV40T-transformed human bronchial epithelial cell line with the phenotypic features of early tumorigenesis at high passage, was cultured and the conditioned media (CM) was used to collect its secretory proteins [17]. Proteins secreted from different passages of M-BE cells were extracted and then separated by 2-DE, followed by Matrix Assisted Laser Desorption Ionization Time-Of-Flight (MALDI-TOF)/TOF mass spectrometry (MS). This resulted in the identification of 47 proteins, including cathepsin D, that exhibited increased abundance in culture media or cells during passaging. Moreover, Xiao et al., analyzed the proteins released into the serum-free medium from the tumor microenvironment with short time-cultured lung cancer and adjacent normal bronchial epithelial cells [18], thus demonstrating the versatility of this approach.

### SUMMARY OF THE DISCLOSURE

A shotgun proteomic analysis of the conditioned media of four lung cancer cell lines of differing histotypes is disclosed herein. The aim was to identify secreted or membrane-bound proteins that are useful as novel lung cancer biomarkers.

In an aspect, the disclosure provides a method of screening for, diagnosing or detecting lung cancer in a subject, the method comprising:

a) determining a level of a biomarker or a plurality of biomarkers in a sample from the subject, wherein the biomarker(s) is/are selected from the biomarkers listed in Table 8 and

b) comparing the level of each biomarker in the sample with a control; wherein an increased level of any one of the biomarkers compared to the control is indicative that the subject has lung cancer.

In another aspect, the disclosure provides a method for screening a subject for the need for follow-up lung cancer testing comprising:

a) determining a level of a biomarker or a plurality of biomarkers in a sample from the subject, wherein the biomarker(s) is/are selected from the biomarkers listed in Table 8; and

b) comparing the level of each biomarker in the sample with a control; wherein an increased level of any one of the biomarkers compared to the control is indicative that the subject is in need for follow-up lung cancer testing.

In a further aspect, the disclosure provides, a method for prognosing lung cancer recurrence in a subject previously having lung cancer, the method comprising:
(a) determining the level of a biomarker or a plurality of biomarkers in a sample from the subject, optionally wherein the sample is obtained after treatment, optionally obtained after surgical resection, wherein the biomarker(s) is/are selected from the biomarkers listed in Table 8; and
(b) comparing the level of each biomarker in the sample with a positive control or a reference level associated with recurrence;
   wherein the disease outcome associated with the positive control reference level most similar to the level of each biomarker in the sample is the predicted prognosis.

Yet a further aspect provides a method of monitoring response to treatment comprising:
a) determining a base-line level of a biomarker or a plurality of biomarkers in a base-line sample from the subject, wherein the biomarker(s) is/are selected from the biomarkers listed in Table 8;
b) determining a level of a biomarker or a plurality of biomarkers in a post-treatment sample from the subject; and
c) comparing the level of each biomarker in the post-treatment sample with the base-line level;
   wherein an increase in the biomarker level in the post-treatment sample compared to the baseline level is indicative the subject is not responding or is responding poorly to treatment, and a decrease in the biomarker level in the post treatment sample compared to the base-line level is indicative that the subject is responding to treatment.

Another aspect provides a method of monitoring disease progression comprising:
a) determining a base-line level of a biomarker or a plurality of biomarkers in a base-line sample from the subject, wherein the biomarker(s) is/are selected from the biomarkers listed in Table 8;
b) determining a level of a biomarker or a plurality of biomarkers in a sample taken subsequent to the base-line sample from the subject; and
c) comparing the level of each biomarker in the sample with the base-line level;

wherein an increase in the biomarker level in the post-base-line sample compared to the base-line level is indicative the disease is progressing, and a decrease in the biomarker level in the post base-line sample compared to the base-line level is indicative that the disease is not progressing.

In a further embodiment, the biomarker(s) is/are selected from a disintegrin and metalloproteinase-17 (ADAM-17), Osteoprotegerin, Pentraxin 3, Follistatin, soluble tumor necrosis factor receptor I (sTNF RI), and/or any combination thereof. In an embodiment, the biomarker is a soluble biomarker. In yet a further embodiment, the soluble biomarker is sADAM-17, sOsteoprotegerin, sPentraxin, sFollistatin and/or sTNF RI.

In another embodiment, the lung cancer is a small cell lung cancer (SCLC). In another embodiment, the lung cancer is a non-small cell lung cancer (NSCLC).

In an embodiment, the sample and/or control comprises serum.

Another aspect provides an immunoassay for detecting a biomarker comprising an antibody immobilized on a solid support, wherein the antibody binds a biomarker, the biomarker selected from a biomarker listed in Table 8, preferably selected from ADAM-17, Osteoprotegerin, or a combination thereof.

A further aspect provides a composition comprising at least two detection agents that bind a biomarker selected from the biomarkers listed in Table 8, preferably selected from ADAM-17, Osteoprotegerin, Pentraxin 3, Follistatin, and sTNF RI.

Another aspect provides a kit for detecting a biomarker comprising:
(a) at least two agents, each of which binds a biomarker selected from a biomarker listed in Table 8, such as ADAM-17, Osteoprotegerin, Pentraxin 3, Follistatin, or sTNF RI, or any combination thereof; and
(b) instructions for use, or a quantity of at least one purified standard, wherein the standard is selected from a Table 8 polypeptide, such as ADAM-17 polypeptide, Osteoprotegerin polypeptide, Pentraxin 3 polypeptide, Follistatin polypeptide or sTNF RI polypeptide.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1. Outline of experimental workflow showing proteins secreted from four lung cancer cell lines into the serum free media were digested with trypsin and subjected to strong cation exchange liquid chromatography followed by LC-MS/MS. The resulting raw mass spectra were analyzed by Mascot and X!Tandem search engines and by Scaffold.

FIG. 2. Number of proteins identified by LC-MS/MS in CM of 4 lung cancer cell lines and their cellular localization. Shown is the overlap of 3 independent replicates (Rep 1-3) and total number of proteins identified. Lower panel depicts cellular localization. Mitoch., mitochondria; Golgi app., Golgi apparatus; ER., endoplasmic reticulum; Other org., other organelles.

FIG. 3. Overlap of proteins identified in CM by LC-MS/MS between each of four lung cancer cell lines. Overlap of total proteins (A, total number in parentheses), extracellular proteins (B) and membrane proteins (C).

FIG. 4. Overlap of proteins identified herein and six other lung-related proteomics studies. Casado et al. [10]; Tyan et al. [11]; Nicholas et al. [9]; Huang et al. [25]; Tian et al. [26]; Xiao et al. [18].

FIG. 5. Detection of Osteoprotegerin, sTNF RI, Follistatin, PTX3 and ADAM-17 in serum. Levels of these candidate biomarkers were measured by ELISA in serum of patients with or without lung cancer. n, number of subjects. Median values are shown by a horizontal line. P values were calculated with the Mann-Whitney test.

FIG. 6. Biological function analyses. The top 10 functions for the extracellular and membrane-bound proteins are shown, as determined by Ingenuity Pathway Analysis (IPA). The y axis shows the negative log of p value.

FIG. 7. Molecular functions related to diseases associated with ADAM-17. The web diagram generated through IPA software depicts the biological functions that ADAM-17 is associated with, in the context of disease.

FIG. 8. Optimization of seeding density for H520.

A, IGFBP2 levels measured in CM at different seeding densities (8, 12 and 16 million cells); B, LDH levels measured in CM at different seeding densities (8, 12 and 16 million cells); C, IGFBP2/LDH ratio calculated at different seeding densities (8, 12 and 16 million cells).

FIG. 9. Optimization of seeding density for H460.

A, IGFBP2 levels measured in CM at different seeding densities (1, 2 and 4 million cells); B, LDH levels measured in CM at different seeding densities (1, 2 and 4 million cells); C, IGFBP2/LDH ratio calculated at different seeding densities (1, 2 and 4 million cells).

FIG. 10. Optimization of seeding density for H23.

A, IGFBP2 levels measured in CM at different seeding densities (2, 4 and 8 million cells); B, LDH levels measured in CM at different seeding densities (2, 4 and 8 million cells); C, IGFBP2/LDH ratio calculated at different seeding densities (2, 4 and 8 million cells).

FIG. 11. Optimization of seeding density for H1688.

A, IGFBP2, KLK11 and KLK14 levels measured in CM at different seeding densities (5 and 10 million cells); B, LDH levels measured in CM at different seeding densities (5 and 10 million cells); C, IGFBP2, KLK11, KLK14/LDH ratio calculated at different seeding densities (5 and 10 million cells).

FIG. 12. Identification of internal control proteins by LC MS/MS.

H1688 expresses IGFBP2, KLK11 and KLK14 in concentrations ranging from approximately 2-35 μg/L, as measured by ELISA. The sequences of the respective proteins are indicated (A) IGFBP2, (B) KLK11, (C) KLK14. The peptides identified by MS in the CM of H1688 are highlighted in yellow.

FIG. 13. Molecular functions related to diseases associated with Follistatin.

The web diagram generated through IPA software depicts the biological functions that Follistatin is associated with, in the context of disease.

FIG. 14. Molecular functions related to diseases associated with PTX3.

The web diagram generated through IPA software depicts the biological functions that PTX3 is associated with, in the context of disease.

FIG. 15. Molecular functions related to diseases associated with TNFRSF1A. The web diagram generated through IPA software depicts the biological functions that TNFRSF1A is associated with, in the context of disease.

FIG. 16. Molecular functions related to diseases associated with Osteoprotegerin (TNFRSF11B). The web diagram generated through IPA software depicts the biological functions that Osteoprotegerin is associated with, in the context of disease.

FIG. 17. Sensitivity and Specificity Analysis for ADAM-17, Osteoprotegerin, Pentraxin 3, sTNF RI and Follistatin calculated using ROC curve analysis.

FIG. 18. Sensitivity and Specificity Analysis for Pentraxin 3 calculated using ROC curve analysis.

A, ROC curve for Pentraxin 3 comparing all cases and all controls; B, ROC curve for Pentraxin 3 comparing all cases and high-risk controls; C, ROC curve for Pentraxin 3 comparing all cases and other cancer controls.

FIG. 19. ROC curve analysis for Pentraxin 3 amongst sub-groups of patients stratified by histology.

A, ROC curve for Pentraxin 3 comparing NSCLC cases and high-risk controls; B, ROC curve for Pentraxin 3 comparing SCLC cases and high-risk controls; C, ROC curve for Pentraxin 3 comparing lung cancer of undetermined histology and high-risk controls; D, ROC curve for Pentraxin 3 comparing squamous cell carcinomas and high-risk controls; E, ROC curve for Pentraxin 3 comparing adenocarcinomas and high-risk controls.

FIG. 20. ROC curve analysis for Pentraxin 3 amongst patients at different clinicopathological stages.

A, ROC curve for Pentraxin 3 comparing pathological stage I lung cancers and high-risk controls; B, ROC curve for Pentraxin 3 comparing pathological stage 11 lung cancers and high-risk controls; C, ROC curve for Pentraxin 3 comparing pathological stage III lung cancers and high-risk controls; D, ROC curve for Pentraxin 3 comparing pathological stage IV lung cancers and high-risk controls; E, ROC curve for Pentraxin 3 comparing pathological or clinical stage I lung cancers and high-risk controls; F, ROC curve for Pentraxin 3 comparing pathological or clinical stage II lung cancers and high-risk controls; G, ROC curve for Pentraxin 3 comparing pathological or clinical stage III lung cancers and high-risk controls; H, ROC curve for Pentraxin 3 comparing pathological or clinical stage IV lung cancers and high-risk controls.

### DETAILED DESCRIPTION OF THE DISCLOSURE

### I. Definitions

The term “lung cancer” as used herein refers to all types of lung cancer, benign to malignant, and includes, but is not limited to the non-small cell lung cancer (NSCLC) and small cell lung cancer (SCLC) and for example the following NSCLC histological backgrounds: adenocarcinoma (ADC), squamous cell carcinoma (SCC), and large cell carcinoma (LCC). The World Health Organization (WHO) histologic classification of lung cancer describes 2 major groupings dependent on cell type: NSCLC and SCLC. The WHO histological classification of lung tumors includes adenosquamous carcinoma, carcinoid tumors, bronchial gland carcinoma, malignant mesothelial tumors and miscellaneous malignant tumors. In addition, lung cancer can be characterized by pathological stage (e.g. based on biopsy staining) and/or clinical stage (e.g. based on imaging), including stage I, stage II, stage III and stage IV. As used herein, a “combined stage” refers to the pathological stage, if available, or the clinical stage if the pathological stage is not available.

The phrase “screening for, diagnosing or detecting lung cancer” refers to a method or process of determining if a subject has or does not have lung cancer. For example, detection of increased levels of biomarker(s) selected from Table 8, 15 and/or of ADAM-17, Osteoprotegerin, Pentraxin 3, Follistatin, or sTNF RI, or any combination thereof, compared to a control is indicative that the subject has lung cancer.

The term “subject” as used herein refers to any member of the animal kingdom, preferably a human being including for example a subject that has or is suspected of having lung cancer.

The term “level” as used herein refers to an amount (e.g. relative amount or concentration) of biomarker that is detectable or measurable in a sample. For example, the level can be a concentration such as μg/L or a relative amount such as 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 10, 15, 20, 25, 30, 40, 60, 80 and/or 100 times a control level, where for example, the control level is the level such as the average or median level in a normal sample (e.g. serum from a subject without lung cancer). The level of biomarker can be, for example, the level of soluble (e.g. cleaved, secreted, released, or shed biomarker) polypeptide biomarker.

The term “cut-off level” as used herein refers to a value corresponding to a level of a biomarker in a sample above which a subject is likely to have lung cancer for a particular specificity and sensitivity and which is used for determining if a subject has or does not have lung cancer. For example, the cut-off level can be the highest value associated with a panel of controls (e.g. 100% specificity). In a further example, the cut-off level can be a relative amount of a biomarker in comparison to a control, such as 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 10, and 40 times a control level.

The term “specificity” as used herein refers to the percentage of subjects without lung cancer that are identified as not having lung cancer based on a biomarker level that is, for example, at or below a control level and/or a cut-off level.

The term “sensitivity” as used herein refers to the percentage of subjects with lung cancer that are identified as having lung cancer based on a biomarker level that is, for example, above a control level and/or a cut-off level.

The term “control” as used herein refers to a sample from an individual or a group of individuals who are known as not having lung cancer or to a biomarker level or value, such as a cut-off value at which or below which individuals are likely to belong to a lung cancer free class. For example, where the control is a value, the value can for example correspond to the level of a biomarker in a control sample or set of samples. For example, the control can be a value (e.g. cut-off level) wherein samples from subjects with a level above the cut-off value have or are likely to have lung cancer. In another example, the control can correspond to the median level of a biomarker in a set of samples from subjects without lung cancer. In addition, the control is optionally derived from tissue of the same type as the sample of the subject being tested. For example, the control can be a serum sample where the sample from the subject being tested (e.g. test sample) is a serum sample.

The term “high risk control” as used herein refers to subjects that have smoked 30 pack years, optionally subjects that are 50 years of age or older and that have smoked 30 pack years with lung lesions observed on a chest X-ray or on a computed tomography (CT) scan that are suspected of being lung cancer but proven not to be lung cancer at 1 year follow up. If at 1 year follow up participant has been diagnosed with a type of cancer other than lung cancer, then the participant is considered an “other cancer” control.

The term “positive control” as used herein refers to a sample of an individual or a group of individuals with lung cancer and/or a value e.g. corresponding to a level of one or more biomarkers associated with the disease class, e.g. lung cancer.

The term “reference level” as used herein refers to the level of one or more biomarkers associated with a particular group, such as a prognostic group, for example recurrence.

The term “reference level associated with recurrence” as used herein refers to a level of a biomarker in subjects associated with recurrence of lung cancer.

The term “baseline level” as used herein refers to a level that is used for comparison to a sample taken at a later time point. For example, in methods related to monitoring response to treatment or disease progression, “base-line level” can refer to a level of a biomarker in a sample taken prior to a subsequent sample, e.g. base line sample is taken before treatment, comparison to which provides an indication of response to treatment.

The term “biomarker” as used herein can be any type of molecule corresponding to a biomarker listed in Table 8, also referred to as “biomarkers of the disclosure”, that can be used to distinguish subjects with or without lung cancer, for example, ADAM-17, Osteoprotegerin, Pentraxin 3, Follistatin, sTNF RI and/or any combination thereof. The term biomarker includes without limitation, a nucleic acid sequence including a gene, or corresponding RNA, or a polypeptide, fragment thereof, or epitope that is differentially present, including differentially modified (e.g. differentially glycosylated), expressed, and/or soluble biomarkers e.g. biomarkers which are detectable in a biological fluid and which are differentially cleaved, secreted, released or shed in subjects with or without lung cancer.

The term “biomarker products” as used herein refer to biomarker gene products such as polypeptides including for example, soluble polypeptides, detectable for example in blood and/or RNA products expressed by and/or corresponding to a biomarker described in the present disclosure.

The term “prognosis” as used herein refers to an expected clinical outcome group such as a poor survival group or a good survival group associated with or reflected by an increased biomarker level or levels when compared to a control, wherein the biomarker(s) is/are selected from the biomarkers listed in Table 8, for example, ADAM-17, Osteoprotegerin, Pentraxin 3, Follistatin, sTNF RI and/or any combinations thereof.

The term “polypeptide biomarker” and/or “polypeptide biomarker product” refers to polypeptide and/or fragments thereof of a biomarker of the present disclosure and includes polypeptides translated from the RNA transcripts of biomarkers described herein or optionally, known in the art associated with lung cancer. Polypeptide biomarkers include modified (e.g. post-translational modifications such as glycosylation), expressed, as well as soluble biomarkers such as secreted, cleaved, released, and shed polypeptide products. The terms “polypeptide” and “protein” are intended to be used interchangeably.

The term “soluble biomarker” as used herein refers to a biomarker, preferably a soluble polypeptide biomarker that is released in any manner from a cell and detectable in a biological fluid, such as blood, serum, plasma, sputum, pleural effusion, nasal lavage fluid, bronchoalveolar lavage (BAL) fluid, saliva or tumor interstitial fluid and/or in fraction thereof. For example, without wishing to be bound to theory, a soluble biomarker can be cleaved, secreted, or shed from a cell, e.g. a tumour cell. Proteins which can serve as biomarkers, become elevated, for example in biological fluid such as serum, through several possible mechanisms. Molecules may be released into the circulation through aberrant shedding and secretion from tumour cells or through destruction of tissue architecture and angiogenesis as the tumour invades. Proteins can also be cleaved from the extracellular surface of tumour cells by proteases and subsequently make their way into the circulation. To this end, it is hypothesized that novel candidate biomarkers can be identified through extensive proteomic analysis of (a) supernatants of human cancer cell lines grown in vitro and/or (b) relevant biological fluids collected from cancer patients. Due to the close proximity of these fluids to tumor cells, it is hypothesized that they are highly enriched sources of proteins secreted, shed, or cleaved from the tumor cells. For example, ADAM-17 is a transmembrane glycoprotein. Soluble ADAM-17 (e.g. sADAM-17) refers to ADAM-17 that is not bound as a transmembrance protein to a cell membrane of a cell and which is detectable, for example, in blood. Accordingly, detecting a level of soluble biomarker, for example sADAM-17 refers to detecting the level of ADAM-17 that is not bound as a transmembrane protein to a cell in a biological fluid, such as blood.

The term “sample” as used herein refers to any biological fluid, cell or tissue sample from a subject which can be assayed for biomarkers (e.g. RNA and/or polypeptide products), such as soluble biomarkers in subjects having or not having lung cancer. For example the sample is optionally or comprises blood, tumor biopsy, serum, plasma, sputum, pleural effusion, nasal lavage fluid, BAL fluid, saliva or tumor interstitial fluid. The sample can for example be a “post-treatment” sample wherein the sample is obtained after one or more treatments, or a “base-line sample” which is for example used as a base line for assessing disease progression.

The term “biological fluid” as used herein refers to any body fluid, which can comprise cells or be substantially cell free, which can be assayed for biomarkers, including for example blood, serum, plasma, sputum, pleural effusion, nasal lavage fluid, bronchoalveolar (BAL) fluid, saliva or tumor interstitial fluid.

The term “antibody” as used herein is intended to include monoclonal antibodies, polyclonal antibodies, and chimeric antibodies. The antibody may be from recombinant sources and/or produced in transgenic animals. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies having specificity for a specific protein, such as the protein product of a biomarker of the disclosure, may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

The term “detection agent” as used herein refers to any molecule or compound that can bind to a biomarker product described herein, including polypeptides such as antibodies, nucleic acids and peptide mimetics. For example, a suitable antibody for detecting the level of a biomarker that is a transmembrane protein includes an antibody that binds an extracellular portion of the protein. The “detection agent” can for example be coupled to or labeled with a detectable marker. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The term “ADAM-17” means a disintegrin and metalloproteinase-17 and includes without limitation, all known ADAM-17 molecules, including naturally occurring variants, and including those deposited in Genbank with accession number NP_{—}003174.3 which is herein incorporated by reference.

The term “Osteoprotegerin” as used herein includes without limitation, all known Osteoprotegerin molecules, including naturally occurring variants, such as Osteoprotegerin precursor, and including those deposited in Genbank with accession number NP_{—}002537.3 which is herein incorporated by reference. Osteoprotegerin is a secreted member of the tumor necrosis factor receptor superfamily and is also known as tumour necrosis factor receptor superfamily member 11B (TNFRSF11B).

The term “Pentraxin 3” as used herein includes without limitation, all known Pentraxin 3 molecules, including naturally occurring variants, and including those deposited in Genbank with accession number NP_{—}002843.2 which is herein incorporated by reference. Pentraxin 3, is also known as tumor necrosis factor-stimulated gene 14 (TSG-14).

The term “Follistatin” includes without limitation, all known Follistatin molecules, including naturally occurring variants, for example, Follistatin isoform FST344 precursor and including those deposited in Genbank, for example, with accession number NP_{—}037541.1 which is herein incorporated by reference.

The term “sTNF RI” as used herein means soluble tumor necrosis factor receptor I and refers to the truncated, cleaved, shed, or non-membrane bound variant of TNFSFRIA and includes without limitation, all known sTNF RI molecules, including naturally occurring variants, and including those deposited in Genbank, for example, a deposit with accession number NP_{—}001056.1, which is herein incorporated by reference.

### II. Methods

The present disclosure pertains to methods for detecting lung cancer using biomarkers, which are differentially present, including soluble biomarkers, in individuals having or not having lung cancer. A cell culture model was employed where cells are grown in serum-free media, coupled with a proteomics approach to identify novel biomarkers associated with lung cancer, including the biomarkers listed in Table 8. Further it is demonstrated herein that detecting a disintegrin and metalloproteinase-17 (ADAM-17), Osteoprotegerin (OPG), Pentraxin 3 (PTX3), Follistatin and/or soluble tumor necrosis factor receptor I (sTNF RI) biomarker products, individually or in any combination, in patient samples, is useful for screening for, diagnosing and/or detecting lung cancer and/or detecting the presence of lung cancer cells. It is also herein demonstrated that levels of soluble biomarkers, ADAM-17, OPG, PTX3, Follistatin and/or sTNF RI are useful for screening for, diagnosing and/or detecting lung cancer and/or the presence of lung cancer cells.

Accordingly, an aspect of the disclosure provides a method of screening for, diagnosing or detecting lung cancer in a subject, the method comprising:
a) determining a level of one or more biomarkers in a sample from the subject, wherein the biomarker(s) is/are selected from the biomarkers listed in Table 8; and
b) comparing the level of each biomarker in the sample with a control,
wherein an increased level of any one of the biomarkers compared to the control is indicative that the subject has lung cancer.

In an embodiment, an increased level of each of the biomarkers compared to the control is indicative that the subject has lung cancer. In an embodiment, an increased level of one or more of ADAM-17, OPG, PTX3, Follistatin and/or sTNF RI compared to a control is indicative the subject has lung cancer. In an embodiment, an increased level of Pentraxin 3 compared to the control is indicative that the subject has lung cancer.

In another aspect, the disclosure provides a method of screening for the need for follow up lung cancer testing, the method comprising:
a) determining a level of one or more biomarkers in a sample from the subject, wherein the biomarker(s) is/are selected from the biomarkers listed in Table 8; and
b) comparing the level of each biomarker in the sample with a control,
wherein an increased level of any one of the biomarkers compared to the control is indicative that the subject is in need for follow up lung cancer testing.

In another embodiment, the control is a value, for example corresponding to a level of biomarker in a sample of a subject who is lung cancer free or an average from samples from a population of subjects who are cancer free. In an embodiment, an increased level of one or more of ADAM-17, OPG, PTX3, Follistatin and/or sTNF RI compared to control is indicative that the subject is in need of follow up lung cancer testing. In a further embodiment, an increased level of Pentraxin 3 compared to the control is indicative that the subject is in need of follow up lung cancer testing. In an embodiment, the follow up testing comprises sputum analysis and/or imaging.

An individual with lung cancer has several treatment options, such as chemotherapy, various surgical options and/or radiotherapy. Recurrence unfortunately is seen in a large percentage of cases. As the increased level of biomarkers is related to for example, shedding, secretion or other manner of release from cancer cells or as a result of cancer cell/host cell interations, it is predictable that the biomarkers described herein are also useful for detecting recurrence, particularly in the initial lung cancer had increased levels of one or more of the biomarkers in Table 8.

Accordingly, another aspect of the disclosure provides a method for prognosing lung cancer recurrence in a subject previously having lung cancer, the method comprising:
a) determining a level of a biomarker or a plurality of biomarkers in a sample from the subject, wherein the biomarker(s) is/are selected from the biomarkers listed in Table 8; and
b) comparing the level of each biomarker in the sample with a control or a reference level associated with recurrence,
wherein the disease outcome associated with the reference level most similar to the level of each biomarker in the sample is the predicted prognosis.

In an embodiment, the sample is obtained after treatment. In another embodiment, the sample is obtained after chemotherapeutic treatment. In another embodiment the sample is obtained after surgical resection of the lung cancer. In yet another embodiment, the method is repeated, for example 6, 9 and/or 12 months after treatment or resection. In an embodiment, the biomarker is selected from one or more of ADAM-17, OPG, PTX3, Follistatin and/or sTNF RI and the level of the one or more biomarkers in the sample from the subject is compared to a control or reference level associated with recurrence, wherein the disease outcome associated with the reference level most similar to the level of the one or more biomarkers in the sample is the predicted prognosis. In a further embodiment, the level of Pentraxin 3 in a sample from the subject is compared to the level of Pentraxin 3 in a control or reference level associated with recurrence, wherein the disease outcome associated with the reference level most similar to the level of Pentraxin 3 in the sample is the predicted prognosis.

Similarly, as it is predictable that increases in tumour burden will correspond to increases in biomarker expression, the biomarkers disclosed herein are useful for monitoring response to treatment and/or monitoring disease progression. Accordingly in another aspect, the disclosure provides a method of monitoring response to treatment comprising:
a) determining a base-line level of a biomarker or a plurality of biomarkers in a base-line sample from the subject, wherein the biomarker(s) is/are selected from the biomarkers listed in Table 8;
b) determining a level of a biomarker or a plurality of biomarkers in a post-treatment sample from the subject; and
c) comparing the level of each biomarker in the post-treatment sample with the base-line level;
   wherein an increase in the biomarker level in the post-treatment sample compared to the baseline level is indicative the subject is not responding or is responding poorly to treatment, and a decrease in the biomarker level in the post treatment sample compared to the base-line level is indicative that the subject is responding to treatment.

In an embodiment, the biomarker is selected from one or more of ADAM-17, OPG, PTX3, Follistatin and/or sTNF RI and an increase in one or more biomarker levels in the post treatment sample compared to the baseline level is indicative he subject is not responding or is responding poorly to treatment, and a decrease in the one or more biomarker levels in the post treatment sample compared to the base-line level is indicative that the subject is responding to treatment. In an embodiment, the biomarker is Pentraxin 3 and an increase in the Pentraxin 3 level in the post-treatment sample compared to the baseline level is indicative the subject is not responding or is responding poorly to treatment, and a decrease in the Pentraxin 3 level in the post treatment sample compared to the base-line level is indicative that the subject is responding to treatment.

In a further aspect, the disclosure provides a method of monitoring disease progression comprising:
d) determining a base-line level of a biomarker or a plurality of biomarkers in a base-line sample from the subject, wherein the biomarker(s) is/are selected from the biomarkers listed in Table 8;
e) determining a level of a biomarker or a plurality of biomarkers in a sample taken subsequent to the base-line sample from the subject; and
f) comparing the level of each biomarker in the sample with the base-line level;
   wherein an increase in the biomarker level in the post-base-line sample compared to the base-line level is indicative the disease is progressing, and a decrease in the biomarker level in the post base-line sample compared to the base-line level is indicative that the disease is not progressing.

In an embodiment, the biomarker is selected from one or more of ADAM-17, OPG, PTX3, Follistatin and/or sTNF RI and an increase in the one or more biomarker levels in the post-base-line sample compared to the base-line level is indicative the disease is progressing, and a decrease in the one or more biomarker levels in the post base-line sample compared to the base-line level is indicative that the disease is not progressing. In an embodiment, an increase in the Pentraxin 3 level in the post-base-line sample compared to the base-line level is indicative the disease is progressing, and a decrease in the Pentraxin 3 level in the post base-line sample compared to the base-line level is indicative that the disease is not progressing.

In yet another embodiment, the biomarkers(s) is/are selected from the biomarkers listed in Table 8, which correspond to proteins found in this study that were not found in previous studies related to lung proteomics.

In an embodiment, the biomarker(s) is/are selected from Table 8 with the proviso that the biomarker(s) is/are not listed in Table 1. In another embodiment, the biomarker is not CEA [27, 28], chromogranin A [29], chromogranin B [30], gastrin releasing peptide [29, 31], kallikrein-related peptidases 11 and 14 [32-34], progranulin, matrix metallopeptidase 1 (MMP1), collagenase [18] and/or neural cell adhesion molecule [35-37].

In another embodiment, the biomarker is not C1 of aldo-keto reductase family 1 (AKR1C1) identified by Huang et al. as dihydrodiol dehydrogenase [25].

In an embodiment, the lung cancer being screened for, diagnosed, detected or screened for the need for follow up testing in a subject is a small cell lung cancer (SCLC) or a non-small cell lung cancer (NSCLC). In a further embodiment, the NSCLC is an adenocarcinoma, a squamous cell carcinoma or a large cell carcinoma. In another embodiment, the lung cancer is lung cancer is stage I, stage II, stage III or stage IV. For example, in an embodiment, the lung cancer is NSCLC stage I, NSCLC state II, NSCLC stage III, or NSCLC stage IV. In an embodiment, the lung cancer is SCLC stage I, SCLC stage II, SCLC stage III, or SCLC stage IV.

In another embodiment, the lung cancer being screened for, diagnosed, detected or screened for the need for follow up testing and/or prognosed for recurrence is SCLC and the biomarker(s) is/are selected from the biomarkers listed in Table 8.

In another embodiment, the lung cancer being screened for, diagnosed, detected or screened for the need for follow up testing and/or prognosed for recurrence is NSCLC and the biomarker(s) is/are selected from the biomarkers listed in Table 8. In another embodiment, the NSCLC is an adenocarcinoma and the biomarkers(s) is/are selected from the biomarkers listed in Table 8. In another embodiment, the NSCLC is a squamous cell carcinoma and the biomarkers(s) is/are selected from the biomarkers listed in Table 8. In a further embodiment, the NSCLC is a large cell carcinoma and the biomarkers(s) is/are selected from the biomarkers listed in Table 8. In yet another embodiment, the lung cancer is a NSCLC and the biomarker(s) is/are selected from the biomarkers listed in Table 8.

In a preferred embodiment, the biomarkers are selected from ADAM-17, Osteoprotegerin, Pentraxin 3, Follistatin, and sTNF RI, and/or any combination thereof. In an embodiment, the biomarker is ADAM-17. In another embodiment, the biomarker is Osteoprotegerin. In an embodiment, the biomarker is Pentraxin 3. In a further embodiment, the biomarker is Follistatin. In yet another embodiment, the biomarker is sTNF RI.

The biomarkers disclosed herein were identified in the culture media of lung cancer cell subtypes and thereby include biomarkers that were in any manner released from the cell e.g. cleaved from membrane, secreted, and/or shed by the lung cancer cells into the culture medium (e.g. soluble biomarker). Further, many of the biomarkers were also found in a plasma proteome database. Accordingly, in an embodiment the level of biomarker(s) determined is soluble biomarker wherein the biomaker is selected from biomarkers listed in Table 8. In another embodiment the biomarker is soluble ADAM-17 (sADAM-17), soluble Osteoprotegerin (sOPG), soluble Pentraxin 3 (sPTX3), soluble Follistatin (sFollistatin), and/or soluble sTNF RI, and/or any combination thereof.

In another embodiment, the biomarker level determined is a polypeptide biomarker level.

In an embodiment, the methods disclosed herein further comprise obtaining a sample from the subject. In an embodiment, the level of biomarker is determined by contacting the sample and/or control with a detection agent.

In another embodiment, the biomarker level determined is a soluble form of a transmembrane protein (e.g. shed or cleaved portion thereof) and the detection agent is an antibody that binds to an extracellular portion of said biomarker.

In a further embodiment, the methods disclosed herein including the method of screening for, diagnosing or detecting lung cancer in a subject, or for screening a subject for the need for follow-up testing, and/or prognosis is used in addition to traditional diagnostic techniques for lung cancer. For example, SCLC and NSCLC are differentiated on the basis of size or appearance of the malignant cells. Accordingly, in an embodiment, cytology (e.g. sputum or biopsy) is also conducted.

In an embodiment, the sample and/or control is, or comprises a biological fluid. In an embodiment, the sample comprises blood, tumor biopsy, serum, plasma, sputum, pleural effusion, nasal lavage fluid, BAL fluid, saliva or tumour interstitial fluid or any fraction thereof. In an embodiment, the sample comprises blood. In another embodiment, the sample comprises a fraction of blood such as serum and/or plasma. In a preferred embodiment, the sample comprises serum. A person skilled in the art is familiar with the techniques for obtaining a serum sample. For example, the sample can be collected in EDTA-containing vacutainer tubes, centrifuged at 3000 rotations per minute for 15 minutes within one hour of collection, and optionally stored at −80 degrees Celsius.

In certain embodiments, the samples are processed prior to detecting the biomarker level. For example, a sample may be fractionated (e.g. by centrifugation or using a column for size exclusion), concentrated or proteolytically processed such as trypsinized, depending on the method of determining the level of biomarker employed.

In an embodiment, the sample and control are the same or similar tissue type, e.g both comprise blood and/or serum. Alternatively, the control is a value that corresponds to a level of biomarker derived from the same or similar type (e.g. tissue) as the sample.

In an embodiment, the control is a value for a biomarker, wherein subjects having a level of biomarker above the control are identified as having for example lung cancer and/or in need of follow up testing. For instance, the median level of ADAM-17, Osteoprotegerin, Pentraxin 3, Follistatin, and sTNF RI in subjects without lung cancer in the group disclosed herein is 12.0 μg/L, 1.84 μg/L, 1.52 ng/mL, 1251 μg/mL and 1.02 μg/L, respectively, whereas in subjects with lung cancer, the median level of ADAM-17, Osteoprotegerin, Pentraxin 3, Follistatin, and sTNF RI in the group disclosed herein is 27.3 μg/L, 4.43 μg/L, 4.91 ng/mL, 3116 pg/mL, and 1.53 μg/L, respectively. In each case, the median level in subjects with lung cancer is significantly increased compared to control subjects without lung cancer. Selecting a value for the control (e.g a cut-off value) wherein subjects having an increased level of one of more biomarkers disclosed herein is useful for identifying subjects as having lung cancer, needing follow testing and/or likely to have recurrence. The value selected will vary with the desired specificity and sensitivity. Accordingly, in an embodiment, wherein the biomarker is or comprises ADAM-17 and the control value is 10 μg/L, 11 μg/L, 13 μg/L, 14 μg/L, 15 μg/L, 16 μg/L, 17 μg/L, 18 μg/L, 19 μg/L, 20 μg/L, 21 μg/L, 22 μg/L, 23 μg/L, 24 μg/L, 25 μg/L, 26 μg/L, 27 μg/L, 28 μg/L, 29 μg/L, 30 μg/L, 31 μg/L, 32 μg/L, 33 μg/L, 34 μg/L, or 35 μg/L.

In another embodiment, the biomarker is or comprises Osteoprotegerin and the control value is 1.8 μg/L, 1.9 μg/L, 2.0 μg/L, 2.1 μg/L, 2.2 μg/L, 2.3 μg/L, 2.4 μg/L, 2.5 μg/L, 2.6 μg/L, 2.7 μg/L, 2.8 μg/L, 2.9 μg/L, 3.0 μg/L, 3.1 μg/L, 3.2 μg/L, 3.3 μg/L, 3.4 μg/L, 3.5 μg/L, 3.6 μg/L, 3.7 μg/L, 3.8 μg/L, 3.9 μg/L, 4.0 μg/L, 4.1 μg/L, 4.2 μg/L, 4.3 μg/L, 4.4 μg/L, 4.5 μg/L, 4.6 μg/L, or 4.7 μg/L.

In a further embodiment, the biomarker is or comprises Pentraxin 3 and the control value is 1.5 ng/mL, 1.6 ng/mL, 1.7 ng/mL, 1.8 ng/mL, 1.9 ng/mL, 2.0 ng/mL, 2.1 ng/mL, 2.2 ng/mL, 2.3 ng/mL, 2.4 ng/mL, 2.5 ng/mL, 2.6 ng/mL, 2.7 ng/mL, 2.8 ng/mL, 2.9 ng/mL, 3.0 ng/mL, 3.1 ng/mL, 3.2 ng/mL, 3.3 ng/mL, 3.4 ng/mL, 3.5 ng/mL, 3.6 ng/mL, 3.7 ng/mL, 3.8 ng/mL, 3.9 ng/mL, 4.0 ng/mL, 4.1 ng/mL, 4.2 ng/mL, 4.3 ng/mL, 4.4 ng/mL, 4.5 ng/mL, 4.6 ng/mL, 4.7 ng/mL, 4.8 ng/mL, 4.9 ng/mL, 5.0 ng/mL, 5.1 ng/mL, or 5.2 ng/mL.

In another embodiment, the biomarker is or comprises Follistatin and the control value is 1100 pg/mL, 1200 pg/mL, 1300 pg/mL, 1400 pg/mL, 1500 pg/mL, 1600 pg/mL 1700 pg/mL, 1800 pg/mL, 1900 pg/mL, 2000 pg/mL, 2100 pg/mL, 2200 pg/mL, 2300 pg/mL, 2400 pg/mL, 2500 pg/mL, 2600 pg/mL, 2700 pg/mL, 2800 pg/mL, 3000 pg/mL, 3200 pg/mL, 3400 pg/mL, 3600 pg/mL, or 3800 pg/mL.

In yet another embodiment, the biomarker is or comprises sTNF RI and the control value is 0.9 μg/L, 1.0 μg/L, 1.05 μg/L, 1.1 μg/L, 1.15 μg/L, 1.2 μg/L, 1.25 μg/L, 1.3 μg/L, 1.35 μg/L, 1.4 μg/L, 1.45 μg/L, 1.5 μg/L, 1.55 μg/L, 1.6 μg/L, 1.65 μg/L, 1.7 μg/L, 1.75 μg/L, or 1.8 μg/L.

In an embodiment, the level of ADAM-17 in the sample that is indicative of lung cancer is at least 28 μg/L, 30 μg/L, 32 μg/L, 34 μg/L, 36 μg/L, 38 μg/L, 40 μg/L, 42 μg/L, 44 μg/L, 46 μg/L, 48 μg/L, 50 μg/L, 60 μg/L, 80 μg/L, 100 μg/L, 200 μg/L, 300 μg/L, 400 μg/L, 500 μg/L, 600 μg/L, 700 μg/L, 800 μg/L, 900 μg/L, 1000 μg/L, 1100 μg/L, or 1200 μg/L. In an embodiment, the sample is serum.

In an embodiment, the level of biomarker in the sample that is indicative of lung cancer, the need for follow up testing and/or recurrence for Osteoprotegerin is at least 4.6 μg/L, 4.8 μg/L, 5.0 μg/L, 5.2 μg/L, 5.4 μg/L, 5.6 μg/L, 5.8 μg/L, 6.0 μg/L, 6.2 μg/L, 6.4 μg/L, 6.6 μg/L, 6.8 μg/L, 7.0 μg/L, 7.2 μg/L, 7.4 μg/L, 7.6 μg/L, 7.8 μg/L, 8.0 μg/L, 8.2 μg/L, 8.4 μg/L, 8.6 μg/L, 8.8 μg/L, 9.0 μg/L, 10 μg/L, 12 μg/L, 14 μg/L, 16 μg/L, 18 μg/L, 20 μg/L, 25 μg/L, 30 μg/L, 35 μg/L or 40 μg/L. In an embodiment, the sample is serum.

In a further embodiment, and the level of Pentraxin 3 in the sample that is indicative of lung cancer is at least 5.0 ng/mL, 5.2 ng/mL, 5.4 ng/mL, 5.6 ng/mL, 5.8 ng/mL, 6.0 ng/mL, 6.2 ng/mL, 6.4 ng/mL, 6.6 ng/mL, 6.8 ng/mL, 7.0 ng/mL, 7.2 ng/mL, 7.4 ng/mL, 7.6 ng/mL, 7.8 ng/mL, 8.0 ng/mL, 8.2 ng/mL, 8.4 ng/mL, 8.6 ng/mL, 8.8 ng/mL, 9.0 ng/mL, 9.2 ng/mL, 9.4 ng/mL, 9.6 ng/mL, 9.8 ng/mL, 10 ng/mL, 11 ng/mL, 12 ng/mL, 13 ng/mL. 14 ng/mL, 15 ng/mL, 16 ng/mL, 17 ng/mL, 18 ng/mL, 19 ng/mL, 20 ng/mL, 25 ng/mL, 30 ng/mL, 40 ng/mL, 50 ng/mL, or 60 ng/mL. In an embodiment, the sample is serum.

In another embodiment, the level of Follistatin in the sample that is indicative of lung cancer is at least 3200 pg/mL, 3300 pg/mL, 3400 pg/mL, 3500 pg/mL, 3600 pg/mL, 3700 pg/mL, 3800 pg/mL, 3900 pg/mL, 4000 pg/mL, 4100 pg/mL, 4200 pg/mL, 4300 pg/mL, 4400 pg/mL, 4500 pg/mL, 4600 pg/mL, 4700 pg/mL, 4800 pg/mL, 4900 pg/mL, 5000 pg/mL, 6000 pg/mL, 7000 pg/mL, 8000 pg/mL, 9000 pg/mL, 10000 pg/mL, or 12000 pg/mL. In an embodiment, the sample is serum.

In another embodiment, the level of sTNF RI in the sample that is indicative of lung cancer is at least 1.5 μg/L, 1.55 μg/L, 1.6 μg/L, 1.65 μg/L, 1.7 μg/L, 1.75 μg/L, 1.8 μg/L, 1.85 μg/L, 1.9 μg/L, 1.95 μg/L, 2.0 μg/L, 2.1 μg/L, 2.2 μg/L, 2.3 μg/L, 2.4 μg/L, 2.5 μg/L, 2.6 μg/L, 2.7 μg/L, 2.8 μg/L, 2.9 μg/L, 3.0 μg/L, 3.1 μg/L, 3.2 μg/L, 3.3 mg/L, 3.4 μg/L, 3.5 μg/L, 3.6 μg/L, 3.7 μg/L, 3.8 μg/L, 3.9 μg/L, 4.0 μg/L, 5.0 μg/L, 6.0 μg/L, 6.5 μg/L, or 7.0 μg/L. In an embodiment, the sample is serum.

A person skilled in the art will recognize that the particular control value (e.g. cut-off value) for each biomarker can be determined for a particular population or set of conditions as demonstrated herein. For example the cut-off value can vary with sample processing, e.g. dilution and/or concentration of the sample. Furthermore, the control values vary for a design, specificity and/or sensitivity. For example, the values in the Example 1 were calculated to provide about 100% specificity. Example 2 describes calculations of cut-off levels for various specificities and sensitivities. The control value is in an embodiment, a value that provides a specificity of at least 70%, 75%, 80%, 85%, 90%, 95%, 98%, 99% and/or 100%. In another embodiment, the control value is a value provides a sensitivity of at least 30%, 35%, 40%, 45%, 50%, 55%, 60%, 65%, 70%, 75%, 80%, 85%, 90%, 95%, 98%, 99% and/or 100%.

The increase in biomarker level(s) that is indicative of lung cancer, the need for follow up testing, prognosis, poor response to treatment and/or disease progression is in an embodiment a fold increase relative to the control and/or base-line level. Accordingly, in an aspect of the disclosure, the increase indicative of lung cancer, the need for follow up testing and/or prognosis in subjects with lung cancer relative to control (and/or base-line level) is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.2, 5.4, 5.6, 5.8, 6.0, 6.5, 7.0, 7.5, 8.0, 8.5, 9.0, 9.5, 10, 15, 20, 30, 40, 50, 60, 80, and 100 fold.

In an embodiment, the level of ADAM-17 in the sample that is indicative of lung cancer, the need for follow up testing, prognosis, poor response to treatment and/or disease progression is, relative to the control (and/or base-line level), at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.2, 4.4, 4.6, 4.8, 5.0, 5.5, 6.0, 6.5, 7.0, 7.5, 8.0, 8.5, 9.0, 9.5, 10, 15, 20, 40, 60, 80, or 100 fold.

In another embodiment, the level of Osteoprotegerin in the sample that is indicative of lung cancer, the need for follow up testing, prognosis, poor response to treatment and/or disease progression is, relative to the control, and/or base-line level at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 5.0, 7.5, 10, 15, or 20 fold.

In a further embodiment, the level of Pentraxin 3 in the sample that is indicative of lung cancer, the need for follow up testing, prognosis, poor response to treatment and/or disease progression is, relative to the control and/or base-line level, at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.2, 5.4, 5.6, 5.8, 6.0, 6.5, 7.0, 7.5, 8.0, 8.5, 9.0, 9.5, 10, 15, 20, or 40 fold.

In yet a further embodiment, the level of Follistatin in the sample that is indicative of lung cancer, the need for follow up testing, prognosis, poor response to treatment and/or disease progression is, relative to the control and/or base-line level, at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 5.0, 6.0, 8.0, or 10 fold.

In another embodiment, the level of sTNF RI in the sample that is indicative of lung cancer, the need for follow up testing, prognosis, poor response to treatment and/or disease progression is, relative to the control and/or base-line level, at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 5.0, 6.0, 8.0, or 10 fold.

In another embodiment, the level of biomarker(s) that is indicative of lung cancer, the need for follow up testing and/or prognosis is the median level in a population of subjects with lung cancer. For example, described herein are methods of determining the median level of a biomarker of the disclosure in subjects with and without lung cancer. In an embodiment, the level of biomarker in the sample is at least the median level of the biomarker in subjects with lung cancer. In another embodiment, the level of biomarker(s) that is indicative of lung cancer, the need for follow up testing and/or prognosis is the average level in a population of subjects with lung cancer.

In an embodiment, the level of biomarker(s) in the sample that is/are indicative of lung cancer, the need for follow up testing and/or prognosis is at least the median level of a biomarker(s) of one or more biomarkers listed in Table 8. In another embodiment, the level of biomarker(s) in the sample that is/are indicative of lung cancer, the need for follow up testing and/or prognosis is at least the average level of a biomarker(s) of one or more biomarkers listed in Table 8.

In a further embodiment, the level of the biomarker(s) in a sample that is indicative of lung cancer, the need for follow up testing, prognosis, poor response to treatment and/or disease progression is a range, for example, 1.1 to 10, 1.1 to 20, 1.1 to 40, 1.1 to 100, 1.5 to 10, 1.5 to 20, 1.5 to 40, 1.5 to 100, 2.0 to 10, 2.0 to 20, 2.0 to 40, 2.0 to 100, 3.0 to 10, 3.0 to 20, 3.0 to 40, or 3.0 to 100 times a control or base-line level.

In certain embodiments, for example, when using Western blot analysis, the value of the level of the biomarker in the sample from the subject and/or a control is normalized to an internal control. For example, the level of a biomarker may be normalized to an internal control such as a polypeptide that is present in the sample type being assayed, for example a house keeping gene protein, such as beta-actin, glyceraldehyde-3-phosphate dehydrogenase, or beta-tubulin, or total protein, e.g. any level which is relatively constant between subjects for a given volume.

In another embodiment, the level of two or more of the biomarkers are determined. In yet a further embodiment, 3, 4, or 5 or more biomarker levels are determined. In yet another embodiment, 6-10, 11-15, 16-20, 21-25, or more biomarker levels, or any number in between, are determined. A person skilled in the art will appreciate that a number of methods can be used to determine the amount of a polypeptide biomarker, including soluble biomarker of the disclosure, including mass spectrometry approaches, such as multiple reaction monitoring (MRM) and product-ion monitoring (PIM), and also including immunoassays such as Western blots, enzyme-linked immunosorbant assay (ELISA), and immunoprecipitation followed by sodium-dodecyl sulfate polyacrylamide gel electrophoresis (SDS-PAGE) immunocytochemistry. Accordingly, in other embodiments, the level determined is a polypeptide product. In certain embodiments, the step of determining the biomarker level comprises using immunohistochemistry and/or an immunoassay. In certain embodiments, the immunoassay is an ELISA. In yet a further embodiment, the ELISA is a sandwich type ELISA.

For example, the Quantikine human sTNF RI Immunoassay can be used to detect sTNF RI. It is a solid phase ELISA designed to measure sTNF RI in cell culture supernates, serum, plasma and urine. It contains E. coli-expressed, recombinant human sTNF RI, as well as antibodies raised against this polypeptide. The recombinant protein represents the non-glycosylated, N-terminal methionyl form of the naturally occurring human soluble Type I receptor for TNF with an apparent molecular weight of approximately 18.6 kDa. The immunoassay has been shown to accurately quantitate the recombinant sTNF RI. In another example, the level of Pentraxin 3 can be determined using a Pentraxin 3 ELISA kit, purchased for example, from R&D Systems. As an example, two antibodies can be employed, one used for capture (e.g. a monoclonal mouse antibody) and one used for detection (e.g. a biotinylated goat polyclonal antibody). Standardization can be achieved by using recombinant, purified Pentraxin 3. Samples can be diluted, for example, diluted 3-fold with a 6% bovine serum albumin solution before analysis. As an example, the sample can be diluted to fall within a linear portion of a standard curve, for example in an Example described herein, the calibration curve was linear from 200 to 20,000 pg/mL and the precision in this range was <10%. Assays may for example be performed in duplicate.

The level of two or more markers can be determined for example using multiple reaction monitoring assays such as “Product-ion monitoring” PIM assays. This method is a hybrid assay wherein an antibody for a biomarker is used to extract and purify the biomarker from a sample e.g. a biological fluid, the biomarker is then trypsinized in a microtitre well and a proteolytic peptide is monitored with a triple-quadrapole mass spectrometer, during peptide fragmentation in the collision cell. More technical details can be found in (74). Biomarker levels for a model biomarker has been quantified as low as 0.1 ng/mL with CVs less than 20%.

Alternatively, it is also possible to quantify analytes present at relatively higher concentration in a biological fluid such as serum (e.g. ≧100 ng/mL) without antibody enrichment. In this case, the biological fluid (e.g. serum) is digested in trypsin and selected proteotypic peptides are monitored for various transitions during fragmentation, as described above. With such assays, multiplexing 5 or more biomarkers is possible.

In an embodiment, antibodies or antibody fragments are used to determine the level of polypeptide of one or more biomarkers of the disclosure. In an embodiment, the antibody or antibody fragment is labeled with a detectable marker. In a further embodiment, the antibody or antibody fragment is, or is derived from, a monoclonal antibody. A person skilled in the art will be familiar with the procedure for determining the level of a polypeptide biomarker by using said antibodies or antibody fragments, for example, by contacting the sample from the subject with an antibody or antibody fragment labeled with a detectable marker, wherein said antibody or antibody fragment forms a complex with the biomarker.

The label is preferably capable of producing, either directly or indirectly, a detectable signal. For example, the label may be radio-opaque or a radioisotope, such as ³H, ¹⁴C, ³²P_{,} ³⁵S_{,} ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In another embodiment, the level of polypeptide biomarker of the disclosure is detectable indirectly. For example, a secondary antibody that is specific for a primary antibody that is in turn specific for the isolated protein of the disclosure wherein the secondary antibody contains a detectable label can be used to detect the target polypeptide biomarker.

### III. Compositions

Another aspect of the disclosure relates to compositions for determining the levels of biomarker products described herein. In an embodiment, the composition comprises at least two detection agents that bind a biomarker selected from the biomarkers listed in Table 8. In an embodiment, the composition comprises at least two detection agents that bind one or more biomarkers selected from ADAM-17, Osteoprotegerin, Pentraxin 3, Follistatin, sTNF RI and/or combinations thereof. In another embodiment the composition comprises at least two detection agents wherein each agent binds a polypeptide biomarker, wherein the biomarkers comprise ADAM-17, Osteoprotegerin, Pentraxin 3, Follistatin, sTNF RI and/or combinations thereof. In a further embodiment, the composition comprises a detection agent which binds soluble biomarker. In an embodiment, the detection agent is an antibody. In an embodiment, the antibody detects ADAM-17, Osteoprotegerin, Pentraxin 3, Follistatin, or sTNF RI. In a further embodiment, the antibody is an antibody described herein. The composition comprises in another embodiment, a suitable carrier, diluent, or additive as are known in the art.

A person skilled in the art will appreciate that the detection agents can be labeled. The label is preferably capable of producing, either directly or indirectly, a detectable signal. For example, the label may be radio-opaque or a radioisotope, such as ³H, ¹⁴C, ³²P, ³⁵S_{,} ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In an embodiment the two detection agents are each isolated polypeptides. In another embodiment, the isolated polypeptide is an antibody and/or an antibody fragment for example, an antibody described herein.

In another embodiment, the detection agent is a nucleic acid that binds or hybridizes a nucleic acid biomarker, for example a nucleic acid that hybridizes a nucleic acid biomarker. In a further embodiment, the agent is a peptide mimetic that binds a biomarker product described herein.

### IV. Immunoassays and Kits

Another aspect of the disclosure provides an immunoassay comprising an antibody optionally immobilized on a solid support, wherein the antibody binds a biomarker of the disclosure. In a further embodiment, the biomarker recognized by the antibody is selected from ADAM-17 and/or Osteoprotegerin. In a preferred embodiment, the biomarker recognized by the antibody is Pentraxin 3. The immunoassay is useful for detecting a level of a biomarker of the disclosure.

Another aspect of the disclosure is a kit for screening for, detecting, or diagnosing lung cancer in a subject and/or determining prognosis of a subject having lung cancer. In an embodiment, the kit comprises one or more detection agents, for example an antibody, specific for a biomarker described herein, for example a biomarker listed in Table 8. In an embodiment, the kit comprises a detection agent specific for ADAM-17, Osteoprotegerin, Pentraxin 3, Follistatin, and/or sTNF RI and instructions for use. In an embodiment, the kit comprises a composition or immunoassay described herein.

The kit can also include a control or reference standard and/or instructions for use thereof. In addition, the kit can include ancillary agents such as vessels for storing or transporting the detection agents and/or buffers or stabilizers.

In another embodiment, the kit comprises an antibody to one or more of ADAM-17, Osteoprotegerin, Pentraxin 3, Follistatin and sTNF RI and a quantity of a purified standard, such as a known quantity of biomarker polypeptide.

In an embodiment, the disclosure provides a kit for detecting a biomarker comprising:

(a) a detection agent that binds a biomarker selected from ADAM-17, Osteoprotegerin, Pentraxin 3, Follistatin, and/or sTNF RI or any combination thereof; and

(b) instructions for use, or a quantity of purified ADAM-17, Osteoprotegerin, Pentraxin 3, Follistatin, or sTNF RI polypeptide.

In a further embodiment, the kit comprises one or more detection agents wherein the detection agent binds to an extracellular portion of a biomarker for example wherein the biomarker is a transmembrane protein.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Sequences associated with accession numbers described herein including for example the Tables, are herein specifically incorporated by reference.

The following non-limiting examples are illustrative of the present disclosure:

### EXAMPLES

### Example 1

### Results

### Optimization of Cell Culture Conditions

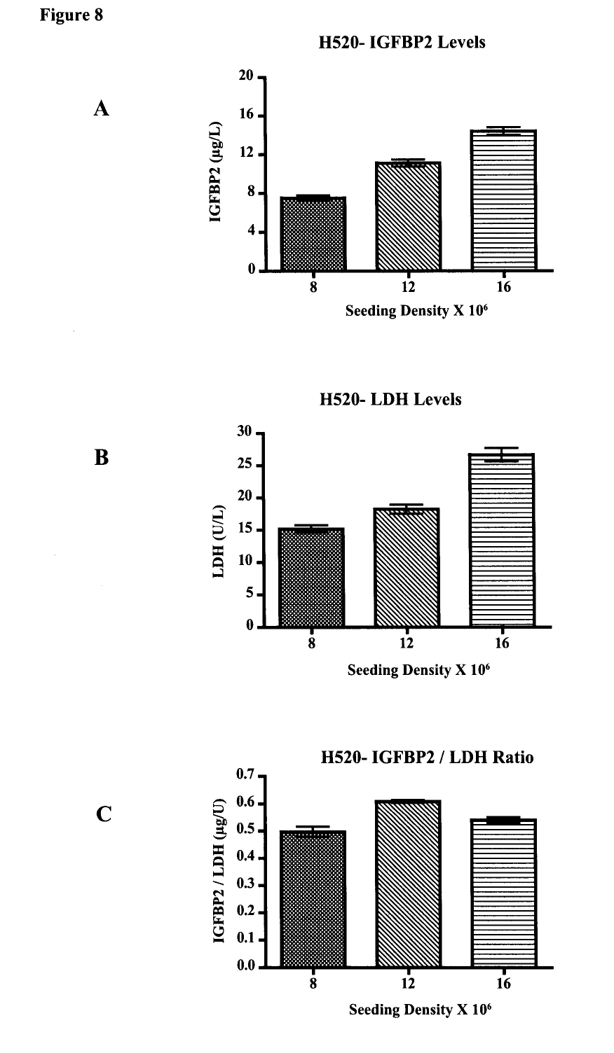

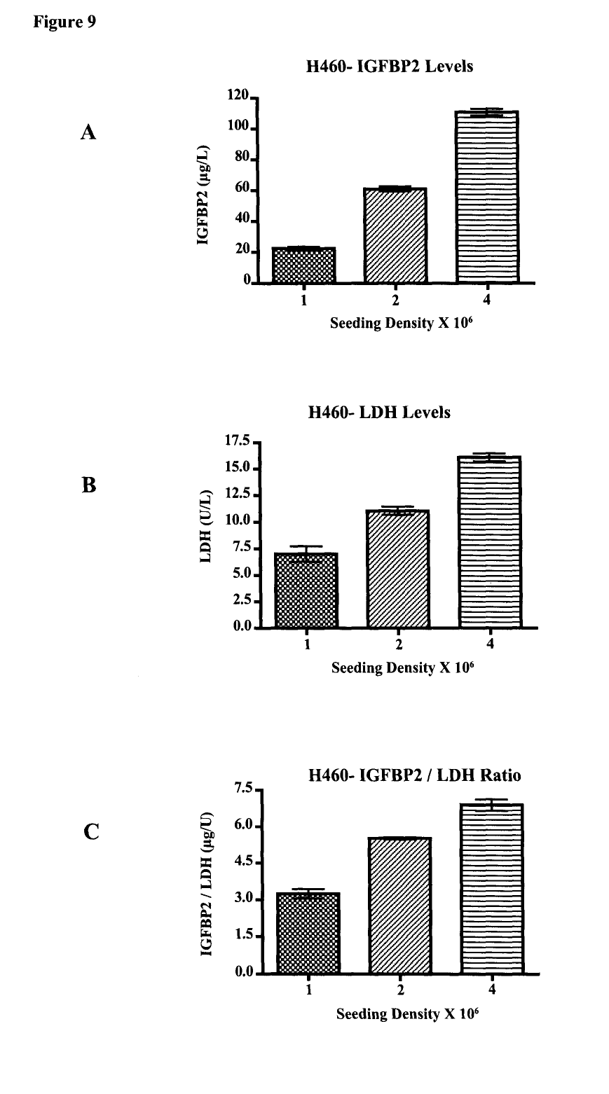

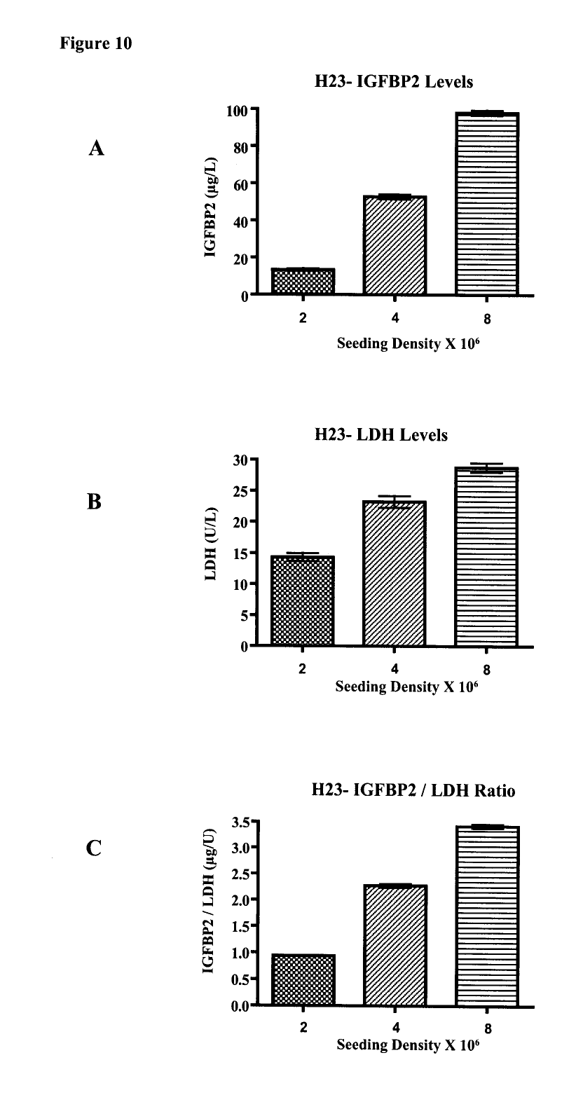

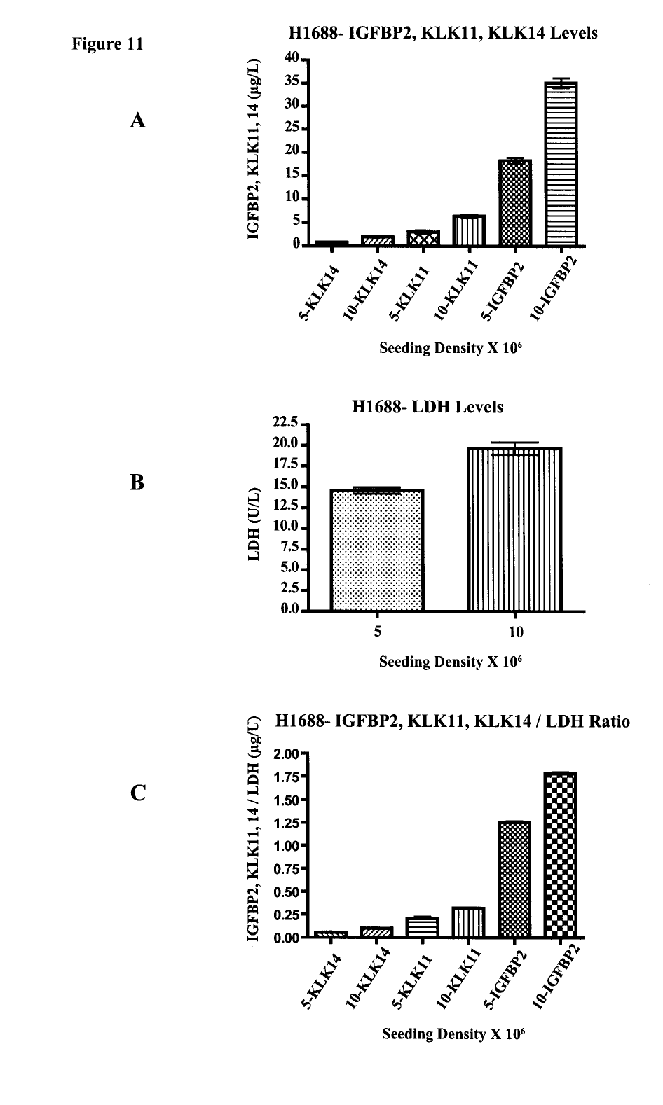

In order to delineate the secretome of the 4 lung cancer cell lines, cell culture conditions were first optimized to minimize cell death and maximize secreted protein concentration. For this purpose, cells were grown in SFM for 48 h at different seeding densities. Total protein, LDH levels and the concentration of IGFBP2 in the CM of H1688, H520, H460 and H23 cells, and KLK11 and KLK14 in the CM of H1688 cells were measured. The ratio of IGFBP2 concentration to LDH levels for each cell culture condition and the ratio of KLK11 and KLK14 concentrations to LDH levels measured in the CM of H1688 cell line were compared (FIGS. 8-11). The following optimal seeding densities were selected for proteomic analysis (4×10⁶ cells for H460, 8×10⁶ cells for H23, 10×10⁶ cells for H1688 and 12×10⁶ cells for H520, respectively), as those gave the highest ratio of IGFBP2 or KLK production (indicator of secreted proteins) to LDH (indicator of cell death).

At the optimized seeding densities, total protein concentration was 38, 15, 14 and 15 μg/mL for H460, H23, H1688 and H520, respectively. Further, proteomic analysis was accomplished with approximately 800 μg to 1 mg of total protein.

### Identification of Proteins by Mass Spectrometry (MS Method)

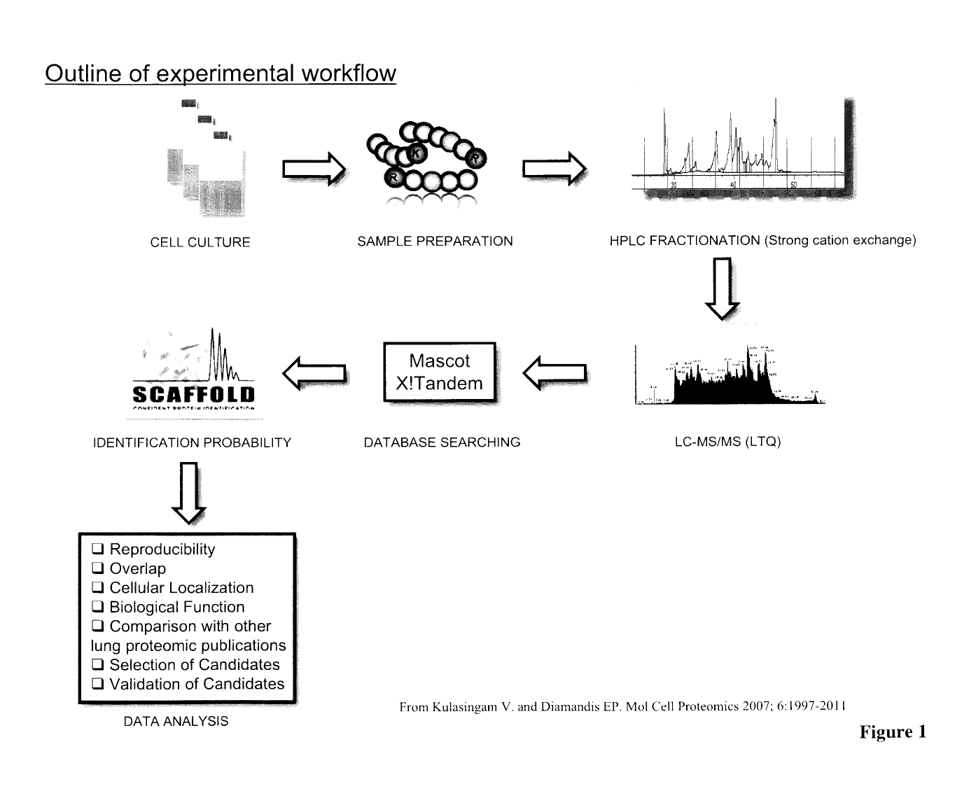

The experimental design for sample preparation, LC/MS/MS and bioinformatic analysis was similar to a design previously described [14] and is outlined in FIG. 1. The conditioned media from each of four lung cancer cell lines grown in SFM was collected, dialyzed, lyophilized and digested with trypsin. The samples were then subjected to strong cation exchange liquid chromatography followed by LC-MS/MS. Mascot and X!Tandem search engines were used to analyze the resulting raw mass spectra. Using Scaffold, which contains Protein and Peptide Prophet software, a list of all proteins with an 80% probability and all peptides with a 95% probability was generated. In total, from the three replicates per cell line, 965, 871, 726 and 847 proteins were identified in the H1688, H23, H460 and H520 cell lines. From the negative control flask that did not contain any cells but treated in the same manner as the CM of the 4 cell lines, a total of 83 proteins were identified. Many of these were fetal bovine serum (FBS)-derived proteins, used to initially culture the cells. These proteins were not considered further in data analysis.

### Overlap of Proteins Between Replicates and Reproducibility of the Method

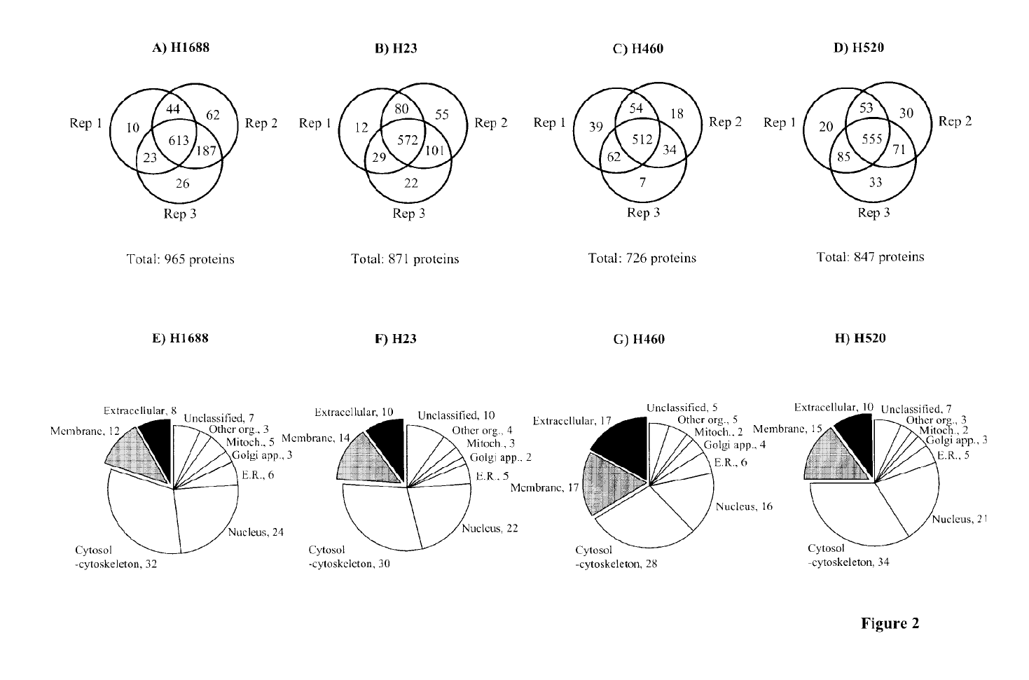

To investigate the reproducibilty of the method, each cell line was cultured in triplicate, providing three independent biological replicates per cell line. FIG. 2 shows the overlap between the 3 replicates of each cell line. For H1688, 965 proteins were identified (FIG. 2A). Of these, 613 were identified in all 3 replicates, yielding a reproducibility of 63.5%. For the H23 cell line, a total of 871 proteins were identified (FIG. 2B), of which 572 were common to all 3 replicates (65.7% reproducibility). Furthermore, 726 proteins were identified in H460 (FIG. 2C), of which 512 were found in all 3 replicates (70.5% reproducibility). Finally, 847 proteins were identified in H520. Of these, 555 were common to all 3 replicates, yielding a reproducibility of 65.5% (FIG. 2D). Approximately 20-26% of proteins were found in two replicates, whereas approximately 10% were exclusive to one replicate.

### Identification of Internal Control Proteins by MS

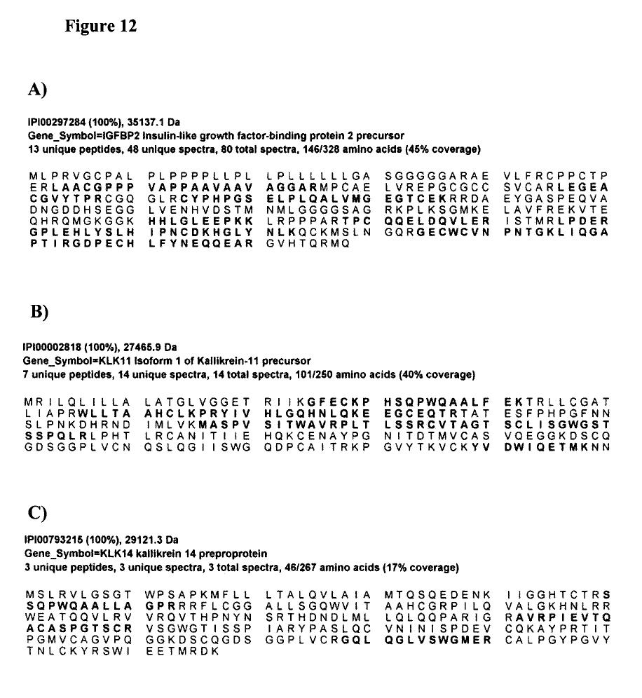

To monitor the cell culture optimization process, the concentration of 2 kallikrein-related peptidases (KLK11 and KLK14) and IGFBP2, which are known to be secreted, was measured by ELISA in the CM of the 4 lung cancer cell lines. All cell lines expressed IGFBP2 (15-110 μg/L), while H1688 was the only cell line expressing KLK11 (6.3 μg/L) and KLK14 (1.9 μg/L) at levels measurable by ELISA. Using the MS approach, KLK11 and KLK14 were identified in the CM of H1688 and IGFBP2 in the CM of all 4 cell lines. FIG. 12 illustrates the sequences of KLK11, KLK14 and IGFBP2 and the peptides identified by MS. IGFBP2 displayed approximately 10 unique peptides in all three replicates of each cell line, covering approximately 40% of its sequence (FIG. 12A). Six (replicate 1) to seven unique peptides (replicates 2 and 3) were identified for KLK11 resulting in a 28 to 41% sequence coverage (FIG. 12B). Furthermore, KLK14 was identified in 2 replicates of H1688 by one and three unique peptides, respectively. This resulted in a 5 to 17% sequence coverage (FIG. 12C). H520, H23 and H460 cells did not secrete any detectable KLK14 by ELISA and, as expected, this kallikrein-related peptidase was not found in their CM by MS.

Thus, successful identification of the selected endogenous internal control proteins by MS, especially those expressed at relatively low levels (KLK11 and KLK14), demonstrated that the detection limit of the MS-method for marker identification was in the low μg/L range.

### Classification of Proteins Identified by MS by Cellular Localization

Each identified protein was classified by its cellular localization using Genome Ontology (GO), Swiss-Prot, Human Protein Reference and Bioinformatic Harvester databases. These categories are non-exclusive since a protein can be classified in more than one cellular compartment. FIG. 2E-H shows the cellular localization of proteins identified in the CM of H1688 (E), H23 (F), H460 (G) and H520 (H). Twenty to 34% of the proteins identified were classified as extracellular or membrane-bound in each cell line. The remainder of the proteins identified in the CM were classified as intracellular [>50% (cytosol-cytoskeleton, nucleus, endoplasmic reticulum, Golgi apparatus, mitochondria and other organelles such as endosomes, lysosomes)], while 5-10% were unclassified.

### Overlap of Proteins Between the Four Lung Cancer Cell Lines

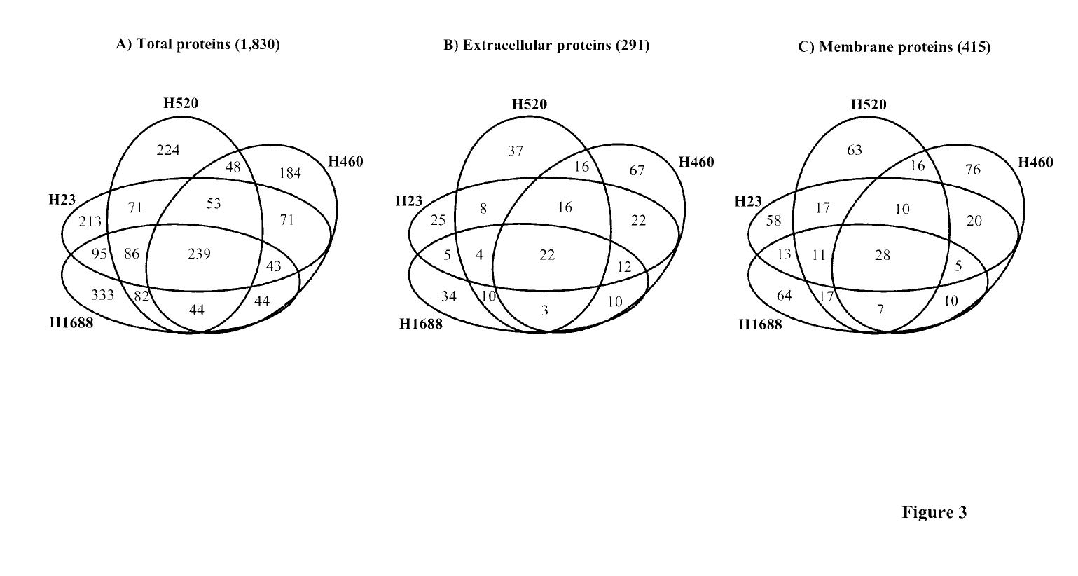

The proteins identified among the 4 lung cancer cell lines were analyzed for overlap, using an in-house developed program (FIG. 3). Out of the 1,830 unique proteins identified in this study, 239 (13%) were common to all 4 cell lines (FIG. 3A). Moreover, 226 (12.4%) and 411 (22.5%) proteins were found in three and two of the cell lines, respectively. Interestingly, about 52% of the proteins identified were unique to one of the cell lines.

FIGS. 3B and 3C display the overlap among the 291 extracellular proteins and the 415 membrane-bound proteins, respectively. The results show 22 (about 8%) of the extracellular proteins and 28 (about 7%) of the membrane-bound proteins were common to all 4 cell lines. A large portion of extracellular proteins (56%) and membrane-associated proteins (63%) were identified in only one cell line. These results illustrate the heterogeneity of lung cancer cell lines and the requirement of analyzing multiple cell lines to better depict the secretome of lung cancer.

### Extracellular and Membrane-bound Proteins Identified by MS

According to GO annotation, 291 proteins (15.9%) were classified as extracellular and 415 proteins (22.7%) as membranous. From the list of extracellular and membrane-bound proteins, some known or putative lung cancer biomarkers were identified. These included CEA [27, 28], chromogranin A [29], chromogranin B [30], gastrin releasing peptide [29, 31], kallikrein-related peptidases 11 and 14 [32-34], matrix metallopeptidase 1 (MMP1), collagenase [18] and neural cell adhesion molecule [35-37] (Table 1). Moreover, all of the extracellular and membrane-bound proteins were compared to the Human Plasma Proteome Database to determine whether they have been previously found in plasma. Of 291 secreted proteins, 129 (44.3%) were identified in human plasma. One hundred and sixty-eight of 415 membranous proteins (40.5%) were also found in human plasmaTables 7A-D contain detailed information on the 5 lung markers, e.g. ADAM-17, Osteoprotegerin, Pentraxin 3, Follistatin and sTNF R1, identified for each of the cell lines, including number of unique peptides, peptide sequences, precursor ion mass and charge states.

Comparison of the Present Proteome with Other Lung Proteomic Publications

Proteins identified in the four lung cancer cell lines were compared with the proteome of lung-related diseases and lung-related biological fluids.

Xiao et al. used proteomic techniques to analyze CM from primary cultures of lung cancer cells and adjacent normal bronchial epithelial cells of 6 lung cancer patients [18]. Using one-dimensional PAGE and nano-ESI-MS/MS, they identified 231 proteins, of which 161 (70%) were also found in the herein described proteomics study. Huang et al. analyzed secreted proteins in the CM of an NSCLC cell line (A549) by 2D-PAGE and MALDI-TOF MS. Fourteen human proteins were identified, of which 11 (79%) were also found using the methods described herein, including alpha enolase, peroxiredoxin 1, Galectin 1, ubiquitin carboxyl-terminal hydrolase (PGP9.5) and dihydrodiol dehydrogenase (DDH) [25]. In addition, comparative proteomic analysis of the two NSCLC cell lines with different metastatic potential was carried out using 2-DE followed by MALDI-TOF/MS and MS/MS analysis. Thirty three differentially expressed proteins were identified, including 16 proteins which were significantly up-regulated and 17 proteins which were down-regulated in highly metastatic cells, compared with non-metastatic cells [26]. Of these 33 proteins reported to be altered, 30 (91%) were also found among the 1,830 proteins in the CM described herein. Importantly, all proteins identified as up-regulated in highly metastatic cells were identified herein. Among these candidates, Tian et al. observed by IHC a correlation between up-regulation of S100A11 expression in NSCLC tissues and higher tumor-node-metastasis (TNM) stage and positive lymph node status [26].

The data shown herein was also compared with proteomic analyses of human-induced sputum [9] and human-induced sputum of chronic bronchitis subjects [10]. With combination of 2-D gel analysis and GeLC-MS/MS, a total of 191 human proteins were confidently assigned in induced-sputum [9], of which 72 were also found by using the methods herein described. Interestingly, several extracellular and membranous proteins such as annexins A1 and A2, cathepsin D, clusterin, cystatins C and SN, IGFBP2, kallikrein-related peptidase 11, prominin 1, gelsolin and lipocalin 1 were present in both studies. However, there was less overlap with the proteome of induced-sputum of chronic bronchitis subjects (22/106 proteins, [10]), likely due to the presence of abundant proteins (including immunoglobulins) in the sputome (38/106, [10]) that were not present in the herein disclosed list of proteins.

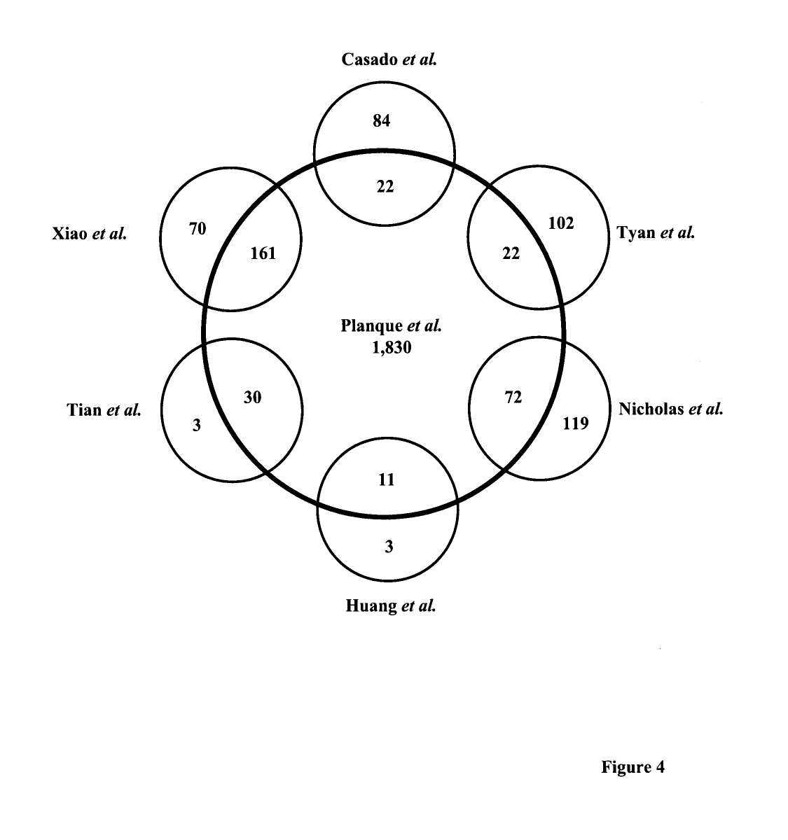

Using 2D nano-HPLC-ESI-MS/MS, Tyan et al. reported identification of 124 proteins from 43 pooled adenocarcinoma patient pleural effusions with high confidence (at least 2 or more unique peptides for each protein identified) [11]. From these, 22 were also identified by the methods disclosed herein, including extracellular lipocalin 1, gelsolin, lumican, pigment epithelium-derived factor, alpha-1-antitrypsin, zinc alpha-2-glycoprotein 1 and apolipoprotein E. FIG. 4 summarizes the overlap between other publications and the data disclosed herein. Table 8 enlists all the biomarkers identified herein that were not found in the published lung proteomic studies.

### Validation of Proteins Identified by MS as Lung Cancer Biomarkers

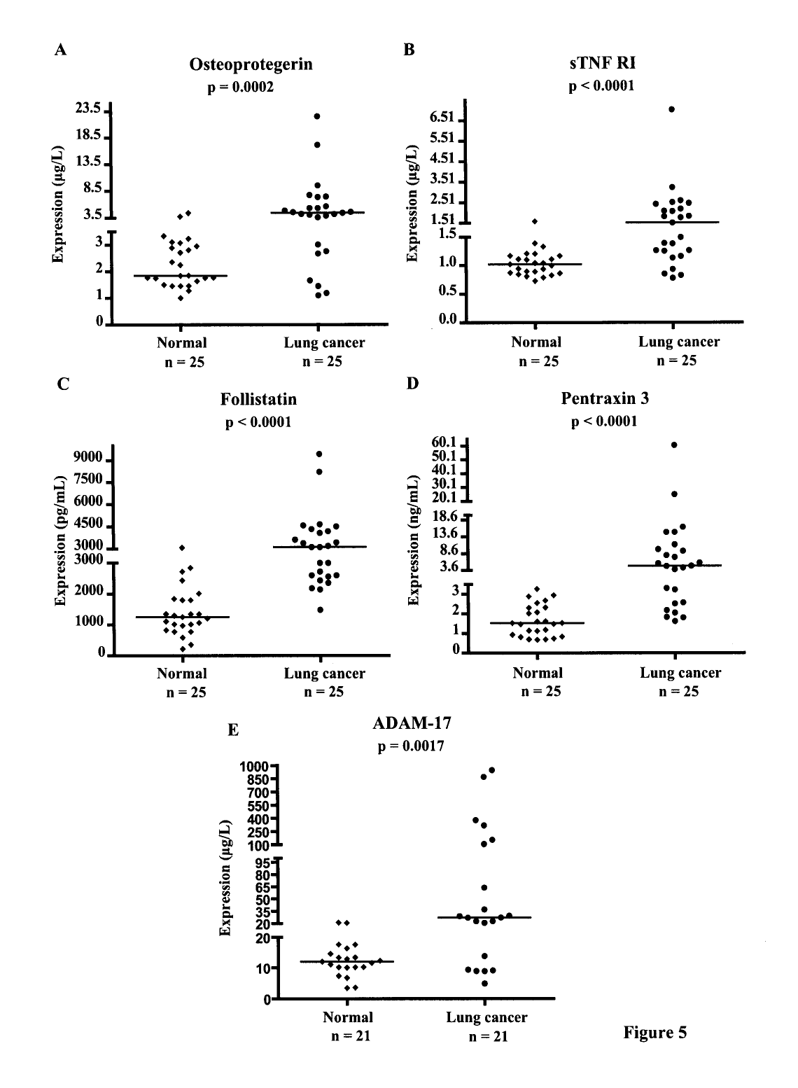

Using commercially available or developed in-house sandwich immunoassays, pre-clinical validation was performed on five candidates, sTNF RI, Follistatin, ADAM-17, Pentraxin 3 and Osteoprotegerin, selected from the list of proteins identified by MS. Candidate biomarker concentration was examined in serum samples from patients with or without lung cancer (FIG. 5, Tables 2-6). Serum levels of osteoprotegerin (OPG) were significantly elevated in patients with lung cancer (median=4.43 μg/L), in comparison to healthy individuals (median=1.84 μg/L) (p=0.0002).

The sTNF RI serum levels in NSCLC were significantly higher (median=1.53 μg/L) than those in healthy controls (median=1.02 μg/L) (p<0.0001).

A significant elevation of Follistatin was observed in serum of lung cancer patients (median=3,116 pg/mL) as compared to healthy volunteers (median=1,251 pg/mL) (p<0.0001).

Pentraxin 3 (PTX3) was identified in all 3 NSCLC cell lines and especially, with higher abundance in the squamous cell carcinoma cell line, with 15 to 16 unique peptides. As demonstrated in FIG. 5D, the distribution of PTX3 between cases and controls was significantly different (p<0.0001). Serum levels of PTX3 were much higher in lung cancer patients (median=4.91 ng/mL), as compared to healthy individuals (median=1.52 ng/mL).

By using an ELISA developed in-house, significant increase of ADAM-17 was observed in serum of patients with NSCLC (median=27.3 μg/L), in comparison to healthy volunteers (median=12.0 μg/L) (p=0.002).

In a very preliminary assessment of these five candidate markers for lung carcinoma, the diagnostic sensitivity (percentage of patients with elevated marker levels) was calculated at 100% specificity (using as cutoff, the highest value in the normal group). These diagnostic sensitivities were: Osteoprotegerin-52%; sTNF R1-52%; Follistatin-56%; Pentraxin 3-68%; ADAM-17-67%.

### Assignments of Biological Function and Network Construction for Biological Processes

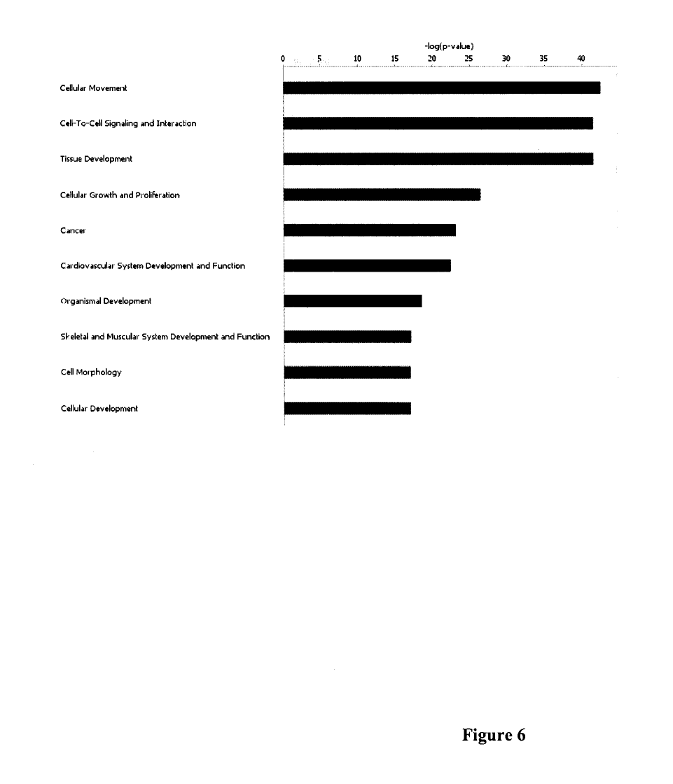

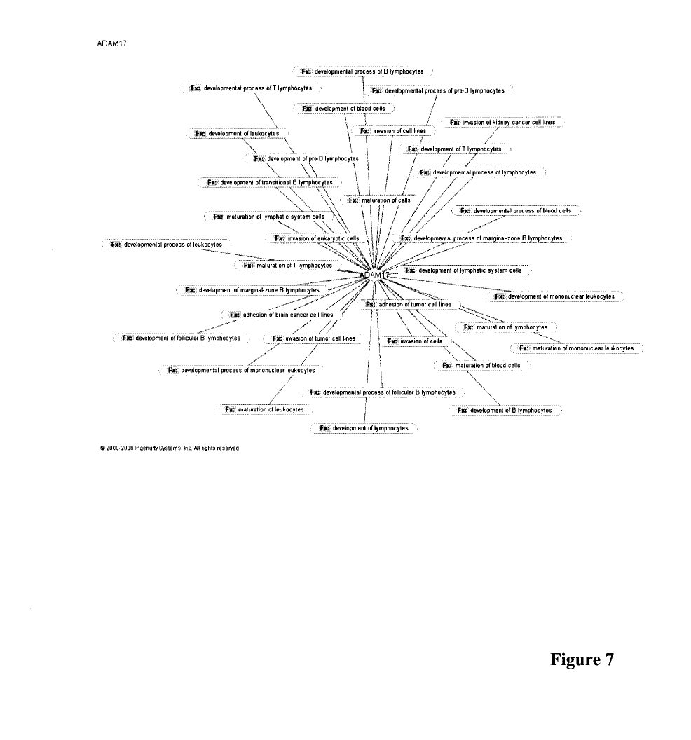

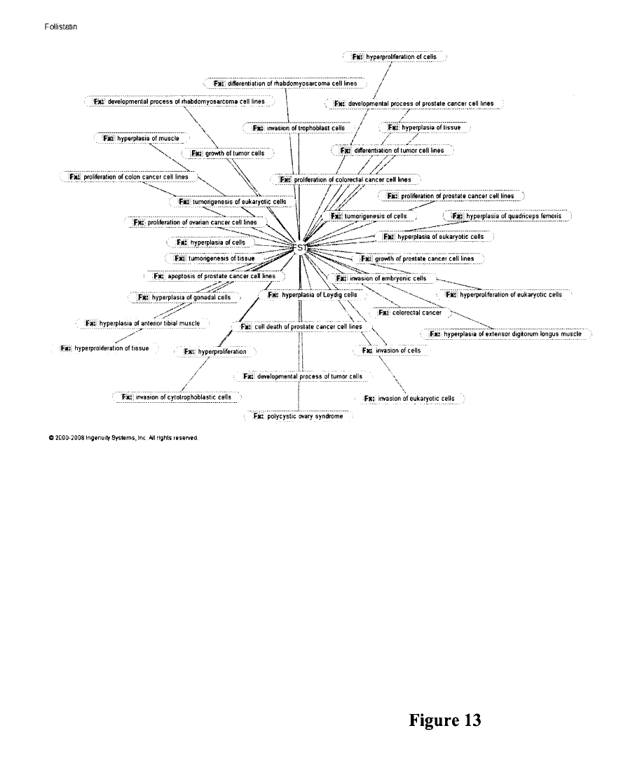

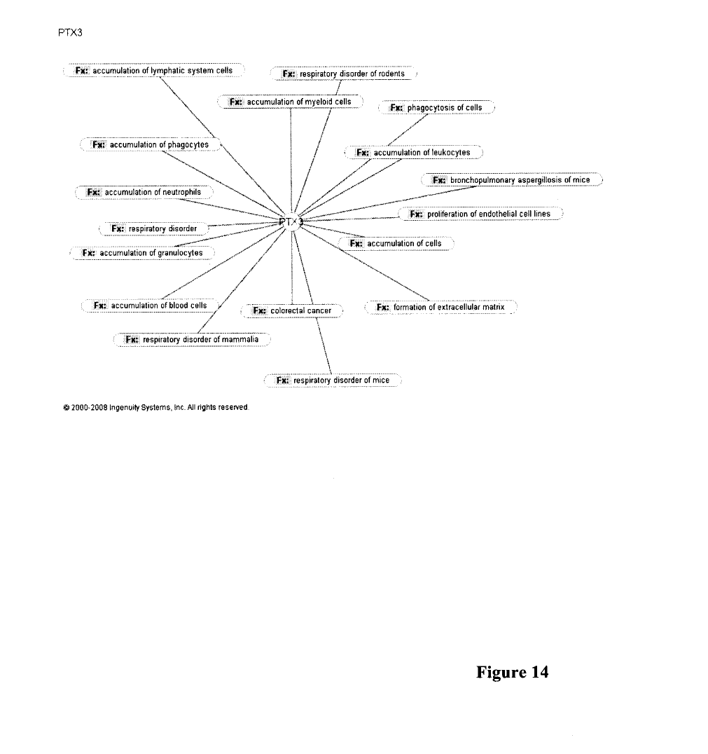

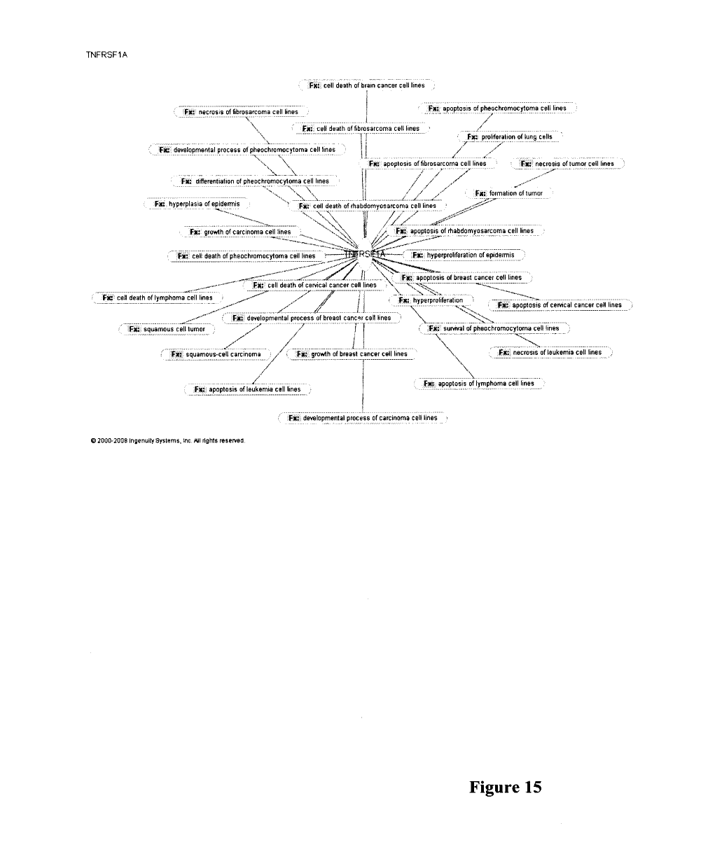

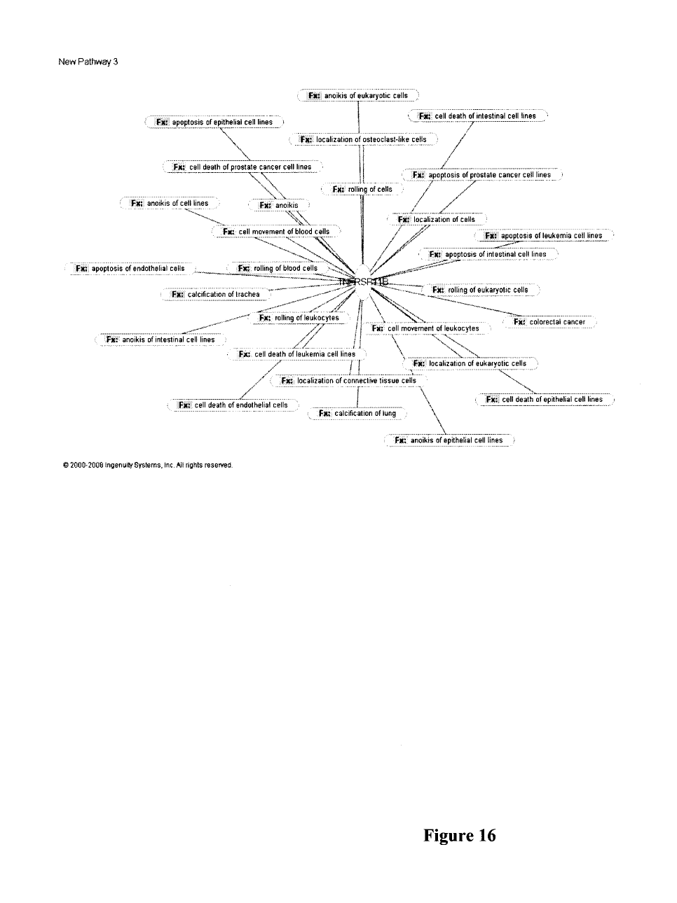

The potential biological functions of extracellular and membrane-bound proteins identified in CM of all cell lines were analyzed using Ingenuity Pathway Analysis. The top 10 functions are illustrated in FIG. 6. Major categories included cellular movement, cell-to-cell signaling and interaction, cellular growth and proliferation and cancer. Ingenuity Pathway Analysis was also used to develop biological networks showing the functions and disease association of each of the five candidates selected for preliminary validation. ADAM-17 plays a significant role in recruitment of immune cells during the inflammatory response. As well, ADAM-17 plays a role in modulating cell adhesion and potentially contributing to the invasiveness of cancer cells. Both of these functions have been well-recognized to play a significant role in tumor progression and invasion. The biological network constructed for ADAM-17 is presented in FIG. 7. Follistatin is associated with various processes involving malignant progression and invasion. As presented in FIG. 13, Follistatin is involved with regulation of cell growth and proliferation of various cancer cell lines. The molecular functions associated with PTX3 are highlighted in FIG. 14. These include participation in mediation of inflammatory response. Interestingly, PTX3 was shown to be involved with respiratory disorders in mice. The protein sTNF RI displays connections with cancer progression. As shown in FIG. 15, networks involved with cancer include apoptosis, malignant progression, cell survival and proliferation. Finally, Osteoprotegerin (TNFRSF11B) has shown to be involved with several molecular networks, including cell adhesion, apoptosis, cell migration, and malignant transformation (FIG. 16).

### Discussion

In proteome projects, the 2-DE approach has been the primary technique of separation and comparison of complex protein mixtures. However, this approach suffers from large variations caused by sample preparation, protein loading and gel staining [38]. Another limit of 2-DE for proteomics concerns the poor recovery of proteins from gel for MS. Methods to supplement or replace 2-DE, such as multidimensional LC (multi-LC) have therefore been sought [39]. Multi-LC-MS/MS analysis allows identification of proteins in a high throughput fashion unlike the rather slow and laborious 2DE-MS/MS methods. This technique has been used to discover cancer biomarkers by analyzing complex protein mixtures such as biological fluids, tissues or cell cultures [14, 15, 40-44]. However, this technology is still challenged in the case of complex mixtures such as serum, that require well-established methodologies for depletion of highly abundant proteins and efficient sample fractionation before proteomic analysis [12, 13].

A 2D-LC-MS/MS strategy was utilized to identify the secretome of four lung cancer cell lines of differing histological subtypes, grown in serum-free media. Since lung cancer is a heterogenous disease, the secretome of cell lines of differing origin was analyzed in order to have a better depiction of the proteome of lung cancer and more chances to discover biomarkers of this pathology. By searching with both Mascot and X!Tandem, over 1,800 proteins were identified in the CM of all four cell lines combined, which represents one of the largest repositories of proteins identified for lung cancer. As reported by Kapp et al., the use of multiple search engines increases confidence of protein identification [45]. These search engines utilize different algorithms and scoring functions to determine if a mass spectrum matches an entry in the database [46]. Moreover, by combining the use of Peptide and ProteinProphet algorithms embedded within Scaffold, an increased confidence of protein identification probabilities is made [23, 24]. Particular attention was placed on extracellular and membrane-bound proteins from the four lung cancer cell lines, because these proteins have the highest probability of being found in the circulation and function as putative biomarkers. Thirty eight percent of identified proteins were classified as extracellular and membrane-bound. Among them, various cytokines, proteases, protease inhibitors, growth factors, extracellular matrix proteins and receptors were identified. In addition, a large number of intracellular proteins were found, including ones classified as nuclear and cytoplasmic by GO annotation. In general, the proteomics data reported herein revealed a similar distribution of proteins by cellular component in each cell line. During the cell culture phase, a small portion of the cell population dies, resulting in the release of intracellular proteins into the conditioned media. Despite efforts to optimize cell culture conditions to minimize cell death and maximize secreted protein concentration, the identification of intracellular proteins in the CM by MS is inevitable because of the high sensitivity of the technique. By using quantitative proteomic techniques (ICAT reagents and MS/MS) to identify secreted and cell surface proteins from a prostate cancer cell line (LNCaP), Martin et al., found that more than 50% of proteins identified in LNCaP-conditioned media were classified as intracellular [42]. However, previous studies using a similar cell-culture-based approach, showed that proteins identified in the cell lysate did not contain as many secreted proteins as the CM for that cell line [14]. Furthermore, the extracellular proteins found in the cell lysate showed minimal overlap with the proteins identified in the CM [14]. This data demonstrates that the strategy used herein significantly enriches for secreted proteins.

Each cell line was cultured in triplicate. Using an in-house developed program, the overlap of identified proteins between the 3 replicates of each cell line was examined. As shown in FIGS. 2, a 63.5 to 70.5% overlap of proteins between the replicates of each cell line was observed, suggesting excellent reproducibility between runs. Due to the nature of mass spectrometric measurements, not all peptides are ionized in each run, and subsequently, different peptides are selected for ionization and finally detected [14]. The diverse steps during sample preparation, including reduction-alkylation, lyophilization, sample fractionation, zip-tipping, can also be important contributing factors to the variations observed between the replicates.

As determined by specific ELISA, the presence of three internal controls (IGFBP-2, KLK11 and KLK14) was confirmed by mass spectrometry in the CM of all lung cancer cell lines. Among them, KLK14 was the less abundant protein (1.9 μg/L, as determined by ELISA) and was detected in two out of the three replicates of H1688 by one and three unique peptides, respectively. It is conceivable that the detection limit of the method described herein is close to this value of 1.5-2 μg/L, as previously reported [14, 15]. Based on these observations, this proteomic strategy can identify proteins in CM in the low μg/L range or higher. With regard to current biomarkers used in the clinic, this is the expected concentration range, giving hope that new lung cancer markers should be detectable in serum. The method described herein successfully identified proteins that are candidate or currently used as biomarkers of lung cancer, including CEA [27, 28], Pro-GRP [29, 31], SCC antigen [47, 48], Tumor M2-PK [49], NCAM [35-37], chromogranin A [29] and chromogranin B [30]. In addition, candidate markers were identified that were previously reported in lung-related proteomic studies such as member C1 of aldo-keto reductase family 1 (AKR1C1) identified by Huang et al. as dihydrodiol dehydrogenase [25] and MMP1 found to be overexpressed in lung cancer patients and especially, in late stage [18]. Furthermore, 129/291 extracellular and 168/415 membranous proteins identified were found in the plasma proteome. These data overall, further support the strategy of using the CM of lung cancer cell lines to discover candidate biomarkers.

From the list of proteins, some arbitrary criteria were used to select the most promising candidates for validation. Given that serological biomarkers identified so far are generally secreted or shed proteins, such as PSA, CA-125 and SCC-Ag in prostate, ovarian and lung cancer, respectively, it was hypothesized that new lung cancer markers might be secreted proteins, or their fragments, originating from cancer cells or their microenvironment and then enter the circulation [50]. Consequently, the focus was on proteins that were classified as extracellular or membrane-bound. As secondary criteria, proteins were selected that showed relatively lung-specific expression at the mRNA or protein level by examining the UniGene expressed tag database and the Human Protein Atlas database (www.proteinatlas.org). Then, literature searches were performed to ensure that these proteins have not been examined as serological markers for lung cancer, and showed biological connections with lung or other cancers. Selected proteins were compared to the proteome of lung-related diseases (lung cancer [18, 25, 26], pleural effusion [11]) or the proteome of a lung-related biological fluid (induced sputum [9, 10]) and serum (http://www.plasmaproteomedatabase.org). Finally, potential candidates that had commercially available antibodies or immunoassays were selected.

From this selection, five candidates were retained for further investigation: ADAM-17, Pentraxin 3, sTNF RI, Osteoprotegerin and Follisatin. Serum levels of each candidate were higher in NSCLC patients in comparison with healthy controls. To examine the putative connections with lung cancer, biological networks were constructed of each candidate in association to functions and diseases (FIGS. 7, 13-16). Each candidate is associated with various processes including tumor development or malignant progression. Previously, ADAM-17 was found to be overexpressed in breast cancer and associated with tumor progression and metastasis [51, 52]. ADAM-17 was also shown to predict adverse outcome in breast cancer [53]. More recently, ADAM-17, a major ErbB ligand sheddase, was found to be upregulated in NSCLC tumor samples and was required not only for heregulin-dependent HER3 signaling, but also for EGFR ligand-dependent signaling in NSCLC cell lines [54]. Pentraxin-3 was the first long pentraxin discovered, initially named TSG-14 and later identified as an IL-1 inducible gene in human umbilical vein endothethelial cells [55]. Despite the fact that PTX3 was reported in preventing infection by certain fungi, bacteria or viruses in the lung, increased expression was also associated with more severe lung injury such as high volume mechanical ventilation or severe bacterial infection [56]. Follistatin showed several links to cancer, such as prostate [15, 57, 58], colon [59] and ovarian cancer [60]. Concerning its link to lung cancer, Follistatin has been suggested to suppress the production of multiple-organ metastasis by small cell lung cancer cells in natural killer cell-depleted severe combined immunodeficiency (SCID) mice, predominantly by inhibiting angiogenesis [61]. As shown in FIG. 15, TNF RI is associated with cancer by participating in apoptosis, malignant progression and proliferation. A spontaneous regression of lung metastasis was observed by Tomita et al. in the absence of tumor necrosis factor receptor p55 [62], suggesting that TNF RI-mediated signals could maintain tumor neovascularization at least partly by inducing HGF expression and, eventually support lung metastasis. Osteoprotegerin, a secreted member of the tumor necrosis factor receptor superfamily, has not been well-documented in lung disease. However, it displays several connections to cancer, such as pancreatic, colorectal [63] and bladder carcinoma [64].

Due to the heterogeneity of lung cancer and the lack of sensitivity and specificity of individual markers, there is a growing consensus that panels of markers can improve screening, diagnosis, prognosis, or monitoring responses to therapy.

In summary, presented herein is one of the most comprehensive proteomic analyses of conditioned media from four lung cancer cell lines for new biomarker discovery. Five candidates have been further validated as serum markers for lung cancer.

### Materials and Methods

### Cell Lines and Cell Culture

The four lung cancer cell lines, H23 (CRL-5800), H520 (HTB-182), H460 (HTB-177) and H1688 (CCL-257) were purchased from the American Type Culture Collection (ATCC, Rockville, Md.). These cell lines represent the four major histological lung cancer subtypes: (i)-NSCLC, adenocarcinoma (H23), squamous cell carcinoma (H520), large cell carcinoma (H460); (ii)-SCLC (H1688). All cell lines were maintained in 75 cm² culture flasks in RPMI 1640 culture medium (BD Biosciences) supplemented with 8% fetal bovine serum (FBS) (Hyclone). All cells were cultured in a humidified incubator at 37° C. and 5% CO₂.

Cells were seeded at different seeding densities (4×10⁶ cells for H460, 8×10⁶ cells for H23, 10×10⁶ cells for H1688 and 12×10⁶ cells for H520, respectively) into six 175 cm² culture flasks per cell line (with the exception of three flasks for H460) and grown for 2 days in 30 ml of RPMI supplemented with 8% FBS. After 2 days, the culture medium was removed and the cells rinsed 3 times with 30 ml of 1× phosphate-buffered saline (PBS) (Invitrogen). Then, 30 ml of chemically-defined Chinese Hamster Ovary (CDCHO) serum-free medium (Invitrogen), supplemented with glutamine (8 mM) (Invitrogen) were added to the flasks and the flasks were incubated for 48 hours. The H520 cell line was grown as described above, except that the cells were incubated for 3 days in RPMI supplemented with 8% FBS, before the medium was changed to CDCHO serum-free medium. All cell lines were grown in triplicate and independently processed and analyzed. The same conditions and procedures were applied to set up a negative control. In this case, 30 ml of RPMI supplemented with 8% FBS were prepared as mentioned above, with no cells added to the 175 cm² culture flask.

After incubation in CDCHO, the conditioned media (CM) were collected and spun down to remove cellular debris. The CM were then frozen at −80° C. until further processing. Aliquots (1 ml) were taken from the CM at the time of harvest for measurement of total protein and lactate dehydrogenase (LDH), as well as kallikrein-related peptidases 11, 14 and insulin-like growth factor binding protein 2 (internal control proteins) by using specific ELISA assays.

### Measurement of Total Protein, Lactate Dehydrogenase, Kallikreins 11, 14 and IGFBP2

Total protein was quantified in the CM using a Coomassie (Bradford) assay (Pierce Biotechnology) according to the manufacturer's instructions. Lactate dehydrogenase (indicator of cell death) was measured in the CM using an enzymatic assay based on lactate to pyruvate conversion and parallel production of NADH from NAD⁺. The production of NADH was monitored at 340 nm using an automated method (Roche Modular Systems). Kallikrein-related peptidases 11 and 14 were measured with in-house enzyme-linked immunosorbent assays (ELISA) as previously described [19-21]. IGFBP2 sandwich ELISA kit, purchased from R&D Systems, was used to measure levels of IGFBP2 in the CM of lung cancer cell lines.

### Conditioned Media Sample Preparation

One CM aliquot (30 ml) was collected for the cell line H460, whereas two-30 ml CM aliquots were combined (60 ml) for the 3 cell lines H23, H1688 and H520. Three biological replicates per cell line were performed. Each replicate contained approximately 800 μg to 1 mg of total protein.

These replicates were dialyzed using a 3.5-kDa molecular mass cutoff membrane (Spectrum Laboratories, Inc., CA, USA). The CM were dialyzed overnight at 4° C. in 5 liters of 1 mM ammonium bicarbonate solution with two buffer changes. The dialyzed CM were frozen and lyophilized to dryness. Following lyophilization, samples were denatured using 8M urea and reduced with DTT (final concentration of 13 mM, Sigma-Aldrich) at 50° C. for 30 min. Then, samples were alkylated with 500 mM iodoacetamide (Sigma-Aldrich) in the dark at room temperature for 1 h and desalted using a NAP5 column (GE Healthcare). The 1 ml final samples were lyophilized and trypsin (Promega)-digested at a molar ratio of 1:50 (trypsin:protein concentration) overnight at 37° C. Finally, the peptides were lyophilized to dryness.

### Strong Cation Exchange Liquid Chromatography

The trypsin-digested lyophilized samples were resuspended in 120 μl of 0.26M formic acid in 10% acetonitrile (ACN; mobile phase A). The samples were fractionated using an Agilent 1100 HPLC system connected to a PolySULFOETHYL A® column with a 200-Å pore size and a diameter of 5 μm (The Nest Group Inc.). A one hour linear gradient was used, with 1M ammonium formate and 0.26M formic acid in 10% acetonitrile (mobile phase B) at a flow rate of 200 μL/min. Fractions were collected via a fraction collector every 5 min (12 fractions per run) and frozen at −80° C. for further use.

A peptide cation exchange standard, consisting of three peptides, was run at the beginning of each day to assess column performance (Bio-Rad).

### Mass Spectrometry (LC-MS/MS)

Of the 12 fractions collected per HPLC run, seven fractions (fractions 5 to 11, containing the bulk of peptides) were analyzed by mass spectrometry. The seven fractions per replicate per cell line were C₁₈-extracted using a ZipTip_{C18} pipette tip (Millipore) and eluted in 4 μL of 90% ACN, 0.1% formic acid, 10% water and 0.02% trifluoroacetic acid (TFA) (Buffer B). Eighty μL of 95% water, 0.1% formic acid, 5% ACN, and 0.02% TFA (Buffer A) were added to this mixture, and 40 μl were injected via an autosampler on an Agilent 1100 HPLC. The peptides were first collected onto a 2-cm C₁₈ trap column (inner diameter, 200 μm), then eluted onto a resolving 5-cm analytical C₁₈ column (inner diameter, 75 μm) with an 8-μm tip (New Objective). The HPLC was coupled online to a 2-D Linear Ion Trap (LTQ, Thermo Inc.) mass spectrometer using a nano-ESI source in data-dependent mode. Each fraction was run with a 120-min gradient. The eluted peptides were subjected to tandem mass spectrometry (MS/MS). DTAs were created using the Mascot Daemon v2.16 and extract_msn (Matrix Science). The parameters for DTA creation were: minimum mass, 300 Da; maximum mass, 4000 Da; automatic precursor charge selection; minimum peaks, 10 per MS/MS scan for acquisition; and minimum scans per group, 1.

### Data Analysis

Mascot (Matrix Science, London; version 2.1.03) and X!Tandem (Global Proteome Machine Manager, Beavis Informatics Ltd; version 2.0.0.4) search engines were used to analyze the resulting raw mass spectra from each fraction. Each fraction was analyzed by both search engines on the International Protein Index (IPI) Human database (version 3.16; >62,000 entries) [22]. One missed cleavage was allowed and searches were performed with fixed carbamidomethylation of cysteines and variable oxidation of methionine residues. A fragment tolerance of 0.4 Da and a parent tolerance of 3.0 Da were used for both search engines with trypsin as the specified digestion enzyme. This operation resulted in seven DAT files (Mascot) and seven XML files (X!Tandem) for each replicate sample per cell line. Scaffold (version Scaffold-01_{—}06_{—}19, Proteome Software Inc., Portland, Oreg.) was utilized to validate MS/MS-based peptide and protein identifications. The cutoffs in Scaffold were set for 95% peptide identification probability as specified by the PeptideProphet algorithm [23] and 80% protein identification probability as assigned by ProteinProphet algorithm [24]. Identifications not meeting these criteria were not included in the displayed results. The DAT and XML files for each cell line plus their respective negative control files (RPMI-1640 culture medium only) were inputted into Scaffold to cross-validate Mascot and X!Tandem data files. Each replicate sample was designated as one biological sample containing both DAT and XML files in Scaffold and searched with MudPIT (Multidimensional Protein Identification Technology) option selected. Using a similar approach of analysis of conditioned media from breast and prostate cancer cell lines, a false positive error rate of 1-2% using the sequence-reversed IPI human database was observed.

The sample reports were exported to Excel, and an in-house developed program was used to extract Genome Ontology (GO) terms for cellular component for each protein and the proportion of each GO term in the dataset. Proteins that were not able to be classified by GO terms were checked with Swiss-Prot entries and against the Human Protein Reference Database and Bioinformatic Harvester to search for cellular component annotations. The overlap between proteins identified from each cell line and between the 3 replicates of each cell line was assessed using an in-house developed program. All extracellular and membrane-bound proteins were also searched against the Plasma Proteome Database. The list of displayed proteins were also compared with those found in other lung-related proteomic studies [9-11, 18, 25, 26]. Finally, the extracellular and membrane proteins identified by cellular function and disease were classified using Ingenuity Pathway Analysis software (Ingenuity Systems). In addition, the molecular functions associated with each of the biomarker candidates were analyzed with the Ingenuity Pathway Analysis software.

### Validation of Lung Biomarker Candidates: Clinical Samples and ELISA Analysis

Samples were collected at the UCLA Medical Centre between October 2004 and March 2006, in accordance with the UCLA Institutional Review Board approval and patient written informed consent from fifty subjects, including 25 cases diagnosed with NSCLC and 25 normal healthy donors. Peripheral blood was collected from patients at least 4 weeks prior to receiving therapy or from patients with advanced disease. In patients who had previously undergone surgical resection, blood was collected after recurrence at least one year following surgery. Plasma was collected in EDTA-containing vacutainer tubes. Samples were centrifuged at 3,000 rpm for 15 minutes within one hour of collection, separated, and stored in aliquots at −80° C. Staging was determined by the American Joint Committee on Cancer Guidelines. Distributions of patients by demographic and clinical characteristics are presented in Tables 2-6 for each of the candidates tested.

Serum levels of Pentraxin-3 (TSG-14), Follistatin and sTNF RI were measured by ELISA, using a commercially available kit (R&D Systems, Minneapolis, USA). Serum levels of Osteoprotegerin and ADAM-17 were measured using an in-house developed ELISA, using commercial antibodies purchased from R&D Systems.

### Statistical Analysis

The differences between groups were evaluated by the Mann-Whitney test using GraphPad Prism version 4 for Windows (GraphPad software, San Diego, Calif., USA). All comparisons were two-tailed, and p values of <0.05 were considered significant.

### Example 2

### Diagnostic Accuracy of Proteins Identified by MS as Lung Cancer Biomarkers

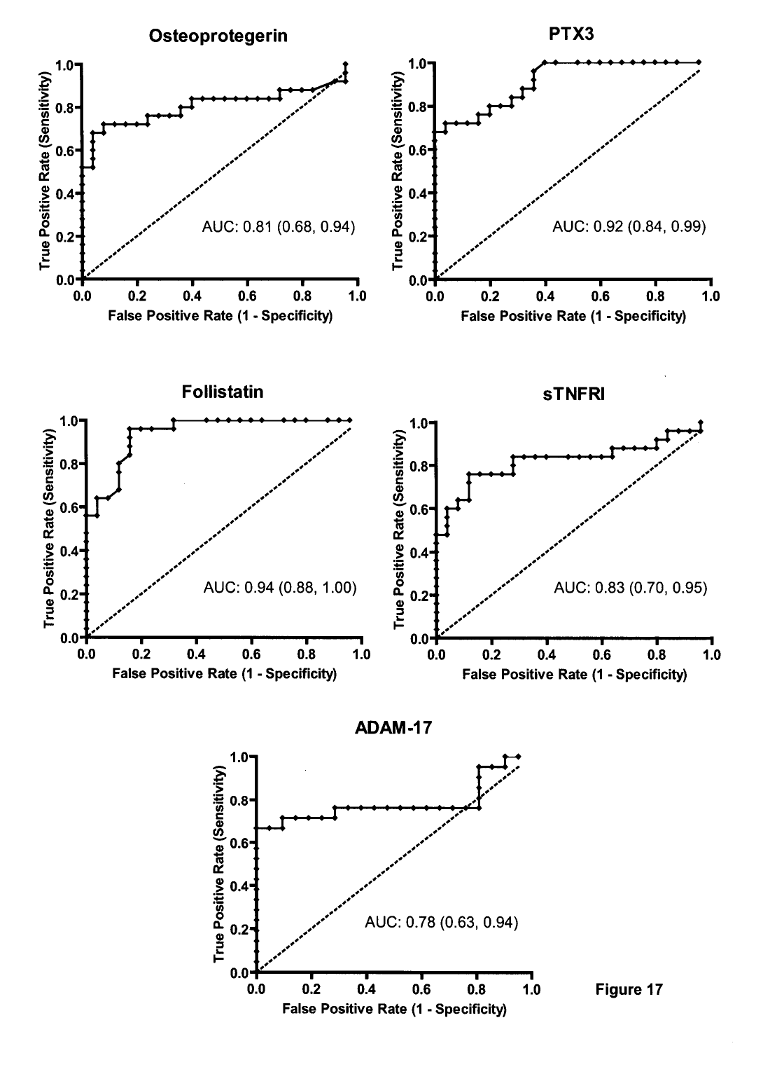

For the samples collected at UCLA, the clinical usefulness of ADAM-17, Osteoprotegerin, Pentraxin 3, sTNF RI and Follistatin in distinguishing samples obtained from subjects with NSCLC (cases) and subjects that were lung cancer free (controls) was investigated using Receiver Operating Characteristic (ROC) curve analysis and the sensitivity and specificity, using each value in the data table as the cut-off value, was calculated using GraphPad Prism version 4 for Windows (FIG. 17). The ROC curve is a plot of the true positive fraction versus the false positive fraction. GraphPad Prism tabulates sensitivity and 1-specificity, with 95% confidence intervals (CI), for all possible cut-off values. Each lung cancer biomarker was useful in discriminating between samples obtained from subjects in the NSCLC or non-lung cancer group, with an area under the curve (AUC) ranging from 0.78 for ADAM-17 to 0.94 for Follistatin (see FIG. 17 and Table 9). Pentraxin 3 and Follistatin showed the highest AUC (>0.90; 95% CI, 0.84-1.00) in differentiating between cases and controls.

### Example 3

### Diagnostic Accuracy of Pentraxin 3, KLK11 and Progranulin as Lung Cancer Biomarkers

The clinical usefulness of Pentraxin 3, KLK11 and progranulin in distinguishing samples obtained from subjects with lung cancer and subjects that were lung cancer free was investigated as described in Example 2 using samples obtained from the Early Detection Research Network (EDRN; http://edrn.nci.nih.gov) of the National Cancer Institute (NCl). These samples consist of a total of 426 samples from 203 patients diagnosed with lung carcinoma (please see below), 180 individuals at high risk for lung cancer due to a history of cigarette smoking, and 43 individuals with cancers other than lung (25 breast cancer, 18 colon cancer). The lung cancer cases and high-risk controls were at least 40 years old, and the high-risk controls had a cigarette smoking history of at least 30 pack-years. Cases and high-risk controls were frequency matched on age, cigarette smoking history, and center where the specimens were collected. The specimens tested represent a copy of the lung cancer “Reference Set A” (“Blood Repository for the Validation of Lung Cancer Biomarkers” Lung Cancer Biomarkers Group, Apr. 14, 2010 (edrn.nci.nih.gov/resources/sample-reference-sets/LCBG %2OAPR %2014%202010.DOC/VIEW created by the EDRN. Specimens in this reference set were contributed by four institutions (MD Anderson Cancer Center, New York University, UCLA, and Vanderbilt University) from archive samples previously collected and stored at −80° C. One aliquot (100 μL of serum) was shipped to the laboratory of Dr. E. P. Diamandis on dry ice. Samples were labeled with a number and they were blinded. The code was broken only after ELISA analysis was completed and the data submitted to a statistician.

In all serum samples, Pentraxin 3, KLK11 and progranulin were quantified by using ELISA methodologies. The ELISA for KLK11 was developed in-house and described elsewhere [20]. The ELISA kit for progranulin was purchased from R&D Systems, Minneapolis, Minn., USA and it was used according to the manufacturer's recommendations. KLK11 and progranulin were found here to be non-informative biomarkers for lung carcinoma.

Pentraxin 3 ELISA kits were purchased from R&D Systems. The assay is based on two antibodies, one used for capture (monoclonal mouse antibody) and one used for detection (biotinylated goat polyclonal antibody). Standardization was achieved by using recombinant, purified Pentraxin 3 provided by the manufacturer. The manufacturer's recommendations and protocol were used and serum samples were diluted 3-fold with a 6% bovine serum albumin solution before analysis. The calibration curve was linear from 200 to 20,000 pg/mL and the precision in this range was <10%. All assays were performed in duplicate.

ROC curves for progranulin and KLK11 for the whole patient group and against all controls, or only the high-risk controls were not informative (the AUCs were close to 0.50 and not statistically significant). For this reason, further statistical analyses for these two biomarkers were not performed.

ROC curves were constructed for the whole group of patients and controls, as well as for cases subgroups stratified by histology type and stage and control subgroups stratified by control type (high-risk versus other cancer). The AUC and the sensitivity of Pentraxin-3 at selected specificity cut-off points were also calculated and confidence intervals for these quantities calculated by bootstrap. Not all patients had complete clinicopathological information and, as deemed necessary, subgroups were combined to increase the statistical power of the calculations. All analyses were performed using Stata Version 11 and the pcvsuite of basic ROC analysis commands created by Dr. M. Pepe [77, 78].

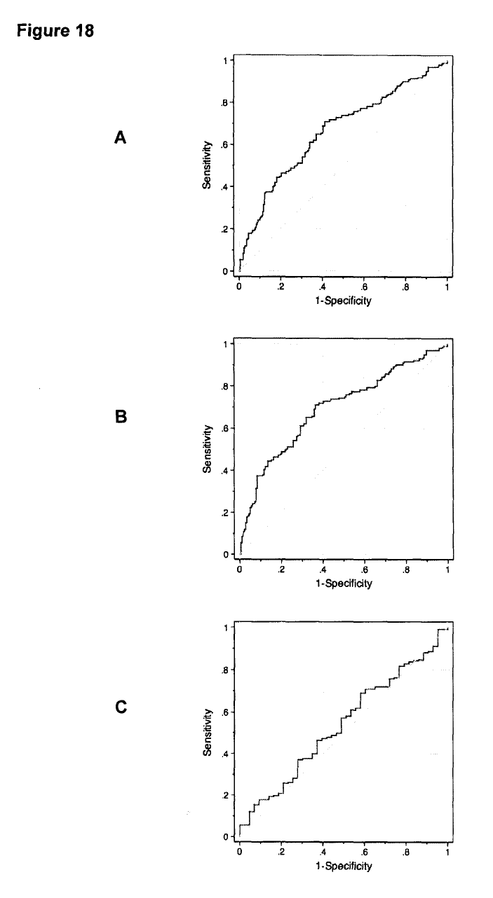

The ROC curve for Pentraxin-3 for all cases (N=203) and all controls (N=223), all cases and high-risk controls (N=180), and all cases and other cancer controls (N=43) are shown in FIG. 18 (panels A, B, and C, respectively). Pentraxin-3 has significant discriminatory value, especially when comparing all patients, to the high-risk controls (which is a relevant group for population screening purposes).

The sensitivity of Pentraxin-3 versus high-risk controls and all controls at various specificity cut-offs is shown in Table 11. At 90% and 80% specificity, the sensitivities versus the high-risk controls were 37% and 48%, respectively.

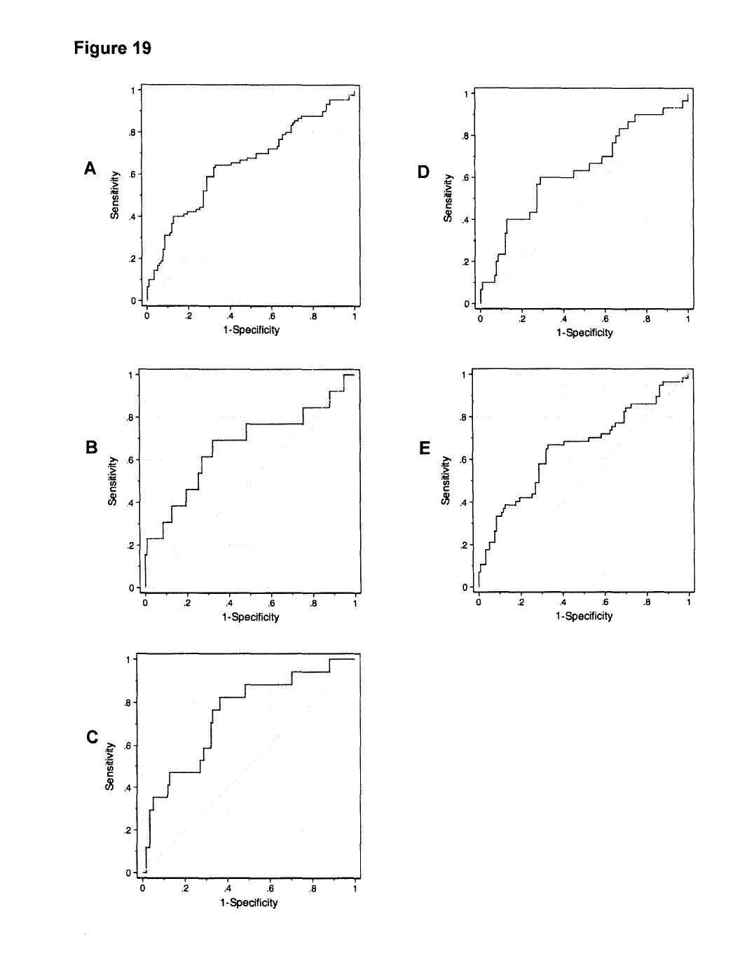

ROC curve analysis was also performed in sub-groups of patients, stratified by histology. Among the patients for which information was available, there were 90 NSCLC cases, 13 SCLC cases and 17 cancers for which classification could not be determined. Among the 90 NSCLC cases, there were 30 squamous cell carcinomas and 57 adenocarcinomas (3 undetermined). The ROC curves for these sub-groups are shown in FIG. 19. A summary of the AUC for each one of the sub-groups is shown in Table 12. The ROC curves and associated AUCs were generally very similar with all sub-groups. It appears that Pentraxin 3 has similar discriminating ability with all of the major sub-types and histotypes of lung cancer.

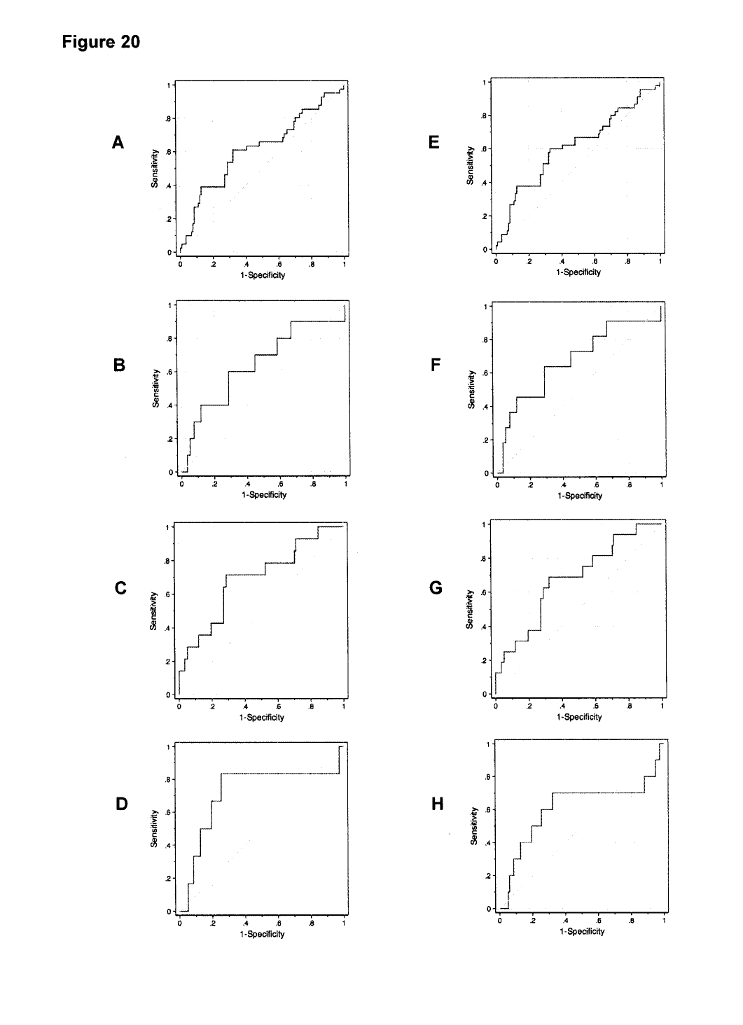

There were only 44 patients with known pathological stage, 29 in stage I, 3 in stage II, 8 in stage III and 4 in stage IV. There was an increase in AUC from stage I to stage IV, as follows: AUC 0.62 for stage I, 0.64 for stage II, 0.69 for stage III and 0.72 for stage IV disease. For some patients, either the pathological or clinical stage was known. When the data was analyzed according to combined stage (either pathological or clinical stage present), the following was found: AUC=0.61 (stage I; N=45), AUC=0.67 (stage II; N=11), AUC=0.68 (stage III; N=16) and AUC=0.61 (stage IV; N=10) (FIG. 20).

### Example 4

### Analysis of Other Lung Cancer Cell Lines

Lung cancer has two major histological types—small cell lung carcinoma (SCLC) and non-small cell lung carcinoma (NSCLC). NSCLC can be further sub-divided into squamous cell carcinoma, adenocarcinoma and large cell lung carcinoma. Thus, twelve lung cancer cell lines representative of each subtype were chosen for analysis. This includes two cell lines derived from normal embryonic lung tissue and adult bronchial tissue.

Four SCLC cell lines were chosen—NCI-H1688, DMS-153, NCI-H146 and NCI-H889, all of which were derived from liver, bone marrow and lymph node metastasis. SCLCs comprise approximately 16% of lung cancers and are known for their aggressiveness. In terms of NSCLC, three adenocarcinoma cell lines (NCI-H2126, NCI-H23 and NCI-H522 ranging from late stage metastasis to early stage), three squamous cell carcinoma cell lines (HTB-58, HBT-182 and NCI-H2066, comprising a pleural effusion metastasis, carcinoma in situ and a mixed squamous/small cell/adenocarcinoma in stage 1, respectively), and one large cell lung cancer cell line (HTB-177, derived from a pleural effusion metastasis) were chosen. Two cell lines derived from lung fibroblasts (WI-38) and bronchus (NL-20), exhibiting properties of normal cells will also be utilized. Four lung cancer cell lines, H23 (CRL-5800), H520 (HTB-182), H460 (HTB-177) and H1688 (CCL-257) were analysed as described in Example 1 and represent the four major histological lung cancer subtypes: (i)-NSCLC, adenocarcinoma (H23), squamous cell carcinoma (H520), large cell carcinoma (H460); (ii)-SCLC (H1688). The remaining 8 cell lines will be analysed as in Example 1.

### Example 5

Complex signalling networks working through protein-protein interactions within cells are essential for proper biological function. A similar phenomenon is seen under pathological conditions. Aberrant signalling is one of the hallmarks of tumorigenesis and cancer progression. Deregulated expression and functioning of proteins under cancerous conditions occurs not only within cancer cells but extends to the tumour microenvironment and surrounding host tissue. As such, the dynamic interplay between tumour cells and the surrounding ‘normal’ host tissue, that is, the ‘tumour-host interface’, significantly influences aspects of tumour growth and maintenance. These biological phenomena are also relevant to biomarker discovery. Aside from secreted and shed proteins, cleavage of transmembrane proteins by proteases found in the tumour microenvironment is an important mechanism by which proteins can enter the circulation and serve as biomarkers. Analysis of tissue culture supernatants of cancer cells, as well as relevant biological fluids in close proximity to the tumour, should capture biomarkers generated by protein secretion, shedding, proteolysis and tumour-host interface.

Much of the past research on proteomics-based biomarker discovery has focused on serum and tissue analysis. Serum analysis, although scientifically sound, is problematic for initial proteomic analysis and discovery of candidates. Serum is a highly heterogeneous fluid and protein concentrations vary from individual to individual. This can potentially confound results during comparative analyses in the discovery phase. Additionally, biomarkers are usually proteins present in low amounts in serum (ng to pg/mL levels) and due to the highly complex nature of serum, there is an increased chance that such low abundance proteins (potential novel biomarkers) are masked by high-abundance proteins (present at ug to mg/mL levels) during high-throughput protein identification. Similar problems apply to tissue proteomics. As per our hypothesis, and based on past research, the majority (if not all) of clinically useful serum biomarkers are secreted or shed proteins. This subset of proteins comprises only 20-25% of all proteins present in a cell. As a result, analysis of the tissue proteome may also result in the masking of potential biomarkers by other, more highly abundant proteins. Instead, enrichment of the secreted/shed subset through analysis of tissue culture supernatants into which tumor cells contribute their secretions, as well as biological fluids found in close proximity to tumour cells, should bypass some of the problems of serum and tissue proteomics. Biological fluid found in close proximity to tumour cells will also be subjected to proteomic analysis. Bronchoalveolar lavage fluid from non-malignant disease [n=5] and lung cancer [n=5] will be analyzed.

The proteome of the biological fluid will be delineated following procedures similar to those described in Example 1. Additional chromatographic purifications (such as gel filtration chromatography) will be incorporated, as necessary, to rid the samples of high abundance proteins.

Fluid samples will be subjected to three 30-minute centrifugations to remove cellular debris and lipids. They will then undergo size exclusion chromatography to remove proteins of high abundance, as previously described for malignant ascites (70). To maximize coverage of the respective biofluid proteomes, centrifugal ultrafiltration with disposable devices (according to manufacturer specifications) is optionally performed to select for proteins ≦30 kDa. Subsequent to these pre-fractionation methods, the samples will be reduced, alkylated and trypsin-digested as per the cell line CM, followed by fractionation on an SCX column. The peptides in the generated fractions will then be concentrated using a C18 Zip Tip and run through an LC-MS/MS system for protein identification [70]. All analyses following the pre-fractionation steps, including bioinformatics, will be similar to those of the cancer cell lines.

### Example 6

Commercially available ELISA assays and quantitative mass spectrometric approaches such as multiple reaction monitoring (MRM) and product-ion monitoring (PIM) will be used to compare concentrations of candidates in serum of normal individuals and patients with benign diseases vs. patients with cancer. For those candidates lacking commercially available antibodies or ELISA kits the corresponding recombinant proteins will be produced and utilized for production of antibodies. The antibodies will then be used to develop sandwich-type ELISA assays for quantification.

Production of recombinant proteins: In order to express the necessary recombinant proteins, plasmids containing the full and verified sequence of the molecules of interest will be obtained, either from commercial sources (such as Origene Technologies; http://www.origene.com) or from the Harvard Institute of Proteomics (www.hip.harvard.edu). The sequences to be expressed will be inserted into Invitrogen's “Gateway Vector System”, which allows convenient sub-cloning into secondary vectors suitable for high-yield expression in E. coli, yeast, baculovirus or mammalian cells. E. coli expression will be used first and, if necessary, yeast, baculovirus and mammalian cells will be tried in this sequence. The goal is to produce mg amounts of each one of these proteins, to be used as immunogens for monoclonal and polyclonal antibody production. Incorporation of a polyhistidine tag in each of the recombinant proteins will help facilitate subsequent purification. After production, the recombinant proteins will be further purified by affinity chromatography on nickel columns and, if necessary, by additional ion-exchange or reverse-phase chromatography. The purity of the final proteins will be assessed by polyacrylamide gel electrophoresis and Coomassie or silver staining and protein identities will be verified by using tandem mass spectrometry, available in-house.

Production of monoclonal antibodies: Mice will be immunized with the recombinant proteins, by using a standardized protocol. After checking for satisfactory polyclonal response by ELISA, the spleens of the animals will be removed and the lymphocytes will be fused by polyethylene glycol with a suitable myeloma partner (e.g. SP 2/0 cells) to produce hybridomas. The hybridomas will be cultured, sub-cloned and screened by using standard procedures to identify clones secreting antibodies which interact specifically with the proteins of interest. This approach usually yields approx. 5-7 promising clones which will be further evaluated for their suitability for constructing ELISA assays. The identified clones will be expanded and monoclonal antibodies will be produced, first in tissue culture, followed by either ascites or hollow fiber bioreactor columns to produce larger amounts. The monoclonal antibodies will be purified by protein A/G affinity chromatography and assessed for specificity by Western blots.

Production of polyclonal antibodies: By using the recombinant proteins as immunogens, two rabbits will be immunized with a standardized protocol, which includes approx. 100 μg of immunogen per animal, every 3 to 4 weeks. The first immunization will be performed with use of complete Freund's adjuvant and subsequent immunizations with incomplete adjuvant. High titers of polyclonal antibodies (working at dilutions from 100,000 to 1,000,000-fold on Western blots) are expected after the 6^{th} immunization. After checking the titers of antibodies during the immunization period by ELISA, rabbits will be sacrificed and approx. 50 ml of antiserum will be obtained. This antiserum will be further purified by protein NG affinity chromatography to obtain an IgG fraction of the polyclonal antibody. The specificity of the polyclonal antibodies will be verified by using Western blot analysis.

Development of ELISA assays: Depending on the availability of suitable antibodies, we will opt to develop either monoclonal/monoclonal or monoclonal/polyclonal antibody-based ELISA assays. In either case, the coating antibodies will be non-covalently immobilized on microtiter plates. The detection antibody (monoclonal or polyclonal) will be biotinylated. Streptavidin-alkaline phosphatase will be used as a linking/detection reagent. For detection, we will utilize our substrate, diflunisal phosphate, in combination with terbium chelates and time-resolved fluorometry as we described elsewhere (71). The developed ELISA assays will be expected to have sensitivities in the low pg/ml concentration, and be free of any interference from other analytes. The developed assays will be calibrated using recombinant proteins. Furthermore, the assays will be subjected to extensive validation before serum analysis, including assessment of reproducibility, cross-reactivity, recovery and parallelism.

Multiple Reaction Monitoring Assays: For analytes for which, either one or more monoclonal or polyclonal antibodies are available, “Product-ion monitoring” (PIM) assays will be performed, after affinity purification of candidate biomarkers by an immobilized antibody, as described in (74). In this “hybrid” assay, the antibody is used to extract and purify the analyte from the biological fluid (eg. serum), followed by trypsin digestion of the analyte in the microtitre well. Then, a “proteotypic peptide” is selected for monitoring with a triple-quadrapole mass spectrometer, during peptide fragmentation in the collision cell. More technical details can be found in (74). By using this assay, and PSA as a model biomarker, PSA was quantified down to 0.1 ng/mL with (CVs) less than 20%.

In addition to this technology, it is also possible to quantify analytes present at relatively higher concentration in serum (e.g. 100 ng/mL) without antibody enrichment. In this case, the biological fluid (e.g. serum) is digested in trypsin and selected proteotypic peptides are monitored for various transitions during fragmentation, as described above. With such assays, multiplexing 5 or more analytes is possible.

Comparative proteomic analysis and absolute vs. relative quantification: compare quantitatively protein amounts in tissue culture supernatants and biological fluids originating from normal/benign or malignant conditions. Briefly, proteins from these fluids will be digested with trypsin and each set of generated peptides (normal/benign/cancer) will be labelled with one of the four available isobaric iTRAQ tags. After mixing of labelled peptides, the composite mixture will be analyzed by tandem mass spectrometry, as described earlier. With this technology, and appropriate software from ABI, it is possible to compare the concentrations of hundreds of proteins, in up to four different biological fluids (newer reagents include 8 instead of 4 isobaric tags), to identify overexpressed or underexpressed proteins.

Alternatively, absolute quantification of proteins in tissue culture supernatants and biological fluids can be achieved by using labelled (“heavy”) peptides of identical sequence as the proteotypic peptides of interest, for construction of calibration curves. One such method, AQUA, has been described recently by S. Gygi and colleagues (72, 73).

Further validation can for example be conducted using the well-accepted and statistically sound criteria, described by Sullivan-Pepe et al. (75, 76).

### Example 7

To diagnose whether or not a patient has lung cancer, a sample is obtained from the patient, such as peripheral blood. The level of one or more biomarkers, such as Pentraxin 3, Follistatin, sTNF RI, Osteoprotegerin and/or ADAM-17, is readily determined, for example, by ELISA, and compared to a control. A control value or cut-off level can be established by a clinical laboratory (for example as provided in Example 2). For example, the clinical laboratory can obtain a set of samples of peripheral blood from subjects for which there is associated clinical data, e.g. lung cancer, from a blood bank. The clinical laboratory can assess the level of the biomarker in samples of subjects with lung cancer and control samples without lung cancer for the conditions in their laboratory. A cut-off value can be determined for a particular observed sensitivity or specificity. The level in the patient sample is measured and compared to the cut-off value, wherein patients with biomarker levels above the cut-off value are identified as having lung cancer or in need of follow up testing.

### Example 8

### Prognostic Value of Biomarkers Listed in Table 8

Samples comprising lung carcinoma, cytosolic extracts and/or serum will be collected and the expression level of lung cancer biomarkers will be measured with quantitative ELISA methodologies and used to determine their prognostic value or combined prognostic value on survival of patients with various forms, and at different stages of lung cancer. The samples may include tissues and/or serum samples obtained at surgery from patients with lung cancer, tissues and/or serum samples obtained at surgery from patients with benign lung tumours, tissues and/or serum samples from patients with non-lung primary tumours that have metastasized to the lung, normal lung tissues and/or serum from healthy individuals. Age distributions will be similar between the different groups. The prognostic value of the lung cancer biomarkers will be examined using standard statistical analyses, including chi-square tests, Cox univariate and multivariate analysis and Kaplan-Meier survival analysis. The lung cancer biomarkers that will be measured include those biomarkers that are listed in Table 8, preferably one or more of Pentraxin 3, Follistatin, Osteoprotegerin ADAM-17 and/or sTNF R1.

### Example 9

### Prognostic Value of Biomarkers Listed in Table 8

A lung cancer tissue microarray (TMA), which consists of samples from patient with various lung cancer pathologies linked to an extensive database containing clinical and pathological information, including information on the outcome, will be used to examine the tissue expression profile of lung cancer biomarkers. The Kruskal-Wallis test will be used to determine whether variables differ across groups. Kaplan-Meier plots will be used to visualize the survival distributions and log-rank tests will be used to test the difference between survival distributions. The Cox proportional hazards model will be used to test the statistical independence and significance of predictors. The lung cancer biomarkers that will be measured include those biomarkers that are listed in Table 8, preferably one or more of Pentraxin 3, Follistatin, Osteoprotegerin, ADAM-17 and/or sTNF R1.

### Example 10

### Recurrence of Lung Cancer

The expression level of one or more biomarkers listed in Table 8 will be determined by ELISA and/or by SDS-PAGE followed by Western blotting, in subjects that have had a recurrence of lung cancer and for which there are samples available, such as peripheral blood and/or BAL fluid, that were obtained, for example, by a blood bank from subjects during (i) a period in which the subject has lung cancer; (ii) a period after (i) and in which the subject is free of lung cancer, such as 3, 6, 9 and/or 12 months after treatment; (iii) a period after (ii) in which the subject has lung cancer; and (iv) optionally, a period before the earliest instance of lung cancer and from subjects that have had lung cancer but no recurrence of lung cancer for at least 12 months after treatment, for example, during (1) a period in which the subject has lung cancer; (2) a period after (1) and in which the subject is free of lung cancer, such as 3, 6, 9 and/or 12 months; and (3) optionally, a period before the earliest instance of lung cancer. From these samples it can be determined whether the expression levels of one or more biomarkers listed in Table 8, preferably one or more of Pentraxin 3, Follistatin, Osteoprotegerin, ADAM-17 and/or sTNF RI, is/are useful for diagnosing whether lung cancer has recurred or is likely to recur in a subject that previously had lung cancer. For example, a cut-off value can be established wherein patients that have had lung cancer that have expression levels of one or more biomarkers listed in Table 8, preferably one or more of Pentraxin 3, Follistatin, Osteoprotegerin, ADAM-17 and/or sTNF RI, above the cut-off value 3, 6, 9 and/or 12 months after treatment for lung cancer and/or after they were determined to be lung cancer-free will be diagnosed as having had lung cancer recur, or likely to recur.

### Example 11

### Monitoring Response to Treatment for Lung Cancer

To determine whether a patient with lung cancer is responding to or likely to respond to treatment, such as chemotherapy and/or surgical resection, a sample is obtained from the patient, such as peripheral blood and/or BAL fluid. The level of one or more biomarkers, preferably one or more of Pentraxin 3, Follistatin, Osteoprotegerin, ADAM-17 and/or sTNF RI, is/are readily determined, for example, by ELISA, MRM and/or PIM, and compared to a control. For the control, a set of samples, for example, 15-20 samples per subject group, of BAL fluid or peripheral blood can be obtained from a blood bank from subjects for which there is associated clinical data, e.g. whether the samples are from subjects diagnosed with lung cancer and associated with or without responsiveness to treatment. The level of said biomarker(s) is readily determined for each sample, for example, by ELISA, MRM and/or PIM, and a suitable cut-off value is defined, wherein patients with biomarker levels below the cut-off value are identified as likely to respond to treatment. In addition, the clinical laboratory can identify a cut-off value for said biomarker(s) from samples associated with subjects without lung cancer or subjects with lung cancer that were responsive to treatment, wherein patients that are above this cut-off value prior to treatment but showing a trend over 3, 6, 9 and/or 12 months after the initiation of treatment towards or below the cut-off value are identified as responding to treatment.

### Example 12

### Prognosis of Patients with Lung Cancer

To determine the prognosis of a patient with lung cancer, a sample is obtained from the patient, such as peripheral blood and/or BAL fluid. The level of one or more biomarkers, preferably one or more of Pentraxin 3, Follistatin, Osteoprotegerin, ADAM-17 and/or sTNF RI, is/are readily determined, for example, by ELISA, MRM and/or PIM, and compared to a control. For the control, a clinical laboratory can obtain a set of samples such as BAL fluid and/or peripheral blood from a blood bank from subjects, for example, 15-20 samples per subject group, for which there is associated clinical data, e.g. whether the samples are from subjects comprising a good or poor survival group, or from subject with benign conditions, or early or late stage lung cancer. The level of said biomarker(s) is readily determined for each sample, for example, by ELISA, MRM and/or PIM, and the clinical laboratory identifies a cut-off value, wherein patients with biomarker levels below or above the cut-off value are identified as a good or poor survival group, respectively. Optionally, the clinical laboratory identifies a control value or range, wherein patients with biomarker levels within the control value or range are likely to have benign conditions, or early or late stage lung cancer.

### Example 13

A kit is used for screening for, detecting, or diagnosing lung cancer in a subject and/or determining prognosis of a subject having lung cancer, wherein a sample is obtained from the subject, such as peripheral blood and/or BAL fluid and the level of one or more biomarkers, preferably one or more of Pentraxin 3, Follistatin, Osteoprotegerin, ADAM-17 and/or sTNF RI, is/are readily determined by using the kit reagents following the instructions for use, and is compared to a control or reference standard. The kit can comprise one or more detection agents, for example an antibody, specific for one of said biomarkers and a control or reference standard and/or instructions for use thereof. The kit can include ancillary agents such as vessels for storing or transporting the detection agents and/or buffers or stabilizers. The kit can comprise a composition comprised of at least two detection agents that bind one of said biomarkers or combinations thereof. The kit can comprise an immunoassay, wherein one or more antibodies are immobilized on a solid support and each antibody is capable of forming a complex with one of said biomarkers. A cut-off value is identified, for example, by a clinical laboratory, which is appropriate for screening for, detecting, or diagnosing lung cancer in a subject and/or determining prognosis of a subject having lung cancer.

### Tables

TABLE 1
Examples of known and putative lung cancer biomarkers identified in the
conditioned media of H1688, H23, H460 and H520 cell lines by LC-MS/MS

| Protein | H1688* | H23* | H460* | H520* | Relationship | References |
|---|---|---|---|---|---|---|
| CEA | 7-8-8 | | | | useful indicator of disease extent and | [27, 28] |
| | | | | | may potentially have important | |
| | | | | | prognostic value for NSCLC patients | |
| | | | | | serum CEA level appears to be | |
| | | | | | closely associated with the presence of | |
| | | | | | EGFR gene mutations in patients with | |
| | | | | | pulmonary adenocarcinomas | |
| Chromogranin-A | 17-15-14 | | 11-10-12 | | high serum levels of CGA before | [29] |
| | | | | | chemotherapy was found as an | |
| | | | | | unfavorable prognostic determinant for | |
| | | | | | NSCLC patients | |
| Chromogranin-B | 30-26-23 | 6-7-7 | | 29-22-31 | marker for the immunohistochemical | [30] |
| | | | | | demonstration of carcinoids and well | |
| | | | | | differentiated neuroendocrine | |
| | | | | | carcinomas | |
| Creatine kinase | 10-13-12 | 1-3-1 | 1-1-2 | 9-12-12 | elevated levels of CK-BB found in | [65, 66] |
| BB | | | | | the serum of SCLC patients | |
| | | | | | relationship found between enhanced | |
| | | | | | levels of CK-BB and the degree of | |
| | | | | | lung carcinoma advance | |
| Progastrin- | 4-3-4** | | | | high serum levels of ProGRP before | [29, 31] |
| releasing | | | | | treatment conferred a survival | |
| peptide | | | | | advantage for NSCLC patients | |
| | | | | | higher serum levels of ProGRP (31-98) | |
| | | | | | in patients with extensive SCLC | |
| | | | | | than in patients with limited disease | |
| | | | | | higher serum levels of ProGRP (31-98) | |
| | | | | | in patients with pure small-cell | |
| | | | | | carcinoma than in patients with mixed | |
| | | | | | small-cell/large cell carcinoma | |
| Kallikrein 11 | 6-7-7 | | | | higher serum levels in NSCLC than | [32, 33] |
| | | | | | in healthy volunteers | |
| | | | | | associated with higher risk of | |
| | | | | | NSCLC | |
| | | | | | KLK11 mRNA overexpression in a | |
| | | | | | subgroup of neuroendocrine tumors | |
| | | | | | with unfavourable outcome | |
| Kallikrein 14 | 1-0-3 | | | | KLK14 mRNA overexpression in | [33, 34] |
| | | | | | lung tumors associated with a positive | |
| | | | | | nodal status of the tumor | |
| | | | | | higher serum levels in NSCLC than | |
| | | | | | in healthy volunteers | |
| | | | | | associated with higher risk of | |
| | | | | | NSCLC | |
| L-lactate | 4-6-6 | 8-8-10 | 8-6-7 | 6-5-7 | elevated serum LDHB correlated | [67] |
| dehydrogenase | | | | | with the clinical stage of lung cancer | |
| B chain | | | | | | |
| MMP1 | 0-4-3 | | 15-15-14 | | elevated serum levels in late stage | [18, 68] |
| collagenase | | | | | lung cancer patients | |
| | | | | | overexpression in tumor tissues and | |
| | | | | | association with tumor invasion and | |
| | | | | | metastasis | |
| Neural cell | 0-1-2 | | 1-0-0 | | elevated serum levels in patients with | [35-37] |
| adhesion | | | | | SCLC | |
| molecule | | | | | patients with pathologic NCAM | |
| | | | | | levels have significantly shorter | |
| | | | | | survival times | |
| | | | | | potential tumor marker for SCLC | |
| | | | | | raised serum NCAM in active SCLC | |
| | | | | | and in patients in relapse suggests that | |
| | | | | | NCAM can be used as a target for | |
| | | | | | antibody-directed therapy of | |
| | | | | | micrometastases | |
| Peroxiredoxin1 | 12-12-9 | 12-12- | 10-10-13 | 16-17-18 | up-regulated in lung cancer and may | [69] |
| | | 11 | | | serve as a prognostic biomarker and | |
| | | | | | therapeutic target in NSCLC | |
| Squamous cell | | | | 3-1-0 | both the preoperative SCC-Ag level | [47, 48] |
| carcinoma | | | | | and its postoperative decrease have | |
| (SCC) antigen | | | | | prognostic significance inferior to | |
| | | | | | stage of disease | |
| | | | | | elevated levels (>1.5 ng/ml) of SCC | |
| | | | | | were observed in 52.7% of squamous | |
| | | | | | cell lung cancer patients, but in only | |
| | | | | | 14.2% of nonsquamous cell lung | |
| | | | | | cancer patients | |
| Tumor M2-PK | 20-21-22 | 14-21- | 22-22-28 | 0-2-0 | elevated serum M2-PK found with | [49] |
| | | 24 | | | progressive lung tumor stages | |

*Each column contains number of unique peptides per protein; 3 values are reported for each protein representing each the 3 replicate samples per cell line.
**Combination of isoforms 1 and 2 of Gastrin-releasing peptide identified in H1688-CM

TABLE 2
Clinical and pathological characteristics of lung cancer patients for
Osteoprotegerin measurement by ELISA

| | Controls | Cases |
|---|---|---|
| Smoking status | | |
| Yes | 07 | 17 |
| No¹ | 17 | 08 |
| x² | 01 | |
| Gender | | |
| Female | 15 | 10 |
| Male | 10 | 15 |
| Age | | |
| Mean | 42 | 62 |
| SD | 09 | 14 |
| Stage | | |
| III | | 03 |
| IV | | 22 |
| Histology³ | | |
| ADC | | 08 |
| SCC | | 06 |
| BAC | | 01 |
| LCC | | 01 |
| Unspecified NSCLC | | 09 |

¹<100 cigarettes/lifetime.
²x, unknown.
³ADC, adenocarcinoma; SCC, squamous cell carcinoma; BAC, bronchioloalveolar carcinoma; LCC, large cell carcinoma; NSCLC, non-small cell lung carcinoma.

TABLE 3
Clinical and pathological characteristics of lung cancer patients
for sTNF RI measurement by ELISA

| | Controls | Cases |
|---|---|---|
| Smoking status | | |
| Yes | 07 | 16 |
| No¹ | 14 | 09 |
| x² | 04 | |
| Gender | | |
| Female | 08 | 11 |
| Male | 17 | 14 |
| Age | | |
| Mean | 36 | 62 |
| SD | 08 | 11 |
| Stage | | |
| III | | 04 |
| IV | | 20 |
| x² | | 01 |
| Histology³ | | |
| ADC | | 14 |
| SCC | | 04 |
| BAC | | 01 |
| Unspecified NSCLC | | 06 |

¹<100 cigarettes/lifetime.
²x, unknown.
³ADC, adenocarcinoma; SCC, squamous cell carcinoma; BAC, bronchioloalveolar carcinoma; NSCLC, non-small cell lung carcinoma.

TABLE 4
Clinical and pathological characteristics of lung cancer patients
for Follistatin measurement by ELISA

| | Controls | Cases |
|---|---|---|
| Smoking status | | |
| Yes | 07 | 17 |
| No¹ | 17 | 08 |
| x² | 01 | |
| Gender | | |
| Female | 15 | 10 |
| Male | 10 | 15 |
| Age | | |
| Mean | 42 | 62 |
| SD | 09 | 14 |
| Stage | | |
| III | | 02 |
| IV | | 23 |
| Histology³ | | |
| ADC | | 08 |
| SCC | | 06 |
| LCC | | 01 |
| Unspecified NSCLC | | 10 |

¹<100 cigarettes/lifetime.
²x, unknown.
³ADC, adenocarcinoma; SCC, squamous cell carcinoma; LCC, large cell carcinoma; NSCLC, non-small cell lung carcinoma.

TABLE 5
Clinical and pathological characteristics of lung cancer patients
for ADAM-17 measurement by ELISA

| | Controls | Cases |
|---|---|---|
| Smoking status | | |
| Yes | 04 | 15 |
| No¹ | 16 | 06 |
| x² | 01 | |
| Gender | | |
| Female | 14 | 08 |
| Male | 07 | 13 |
| Age | | |
| Mean | 41 | 61 |
| SD | 09 | 13 |
| Stage | | |
| III | | 03 |
| IV | | 18 |
| Histology³ | | |
| ADC | | 07 |
| SCC | | 06 |
| BAC | | 01 |
| LCC | | 01 |
| Unspecified NSCLC | | 06 |

¹<100 cigarettes/lifetime.
²x, unknown.
³ADC, adenocarcinoma; SCC, squamous cell carcinoma; BAC, bronchioloalveolar carcinoma; LCC, large cell carcinoma; NSCLC, non-small cell lung carcinoma.

TABLE 6
Clinical and pathological characteristics of lung cancer patients
for Pentraxin 3 by ELISA

| | Controls | Cases |
|---|---|---|
| Smoking status | | |
| Yes | 08 | 19 |
| No¹ | 15 | 06 |
| x² | 02 | |
| Gender | | |
| Female | 08 | 12 |
| Male | 17 | 13 |
| Age | | |
| Mean | 34 | 64 |
| SD | 09 | 10 |
| Stage | | |
| III | | 05 |
| IV | | 19 |
| x² | | 01 |
| Histology³ | | |
| ADC | | 10 |
| SCC | | 06 |
| Unspecified NSCLC | | 09 |

¹<100 cigarettes/lifetime.
²x, unknown.
³ADC, adenocarcinoma; SCC, squamous cell carcinoma; NSCLC, non-small cell lung carcinoma.

TABLE 7A
Detailed information for Follistatin, Osteoprotegerin and ADAM-17 identified in H1688 cell line

| | | | | Number | Number | | | | Best | Best | Best | Best XI | Calculated | | |
|---|---|---|---|---|---|---|---|---|---|---|---|---|---|---|---|
| Biological | | | Protein | of | of | Number | Percentage | | Peptide | Mascot | Mascot | Tandem | Peptide | Peptide | Peptide |
| sample | | Protein accession | identification | unique | unique | of total | sequence | Peptide | identification | ion | identity | −log(e) | Mass | start | stop |
| name | Protein name | numbers | probability | peptides | spectra | spectra | coverage | sequence | probability | score | score | score | (AMU) | index | index |
| S1 | FST Isoform 1 of | IPI00021081, IPI00217070, IPI00217071 | 100.00% | 4 | 5 | 5 | 13.90% | CKEQPEL | 95.00% | 0 | 0 | 4.68 | 1746.812 | 150 | 163 |
| | Follistatin | | | | | | | EVQYQGR | | | | | | | |
| | precursor | | | | | | | | | | | | | | |
| S1 | FST Isoform 1 of | IPI00021081, IPI00217070, IPI00217071 | 100.00% | 4 | 5 | 5 | 13.90% | EAACSS | 95.00% | 64.2 | 41.7 | 4.59 | 1362.694 | 299 | 311 |
| | Follistatin | | | | | | | GVLLEVK | | | | | | | |
| | precursor | | | | | | | | | | | | | | |
| S1 | FST Isoform 1 of | IPI00021081, IPI00217070, IPI00217071 | 100.00% | 4 | 5 | 5 | 13.90% | EQPELEV | 95.00% | 49.9 | 41.5 | 4.35 | 1475.713 | 152 | 163 |
| | Follistatin | | | | | | | QYQGR | | | | | | | |
| | precursor | | | | | | | | | | | | | | |
| S1 | FST Isoform 1 of | IPI00021081, IPI00217070, IPI00217071 | 100.00% | 4 | 5 | 5 | 13.90% | LSTSWTE | 95.00% | 29.3 | 40 | 4.82 | 1998.93 | 61 | 77 |
| | Follistatin | | | | | | | EDVNDN | | | | | | | |
| | precursor | | | | | | | TLFK | | | | | | | |
| S1 | TNFRSF11B | IPI00298362 | 100.00% | 6 | 10 | 10 | 22.70% | FTPNWL | 95.00% | 46.3 | 40.2 | 7.64 | 1901.018 | 215 | 231 |
| | Tumor necrosis | | | | | | | SVLVDNL | | | | | | | |
| | factor receptor | | | | | | | PGTK | | | | | | | |
| | superfamily | | | | | | | | | | | | | | |
| | member 11B | | | | | | | | | | | | | | |
| | precursor | | | | | | | | | | | | | | |
| S1 | TNFRSF11B | IPI00298362 | 100.00% | 6 | 10 | 10 | 22.70% | HTNCSVF | 95.00% | 37.1 | 41 | 4.72 | 1617.843 | 163 | 176 |
| | Tumor necrosis | | | | | | | GLLLTQK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S1 | TNFRSF11B | IPI00298362 | 100.00% | 6 | 10 | 10 | 22.70% | IIQDIDLC | 95.00% | 25.6 | 40.7 | 5.24 | 1702.844 | 270 | 283 |
| | Tumor necrosis | | | | | | | ENSVQR | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S1 | TNFRSF11B | IPI00298362 | 100.00% | 6 | 10 | 10 | 22.70% | LFLEMIG | 95.00% | 27.5 | 40.9 | 3.27 | 1605.868 | 384 | 397 |
| | Tumor necrosis | | | | | | | NQVQSVK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S1 | TNFRSF11B | IPI00298362 | 100.00% | 6 | 10 | 10 | 22.70% | SCPPGF | 95.00% | 27.2 | 40.8 | 1.89 | 1658.796 | 123 | 138 |
| | Tumor necrosis | | | | | | | GVVQAG | | | | | | | |
| | factor receptor | | | | | | | TPER | | | | | | | |
| | superfamily | | | | | | | | | | | | | | |
| | member 11B | | | | | | | | | | | | | | |
| | precursor | | | | | | | | | | | | | | |
| S1 | TNFRSF11B | IPI00298362 | 100.00% | 6 | 10 | 10 | 22.70% | YLHYDEE | 95.00% | 79.4 | 39.6 | 11.8 | 2050.918 | 28 | 43 |
| | Tumor necrosis | | | | | | | TSHQLLC | | | | | | | |
| | factor receptor | | | | | | | DK | | | | | | | |
| | superfamily | | | | | | | | | | | | | | |
| | member 11B | | | | | | | | | | | | | | |
| | precursor | | | | | | | | | | | | | | |
| S2 | FST Isoform 1 of | IPI00021081, IPI00217070, IPI00217071 | 100.00% | 3 | 5 | 6 | 7.85% | CKEQPEL | 95.00% | 0 | 0 | 5 | 1746.812 | 150 | 163 |
| | Follistatin | | | | | | | EVQYQGR | | | | | | | |
| | precursor | | | | | | | | | | | | | | |
| S2 | FST Isoform 1 of | IPI00021081, IPI00217070, IPI00217071 | 100.00% | 3 | 5 | 6 | 7.85% | EAACSS | 95.00% | 70.2 | 41.7 | 4.6 | 1362.694 | 299 | 311 |
| | Follistatin | | | | | | | GVLLEVK | | | | | | | |
| | precursor | | | | | | | | | | | | | | |
| S2 | FST Isoform 1 of | IPI00021081, IPI00217070, IPI00217071 | 100.00% | 3 | 5 | 6 | 7.85% | EQPELEV | 95.00% | 47.7 | 41.4 | 5.77 | 1475.713 | 152 | 163 |
| | Follistatin | | | | | | | QYQGR | | | | | | | |
| | precursor | | | | | | | | | | | | | | |
| S2 | TNFRSF11B | IPI00298362 | 100.00% | 10 | 18 | 21 | 35.20% | CGIDVTL | 95.00% | 30.7 | 40.8 | 4.15 | 1716.773 | 195 | 208 |
| | Tumor necrosis | | | | | | | CEEAFFR | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 10 | 18 | 21 | 35.20% | FLHSFTM | 95.00% | 30.3 | 42.3 | 2.77 | 1173.577 | 371 | 379 |
| | Tumor necrosis | | | | | | | YK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 10 | 18 | 21 | 35.20% | FTPNWL | 95.00% | 34.1 | 40.2 | 3.36 | 1901.018 | 215 | 231 |
| | Tumor necrosis | | | | | | | SVLVDNL | | | | | | | |
| | factor receptor | | | | | | | PGTK | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 10 | 18 | 21 | 35.20% | HTNCSVF | 95.00% | 51.3 | 41 | 5.5 | 1617.843 | 163 | 176 |
| | Tumor necrosis | | | | | | | GLLLTQK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 10 | 18 | 21 | 35.20% | IIQDIDLC | 95.00% | 57.4 | 40.7 | 8.85 | 1702.844 | 270 | 283 |
| | Tumor necrosis | | | | | | | ENSVQR | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 10 | 18 | 21 | 35.20% | KHTNCS | 95.00% | 15.9 | 40.6 | 2.19 | 1745.938 | 162 | 176 |
| | Tumor necrosis | | | | | | | VFGLLLT | | | | | | | |
| | factor receptor | | | | | | | QK | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 10 | 18 | 21 | 35.20% | LFLEMIG | 95.00% | 27.2 | 41 | 0.538 | 1605.868 | 384 | 397 |
| | Tumor necrosis | | | | | | | NQVQSVK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 10 | 18 | 21 | 35.20% | QHSSQE | 95.00% | 30.4 | 41.1 | 2.41 | 1573.798 | 243 | 255 |
| | Tumor necrosis | | | | | | | QTFQLLK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 10 | 18 | 21 | 35.20% | TVCAPCP | 95.00% | 47.4 | 35.9 | 7.92 | 3608.433 | 60 | 88 |
| | Tumor necrosis | | | | | | | DHYYTD | | | | | | | |
| | factor receptor | | | | | | | SWHTSD | | | | | | | |
| | superfamily | | | | | | | ECLYCSP | | | | | | | |
| | member 11B | | | | | | | VCK | | | | | | | |
| | precursor | | | | | | | | | | | | | | |
| S2 | TNFRSF11B | IPI00298362 | 100.00% | 10 | 18 | 21 | 35.20% | YLHYDEE | 95.00% | 78.9 | 39.6 | 14.6 | 2050.918 | 28 | 43 |
| | Tumor necrosis | | | | | | | TSHQLLC | | | | | | | |
| | factor receptor | | | | | | | DK | | | | | | | |
| | superfamily | | | | | | | | | | | | | | |
| | member 11B | | | | | | | | | | | | | | |
| | precursor | | | | | | | | | | | | | | |
| S2 | ADAM17 | IPI00029606, IPI00288894 | 99.80% | 2 | 2 | 2 | 3.76% | INTDGAE | 95.00% | 24.4 | 40.5 | 1.75 | 1790.872 | 140 | 154 |
| | Isoform B of | | | | | | | YNIEPLWR | | | | | | | |
| | ADAM 17 | | | | | | | | | | | | | | |
| | precursor | | | | | | | | | | | | | | |
| S2 | ADAM17 | IPI00029606, IPI00288894 | 99.80% | 2 | 2 | 2 | 3.76% | WQDFFT | 95.00% | 13.9 | 40.3 | 2.51 | 1876.862 | 111 | 126 |
| | Isoform B of | | | | | | | GHVVGE | | | | | | | |
| | ADAM 17 | | | | | | | PDSR | | | | | | | |
| | precursor | | | | | | | | | | | | | | |
| S3 | FST Isoform 1 of | IPI00021081, IPI00217070, IPI00217071 | 100.00% | 5 | 7 | 7 | 16.60% | CKEQPEL | 95.00% | 0 | 0 | 7.5 | 1746.812 | 150 | 163 |
| | Follistatin | | | | | | | EVQYQGR | | | | | | | |
| | precursor | | | | | | | | | | | | | | |
| S3 | FST Isoform 1 of | IPI00021081, IPI00217070, IPI00217071 | 100.00% | 5 | 7 | 7 | 16.60% | CVCAPD | 95.00% | 21.2 | 41 | 1.85 | 1610.677 | 116 | 128 |
| | Follistatin | | | | | | | CSNITWK | | | | | | | |
| | precursor | | | | | | | | | | | | | | |
| S3 | FST Isoform 1 of | IPI00021081, IPI00217070, IPI00217071 | 100.00% | 5 | 7 | 7 | 16.60% | EAACSS | 95.00% | 77.1 | 41.7 | 4.04 | 1362.694 | 299 | 311 |
| | Follistatin | | | | | | | GVLLEVK | | | | | | | |
| | precursor | | | | | | | | | | | | | | |
| S3 | FST Isoform 1 of | IPI00021081, IPI00217070, IPI00217071 | 100.00% | 5 | 7 | 7 | 16.60% | EQPELEV | 95.00% | 53 | 41.4 | 5.92 | 1475.713 | 152 | 163 |
| | Follistatin | | | | | | | QYQGR | | | | | | | |
| | precursor | | | | | | | | | | | | | | |
| S3 | FST Isoform 1 of | IPI00021081, IPI00217070, IPI00217071 | 100.00% | 5 | 7 | 7 | 16.60% | LSTSWTE | 95.00% | 85.1 | 40 | 9.62 | 1998.93 | 61 | 77 |
| | Follistatin | | | | | | | EDVNDN | | | | | | | |
| | precursor | | | | | | | TLFK | | | | | | | |
| S3 | TNFRSF11B | IPI00298362 | 100.00% | 8 | 13 | 14 | 28.40% | FLHSFTM | 95.00% | 30.9 | 42.2 | 1.55 | 1173.577 | 371 | 379 |
| | Tumor necrosis | | | | | | | YK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 8 | 13 | 14 | 28.40% | FTPNWL | 95.00% | 20.1 | 40.2 | 3.18 | 1901.018 | 215 | 231 |
| | Tumor necrosis | | | | | | | SVLVDNL | | | | | | | |
| | factor receptor | | | | | | | PGTK | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 8 | 13 | 14 | 28.40% | HTNCSVF | 95.00% | 38.7 | 41.1 | 4.7 | 1617.843 | 163 | 176 |
| | Tumor necrosis | | | | | | | GLLLTQK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 8 | 13 | 14 | 28.40% | IIQDIDLC | 95.00% | 21.9 | 40.7 | 2.4 | 1702.844 | 270 | 283 |
| | Tumor necrosis | | | | | | | ENSVQR | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 8 | 13 | 14 | 28.40% | KIIQDIDL | 95.00% | 53.3 | 40.4 | 3.66 | 1830.939 | 269 | 283 |
| | Tumor necrosis | | | | | | | CENSVQR | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 8 | 13 | 14 | 28.40% | LFLEMIG | 95.00% | 38.2 | 41 | 3.55 | 1605.868 | 384 | 397 |
| | Tumor necrosis | | | | | | | NQVQSVK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 8 | 13 | 14 | 28.40% | TVCAPCP | 95.00% | 56.4 | 35.8 | 9.46 | 3608.433 | 60 | 88 |
| | Tumor necrosis | | | | | | | DHYYTD | | | | | | | |
| | factor receptor | | | | | | | SWHTSD | | | | | | | |
| | superfamily | | | | | | | ECLYCSP | | | | | | | |
| | member 11B | | | | | | | VCK | | | | | | | |
| | precursor | | | | | | | | | | | | | | |
| S3 | TNFRSF11B | IPI00298362 | 100.00% | 8 | 13 | 14 | 28.40% | YLHYDEE | 95.00% | 89.2 | 39.6 | 13.1 | 2050.918 | 28 | 43 |
| | Tumor necrosis | | | | | | | TSHQLLC | | | | | | | |
| | factor receptor | | | | | | | DK | | | | | | | |
| | superfamily | | | | | | | | | | | | | | |
| | member 11B | | | | | | | | | | | | | | |
| | precursor | | | | | | | | | | | | | | |

TABLE 7B
Detailed information for Osteoprotegerin, Pentraxin 3, sTNF RI and ADAM-17 identified in H23 cell line

| | | | | Number | Number | | | | Best | Best | Best | Best XI | Calculated | | |
|---|---|---|---|---|---|---|---|---|---|---|---|---|---|---|---|
| Biological | | | Protein | of | of | Number | Percentage | | Peptide | Mascot | Mascot | Tandem | Peptide | Peptide | Peptide |
| sample | | Protein accession | identification | unique | unique | of total | sequence | Peptide | identification | ion | identity | −log(e) | Mass | start | stop |
| name | Protein name | numbers | probability | peptides | spectra | spectra | coverage | sequence | probability | score | score | score | (AMU) | index | index |
| S1 | TNFRSF11B | IPI00298362 | 100.00% | 5 | 9 | 9 | 22.40% | FTPNWL | 95.00% | 16.7 | 40.2 | 1.85 | 1901.018 | 215 | 231 |
| | Tumor necrosis | | | | | | | SVLVDNL | | | | | | | |
| | factor receptor | | | | | | | PGTK | | | | | | | |
| | superfamily | | | | | | | | | | | | | | |
| | member 11B | | | | | | | | | | | | | | |
| | precursor | | | | | | | | | | | | | | |
| S1 | TNFRSF11B | IPI00298362 | 100.00% | 5 | 9 | 9 | 22.40% | HTNCSVF | 95.00% | 61.5 | 40.9 | 7.05 | 1617.843 | 163 | 176 |
| | Tumor necrosis | | | | | | | GLLLTQK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S1 | TNFRSF11B | IPI00298362 | 100.00% | 5 | 9 | 9 | 22.40% | IIQDIDLC | 95.00% | 59.1 | 40.7 | 8.8 | 1702.844 | 270 | 283 |
| | Tumor necrosis | | | | | | | ENSVQR | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S1 | TNFRSF11B | IPI00298362 | 100.00% | 5 | 9 | 9 | 22.40% | TVCAPCP | 95.00% | 20.4 | 35.8 | 6.6 | 3608.433 | 60 | 88 |
| | Tumor necrosis | | | | | | | DHYYTD | | | | | | | |
| | factor receptor | | | | | | | SWHTSD | | | | | | | |
| | superfamily | | | | | | | ECLYCSP | | | | | | | |
| | member 11B | | | | | | | VCK | | | | | | | |
| | precursor | | | | | | | | | | | | | | |
| S1 | TNFRSF11B | IPI00298362 | 100.00% | 5 | 9 | 9 | 22.40% | YLHYDEE | 95.00% | 28.4 | 39.8 | 5.66 | 2050.918 | 28 | 43 |
| | Tumor necrosis | | | | | | | TSHQLLC | | | | | | | |
| | factor receptor | | | | | | | DK | | | | | | | |
| | superfamily | | | | | | | | | | | | | | |
| | member 11B | | | | | | | | | | | | | | |
| | precursor | | | | | | | | | | | | | | |
| S1 | ADAM17 | IPI00288894 | 100.00% | 2 | 2 | 2 | 4.13% | DLQTSTH | 95.00% | 26.6 | 39.8 | 1.85 | 2004.066 | 59 | 76 |
| | Isoform A of | | | | | | | VETLLTF | | | | | | | |
| | ADAM 17 | | | | | | | SALK | | | | | | | |
| | precursor | | | | | | | | | | | | | | |
| S1 | ADAM17 | IPI00288894 | 100.00% | 2 | 2 | 2 | 4.13% | WQDFFT | 95.00% | 14.5 | 40.3 | 2.62 | 1876.862 | 111 | 126 |
| | Isoform A of | | | | | | | GHVVGE | | | | | | | |
| | ADAM 17 | | | | | | | PDSR | | | | | | | |
| | precursor | | | | | | | | | | | | | | |
| S1 | PTX3 Pentraxin- | IPI00029568 | 100.00% | 5 | 7 | 7 | 19.90% | ADLHAV | 95.00% | 39.4 | 41.7 | 5.68 | 1294.666 | 161 | 172 |
| | related protein | | | | | | | QGWAAR | | | | | | | |
| | PTX3 precursor | | | | | | | | | | | | | | |
| S1 | PTX3 Pentraxin- | IPI00029568 | 100.00% | 5 | 7 | 7 | 19.90% | LTGFNIW | 95.00% | 105 | 40 | 9.66 | 1993.003 | 333 | 349 |
| | related protein | | | | | | | DSVLSNE | | | | | | | |
| | PTX3 precursor | | | | | | | EIR | | | | | | | |
| S1 | PTX3 Pentraxin- | IPI00029568 | 100.00% | 5 | 7 | 7 | 19.90% | LTSALDE | 95.00% | 86.7 | 41.5 | 8 | 1430.786 | 113 | 125 |
| | related protein | | | | | | | LLQATR | | | | | | | |
| | PTX3 precursor | | | | | | | | | | | | | | |
| S1 | PTX3 Pentraxin- | IPI00029568 | 100.00% | 5 | 7 | 7 | 19.90% | NGCCVG | 95.00% | 55.3 | 40.2 | 0 | 1903.807 | 315 | 332 |
| | related protein | | | | | | | GGFDETL | | | | | | | |
| | PTX3 precursor | | | | | | | AFSGR | | | | | | | |
| S1 | PTX3 Pentraxin- | IPI00029568 | 100.00% | 5 | 7 | 7 | 19.90% | SWLPAG | 95.00% | 68.8 | 40.4 | 7.72 | 1848.914 | 173 | 188 |
| | related protein | | | | | | | CETAILF | | | | | | | |
| | PTX3 precursor | | | | | | | PMR | | | | | | | |
| S1 | TNFRSFIA | IPI00018880, IPI00796532 | 99.60% | 1 | 1 | 1 | 3.05% | EMGQVEI | 95.00% | 20.4 | 41 | 1.6 | 1610.716 | 112 | 125 |
| | Tumor necrosis | | | | | | | SSCTVDR | | | | | | | |
| | factor receptor | | | | | | | | | | | | | | |
| | superfamily | | | | | | | | | | | | | | |
| | member 1A | | | | | | | | | | | | | | |
| | precursor | | | | | | | | | | | | | | |
| S2 | TNFRSF11B | IPI00298362 | 100.00% | 7 | 11 | 11 | 29.90% | CGIDVTL | 95.00% | 9.24 | 40.8 | 3.17 | 1716.773 | 195 | 208 |
| | Tumor necrosis | | | | | | | CEEAFFR | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 7 | 11 | 11 | 29.90% | FTPNWL | 95.00% | 63.4 | 40.2 | 9.44 | 1901.018 | 215 | 231 |
| | Tumor necrosis | | | | | | | SVLVDNL | | | | | | | |
| | factor receptor | | | | | | | PGTK | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 7 | 11 | 11 | 29.90% | HTNCSVF | 95.00% | 54.4 | 41 | 8.42 | 1617.843 | 163 | 176 |
| | Tumor necrosis | | | | | | | GLLLTQK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 7 | 11 | 11 | 29.90% | IIQDIDLC | 95.00% | 54.5 | 40.7 | 5.36 | 1702.844 | 270 | 283 |
| | Tumor necrosis | | | | | | | ENSVQR | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 7 | 11 | 11 | 29.90% | SCPPGF | 95.00% | 34.9 | 40.8 | 2.14 | 1658.796 | 123 | 138 |
| | Tumor necrosis | | | | | | | GVVQAG | | | | | | | |
| | factor receptor | | | | | | | TPER | | | | | | | |
| | superfamily | | | | | | | | | | | | | | |
| | member 11B | | | | | | | | | | | | | | |
| | precursor | | | | | | | | | | | | | | |
| S2 | TNFRSF11B | IPI00298362 | 100.00% | 7 | 11 | 11 | 29.90% | TVCAPCP | 95.00% | 41.5 | 35.9 | 5.7 | 3608.433 | 60 | 88 |
| | Tumor necrosis | | | | | | | DHYYTD | | | | | | | |
| | factor receptor | | | | | | | SWHTSD | | | | | | | |
| | superfamily | | | | | | | ECLYCSP | | | | | | | |
| | member 11B | | | | | | | VCK | | | | | | | |
| | precursor | | | | | | | | | | | | | | |
| S2 | TNFRSF11B | IPI00298362 | 100.00% | 7 | 11 | 11 | 29.90% | YLHYDEE | 95.00% | 68.9 | 39.6 | 10.7 | 2050.918 | 28 | 43 |
| | Tumor necrosis | | | | | | | TSHQLLC | | | | | | | |
| | factor receptor | | | | | | | DK | | | | | | | |
| | superfamily | | | | | | | | | | | | | | |
| | member 11B | | | | | | | | | | | | | | |
| | precursor | | | | | | | | | | | | | | |
| S2 | ADAM17 | IPI00288894 | 100.00% | 3 | 4 | 4 | 5.34% | INTDGAE | 95.00% | 62.6 | 40.6 | 3.51 | 1790.872 | 140 | 154 |
| | Isoform A of | | | | | | | YNIEPLWR | | | | | | | |
| | ADAM 17 | | | | | | | | | | | | | | |
| | precursor | | | | | | | | | | | | | | |
| S2 | ADAM17 | IPI00288894 | 100.00% | 3 | 4 | 4 | 5.34% | VLAHIRD | 95.00% | 24.6 | 41.1 | 0.886 | 1534.871 | 127 | 139 |
| | Isoform A of | | | | | | | DDVIIR | | | | | | | |
| | ADAM 17 | | | | | | | | | | | | | | |
| | precursor | | | | | | | | | | | | | | |
| S2 | ADAM17 | IPI00288894 | 100.00% | 3 | 4 | 4 | 5.34% | WQDFFT | 95.00% | 73.1 | 40.3 | 9.35 | 1876.862 | 111 | 126 |
| | Isoform A of | | | | | | | GHVVGE | | | | | | | |
| | ADAM 17 | | | | | | | PDSR | | | | | | | |
| | precursor | | | | | | | | | | | | | | |
| S2 | PTX3 Pentraxin- | IPI00029568 | 100.00% | 5 | 7 | 8 | 18.40% | ADLHAV | 95.00% | 44 | 41.7 | 5.44 | 1294.666 | 161 | 172 |
| | related protein | | | | | | | QGWAAR | | | | | | | |
| | PTX3 precursor | | | | | | | | | | | | | | |
| S2 | PTX3 Pentraxin- | IPI00029568 | 100.00% | 5 | 7 | 8 | 18.40% | LAESLAR | 95.00% | 13.9 | 40.5 | 2.75 | 1836.939 | 95 | 112 |
| | related protein | | | | | | | PCAPGA | | | | | | | |
| | PTX3 precursor | | | | | | | PAEAR | | | | | | | |
| S2 | PTX3 Pentraxin- | IPI00029568 | 100.00% | 5 | 7 | 8 | 18.40% | LFIMLEN | 95.00% | 40.4 | 41.6 | 5.3 | 1381.697 | 59 | 69 |
| | related protein | | | | | | | SQMR | | | | | | | |
| | PTX3 precursor | | | | | | | | | | | | | | |
| S2 | PTX3 Pentraxin- | IPI00029568 | 100.00% | 5 | 7 | 8 | 18.40% | LTSALDE | 95.00% | 91 | 41.4 | 6.66 | 1430.786 | 113 | 125 |
| | related protein | | | | | | | LLQATR | | | | | | | |
| | PTX3 precursor | | | | | | | | | | | | | | |
| S2 | PTX3 Pentraxin- | IPI00029568 | 100.00% | 5 | 7 | 8 | 18.40% | SWLPAG | 95.00% | 26.9 | 40.4 | 4.72 | 1848.914 | 173 | 188 |
| | related protein | | | | | | | CETAILF | | | | | | | |
| | PTX3 precursor | | | | | | | PMR | | | | | | | |
| S2 | TNFRSF1A | IPI00018880, IPI00796532 | 100.00% | 3 | 4 | 4 | 10.30% | ECESGS | 95.00% | 55.7 | 40.7 | 6.08 | 1723.735 | 83 | 97 |
| | Tumor necrosis | | | | | | | FTASENH | | | | | | | |
| | factor receptor | | | | | | | LR | | | | | | | |
| | superfamily | | | | | | | | | | | | | | |
| | member 1A | | | | | | | | | | | | | | |
| | precursor | | | | | | | | | | | | | | |
| S2 | TNFRSF1A | IPI00018880, IPI00796532 | 100.00% | 3 | 4 | 4 | 10.30% | GTYLYND | 95.00% | 90.1 | 39.8 | 10.3 | 2088.839 | 65 | 82 |
| | Tumor necrosis | | | | | | | CPGPGQ | | | | | | | |
| | factor receptor | | | | | | | DTDCR | | | | | | | |
| | superfamily | | | | | | | | | | | | | | |
| | member 1A | | | | | | | | | | | | | | |
| | precursor | | | | | | | | | | | | | | |
| S2 | TNFRSF1A | IPI00018880, IPI00796532 | 100.00% | 3 | 4 | 4 | 10.30% | QNTVCT | 95.00% | 0 | 0 | 3.51 | 1693.758 | 162 | 175 |
| | Tumor necrosis | | | | | | | CHAGFFLR | | | | | | | |
| | factor receptor | | | | | | | | | | | | | | |
| | superfamily | | | | | | | | | | | | | | |
| | member 1A | | | | | | | | | | | | | | |
| | precursor | | | | | | | | | | | | | | |
| S3 | TNFRSF11B | IPI00298362 | 100.00% | 7 | 11 | 12 | 30.20% | FTPNWL | 95.00% | 20.8 | 40.2 | 5.06 | 1901.018 | 215 | 231 |
| | Tumor necrosis | | | | | | | SVLVDNL | | | | | | | |
| | factor receptor | | | | | | | PGTK | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 7 | 11 | 12 | 30.20% | IIQDIDLC | 95.00% | 63.2 | 40.7 | 8.5 | 1702.844 | 270 | 283 |
| | Tumor necrosis | | | | | | | ENSVQR | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 7 | 11 | 12 | 30.20% | KHTNCS | 95.00% | 22.6 | 40.5 | 1.48 | 1745.938 | 162 | 176 |
| | Tumor necrosis | | | | | | | VFGLLLT | | | | | | | |
| | factor receptor | | | | | | | QK | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 7 | 11 | 12 | 30.20% | LFLEMIG | 95.00% | 31.3 | 41 | 2 | 1605.868 | 384 | 397 |
| | Tumor necrosis | | | | | | | NQVQSVK | | | | | | | |
| | factor receptor | | | | | | | | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 7 | 11 | 12 | 30.20% | SCPPGF | 95.00% | 42.7 | 40.8 | 0 | 1658.796 | 123 | 138 |
| | Tumor necrosis | | | | | | | GVVQAG | | | | | | | |
| | factor receptor | | | | | | | TPER | | | | | | | |
| | superfamily | | | | | | | | | | | | | | |
| | member 11B | | | | | | | | | | | | | | |
| | precursor | | | | | | | | | | | | | | |
| S3 | TNFRSF11B | IPI00298362 | 100.00% | 7 | 11 | 12 | 30.20% | TVCAPCP | 95.00% | 43.1 | 35.8 | 5.6 | 3608.433 | 60 | 88 |
| | Tumor necrosis | | | | | | | DHYYTD | | | | | | | |
| | factor receptor | | | | | | | SWHTSD | | | | | | | |
| | superfamily | | | | | | | ECLYCSP | | | | | | | |
| | member 11B | | | | | | | VCK | | | | | | | |
| | precursor | | | | | | | | | | | | | | |
| S3 | TNFRSF11B | IPI00298362 | 100.00% | 7 | 11 | 12 | 30.20% | YLHYDEE | 95.00% | 47 | 39.6 | 10.7 | 2050.918 | 28 | 43 |
| | Tumor necrosis | | | | | | | TSHQLLC | | | | | | | |
| | factor receptor | | | | | | | DK | | | | | | | |
| | superfamily | | | | | | | | | | | | | | |
| | member 11B | | | | | | | | | | | | | | |
| | precursor | | | | | | | | | | | | | | |
| S3 | ADAM17 | IPI00288894 | 100.00% | 3 | 3 | 3 | 5.34% | INTDGAE | 95.00% | 51.8 | 40.5 | 4.7 | 1790.872 | 140 | 154 |
| | Isoform A of | | | | | | | YNIEPLWR | | | | | | | |
| | ADAM 17 | | | | | | | | | | | | | | |
| | precursor | | | | | | | | | | | | | | |
| S3 | ADAM17 | IPI00288894 | 100.00% | 3 | 3 | 3 | 5.34% | VLAHIRD | 95.00% | 25.6 | 41.1 | 1.59 | 1534.871 | 127 | 139 |
| | Isoform A of | | | | | | | DDVIIR | | | | | | | |
| | ADAM 17 | | | | | | | | | | | | | | |
| | precursor | | | | | | | | | | | | | | |
| S3 | ADAM17 | IPI00288894 | 100.00% | 3 | 3 | 3 | 5.34% | WQDFFT | 95.00% | 70.3 | 40.3 | 10.3 | 1876.862 | 111 | 126 |
| | Isoform A of | | | | | | | GHVVGE | | | | | | | |
| | ADAM 17 | | | | | | | PDSR | | | | | | | |
| | precursor | | | | | | | | | | | | | | |
| S3 | PTX3 Pentraxin- | IPI00029568 | 100.00% | 6 | 8 | 8 | 23.60% | ADLHAV | 95.00% | 34.2 | 41.7 | 4.19 | 1294.666 | 161 | 172 |
| | related protein | | | | | | | QGWAAR | | | | | | | |
| | PTX3 precursor | | | | | | | | | | | | | | |
| S3 | PTX3 Pentraxin- | IPI00029568 | 100.00% | 6 | 8 | 8 | 23.60% | ALAAVLE | 95.00% | 59.1 | 42.3 | 3.33 | 1084.637 | 148 | 157 |
| | related protein | | | | | | | ELR | | | | | | | |
| | PTX3 precursor | | | | | | | | | | | | | | |
| S3 | PTX3 Pentraxin- | IPI00029568 | 100.00% | 6 | 8 | 8 | 23.60% | GNIVGW | 95.00% | 38.1 | 39.5 | 5.89 | 2169.073 | 361 | 381 |
| | related protein | | | | | | | GVTEIQP | | | | | | | |
| | PTX3 precursor | | | | | | | HGGAQY | | | | | | | |
| | | | | | | | | VS | | | | | | | |
| S3 | PTX3 Pentraxin- | IPI00029568 | 100.00% | 6 | 8 | 8 | 23.60% | LAESLAR | 95.00% | 35.1 | 40.6 | 2.64 | 1836.939 | 95 | 112 |
| | related protein | | | | | | | PCAPGA | | | | | | | |
| | PTX3 precursor | | | | | | | PAEAR | | | | | | | |
| S3 | PTX3 Pentraxin- | IPI00029568 | 100.00% | 6 | 8 | 8 | 23.60% | LTSALDE | 95.00% | 32.7 | 41.5 | 3.92 | 1430.786 | 113 | 125 |
| | related protein | | | | | | | LLQATR | | | | | | | |
| | PTX3 precursor | | | | | | | | | | | | | | |
| S3 | PTX3 Pentraxin- | IPI00029568 | 100.00% | 6 | 8 | 8 | 23.60% | SWLPAG | 95.00% | 32.9 | 40.4 | 4.89 | 1848.914 | 173 | 188 |
| | related protein | | | | | | | CETAILF | | | | | | | |
| | PTX3 precursor | | | | | | | PMR | | | | | | | |

TABLE 7C
Detailed information for Osteoprotegerin, ADAM-17, Follistatin and Pentraxin 3 identified in H460 cell line

| | | | | Number | Number | | | | Best | Best | Best | Best XI | Calculated | | |
|---|---|---|---|---|---|---|---|---|---|---|---|---|---|---|---|
| Biological | | Protein | Protein | of | of | Number | Percentage | | Peptide | Mascot | Mascot | Tandem | Peptide | Peptide | Peptide |
| sample | | accession | identification | unique | unique | of total | sequence | Peptide | identification | ion | identity | −log(e) | Mass | start | stop |
| name | Protein name | numbers | probability | peptides | spectra | spectra | coverage | sequence | probability | score | score | score | (AMU) | index | index |
| S1 | TNFRSF11B Tumor | IPI00298362 | 99.90% | 2 | 6 | 6 | 8.23% | FTPNWL | 95.00% | 51.8 | 40.2 | 6.52 | 1901.018 | 215 | 231 |
| | necrosis factor | | | | | | | SVLVDNL | | | | | | | |
| | receptor superfamily | | | | | | | PGTK | | | | | | | |
| | member 11B | | | | | | | | | | | | | | |
| | precursor | | | | | | | | | | | | | | |
| S1 | TNFRSF11B Tumor | IPI00298362 | 99.90% | 2 | 6 | 6 | 8.23% | YLHYDEE | 95.00% | 54.3 | 39.8 | 10.5 | 2050.918 | 28 | 43 |
| | necrosis factor | | | | | | | TSHQLLC | | | | | | | |
| | receptor superfamily | | | | | | | DK | | | | | | | |
| | member 11B | | | | | | | | | | | | | | |
| | precursor | | | | | | | | | | | | | | |
| S1 | ADAM17 Isoform B | IPI00029606, | 100.00% | 4 | 7 | 7 | 9.51% | INTDGAE | 95.00% | 48.1 | 40.5 | 2.8 | 1790.872 | 140 | 154 |
| | of ADAM 17 | IPI00288894 | | | | | | YNIEPLWR | | | | | | | |
| | precursor | | | | | | | | | | | | | | |
| S1 | ADAM17 Isoform B | IPI00029606, | 100.00% | 4 | 7 | 7 | 9.51% | LDSLLSD | 95.00% | 26.3 | 38.5 | 1.18 | 2516.3 | 35 | 56 |
| | of ADAM 17 | IPI00288894 | | | | | | YDILSLS | | | | | | | |
| | precursor | | | | | | | NIQQHSVR | | | | | | | |
| S1 | ADAM17 Isoform B | IPI00029606, | 100.00% | 4 | 7 | 7 | 9.51% | VLAHIRD | 95.00% | 41.6 | 41.1 | 3.22 | 1534.871 | 127 | 139 |
| | of ADAM 17 | IPI00288894 | | | | | | DDVIIR | | | | | | | |
| | precursor | | | | | | | | | | | | | | |
| S1 | ADAM17 Isoform B | IPI00029606, | 100.00% | 4 | 7 | 7 | 9.51% | WQDFFT | 95.00% | 67.7 | 40.3 | 11.6 | 1876.862 | 111 | 126 |
| | of ADAM 17 | IPI00288894 | | | | | | GHVVGE | | | | | | | |
| | precursor | | | | | | | PDSR | | | | | | | |
| S1 | FST Isoform 1 of | IPI00021081, | 100.00% | 5 | 11 | 12 | 19.50% | CKEQPEL | 95.00% | 0 | 0 | 5.6 | 1746.812 | 150 | 163 |
| | Follistatin precursor | IPI00217070, | | | | | | EVQYQGR | | | | | | | |
| | | IPI00217071 | | | | | | | | | | | | | |
| S1 | FST Isoform 1 of | IPI00021081, | 100.00% | 5 | 11 | 12 | 19.50% | CVCAPD | 95.00% | 47.6 | 41 | 3.89 | 1610.677 | 116 | 128 |
| | Follistatin precursor | IPI00217070, | | | | | | CSNITWK | | | | | | | |
| | | IPI00217071 | | | | | | | | | | | | | |
| S1 | FST Isoform 1 of | IPI00021081, | 100.00% | 5 | 11 | 12 | 19.50% | EAACSS | 95.00% | 44.6 | 41.7 | 2.66 | 1362.694 | 299 | 311 |
| | Follistatin precursor | IPI00217070, | | | | | | GVLLEVK | | | | | | | |
| | | IPI00217071 | | | | | | | | | | | | | |
| S1 | FST Isoform 1 of | IPI00021081, | 100.00% | 5 | 11 | 12 | 19.50% | EQPELEV | 95.00% | 54.8 | 41.5 | 5.8 | 1475.713 | 152 | 163 |
| | Follistatin precursor | IPI00217070, | | | | | | QYQGR | | | | | | | |
| | | IPI00217071 | | | | | | | | | | | | | |
| S1 | FST Isoform 1 of | IPI00021081, | 100.00% | 5 | 11 | 12 | 19.50% | ICPEPAS | 95.00% | 33.9 | 37.3 | 0 | 3071.33 | 195 | 221 |
| | Follistatin precursor | IPI00217070, | | | | | | SEQYLC | | | | | | | |
| | | IPI00217071 | | | | | | GNDGVT | | | | | | | |
| | | | | | | | | YSSACHLR | | | | | | | |
| S1 | PTX3 Pentraxin- | IPI00029568 | 100.00% | 3 | 3 | 3 | 10.80% | LTGFNIW | 95.00% | 74 | 39.9 | 7.21 | 1993.003 | 333 | 349 |
| | related protein | | | | | | | DSVLSNE | | | | | | | |
| | PTX3 precursor | | | | | | | EIR | | | | | | | |
| S1 | PTX3 Pentraxin- | IPI00029568 | 100.00% | 3 | 3 | 3 | 10.80% | LTSALDE | 95.00% | 70.9 | 41.4 | 4.09 | 1430.786 | 113 | 125 |
| | related protein | | | | | | | LLQATR | | | | | | | |
| | PTX3 precursor | | | | | | | | | | | | | | |
| S1 | PTX3 Pentraxin- | IPI00029568 | 100.00% | 3 | 3 | 3 | 10.80% | MLLQATD | 95.00% | 19.5 | 41.9 | 1.82 | 1274.678 | 72 | 82 |
| | related protein | | | | | | | DVLR | | | | | | | |
| | PTX3 precursor | | | | | | | | | | | | | | |
| S2 | TNFRSF11B Tumor | IPI00298362 | 100.00% | 5 | 9 | 9 | 19.20% | FTPNWL | 95.00% | 27.9 | 40.2 | 5.02 | 1901.018 | 215 | 231 |
| | necrosis factor | | | | | | | SVLVDNL | | | | | | | |
| | receptor superfamily | | | | | | | PGTK | | | | | | | |
| | member 11B | | | | | | | | | | | | | | |
| | precursor | | | | | | | | | | | | | | |
| S2 | TNFRSF11B Tumor | IPI00298362 | 100.00% | 5 | 9 | 9 | 19.20% | HTNCSVF | 95.00% | 48.4 | 41 | 5.5 | 1617.843 | 163 | 176 |
| | necrosis factor | | | | | | | GLLLTQK | | | | | | | |
| | receptor superfamily | | | | | | | | | | | | | | |
| | member 11B | | | | | | | | | | | | | | |
| | precursor | | | | | | | | | | | | | | |
| S2 | TNFRSF11B Tumor | IPI00298362 | 100.00% | 5 | 9 | 9 | 19.20% | IIQDIDLC | 95.00% | 72.6 | 40.7 | 9.2 | 1702.844 | 270 | 283 |
| | necrosis factor | | | | | | | ENSVQR | | | | | | | |
| | receptor superfamily | | | | | | | | | | | | | | |
| | member 11B | | | | | | | | | | | | | | |
| | precursor | | | | | | | | | | | | | | |
| S2 | TNFRSF11B Tumor | IPI00298362 | 100.00% | 5 | 9 | 9 | 19.20% | SCPPGF | 95.00% | 62.9 | 40.8 | 0 | 1658.796 | 123 | 138 |
| | necrosis factor | | | | | | | GVVQAG | | | | | | | |
| | receptor superfamily | | | | | | | TPER | | | | | | | |
| | member 11B | | | | | | | | | | | | | | |
| | precursor | | | | | | | | | | | | | | |
| S2 | TNFRSF11B Tumor | IPI00298362 | 100.00% | 5 | 9 | 9 | 19.20% | YLHYDEE | 95.00% | 31 | 39.8 | 6.89 | 2050.918 | 28 | 43 |
| | necrosis factor | | | | | | | TSHQLLC | | | | | | | |
| | receptor superfamily | | | | | | | DK | | | | | | | |
| | member 11B | | | | | | | | | | | | | | |
| | precursor | | | | | | | | | | | | | | |
| S2 | ADAM17 Isoform B | IPI00288894 | 100.00% | 3 | 4 | 4 | 5.34% | INTDGAE | 95.00% | 30.7 | 40.5 | 2.8 | 1790.872 | 140 | 154 |
| | of ADAM 17 | | | | | | | YNIEPLWR | | | | | | | |
| | precursor | | | | | | | | | | | | | | |
| S2 | ADAM17 Isoform B | IPI00288894 | 100.00% | 3 | 4 | 4 | 5.34% | VLAHIRD | 95.00% | 35.1 | 41.1 | 3.07 | 1534.871 | 127 | 139 |
| | of ADAM 17 | | | | | | | DDVIIR | | | | | | | |
| | precursor | | | | | | | | | | | | | | |
| S2 | ADAM17 Isoform B | IPI00288894 | 100.00% | 3 | 4 | 4 | 5.34% | WQDFFT | 95.00% | 78.7 | 40.3 | 10.2 | 1876.862 | 111 | 126 |
| | of ADAM 17 | | | | | | | GHVVGE | | | | | | | |
| | precursor | | | | | | | PDSR | | | | | | | |
| S2 | FST Isoform 1 of | IPI00021081, | 100.00% | 6 | 7 | 8 | 23.00% | CKEQPEL | 95.00% | 0 | 0 | 3.66 | 1746.812 | 150 | 163 |
| | Follistatin precursor | IPI00217070, | | | | | | EVQYQGR | | | | | | | |
| | | IPI00217071 | | | | | | | | | | | | | |
| S2 | FST Isoform 1 of | IPI00021081, | 100.00% | 6 | 7 | 8 | 23.00% | CSLCDEL | 95.00% | 37.7 | 41.3 | 1.14 | 1483.587 | 267 | 278 |
| | Follistatin precursor | IPI00217070, | | | | | | CPDSK | | | | | | | |
| | | IPI00217071 | | | | | | | | | | | | | |
| S2 | FST Isoform 1 of | IPI00021081, | 100.00% | 6 | 7 | 8 | 23.00% | CVCAPD | 95.00% | 32.2 | 41 | 4.35 | 1610.677 | 116 | 128 |
| | Follistatin precursor | IPI00217070, | | | | | | CSNITWK | | | | | | | |
| | | IPI00217071 | | | | | | | | | | | | | |
| S2 | FST Isoform 1 of | IPI00021081, | 100.00% | 6 | 7 | 8 | 23.00% | EAACSS | 95.00% | 66.2 | 41.4 | 3.96 | 1362.694 | 299 | 311 |
| | Follistatin precursor | IPI00217070, | | | | | | GVLLEVK | | | | | | | |
| | | IPI00217071 | | | | | | | | | | | | | |
| S2 | FST Isoform 1 of | IPI00021081, | 100.00% | 6 | 7 | 8 | 23.00% | EQPELEV | 95.00% | 42.2 | 41.5 | 3.96 | 1475.713 | 152 | 163 |
| | Follistatin precursor | IPI00217070, | | | | | | QYQGR | | | | | | | |
| | | IPI00217071 | | | | | | | | | | | | | |
| S2 | FST Isoform 1 of | IPI00021081, | 100.00% | 6 | 7 | 8 | 23.00% | ICPEPAS | 95.00% | 39.4 | 37.3 | 0 | 3071.33 | 195 | 221 |
| | Follistatin precursor | IPI00217070, | | | | | | SEQYLC | | | | | | | |
| | | IPI00217071 | | | | | | GNDGVT | | | | | | | |
| | | | | | | | | YSSACHLR | | | | | | | |
| S2 | PTX3 Pentraxin- | IPI00029568 | 99.80% | 2 | 2 | 2 | 7.09% | ALAAVLE | 95.00% | 25 | 42.3 | 1.39 | 1084.637 | 148 | 157 |
| | related protein | | | | | | | ELR | | | | | | | |
| | PTX3 precursor | | | | | | | | | | | | | | |
| S2 | PTX3 Pentraxin- | IPI00029568 | 99.80% | 2 | 2 | 2 | 7.09% | LTGFNIW | 95.00% | 74.5 | 39.9 | 6.8 | 1993.003 | 333 | 349 |
| | related protein | | | | | | | DSVLSNE | | | | | | | |
| | PTX3 precursor | | | | | | | EIR | | | | | | | |
| S3 | TNFRSF11B Tumor | IPI00298362 | 100.00% | 3 | 5 | 5 | 11.70% | FTPNWL | 95.00% | 38.5 | 40.2 | 2.06 | 1901.018 | 215 | 231 |
| | necrosis factor | | | | | | | SVLVDNL | | | | | | | |
| | receptor superfamily | | | | | | | PGTK | | | | | | | |
| | member 11B | | | | | | | | | | | | | | |
| | precursor | | | | | | | | | | | | | | |
| S3 | TNFRSF11B Tumor | IPI00298362 | 100.00% | 3 | 5 | 5 | 11.70% | IIQDIDLC | 95.00% | 22.4 | 40.6 | 2.96 | 1702.844 | 270 | 283 |
| | necrosis factor | | | | | | | ENSVQR | | | | | | | |
| | receptor superfamily | | | | | | | | | | | | | | |
| | member 11B | | | | | | | | | | | | | | |
| | precursor | | | | | | | | | | | | | | |
| S3 | TNFRSF11B Tumor | IPI00298362 | 100.00% | 3 | 5 | 5 | 11.70% | YLHYDEE | 95.00% | 21.2 | 39.6 | 2.59 | 2050.918 | 28 | 43 |
| | necrosis factor | | | | | | | TSHQLLC | | | | | | | |
| | receptor superfamily | | | | | | | DK | | | | | | | |
| | member 11B | | | | | | | | | | | | | | |
| | precursor | | | | | | | | | | | | | | |
| S3 | ADAM17 Isoform B | IPI00029606, | 100.00% | 3 | 4 | 4 | 5.34% | INTDGAE | 95.00% | 71.3 | 40.6 | 3.8 | 1790.872 | 140 | 154 |
| | of ADAM 17 | IPI00288894 | | | | | | YNIEPLWR | | | | | | | |
| | precursor | | | | | | | | | | | | | | |
| S3 | ADAM17 Isoform B | IPI00029606, | 100.00% | 3 | 4 | 4 | 5.34% | VLAHIRD | 95.00% | 47.7 | 41.1 | 3.08 | 1534.871 | 127 | 139 |
| | of ADAM 17 | IPI00288894 | | | | | | DDVIIR | | | | | | | |
| | precursor | | | | | | | | | | | | | | |
| S3 | ADAM17 Isoform B | IPI00029606, | 100.00% | 3 | 4 | 4 | 5.34% | WQDFFT | 95.00% | 73.8 | 40.3 | 11.2 | 1876.862 | 111 | 126 |
| | of ADAM 17 | IPI00288894 | | | | | | GHVVGE | | | | | | | |
| | precursor | | | | | | | PDSR | | | | | | | |
| S3 | FST Isoform 1 of | IPI00021081, | 100.00% | 3 | 4 | 4 | 12.20% | CSLCDEL | 95.00% | 49.1 | 41.3 | 4.7 | 1483.587 | 267 | 278 |
| | Follistatin precursor | IPI00217070, | | | | | | CPDSK | | | | | | | |
| | | IPI00217071 | | | | | | | | | | | | | |
| S3 | FST Isoform 1 of | IPI00021081, | 100.00% | 3 | 4 | 4 | 12.20% | EAACSS | 95.00% | 30.5 | 41.7 | 0.328 | 1362.694 | 299 | 311 |
| | Follistatin precursor | IPI00217070, | | | | | | GVLLEVK | | | | | | | |
| | | IPI00217071 | | | | | | | | | | | | | |
| S3 | FST Isoform 1 of | IPI00021081, | 100.00% | 3 | 4 | 4 | 12.20% | LSTSWTE | 95.00% | 103 | 40 | 9.62 | 1998.93 | 61 | 77 |
| | Follistatin precursor | IPI00217070, | | | | | | EDVNDN | | | | | | | |
| | | IPI00217071 | | | | | | TLFK | | | | | | | |

TABLE 7D
Detailed information for Pentraxin 3 and ADAM-17 identified in H520 cell line

| | | | | | | | | | Best | | Best | Best XI | Calculated | | |
|---|---|---|---|---|---|---|---|---|---|---|---|---|---|---|---|
| Biological | | Protein | Protein | Number of | Number of | Number | Percentage | | Peptide | Best | Mascot | Tandem | Peptide | | |
| sample | | accession | identification | unique | unique | of total | sequence | Peptide | identification | Mascot | identity | −log(e) | Mass | Peptide | Peptide |
| name | Protein name | numbers | probability | peptides | spectra | spectra | coverage | sequence | probability | ion score | score | score | (AMU) | start index | stop index |
| S1 | PTX3 Pentraxin- | IPI00029568 | 100.00% | 16 | 58 | 111 | 54.10% | ADLHAV | 95.00% | 50 | 41.8 | 0 | 1294.666 | 161 | 172 |
| | related protein | | | | | | | QGWAAR | | | | | | | |
| | PTX3 precursor | | | | | | | | | | | | | | |
| S1 | PTX3 Pentraxin- | IPI00029568 | 100.00% | 16 | 58 | 111 | 54.10% | ALAAVLE | 95.00% | 63.1 | 42.1 | 0 | 1084.637 | 148 | 157 |
| | related protein | | | | | | | ELR | | | | | | | |
| | PTX3 precursor | | | | | | | | | | | | | | |
| S1 | PTX3 Pentraxin- | IPI00029568 | 100.00% | 16 | 58 | 111 | 54.10% | GNIVGW | 95.00% | 44.5 | 39.4 | 7.13 | 2169.073 | 361 | 381 |
| | related protein | | | | | | | GVTEIQP | | | | | | | |
| | PTX3 precursor | | | | | | | HGGAQY | | | | | | | |
| | | | | | | | | VS | | | | | | | |
| S1 | PTX3 Pentraxin- | IPI00029568 | 100.00% | 16 | 58 | 111 | 54.10% | IFGSVHP | 95.00% | 19.8 | 41.4 | 1.77 | 1395.768 | 192 | 203 |
| | related protein | | | | | | | VRPMR | | | | | | | |
| | PTX3 precursor | | | | | | | | | | | | | | |
| S1 | PTX3 Pentraxin- | IPI00029568 | 100.00% | 16 | 58 | 111 | 54.10% | LAESLAR | 95.00% | 39.8 | 40.6 | 0 | 1836.939 | 95 | 112 |
| | related protein | | | | | | | PCAPGA | | | | | | | |
| | PTX3 precursor | | | | | | | PAEAR | | | | | | | |
| S1 | PTX3 Pentraxin- | IPI00029568 | 100.00% | 16 | 58 | 111 | 54.10% | LESFSAC | 95.00% | 21.7 | 41.6 | 2.07 | 1339.672 | 204 | 214 |
| | related protein | | | | | | | IWVK | | | | | | | |
| | PTX3 precursor | | | | | | | | | | | | | | |
| S1 | PTX3 Pentraxin- | IPI00029568 | 100.00% | 16 | 58 | 111 | 54.10% | LFIMLEN | 95.00% | 48.5 | 41.6 | 6.77 | 1381.697 | 59 | 69 |
| | related protein | | | | | | | SQMR | | | | | | | |
| | PTX3 precursor | | | | | | | | | | | | | | |
| S1 | PTX3 Pentraxin- | IPI00029568 | 100.00% | 16 | 58 | 111 | 54.10% | LTGFNIW | 95.00% | 118 | 39.9 | 11.8 | 1993.003 | 333 | 349 |
| | related protein | | | | | | | DSVLSNE | | | | | | | |
| | PTX3 precursor | | | | | | | EIR | | | | | | | |
| S1 | PTX3 Pentraxin- | IPI00029568 | 100.00% | 16 | 58 | 111 | 54.10% | LTGFNIW | 95.00% | 92.7 | 36.9 | 0 | 3190.523 | 333 | 360 |
| | related protein | | | | | | | DSVLSNE | | | | | | | |
| | PTX3 precursor | | | | | | | EIRETGG | | | | | | | |
| | | | | | | | | AESCHIR | | | | | | | |
| S1 | PTX3 Pentraxin- | IPI00029568 | 100.00% | 16 | 58 | 111 | 54.10% | LTSALDE | 95.00% | 24.2 | 41.5 | 3.43 | 1430.786 | 113 | 125 |
| | related protein | | | | | | | LLQATR | | | | | | | |
| | PTX3 precursor | | | | | | | | | | | | | | |
| S1 | PTX3 Pentraxin- | IPI00029568 | 100.00% | 16 | 58 | 111 | 54.10% | LVAEAMV | 95.00% | 80.2 | 42.3 | 5.44 | 1145.635 | 256 | 266 |
| | related protein | | | | | | | SLGR | | | | | | | |
| | PTX3 precursor | | | | | | | | | | | | | | |
| S1 | PTX3 Pentraxin- | IPI00029568 | 100.00% | 16 | 58 | 111 | 54.10% | MLLQATD | 95.00% | 46.5 | 41.9 | 2.14 | 1274.678 | 72 | 82 |
| | related protein | | | | | | | DVLR | | | | | | | |
| | PTX3 precursor | | | | | | | | | | | | | | |
| S1 | PTX3 Pentraxin- | IPI00029568 | 100.00% | 16 | 58 | 111 | 54.10% | MLLQATD | 95.00% | 36.9 | 40.4 | 3.55 | 1857.986 | 72 | 87 |
| | related protein | | | | | | | DVLRGEL | | | | | | | |
| | PTX3 precursor | | | | | | | QR | | | | | | | |
| S1 | PTX3 Pentraxin- | IPI00029568 | 100.00% | 16 | 58 | 111 | 54.10% | NGCCVG | 95.00% | 54.3 | 40.2 | 0 | 1903.807 | 315 | 332 |
| | related protein | | | | | | | GGFDETL | | | | | | | |
| | PTX3 precursor | | | | | | | AFSGR | | | | | | | |
| S1 | PTX3 Pentraxin- | IPI00029568 | 100.00% | 16 | 58 | 111 | 54.10% | SWLPAG | 95.00% | 58.3 | 40.4 | 0 | 1848.914 | 173 | 188 |
| | related protein | | | | | | | CETAILF | | | | | | | |
| | PTX3 precursor | | | | | | | PMR | | | | | | | |
| S1 | PTX3 Pentraxin- | IPI00029568 | 100.00% | 16 | 58 | 111 | 54.10% | TILFSYG | 95.00% | 44.9 | 42.3 | 0.699 | 1029.562 | 222 | 230 |
| | related protein | | | | | | | TK | | | | | | | |
| | PTX3 precursor | | | | | | | | | | | | | | |
| S1 | ADAM17 Isoform | IPI00288894 | 99.80% | 2 | 3 | 3 | 3.76% | INTDGAE | 95.00% | 46.3 | 40.5 | 3.39 | 1790.872 | 140 | 154 |
| | A of ADAM 17 | | | | | | | YNIEPLWR | | | | | | | |
| | precursor | | | | | | | | | | | | | | |
| S1 | ADAM17 Isoform | IPI00288894 | 99.80% | 2 | 3 | 3 | 3.76% | WQDFFT | 95.00% | 26.8 | 40.2 | 6.23 | 1876.862 | 111 | 126 |
| | A of ADAM 17 | | | | | | | GHVVGE | | | | | | | |
| | precursor | | | | | | | PDSR | | | | | | | |
| S2 | PTX3 Pentraxin- | IPI00029568 | 100.00% | 15 | 51 | 90 | 52.80% | ADLHAV | 95.00% | 54.2 | 41.6 | 0 | 1294.666 | 161 | 172 |
| | related protein | | | | | | | QGWAAR | | | | | | | |
| | PTX3 precursor | | | | | | | | | | | | | | |
| S2 | PTX3 Pentraxin- | IPI00029568 | 100.00% | 15 | 51 | 90 | 52.80% | ALAAVLE | 95.00% | 55.7 | 42.3 | 3.38 | 1084.637 | 148 | 157 |
| | related protein | | | | | | | ELR | | | | | | | |
| | PTX3 precursor | | | | | | | | | | | | | | |
| S2 | PTX3 Pentraxin- | IPI00029568 | 100.00% | 15 | 51 | 90 | 52.80% | GNIVGW | 95.00% | 50.6 | 39.5 | 6.59 | 2169.073 | 361 | 381 |
| | related protein | | | | | | | GVTEIQP | | | | | | | |
| | PTX3 precursor | | | | | | | HGGAQY | | | | | | | |
| | | | | | | | | VS | | | | | | | |
| S2 | PTX3 Pentraxin- | IPI00029568 | 100.00% | 15 | 51 | 90 | 52.80% | IFGSVHP | 95.00% | 23.6 | 41.4 | 2 | 1395.768 | 192 | 203 |
| | related protein | | | | | | | VRPMR | | | | | | | |
| | PTX3 precursor | | | | | | | | | | | | | | |
| S2 | PTX3 Pentraxin- | IPI00029568 | 100.00% | 15 | 51 | 90 | 52.80% | LAESLAR | 95.00% | 51 | 40.6 | 0 | 1836.939 | 95 | 112 |
| | related protein | | | | | | | PCAPGA | | | | | | | |
| | PTX3 precursor | | | | | | | PAEAR | | | | | | | |
| S2 | PTX3 Pentraxin- | IPI00029568 | 100.00% | 15 | 51 | 90 | 52.80% | LESFSAC | 95.00% | 28.1 | 41.6 | 1.85 | 1339.672 | 204 | 214 |
| | related protein | | | | | | | IWVK | | | | | | | |
| | PTX3 precursor | | | | | | | | | | | | | | |
| S2 | PTX3 Pentraxin- | IPI00029568 | 100.00% | 15 | 51 | 90 | 52.80% | LFIMLEN | 95.00% | 77 | 41.6 | 6.17 | 1381.697 | 59 | 69 |
| | related protein | | | | | | | SQMR | | | | | | | |
| | PTX3 precursor | | | | | | | | | | | | | | |
| S2 | PTX3 Pentraxin- | IPI00029568 | 100.00% | 15 | 51 | 90 | 52.80% | LTGFNIW | 95.00% | 36.7 | 39.9 | 6.51 | 1993.003 | 333 | 349 |
| | related protein | | | | | | | DSVLSNE | | | | | | | |
| | PTX3 precursor | | | | | | | EIR | | | | | | | |
| S2 | PTX3 Pentraxin- | IPI00029568 | 100.00% | 15 | 51 | 90 | 52.80% | LTGFNIW | 95.00% | 83.1 | 36.9 | 7.59 | 3190.523 | 333 | 360 |
| | related protein | | | | | | | DSVLSNE | | | | | | | |
| | PTX3 precursor | | | | | | | EIRETGG | | | | | | | |
| | | | | | | | | AESCHIR | | | | | | | |
| S2 | PTX3 Pentraxin- | IPI00029568 | 100.00% | 15 | 51 | 90 | 52.80% | LTSALDE | 95.00% | 86.1 | 41.4 | 7.26 | 1430.786 | 113 | 125 |
| | related protein | | | | | | | LLQATR | | | | | | | |
| | PTX3 precursor | | | | | | | | | | | | | | |
| S2 | PTX3 Pentraxin- | IPI00029568 | 100.00% | 15 | 51 | 90 | 52.80% | LVAEAMV | 95.00% | 36.4 | 42.3 | 2.8 | 1145.635 | 256 | 266 |
| | related protein | | | | | | | SLGR | | | | | | | |
| | PTX3 precursor | | | | | | | | | | | | | | |
| S2 | PTX3 Pentraxin- | IPI00029568 | 100.00% | 15 | 51 | 90 | 52.80% | MLLQATD | 95.00% | 50.9 | 41.8 | 2.62 | 1274.678 | 72 | 82 |
| | related protein | | | | | | | DVLR | | | | | | | |
| | PTX3 precursor | | | | | | | | | | | | | | |
| S2 | PTX3 Pentraxin- | IPI00029568 | 100.00% | 15 | 51 | 90 | 52.80% | NGCCVG | 95.00% | 67 | 40.2 | 5.89 | 1903.807 | 315 | 332 |
| | related protein | | | | | | | GGFDETL | | | | | | | |
| | PTX3 precursor | | | | | | | AFSGR | | | | | | | |
| S2 | PTX3 Pentraxin- | IPI00029568 | 100.00% | 15 | 51 | 90 | 52.80% | SWLPAG | 95.00% | 46.6 | 40.4 | 5.92 | 1848.914 | 173 | 188 |
| | related protein | | | | | | | CETAILF | | | | | | | |
| | PTX3 precursor | | | | | | | PMR | | | | | | | |
| S2 | PTX3 Pentraxin- | IPI00029568 | 100.00% | 15 | 51 | 90 | 52.80% | TILFSYG | 95.00% | 40.5 | 42.5 | 0.409 | 1029.562 | 222 | 230 |
| | related protein | | | | | | | TK | | | | | | | |
| | PTX3 precursor | | | | | | | | | | | | | | |
| S2 | ADAM17 Isoform | IPI00288894 | 99.80% | 2 | 2 | 2 | 3.40% | INTDGAE | 95.00% | 67.1 | 40.6 | 5.2 | 1790.872 | 140 | 154 |
| | A of ADAM 17 | | | | | | | YNIEPLWR | | | | | | | |
| | precursor | | | | | | | | | | | | | | |
| S2 | ADAM17 Isoform | IPI00288894 | 99.80% | 2 | 2 | 2 | 3.40% | VLAHIRD | 95.00% | 29.6 | 41.2 | 3.41 | 1534.871 | 127 | 139 |
| | A of ADAM 17 | | | | | | | DDVIIR | | | | | | | |
| | precursor | | | | | | | | | | | | | | |
| S3 | PTX3 Pentraxin- | IPI00029568 | 100.00% | 16 | 50 | 128 | 54.10% | ADLHAV | 95.00% | 69.6 | 41.6 | 0 | 1294.666 | 161 | 172 |
| | related protein | | | | | | | QGWAAR | | | | | | | |
| | PTX3 precursor | | | | | | | | | | | | | | |
| S3 | PTX3 Pentraxin- | IPI00029568 | 100.00% | 16 | 50 | 128 | 54.10% | ALAAVLE | 95.00% | 66.2 | 42.1 | 4.31 | 1084.637 | 148 | 157 |
| | related protein | | | | | | | ELR | | | | | | | |
| | PTX3 precursor | | | | | | | | | | | | | | |
| S3 | PTX3 Pentraxin- | IPI00029568 | 100.00% | 16 | 50 | 128 | 54.10% | GNIVGW | 95.00% | 33.7 | 39.4 | 4.33 | 2169.073 | 361 | 381 |
| | related protein | | | | | | | GVTEIQP | | | | | | | |
| | PTX3 precursor | | | | | | | HGGAQY | | | | | | | |
| | | | | | | | | VS | | | | | | | |
| S3 | PTX3 Pentraxin- | IPI00029568 | 100.00% | 16 | 50 | 128 | 54.10% | IFGSVHP | 95.00% | 23.7 | 41.4 | 2.43 | 1395.768 | 192 | 203 |
| | related protein | | | | | | | VRPMR | | | | | | | |
| | PTX3 precursor | | | | | | | | | | | | | | |
| S3 | PTX3 Pentraxin- | IPI00029568 | 100.00% | 16 | 50 | 128 | 54.10% | LAESLAR | 95.00% | 50.7 | 40.6 | 6.51 | 1836.939 | 95 | 112 |
| | related protein | | | | | | | PCAPGA | | | | | | | |
| | PTX3 precursor | | | | | | | PAEAR | | | | | | | |
| S3 | PTX3 Pentraxin- | IPI00029568 | 100.00% | 16 | 50 | 128 | 54.10% | LESFSAC | 95.00% | 23.4 | 41.6 | 1.21 | 1339.672 | 204 | 214 |
| | related protein | | | | | | | IWVK | | | | | | | |
| | PTX3 precursor | | | | | | | | | | | | | | |
| S3 | PTX3 Pentraxin- | IPI00029568 | 100.00% | 16 | 50 | 128 | 54.10% | LFIMLEN | 95.00% | 33.6 | 41.5 | 6.03 | 1381.697 | 59 | 69 |
| | related protein | | | | | | | SQMR | | | | | | | |
| | PTX3 precursor | | | | | | | | | | | | | | |
| S3 | PTX3 Pentraxin- | IPI00029568 | 100.00% | 16 | 50 | 128 | 54.10% | LTGFNIW | 95.00% | 106 | 39.9 | 9.96 | 1993.003 | 333 | 349 |
| | related protein | | | | | | | DSVLSNE | | | | | | | |
| | PTX3 precursor | | | | | | | EIR | | | | | | | |
| S3 | PTX3 Pentraxin- | IPI00029568 | 100.00% | 16 | 50 | 128 | 54.10% | LTGFNIW | 95.00% | 88.8 | 36.9 | 8.47 | 3190.523 | 333 | 360 |
| | related protein | | | | | | | DSVLSNE | | | | | | | |
| | PTX3 precursor | | | | | | | EIRETGG | | | | | | | |
| | | | | | | | | AESCHIR | | | | | | | |
| S3 | PTX3 Pentraxin- | IPI00029568 | 100.00% | 16 | 50 | 128 | 54.10% | LTSALDE | 95.00% | 61.5 | 41.5 | 3.92 | 1430.786 | 113 | 125 |
| | related protein | | | | | | | LLQATR | | | | | | | |
| | PTX3 precursor | | | | | | | | | | | | | | |
| S3 | PTX3 Pentraxin- | IPI00029568 | 100.00% | 16 | 50 | 128 | 54.10% | LVAEAMV | 95.00% | 63.5 | 42.3 | 4.4 | 1161.63 | 256 | 266 |
| | related protein | | | | | | | SLGR | | | | | | | |
| | PTX3 precursor | | | | | | | | | | | | | | |
| S3 | PTX3 Pentraxin- | IPI00029568 | 100.00% | 16 | 50 | 128 | 54.10% | MLLQATD | 95.00% | 52.9 | 41.9 | 3.4 | 1274.678 | 72 | 82 |
| | related protein | | | | | | | DVLR | | | | | | | |
| | PTX3 precursor | | | | | | | | | | | | | | |
| S3 | PTX3 Pentraxin- | IPI00029568 | 100.00% | 16 | 50 | 128 | 54.10% | MLLQATD | 95.00% | 28.8 | 40.4 | 3.05 | 1857.986 | 72 | 87 |
| | related protein | | | | | | | DVLRGEL | | | | | | | |
| | PTX3 precursor | | | | | | | QR | | | | | | | |
| S3 | PTX3 Pentraxin- | IPI00029568 | 100.00% | 16 | 50 | 128 | 54.10% | NGCCVG | 95.00% | 92.7 | 40.2 | 10.6 | 1903.807 | 315 | 332 |
| | related protein | | | | | | | GGFDETL | | | | | | | |
| | PTX3 precursor | | | | | | | AFSGR | | | | | | | |
| S3 | PTX3 Pentraxin- | IPI00029568 | 100.00% | 16 | 50 | 128 | 54.10% | SWLPAG | 95.00% | 61.9 | 40.4 | 7.01 | 1848.914 | 173 | 188 |
| | related protein | | | | | | | CETAILF | | | | | | | |
| | PTX3 precursor | | | | | | | PMR | | | | | | | |
| S3 | PTX3 Pentraxin- | IPI00029568 | 100.00% | 16 | 50 | 128 | 54.10% | TILFSYG | 95.00% | 29.5 | 42.5 | 0.244 | 1029.562 | 222 | 230 |
| | related protein | | | | | | | TK | | | | | | | |
| | PTX3 precursor | | | | | | | | | | | | | | |

TABLE 8
Proteins identified in this study that were not found in the 6 previous studies related
to lung proteomics.

| Protein name | Accession numbers | Protein name | Accession numbers |
|---|---|---|---|
| 105 kDa protein | IPI00794900 | KRT19 Keratin, type I cytoskeletal 19 | IPI00479145 |
| 12 kDa protein | IPI00797738 | KRT3 Keratin, type II cytoskeletal 3 | IPI00290857 |
| 19 kDa protein | IPI00795717 | KTN1 Isoform 1 of Kinectin | IPI00328753, IPI00337736 |
| 21 kDa protein | IPI00478011 | L1CAM Isoform 1 of Neural cell adhesion molecule L1 | IPI00027087, IPI00334532, |
| | | precursor | IPI00646281 |
| 23 kDa protein | IPI00647593 | LAMA1 Laminin subunit alpha-1 precursor | IPI00375294 |
| 25 kDa protein | IPI00375127 | LAMA2 laminin alpha 2 subunit isoform b precursor | IPI00218725, IPI00479834 |
| 27 kDa protein | IPI00455527 | LAMA5 400 kDa protein | IPI00641693 |
| 30 kDa protein | IPI00472119 | LAMA5 Laminin subunit alpha-5 precursor | IPI00783665 |
| 31 kDa protein | IPI00479366 | LAMB2 Laminin subunit beta-2 precursor | IPI00296922 |
| 35 kDa protein | IPI00738677 | LAMP2 Isoform LAMP-2A of Lysosome-associated | IPI00009030, IPI00216172, |
| | | membrane glycoprotein 2 precursor | IPI00739827 |
| 52 kDa protein | IPI00795769 | LAP3 Isoform 1 of Cytosol aminopeptidase | IPI00419237 |
| 64 kDa protein | IPI00175126 | LARS Leucyl-tRNA synthetase, cytoplasmic | IPI00103994 |
| 67 kDa protein | IPI00020513, IPI00797866 | LEMD3 Inner nuclear membrane protein Man1 | IPI00032491 |
| 71 kDa protein | IPI00783641 | LEPRE1 Isoform 3 of Prolyl 3-hydroxylase 1 precursor | IPI00045839 |
| CD68 antigen variant (Fragment) | IPI00555602 | LFNG Isoform 1 of Beta-1,3-N- | IPI00455739 |
| | | acetylglucosaminyltransferase lunatic fringe | |
| DAZAP1/MEF2D fusion protein | IPI00785057 | LGALS3 Galectin-3 | IPI00465431 |
| Eukaryotic translation elongation factor 1 alpha-like 3 | IPI00472724 | LGMN Legumain precursor | IPI00293303 |
| HDCMB21P | IPI00384863 | LIMA1 Isoform Beta of LIM domain and actin-binding protein 1 | IPI00008918, IPI00220465, |
| | | | IPI00796222, IPI00796705 |
| Novel protein similar to Pre-B cell enhancing factor | IPI00472879 | LIPA CDNA FLJ43203 fis, clone FEBRA2008468, highly | IPI00748567 |
| | | similar to LYSOSOMAL ACID LIPASE/CHOLESTERYL | |
| | | ESTER HYDROLASE | |
| P37 AUF1 | IPI00382617 | LIPA Isoform 1 of Lysosomal acid lipase/cholesteryl ester | IPI00007207, IPI00446007, |
| | | hydrolase precursor | IPI00748567 |
| P40 | IPI00164951, IPI00298454, IPI00477094, | LIPG Isoform 1 of Endothelial lipase precursor | IPI00005686 |
| | IPI00477666, IPI00479567, | | |
| | IPI00479700, IPI00749461, | | |
| | IPI00784621 | | |
| Protein | IPI00789847 | LMAN1 ERGIC-53 protein precursor | IPI00026530 |
| Trypsinogen C | IPI00169276 | LMAN2 Vesicular integral-membrane protein VIP36 | IPI00009950 |
| | | precursor | |
| AARS 107 kDa protein | IPI00784131 | LMNB1 Lamin-B1 | IPI00217975 |
| AARS Alanyl-tRNA synthetase, cytoplasmic | IPI00027442, IPI00784131 | LMNB2 Lamin B2 | IPI00009771 |
| ABCE1 ATP-binding cassette sub-family E member 1 | IPI00303207 | LOC196463 Hypothetical protein LOC196463 | IPI00169285 |
| ABCF1 Isoform 2 of ATP-binding cassette sub-family F | IPI00013495, IPI00302146, IPI00642960, | LOC253012 WLKV305 | IPI00377047 |
| member 1 | IPI00792186 | | |
| ABHD14B Isoform 1 of Abhydrolase domain-containing | IPI00063827, IPI00747859 | LOC388720 similar to ubiquitin and ribosomal protein S27a | IPI00397808 |
| protein 14B | | precursor | |
| ABP1 Isoform 1 of Amiloride-sensitive amine oxidase | IPI00020982, IPI00219832 | LOC389842 similar to Ran-specific GTPase-activating | IPI00399212, IPI00414127 |
| [copper-containing] precursor | | protein | |
| ACAT1 ACAT1 protein | IPI00440499 | LOC440055 similar to ribosomal protein S12 | IPI00456898 |
| ACAT2 Acetyl-CoA acetyltransferase, cytosolic | IPI00291419 | LOC51035 Isoform 1 of SAPK substrate protein 1 | IPI00027378 |
| ACBD3 Golgi resident protein GCP60 | IPI00009315 | LOC646993 similar to high-mobility group box 3 | IPI00217477, IPI00376756, |
| | | | IPI00411540, IPI00640781, |
| | | | IPI00643317 |
| ACE Angiotensin-converting enzyme, somatic isoform | IPI00437751 | LOC652595 BA395L14.12 | IPI00183920, IPI00297477 |
| precursor | | | |
| ACIN1 Isoform 1 of Apoptotic chromatin condensation | IPI00007334 | LOC653269 similar to Prostate, ovary, testis expressed | IPI00740545 |
| inducer in the nucleus | | protein on chromosome 2 isoform 2 | |
| ACLY ATP citrate lyase isoform 2 | IPI00394838 | LOC653994; EIF4H Isoform Long of Eukaryotic translation | IPI00014263, IPI00220894 |
| | | initiation factor 4H | |
| ACLY ATP-citrate synthase | IPI00021290 | LOC728641; FABP5; LOC731043 Fatty acid-binding protein, | IPI00007797 |
| | | epidermal | |
| ACOT7 Isoform 2 of Cytosolic acyl coenzyme A thioester | IPI00395469 | LOC731751 similar to protein kinase, DNA-activated, | IPI00786995 |
| hydrolase | | catalytic polypeptide | |
| ACP1 Isoform 1 of Low molecular weight phosphotyrosine | IPI00219861 | LOC84661 Dpy-30-like protein | IPI00028109 |
| protein phosphatase | | | |
| ACP5 Tartrate-resistant acid phosphatase type 5 precursor | IPI00419240 | LOXL2 Lysyl oxidase-like 2 variant | IPI00294839, IPI00782994 |
| ACTA1 Actin, alpha skeletal muscle | IPI00021428 | LOXL2; ENTPD4 Lysyl oxidase homolog 2 precursor | IPI00782994 |
| ACTR2 Actin-like protein 2 | IPI00005159, IPI00470573, IPI00749250 | LPHN1 Isoform 1 of Latrophilin-1 precursor | IPI00183445, IPI00410210 |
| ACTR2 actin-related protein 2 isoform a | IPI00470573 | LPP Lipoma-preferred partner | IPI00023704 |
| ADA Adenosine deaminase | IPI00296441 | LRBA Lipopolysaccharide-responsive and beige-like anchor | IPI00002255, IPI00477088 |
| | | protein | |
| ADAM10 ADAM 10 precursor | IPI00013897 | LRP11 Isoform 1 of Low-density lipoprotein receptor-related | IPI00045841 |
| | | protein 11 precursor | |
| ADAM15 a disintegrin and metalloproteinase domain 15 | IPI00420069 | LRP2 Low-density lipoprotein receptor-related protein 2 | IPI00024292 |
| isoform 3 preproprotein | | precursor | |
| ADAM15 a disintegrin and metalloproteinase domain 15 | IPI00420067 | LRP8 Isoform 1 of Low-density lipoprotein receptor-related | IPI00005774 |
| isoform 4 preproprotein | | protein 8 precursor | |
| ADAM17 Isoform A of ADAM 17 precursor | IPI00288894 | LRRC47 Leucine-rich repeat-containing protein 47 | IPI00170935 |
| ADAM17 Isoform B of ADAM 17 precursor | IPI00029606, IPI00288894 | LRRC59 Leucine-rich repeat-containing protein 59 | IPI00396321 |
| ADAM19 Isoform A of ADAM 19 precursor | IPI00011901, IPI00249735 | LRRFIP1 Isoform 1 of Leucine-rich repeat flightless- | IPI00785113 |
| | | interacting protein 1 | |
| ADAM23 Isoform Alpha of ADAM 23 precursor | IPI00021903 | LRRFIP1 Isoform 2 of Leucine-rich repeat flightless- | IPI00006207, IPI00382733 |
| | | interacting protein 1 | |
| ADAM9 Isoform 1 of ADAM 9 precursor | IPI00440932, IPI00747759 | LRRTM1 Leucine-rich repeat transmembrane neuronal | IPI00328716 |
| | | protein 1 precursor | |
| ADAMTS1 ADAMTS-1 precursor | IPI00005908 | LSM8 U6 snRNA-associated Sm-like protein LSm8 | IPI00219871 |
| ADAMTS12 ADAM metallopeptidase with thrombospondin | IPI00036578 | LSR Isoform 1 of Lipolysis-stimulated lipoprotein receptor | IPI00409640, IPI00409641, |
| type 1 motif, 12 preproprotein | | | IPI00641640 |
| ADAMTS19 ADAMTS-19 precursor | IPI00152639 | LSR Isoform 2 of Lipolysis-stimulated lipoprotein receptor | IPI00328218, IPI00409640, |
| | | | IPI00409641, IPI00641640 |
| ADAMTSL1 ADAMTS-like 1 isoform 1 | IPI00157513 | LTA4H Isoform 1 of Leukotriene A-4 hydrolase | IPI00219077, IPI00514090 |
| ADAMTSL2 ADAMTS-like 2 | IPI00790458 | LTA4H Isoform 2 of Leukotriene A-4 hydrolase | IPI00514090 |
| ADAR Isoform 2 of Double-stranded RNA-specific | IPI00025057, IPI00025058, IPI00394665 | LTB4DH NADP-dependent leukotriene B4 12- | IPI00292657 |
| adenosine deaminase | | hydroxydehydrogenase | |
| ADAR Isoform 4 of Double-stranded RNA-specific | IPI00394668 | LTBP3 Isoform 1 of Latent-transforming growth factor beta- | IPI00073196, IPI00398794 |
| adenosine deaminase | | binding protein 3 precursor | |
| ADCYAP1 Pituitary adenylate cyclase-activating polypeptide | IPI00000027 | LTBP4 latent transforming growth factor beta binding | IPI00783492 |
| precursor | | protein 4 isoform c | |
| ADH5 Class III alCohol dehydrogenase 5 Chi subunit | IPI00746777 | LUC7L2 Isoform 1 of Putative RNA-binding protein Luc7-like 2 | IPI00006932 |
| ADI1 Isoform 1 of 1,2-dihydroxy-3-keto-5-methylthiopentene | IPI00651738 | LUC7L2 Isoform 2 of Putative RNA-binding protein Luc7-like 2 | IPI00216804 |
| dioxygenase | | | |
| ADK Isoform Short of Adenosine kinase | IPI00234368 | LYPLA3 1-O-acylceramide synthase precursor | IPI00301459 |
| ADRM1 Adhesion-regulating molecule 1 precursor | IPI00033030, IPI00470921 | M6PRBP1 Isoform B of Mannose-6-phosphate receptor- | IPI00303882 |
| | | binding protein 1 | |
| ADSS Adenylosuccinate synthetase isozyme 2 | IPI00026833 | MACF1 Isoform 2 of Microtubule-actin cross-linking factor 1, | IPI00256861, IPI00478226, |
| | | isoforms 1/2/3/5 | IPI00513991, IPI00550385, |
| | | | IPI00744472 |
| AGT Angiotensinogen precursor | IPI00032220 | MACF1 Microtubule-actin cross-linking factor 1, isoform 4 | IPI00432363, IPI00514468 |
| AHCY Adenosylhomocysteinase | IPI00012007 | MAGEA10 Melanoma-associated antigen 10 | IPI00301104 |
| AHNAK 313 kDa protein | IPI00555610 | MAGEA4 Melanoma-associated antigen 4 | IPI00018042 |
| AHSA1 Activator of 90 kDa heat shock protein ATPase | IPI00030706 | MAGED2 Isoform 1 of Melanoma-associated antigen D2 | IPI00009542, IPI00477809 |
| homolog 1 | | | |
| AIP AH receptor-interacting protein | IPI00010460 | MAN1A1 Mannosyl-oligosaccharide 1,2-alpha-mannosidase | IPI00439446 |
| | | IA | |
| AK2 Isoform 1 of Adenylate kinase isoenzyme 2, | IPI00215901, IPI00218988 | MAN1A2 Mannosyl-oligosaccharide 1,2-alpha-mannosidase | IPI00009145 |
| mitochondrial | | IB | |
| AKAP12 A-kinase anchor protein 12 isoform 2 | IPI00217683 | MAN1B1 Endoplasmic reticulum mannosyl-oligosaccharide | IPI00008207 |
| | | 1,2-alpha-mannosidase | |
| AKAP12 Isoform 1 of A-kinase anchor protein 12 | IPI00237884 | MAN2A1 Alpha-mannosidase 2 | IPI00003802 |
| AKR1B1 Aldose reductase | IPI00413641 | MAP2 Isoform 1 of Microtubule-associated protein 2 | IPI00003842, IPI00010728, |
| | | | IPI00472094 |
| AKR1B10 Aldo-keto reductase family 1 member B10 | IPI00105407 | MAP2K1 Dual specificity mitogen-activated protein kinase | IPI00219604 |
| | | kinase 1 | |
| AKR1C2 Aldo-keto reductase family 1 member C2 | IPI00005668 | MAP4 Isoform 1 of Microtubule-associated protein 4 | IPI00396171, IPI00745518 |
| AKR1C3 Aldo-keto reductase family 1 member C3 | IPI00291483 | MAP4 Isoform 2 of Microtubule-associated protein 4 | IPI00220113, IPI00396171, |
| | | | IPI00745518 |
| AKR7A2 Aflatoxin B1 aldehyde reductase member 2 | IPI00305978 | MAP4 Microtubule-associated protein 4 isoform 1 variant | IPI00745518 |
| | | (Fragment) | |
| AKR7A3 Aflatoxin B1 aldehyde reductase member 3 | IPI00293721 | MAPK1 Mitogen-activated protein kinase 1 | IPI00003479 |
| ALDH1A1 Retinal dehydrogenase 1 | IPI00218914 | MAPRE1 Microtubule-associated protein RP/EB family | IPI00017596 |
| | | member 1 | |
| ALDH3A2 Isoform 1 of Fatty aldehyde dehydrogenase | IPI00333619, IPI00394758 | MARCKS Myristoylated alanine-rich C-kinase substrate | IPI00219301 |
| ALDH9A1 aldehyde dehydrogenase 9A1 | IPI00479877 | MARCKSL1 MARCKS-related protein | IPI00641181 |
| AMBP AMBP protein precursor | IPI00022426 | MARS Methionyl-tRNA synthetase, cytoplasmic | IPI00008240 |
| ANGPTL4 Angiopoietin-related protein 4 precursor | IPI00153060, IPI00740170 | MAT2A S-adenosylmethionine synthetase isoform type-2 | IPI00010157 |
| ANLN Isoform 2 of Actin-binding protein anillin | IPI00032958, IPI00657687, IPI00743594 | MAT2B methionine adenosyltransferase II, beta isoform 1 | IPI00002324, IPI00181717 |
| ANP32A Acidic leucine-rich nuclear phosphoprotein 32 | IPI00025849, IPI00449263 | MATN2 Isoform 2 of Matrilin-2 precursor | IPI00168520, IPI00473118, |
| family member A | | | IPI00554542 |
| ANP32B Isoform 1 of Acidic leucine-rich nuclear | IPI00007423, IPI00759824 | MATN3 Matrilin-3 precursor | IPI00005690 |
| phosphoprotein 32 family member B | | | |
| ANP32E Acidic leucine-rich nuclear phosphoprotein 32 | IPI00165393, IPI00640833 | MATR3 100 kDa protein | IPI00789551 |
| family member E | | | |
| ANTXR1 Isoform 1 of Anthrax toxin receptor 1 precursor | IPI00030431 | MATR3 Matrin-3 | IPI00017297, IPI00789551 |
| ANXA11 Annexin A11 | IPI00414320 | MBTPS1 MBTPS1 protein | IPI00397466 |
| AP3D1 Isoform 1 of AP-3 complex subunit delta-1 | IPI00411453, IPI00477622, IPI00719680 | MCAM Isoform 1 of Cell surface glycoprotein MUC18 | IPI00016334 |
| | | precursor | |
| APEH Acylamino-acid-releasing enzyme | IPI00337741 | MCM3 DNA replication licensing factor MCM3 | IPI00013214 |
| APEX1 DNA-(apurinic or apyrimidinic site) lyase | IPI00215911 | MCM4 DNA replication licensing factor MCM4 | IPI00018349 |
| API5 Isoform 4 of Apoptosis inhibitor 5 | IPI00006684, IPI00554742, IPI00555572 | MCM6 DNA replication licensing factor MCM6 | IPI00031517 |
| APLP1 Amyloid-like protein 1 precursor | IPI00020012, IPI00796118 | MCM7 Isoform 1 of DNA replication licensing factor MCM7 | IPI00299904, IPI00376143 |
| APLP2 Isoform 1 of Amyloid-like protein 2 precursor | IPI00031030 | MDC1 Isoform 1 of Mediator of DNA damage checkpoint | IPI00552897 |
| | | protein 1 | |
| APOA1BP Apolipoprotein A-I binding protein | IPI00514157 | MDK Midkine precursor | IPI00010333 |
| APOA1BP apolipoprotein A-I binding protein precursor | IPI00168479 | ME1 NADP-dependent malic enzyme | IPI00008215 |
| APRT Adenine phosphoribosyltransferase | IPI00218693 | MESDC2 Mesoderm development candidate 2 | IPI00399089 |
| ARCN1 Coatomer subunit delta | IPI00514053 | MET Isoform 1 of Hepatocyte growth factor receptor | IPI00029273, IPI00294528, |
| | | precursor | IPI00792387 |
| ARF1 ADP-ribosylation factor 1 | IPI00215914, IPI00215917 | METAP2 CDNA FLJ34411 fis, clone HEART2002220, | IPI00300763, IPI00789396 |
| | | highly similar to METHIONINE AMINOPEPTIDASE 2 | |
| ARF3 ADP-ribosylation factor 3 | IPI00215917 | METAP2 Similar to methionyl aminopeptidase 2 | IPI00789396 |
| ARFIP1 Isoform A of Arfaptin-1 | IPI00216520 | MFAP2 Microfibrillar-associated protein 2 precursor | IPI00022621, IPI00644827 |
| ARL3 ADP-ribosylation factor-like protein 3 | IPI00003327 | MFGE8 Lactadherin precursor | IPI00002236 |
| ARMET ARMET protein precursor | IPI00328748 | MGAT2 Alpha-1,6-mannosyl-glycoprotein 2-beta-N- | IPI00025809 |
| | | acetylglucosaminyltransferase | |
| ARPC4 72 kDa protein | IPI00790262 | MGAT4A mannosyl (alpha-1,3-)-glycoprotein beta-1,4-N- | IPI00016743 |
| | | acetylglucosaminyltransferase, isoenzyme A | |
| ASAH1 N-acylsphingosine amidohydrolase (acid | IPI00418446 | MGAT4B mannosyl (alpha-1,3-)-glycoprotein beta-1,4-N- | IPI00395751, IPI00744230 |
| ceramidase) 1 isoform b | | acetylglucosaminyltransferase, isoenzyme B isoform 2 | |
| ASCC3L1 U5 small nuclear ribonucleoprotein 200 kDa | IPI00420014 | MGAT5 Alpha-1,6-mannosylglycoprotein 6-beta-N- | IPI00020407 |
| helicase | | acetylglucosaminyltransferase V | |
| ASPH 26 kDa protein | IPI00024572, IPI00032450, IPI00294834, | MGC11102 CDNA FLJ36810 fis, clone ASTRO2001249 | IPI00298618 |
| | IPI00783284, IPI00783617 | | |
| ASPH Aspartyl/asparaginyl beta-hydroxylase | IPI00783284 | MIA3 similar to melanoma inhibitory activity 3 isoform 1 | IPI00374065, IPI00455473, |
| | | | IPI00739902, IPI00740837, |
| | | | IPI00741107 |
| ASRGL1 asparaginase-like 1 protein | IPI00555734 | MICB MHC class I antigen | IPI00640150, IPI00647961 |
| ASS1 ArgininosuccinAte synthetAse | IPI00020632 | MIF Macrophage migration inhibitory factor | IPI00293276 |
| ATIC Bifunctional purine biosynthesis protein PURH | IPI00289499 | MINPP1 Isoform 1 of Multiple inositol polyphosphate | IPI00293748 |
| | | phosphatase 1 precursor | |
| ATOX1 Copper transport protein ATOX1 | IPI00010863 | MKI67 Isoform Long of Antigen KI-67 | IPI00004233 |
| ATP1A1 Isoform Long of Sodium/potassium-transporting | IPI00006482 | MLLT4 Isoform 4 of Afadin | IPI00023461, IPI00759546 |
| ATPase alpha-1 chain precursor | | | |
| ATP1A2 Sodium/potassium-transporting ATPase alpha-2 | IPI00003021, IPI00302840, IPI00640401, | MMP10 Stromelysin-2 precursor | IPI00013405 |
| chain precursor | IPI00788782 | | |
| ATP1B3 Sodium/potassium-transporting ATPase subunit | IPI00008167 | MMP11 Stromelysin-3 precursor | IPI00306778 |
| beta-3 | | | |
| ATP2A2 115 kDa protein | IPI00747443 | MMP2 72 kDa type IV collagenase precursor | IPI00027780 |
| ATP5A1 ATP synthase subunit alpha, mitochondrial | IPI00440493 | MRC2 Macrophage mannose receptor 2 precursor | IPI00005707 |
| precursor | | | |
| ATP5B ATP synthase subunit beta, mitochondrial precursor | IPI00303476 | MRPL12 39S ribosomal protein L12, mitochondrial | IPI00005537 |
| | | precursor | |
| ATP5J ATP synthase coupling factor 6, mitochondrial | IPI00002521, IPI00456008 | MRPS16; DNAJC9 DnaJ homolog subfamily C member 9 | IPI00154975 |
| precursor | | | |
| ATP6AP1 53 kDa protein | IPI00020430, IPI00784119, IPI00794988 | MTA2 Metastasis-associated protein MTA2 | IPI00171798 |
| ATP6AP2 Protein | IPI00168884, IPI00828107 | MTAP S-methyl-5-thioadenosine phosphorylase | IPI00011876 |
| ATP6AP2 Renin receptor precursor | IPI00168884 | MTHFD1 C-1-tetrahydrofolate synthase, cytoplasmic | IPI00218342, IPI00794900 |
| ATP6V1A Vacuolar ATP synthase catalytic subunit A, | IPI00007682 | MTPN Myotrophin | IPI00179589 |
| ubiquitous isoform | | | |
| ATP6V1B2 Vacuolar ATP synthase subunit B, brain isoform | IPI00007812 | MUC13 Mucin-13 precursor | IPI00011448 |
| ATP6V1G2; BAT1 BAT1 protein | IPI00641829 | MYH9 Myosin-9 | IPI00019502 |
| ATRN Isoform 1 of Attractin precursor | IPI00027235, IPI00478671 | MYL6 19 kDa protein | IPI00797001 |
| AXL AXL receptor tyrosine kinase isoform 1 | IPI00296992, IPI00397361 | MYL6 Isoform Non-muscle of Myosin light polypeptide 6 | IPI00335168, IPI00413922, |
| | | | IPI00744444, IPI00789605, |
| | | | IPI00795576, IPI00796366, |
| | | | IPI00797001, IPI00797626 |
| B3GALT6 Beta-1,3-galactosyltransferase 6 | IPI00064848 | MYL6 Isoform Smooth muscle of Myosin light polypeptide 6 | IPI00789605 |
| B3GAT3 B3GAT3 protein (Fragment) | IPI00477470 | MYO1C myosin IC isoform a | IPI00743335 |
| B3GAT3 Galactosylgalactosylxylosylprotein 3-beta- | IPI00304331, IPI00477470 | MYO6 Isoform 2 of Myosin-VI | IPI00008455, IPI00069126, |
| glucuronosyltransferase 3 | | | IPI00642722, IPI00816452, |
| | | | IPI00816461 |
| B3GNT1 N-acetyllactosaminide beta-1,3-N- | IPI00009997 | NACA Similar to Nascent polypeptide associated complex | IPI00023748, IPI00797126 |
| acetylglucosaminyltransferase | | alpha subunit | |
| B3GNT2 Isoform 2 of UDP-GlcNAc:betaGal beta-1,3-N- | IPI00217345 | NAGLU Alpha-N-acetylglucosaminidase precursor | IPI00008787 |
| acetylglucosaminyltransferase 2 | | | |
| B4GALT3 44 kDa protein | IPI00746600 | NANS Sialic acid synthase | IPI00147874 |
| B4GALT4 Beta-1,4-galactosyltransferase 4 | IPI00030128 | NAP1L1 Nucleosome assembly protein 1-like 1 | IPI00023860, IPI00789029, |
| | | | IPI00791065, IPI00793389 |
| BAG2 BAG family molecular chaperone regulator 2 | IPI00000643 | NAP1L4 Nucleosome assembly protein 1-like 4 | IPI00017763 |
| BAG3 BAG family molecular chaperone regulator 3 | IPI00641582 | NAPG Gamma-soluble NSF attachment protein | IPI00293817 |
| BANF1 Barrier-to-autointegration factor | IPI00026087 | NARS Asparaginyl-tRNA synthetase, cytoplasmic | IPI00306960, IPI00646656 |
| BASP1 Brain acid soluble protein 1 | IPI00299024 | NASP Isoform 1 of Nuclear autoantigenic sperm protein | IPI00179953 |
| BAT2 Isoform 1 of Large proline-rich protein BAT2 | IPI00010700, IPI00642817 | NCAM1 Neural cell adhesion molecule 1, 120 kDa isoform | IPI00555628 |
| | | variant (Fragment) | |
| BCCIP BRCA2 and CDKN1A-interacting protein isoform C | IPI00068254 | NCAM1 Neural cell adhesion molecule 1, 140 kDa isoform | IPI00435020, IPI00795918 |
| | | precursor | |
| BCCIP Isoform 2 of BRCA2 and CDKN1A-interacting protein | IPI00220152 | NCBP1 Nuclear cap-binding protein subunit 1 | IPI00019380 |
| BCHE Cholinesterase precursor | IPI00025864 | NCL Isoform 1 of Nucleolin | IPI00604620 |
| BCLAF1 Isoform 1 of Bcl-2-associated transcription factor 1 | IPI00006079, IPI00413671 | NCL Isoform 2 of Nucleolin | IPI00827674 |
| BDNF Brain-derived neurotrophic factor precursor | IPI00012058 | NCOA5 Nuclear receptor coactivator 5 | IPI00288941 |
| BGN Biglycan precursor | IPI00010790, IPI00643384 | NDRG1 Protein NDRG1 | IPI00022078, IPI00183085, |
| | | | IPI00783586 |
| BID Isoform 1 of BH3-interacting domain death agonist | IPI00413587, IPI00420084 | NDUFV2 NADH dehydrogenase [ubiquinone] flavoprotein 2, | IPI00291328, IPI00412122, |
| | | mitochondrial precursor | IPI00646556 |
| BLMH Bleomycin hydrolase | IPI00219575 | NEDD8 NEDD8 precursor | IPI00020008 |
| BLVRA Biliverdin reductase A precursor | IPI00294158 | NELL1 Protein kinase C-binding protein NELL1 precursor | IPI00023754 |
| BMP1 Isoform BMP1-1 of Bone morphogenetic protein 1 | IPI00014021 | NELL2 Cerebral protein-12 | IPI00795624 |
| precursor | | | |
| BMP1 Isoform BMP1-5 of Bone morphogenetic protein 1 | IPI00218042 | NENF Neudesin precursor | IPI00002525 |
| precursor | | | |
| BOLA2B; BOLA2 BolA-like protein 2 isoform a | IPI00301434 | NEO1 Isoform 1 of Neogenin precursor | IPI00023814, IPI00217291, |
| | | | IPI00472011 |
| BPNT1 Isoform 1 of 3′(2′),5′-bisphosphate nucleotidase 1 | IPI00410214 | NEO1 Isoform 2 of Neogenin precursor | IPI00217291 |
| BSG 46 kDa protein | IPI00795150 | NEU1 Sialidase-1 precursor | IPI00029817 |
| BSG Isoform 2 of Basigin precursor | IPI00019906, IPI00218019, IPI00795150 | NFASC Isoform 7 of Neurofascin precursor | IPI00384998, IPI00470575, |
| | | | IPI00513695, IPI00513705, |
| | | | IPI00655702, IPI00655890, |
| | | | IPI00656129 |
| BST1 ADP-ribosyl cyclase 2 precursor | IPI00026240, IPI00657860 | NHP2L1 NHP2-like protein 1 | IPI00026167 |
| BTD biotinidase precursor | IPI00218413 | NID1 Isoform 1 of Nidogen-1 precursor | IPI00026944 |
| BTF3 Isoform 2 of Transcription factor BTF3 | IPI00419473 | NID2 Nidogen-2 precursor | IPI00028908 |
| BUB3 Mitotic checkpoint protein BUB3 | IPI00013468, IPI00514701, IPI00644108 | NIPSNAP1 Protein NipSnap1 | IPI00304435 |
| C11orf58 Small acidic protein | IPI00003419 | NIPSNAP3A Protein NipSnap3A | IPI00004845 |
| C12orf39 Uncharacterized protein C12orf39 precursor | IPI00012236 | NIT1 Isoform 2 of Nitrilase homolog 1 | IPI00023779, IPI00456663, |
| | | | IPI00456664, IPI00456665, |
| | | | IPI00646277 |
| C12orf5 Uncharacterized protein C12orf5 | IPI00006907 | NIT2 Nitrilase family member 2 | IPI00549467 |
| C13orf8 Zinc finger protein KIAA1802 | IPI00064212 | NMB Isoform 1 of Neuromedin-B precursor | IPI00031753, IPI00220630 |
| C14orf141; LTBP2 Latent-transforming growth factor beta- | IPI00292150 | NNT NAD(P) transhydrogenase, mitochondrial precursor | IPI00337541 |
| binding protein 2 precursor | | | |
| C14orf156 SRA stem-loop-interacting RNA-binding protein, | IPI00009922 | NOL1 Isoform 1 of Putative RNA methyltransferase NOL1 | IPI00654555 |
| mitochondrial precursor | | | |
| C14orf78 similar to AHNAK nucleoprotein isoform 1 | IPI00479767 | NOL5A Nucleolar protein Nop56 | IPI00411937 |
| C19orf10 Uncharacterized protein C19orf10 precursor | IPI00056357 | NOMO1 Nodal modulator 1 precursor | IPI00329352 |
| C1QBP Complement component 1 Q subcomponent- | IPI00014230 | NONO Non-POU domain-containing octamer-binding | IPI00304596 |
| binding protein, mitochondrial precursor | | protein | |
| C1QTNF3; AMACR Alpha-methyl-acyl-CoA racemase | IPI00005918 | NOP5/NOP58 Nucleolar protein NOP5 | IPI00006379 |
| C1R Complement C1r subcomponent precursor | IPI00296165 | NOTCH2 Neurogenic locus notch homolog protein 2 | IPI00297655 |
| | | precursor | |
| C1RL Complement C1r-like protein | IPI00009793, IPI00795055 | NOTCH3 Neurogenic locus notch homolog protein 3 | IPI00029819 |
| | | precursor | |
| C1S Complement C1s subcomponent precursor | IPI00017696 | NOTUM IMP dehydrogenase/GMP reductase family protein | IPI00465159 |
| C20orf77 Uncharacterized protein C20orf77 | IPI00009659 | NOV Protein NOV homolog precursor | IPI00011140 |
| C21orf33 Isoform Long of ES1 protein homolog, | IPI00024913, IPI00218482, IPI00784162, | NPC2 Epididymal secretory protein E1 precursor | IPI00301579 |
| mitochondrial precursor | IPI00784277, IPI00793677 | | |
| C22orf28 UPF0027 protein C22orf28 | IPI00550689 | NPM1 Isoform 1 of Nucleophosmin | IPI00549248 |
| C3orf17 Isoform 1 of Uncharacterized protein C3orf17 | IPI00295519, IPI00607570 | NPM1 Isoform 2 of Nucleophosmin | IPI00220740, IPI00549248 |
| C4orf18 Uncharacterized protein C4orf18 | IPI00165044, IPI00414183 | NPTX1 Neuronal pentraxin-1 precursor | IPI00220562 |
| C4orf31 hypothetical protein LOC79625 | IPI00152148 | NPTX1 similar to neuronal pentraxin I precursor | IPI00787050 |
| C5 Complement component 5 variant (Fragment) | IPI00816741 | NPTX2 Neuronal pentraxin-2 precursor | IPI00026946 |
| C6orf108 c-Myc-responsive protein Rcl | IPI00007926 | NQO1 Hypothetical protein (Fragment) | IPI00619898 |
| C6orf15 Uncharacterized protein C6orf15 precursor | IPI00022381 | NQO1 NAD | IPI00012069, IPI00619898, |
| | | | IPI00619966 |
| C7orf24 Uncharacterized protein C7orf24 | IPI00031564 | NQO2 Ribosyldihydronicotinamide dehydrogenase | IPI00219129, IPI00515016 |
| CA2 Carbonic anhydrase 2 | IPI00218414 | NRCAM Isoform 1 of Neuronal cell adhesion molecule | IPI00333776, IPI00655818 |
| | | precursor | |
| CACYBP Isoform 1 of Calcyclin-binding protein | IPI00395627, IPI00552308, IPI00745851 | NRCAM Isoform 3 of Neuronal cell adhesion molecule | IPI00333778 |
| | | precursor | |
| CAD CAD protein | IPI00301263 | NRCAM NRCAM protein | IPI00783655 |
| CADM1 Nectin-like protein 2 | IPI00003813, IPI00166392 | NRP1 Isoform 1 of Neuropilin-1 precursor | IPI00299594, IPI00398715 |
| CALCA Isoform 1 of Calcitonin precursor | IPI00000914 | NRP1 Muscle type neuropilin 1 | IPI00165438, IPI00299594, |
| | | | IPI00398715, IPI00549340, |
| | | | IPI00607733, IPI00639917, |
| | | | IPI00749187 |
| CALD1 Isoform 4 of Caldesmon | IPI00218696, IPI00333771 | NRP2 neuropilin 2 isoform 4 precursor | IPI00300890, IPI00641131 |
| CALM3; CALM2; CALM1 Calmodulin | IPI00075248, IPI00386621, IPI00794543 | NSF Vesicle-fusing ATPase | IPI00006451 |
| CALU Calumenin precursor | IPI00789155 | NSFL1C Isoform 1 of NSFL1 cofactor p47 | IPI00100197, IPI00397571 |
| CALU isoform 1 of Calumenin precursor | IPI00014537, IPI00789155 | NSFL1C Isoform 2 of NSFL1 cofactor p47 | IPI00022830, IPI00100197, |
| | | | IPI00376904, IPI00397571 |
| CAMK2D Isoform Delta 6 of Calcium/calmodulin-dependent | IPI00172636, IPI00430291, IPI00827573, | NSFL1C p47 protein isoform c | IPI00376904 |
| protein kinase type II delta chain | IPI00827625, IPI00828081, | | |
| | IPI00828139, IPI00828178 | | |
| CAND1 Isoform 1 of Cullin-associated NEDD8-dissociated | IPI00100160, IPI00604431 | NT5C3 Isoform 2 of Cytosolic 5′-nucleotidase III | IPI00807412 |
| protein 1 | | | |
| CANT1 Isoform 1 of Soluble calcium-activated nucleotidase 1 | IPI00103175 | NTN4 Isoform 3 of Netrin-4 precursor | IPI00385236 |
| CANX Calnexin precursor | IPI00020984 | NUCB2 Nucleobindin-2 precursor | IPI00009123, IPI00746961 |
| CAPN1 Calpain-1 catalytic subunit | IPI00011285 | NUCKS1 Isoform 1 of Nuclear ubiquitous casein and cyclin- | IPI00022145 |
| | | dependent kinases substrate | |
| CARHSP1 Calcium-regulated heat stable protein 1 | IPI00304409 | NUDC Nuclear migration protein nudC | IPI00550746, IPI00646767, |
| | | | IPI00647418 |
| CARS cysteinyl-tRNA synthetase isoform c | IPI00027443, IPI00158625, IPI00556365, | NUDT21 Cleavage and polyadenylation specificity factor 5 | IPI00646917 |
| | IPI00556541 | | |
| CAST calpastatin isoform e | IPI00760715, IPI00761035, IPI00761069, | NUDT5 ADP-sugar pyrophosphatase | IPI00296913, IPI00646762 |
| | IPI00761140, IPI00761160 | | |
| CAST Isoform 2 of Calpastatin | IPI00220857, IPI00305750, IPI00760715, | NUDT9 Isoform 1 of ADP-ribose pyrophosphatase, | IPI00031558 |
| | IPI00761035, IPI00761069, | mitochondrial precursor | |
| | IPI00761140, IPI00761160 | | |
| CBFB Core-binding factor subunit beta | IPI00016746, IPI00024871 | NUMA1 Isoform 2 of Nuclear mitotic apparatus protein 1 | IPI00006196, IPI00292771 |
| CBR1 Carbonyl reductase [NADPH] 1 | IPI00295386 | NUTF2 Nuclear transport factor 2 | IPI00009901 |
| CBX1 Chromobox protein homolog 1 | IPI00010320 | OAF Protein OAF homolog | IPI00328703 |
| CBX3; LOC653972 Chromobox protein homolog 3 | IPI00297579 | OCIAD1 OCIA domain containing 1 isoform 1 | IPI00016405 |
| CBX5 Chromobox protein homolog 5 | IPI00024662 | OLFM1 Isoform 1 of Noelin precursor | IPI00017841, IPI00419820, |
| | | | IPI00472517, IPI00550145 |
| CCAR1 Cell division cycle and apoptosis regulator protein 1 | IPI00217357 | OLFML2A CDNA FLJ90228 fis, clone NT2RM2000241 | IPI00184375 |
| | | (Fragment) | |
| CCDC25 CCDC25 protein | IPI00797903 | OLFML2A olfactomedin-like 2A | IPI00385326 |
| CCDC80 steroid-sensitive protein 1 | IPI00260630 | OLFML3 Isoform 1 of Olfactomedin-like protein 3 precursor | IPI00024621, IPI00607652 |
| CCL2 Small inducible cytokine A2 precursor | IPI00009308 | OPA1 Isoform 1 of Dynamin-like 120 kDa protein, | IPI00006721, IPI00107749, |
| | | mitochondrial precursor | IPI00107750, IPI00107751, |
| | | | IPI00107752, IPI00107753, |
| | | | IPI00375149, IPI00375150, |
| | | | IPI00789199, |
| | | | IPI00797488 |
| CCT2 T-complex protein 1 subunit beta | IPI00297779 | OS9 76 kDa protein | IPI00329760, IPI00604451, |
| | | | IPI00784387 |
| CCT3 chaperonin containing TCP1, subunit 3 isoform b | IPI00290770, IPI00553185, IPI00744315 | OS9 Isoform OS-9-2 of Protein OS-9 precursor | IPI00186581 |
| CCT4 T-complex protein 1 subunit delta | IPI00302927 | 0S9 Isoform OS-9-3 of Protein OS-9 precursor | IPI00398855 |
| CCT5 T-complex protein 1 subunit epsilon | IPI00010720 | OTUB1 Hypothetical protein DKFZp564E242 | IPI00000581, IPI00409750 |
| CCT6A T-complex protein 1 subunit zeta | IPI00027626 | OTUB1 Isoform 2 of Ubiquitin thioesterase protein OTUB1 | IPI00409750 |
| CCT7 T-complex protein 1 subunit eta | IPI00018465 | P4HA1 Isoform 1 of Prolyl 4-hydroxylase subunit alpha-1 | IPI00009923 |
| | | precursor | |
| CCT8 Chaperonin containing TCP1, subunit 8 (Theta) | IPI00302925, IPI00784090 | P704P similar to actin-like protein | IPI00748022, IPI00786945 |
| variant | | | |
| CD109 Isoform 1 of CD109 antigen precursor | IPI00152540 | PA2G4 Proliferation-associated protein 2G4 | IPI00299000, IPI00794875 |
| CD14 Monocyte differentiation antigen CD14 precursor | IPI00029260 | PABPC1 70 kDa protein | IPI00796945 |
| CD276 Isoform 1 of CD276 antigen precursor | IPI00410488, IPI00441094, IPI00719044, | PABPC1 Isoform 1 of Polyadenylate-binding protein 1 | IPI00008524, IPI00410017, |
| | IPI00793688 | | IPI00478522, IPI00796945 |
| CD3EAP Isoform 2 of RNA polymerase I-associated factor | IPI00012788, IPI00645816 | PABPC4 Isoform 2 of Polyadenylate-binding protein 4 | IPI00555747 |
| PAF49 | | | |
| CD44 Isoform 12 of CD44 antigen precursor | IPI00297160, IPI00305064, IPI00418465, | PABPN1 Isoform 1 of Polyadenylate-binding protein 2 | IPI00005792, IPI00414963 |
| | IPI00419219, IPI00827650, | | |
| | IPI00827658, IPI00827795, | | |
| | IPI00827893, IPI00827937, | | |
| | IPI00827982, IPI00828056, | | |
| | IPI00828064, IPI00828117, IPI00828192 | | |
| CD44 Isoform 4 of CD44 antigen precursor | IPI00305064, IPI00418465, IPI00827555, | PAFAH1B1 Isoform 1 of Platelet-activating factor | IPI00218728 |
| | IPI00827650, IPI00827658, | acetylhydrolase IB subunit alpha | |
| | IPI00827795, IPI00828056, | | |
| | IPI00828117 | | |
| CD44 Isoform CD44 of CD44 antigen precursor | IPI00305064, IPI00418465, IPI00419219, | PAFAH1B2 Platelet-activating factor acetylhydrolase IB | IPI00026546 |
| | IPI00827555, IPI00827650, | subunit beta | |
| | IPI00827658, IPI00827795, | | |
| | IPI00827893, IPI00828056, | | |
| | IPI00828064, IPI00828117, | | |
| | IPI00828192 | | |
| CD46 CD46 antigen, complement regulatory protein isoform | IPI00219853, IPI00374176, IPI00374177, | PAFAH1B3 Platelet-activating factor acetylhydrolase IB | IPI00014808 |
| 13 precursor | IPI00398353, IPI00456644 | subunit gamma | |
| CD55 Isoform 1 of Complement decay-accelerating factor | IPI00216550 | PAGE2B Putative G antigen family E member 3 | IPI00402613, IPI00640518, |
| precursor | | | IPI00647526 |
| CD70 Tumor necrosis factor ligand superfamily member 7 | IPI00031713 | PAICS Multifunctional protein ADE2 | IPI00217223 |
| CD81 CD81 antigen | IPI00000190 | PAK2 Serine/threonine-protein kinase PAK 2 | IPI00419979 |
| CDC37 Hsp90 co-chaperone Cdc37 | IPI00013122 | PAK2 similar to p21-activated kinase 2 | IPI00787208 |
| CDC42 Isoform 1 of Cell division control protein 42 homolog | IPI00007189 | PAM Isoform 1 of Peptidyl-glycine alpha-amidating | IPI00177543, IPI00219041, |
| precursor | | monooxygenase precursor | IPI00219042, IPI00219043, |
| | | | IPI00749176 |
| CDC5L Cell division cycle 5-like protein | IPI00465294 | PAM Isoform 3 of Peptidyl-glycine alpha-amidating | IPI00219042 |
| | | monooxygenase precursor | |
| CDH10 Cadherin-10 precursor | IPI00295399 | PAPPA Pappalysin-1 precursor | IPI00001869, IPI00513756 |
| CDH11 Isoform 2 of Cadherin-11 precursor | IPI00293539 | PAPSS1 Bifunctional 3′-phosphoadenosine 5′- | IPI00011619 |
| | | phosphosulfate synthetase 1 | |
| CDH13 Cadherin-13 precursor | IPI00024046 | PARK7 Protein DJ-1 | IPI00298547 |
| CDH17 Cadherin-17 precursor | IPI00290089 | PARP1 Poly [ADP-ribose] polymerase 1 | IPI00449049 |
| CDH2 Cadherin-2 precursor | IPI00290085 | PBEF1 Isoform 1 of Nicotinamide | IPI00018873, IPI00472879 |
| | | phosphoribosyltransferase | |
| CDH3 Cadherin-3 precursor | IPI00216677, IPI00645614, IPI00747243 | PCBD1 Pterin-4-alpha-carbinolamine dehydratase | IPI00218568 |
| CDH3 CDH3 protein | IPI00645614, IPI00747243 | PCBP1 Poly(rC)-binding protein 1 | IPI00016610 |
| CDV3 Protein CDV3 homolog | IPI00014197, IPI00787933 | PCBP2 poly(rC)-binding protein 2 isoform b | IPI00012066, IPI00216689, |
| | | | IPI00796337 |
| CEACAM5 Carcinoembryonic antigen-related cell adhesion | IPI00027486 | PCDH1 protocadherin 1 isoform 2 precursor | IPI00176458, IPI00215992 |
| molecule 5 precursor | | | |
| CEL Carboxyl ester lipase | IPI00099670 | PCDH17 Isoform 1 of Protocadherin-17 precursor | IPI00645206 |
| CES1 Isoform 2 of Liver carboxylesterase 1 precursor | IPI00607801 | PCDH19 Isoform 1 of Protocadherin-19 precursor | IPI00552819 |
| CFD complement factor D preproprotein | IPI00165972 | PCDH7 Isoform B of Protocadherin-7 precursor | IPI00215946 |
| CFDP1 Isoform 1 of Craniofacial development protein 1 | IPI00007306 | PCDHB10 Protocadherin beta 10 precursor | IPI00009034 |
| CFHR3 Complement factor H-related 3 | IPI00654723 | PCDHB5 Protocadherin beta 5 precursor | IPI00001428 |
| CGREF1 Cell growth regulator with EF hand domain protein 1 | IPI00783540 | PCID1 PNAS-125 | IPI00000495 |
| CHERP calcium homeostasis endoplasmic reticulum protein | IPI00333010 | PCK2 Phosphoenolpyruvate carboxykinase [GTP], | IPI00294380, IPI00797038 |
| | | mitochondrial precursor | |
| CHGA chromogranin A precursor | IPI00746813 | PCMT1 Isoform 1 of Protein-L-isoaspartate(D-aspartate) O- | IPI00411680, IPI00828189 |
| | | methyltransferase | |
| CHGB Secretogranin-1 precursor | IPI00006601 | PCMT1 Protein-L-isoaspartate (D-aspartate) O- | IPI00024989 |
| | | methyltransferase | |
| ChGn Isoform 1 of Chondroitin beta-1,4-N- | IPI00216738 | PCNA Proliferating cell nuclear antigen | IPI00021700 |
| acetylgalactosaminyltransferase 1 | | | |
| CHID1 Isoform 2 of Chitinase domain-containing protein 1 | IPI00301185, IPI00306719 | PCOLCE Procollagen C-endopeptidase enhancer 1 | IPI00299738 |
| precursor | | precursor | |
| CHL1 Isoform 1 of Neural cell adhesion molecule L1-like | IPI00783390 | PCOLCE2 Procollagen C-endopeptidase enhancer 2 | IPI00002543 |
| protein precursor | | precursor | |
| CHL1 Isoform 2 of Neural cell adhesion molecule L1-like | IPI00299059 | PCSK1 Neuroendocrine convertase 1 precursor | IPI00301961 |
| protein precursor | | | |
| CHORDC1 Cysteine and histidine-rich domain-containing | IPI00015897 | PCSK1N ProSAAS precursor | IPI00002280 |
| protein 1 | | | |
| CHPF Chondroitin sulfate synthase 2 | IPI00465319 | PCSK2 Neuroendocrine convertase 2 precursor | IPI00029131, IPI00643663 |
| CHRDL1 Ventroptin (Fragment) | IPI00478414, IPI00654588 | PCSK5 Proprotein convertase subtilisin/kexin type 5 | IPI00165229, IPI00294862 |
| CHST11 Isoform 1 of Carbohydrate sulfotransferase 11 | IPI00099831, IPI00554485 | PCSK6 Isoform PACE4E-II of Proprotein convertase | IPI00238849 |
| | | subtilisin/kexin type 6 precursor | |
| CIAPIN1 Isoform 3 of Anamorsin | IPI00025333, IPI00387130 | PCSK9 Isoform 1 of Proprotein convertase subtilisin/kexin | IPI00387168 |
| | | type 9 precursor | |
| CILP2 Similar to Cartilage intermediate layer protein | IPI00216780 | PDAP1 28 kDa heat- and acid-stable phosphoprotein | IPI00013297 |
| CIRBP 32 kDa protein | IPI00180954, IPI00641579, IPI00646241 | PDCD6 Programmed cell death protein 6 | IPI00025277 |
| CKB Creatine kinase B-type | IPI00022977 | PDCD6IP PDCD6IP protein | IPI00246058 |
| CKMT1B; CKMT1A Creatine kinase, ubiquitous | IPI00658109 | PDGFC platelet-derived growth factor C precursor | IPI00099977 |
| mitochondrial precursor | | | |
| CLEC11A C-type lectin domain family 11 member A | IPI00033466 | PDGFD Isoform 2 of Platelet-derived growth factor D | IPI00011865, IPI00018822 |
| precursor | | precursor | |
| CLIC4 Chloride intracellular channel protein 4 | IPI00001960 | PDGFRL Platelet-derived growth factor receptor-like protein | IPI00006236 |
| | | precursor | |
| CLINT1 Isoform 1 of Clathrin interactor 1 | IPI00291930, IPI00397519 | PDIA4 Protein disulfide-isomerase A4 precursor | IPI00009904 |
| CLIP1 CLIP1 protein | IPI00013455, IPI00027172, IPI00217113 | PDLIM5 PDZ and LIM domain protein 5 | IPI00007935 |
| CLSTN2 Calsyntenin-2 precursor | IPI00005491 | PEA15 Astrocytic phosphoprotein PEA-15 | IPI00014850, IPI00643342 |
| CLSTN3 Alcadein beta | IPI00396423, IPI00747063 | PENK Proenkephalin A precursor | IPI00000828 |
| CLSTN3 Calsyntenin-3 precursor | IPI00747063 | PEPD Xaa-Pro dipeptidase | IPI00257882 |
| CMPK cytidylate kinase | IPI00219953 | PFAS Phosphoribosylformylglycinamidine synthase | IPI00004534 |
| CNBP Isoform 2 of Cellular nucleic acid-binding protein | IPI00430813, IPI00430814 | PFDN4 Prefoldin subunit 4 | IPI00015891 |
| CNBP Zinc finger protein 9 | IPI00430812 | PFKP Phosphofructokinase, platelet | IPI00643196 |
| CNN3 Calponin-3 | IPI00216682 | PFN2 Isoform IIb of Profilin-2 | IPI00107555, IPI00219468 |
| CNTN1 Isoform 1 of Contactin-1 precursor | IPI00029751 | PGM2 Phosphoglucomutase-2 | IPI00550364 |
| COCH Cochlin precursor | IPI00012386 | PGRMC1 Membrane-associated progesterone receptor | IPI00220739 |
| | | component 1 | |
| COL12A1 316 kDa protein | IPI00827558 | PHGDH D-3-phosphoglycerate dehydrogenase | IPI00011200 |
| COL4A1 Collagen alpha-1(IV) chain precursor | IPI00743696 | PIN1 Peptidyl-prolyl cis-trans isomerase NIMA-interacting 1 | IPI00013723 |
| COL4A2 Collagen alpha-2(IV) chain precursor | IPI00306322 | PITX1; H2AFY H2A histone family, member Y isoform 2 | IPI00059366, IPI00304171, |
| | | | IPI00744148 |
| COL4A6 Isoform B of Collagen alpha-6(IV) chain precursor | IPI00472200 | PLA2G3 Group 3 secretory phospholipase A2 precursor | IPI00024578 |
| COL5A1 Collagen alpha-1(V) chain precursor | IPI00477611 | PLA2G7 Platelet-activating factor acetylhydrolase precursor | IPI00011588 |
| COL5A2 alpha 2 type V collagen preproprotein | IPI00748917 | PLAT Isoform 1 of Tissue-type plasminogen activator | IPI00019590 |
| | | precursor | |
| COL6A2 Isoform 2C2A of Collagen alpha-2(VI) chain | IPI00220613, IPI00304840 | PLAUR Isoform 1 of Urokinase plasminogen activator | IPI00010676, IPI00215706 |
| precursor | | surface receptor precursor | |
| COL8A1 Cell proliferation-inducing protein 41 | IPI00219000 | PLG Plasminogen precursor | IPI00019580 |
| COPA Coatomer subunit alpha | IPI00295857, IPI00646493 | PLOD2 Isoform 2 of Procollagen-lysine, 2-oxoglutarate 5- | IPI00337495, IPI00472165 |
| | | dioxygenase 2 precursor | |
| COPB1 Coatomer subunit beta | IPI00295851 | PLOD3 Procollagen-lysine, 2-oxoglutarate 5-dioxygenase 3 | IPI00030255 |
| | | precursor | |
| COPB2 Coatomer subunit beta′ | IPI00220219 | PLRG1 Isoform 1 of Pleiotropic regulator 1 | IPI00002624 |
| COPE epsilon subunit of coatomer protein complex isoform b | IPI00399318 | PLS3 plastin 3 | IPI00216694 |
| COPG 98 kDa protein | IPI00001890, IPI00783982 | PLXDC2 Isoform 1 of Plexin domain-containing protein 2 | IPI00044369, IPI00073777, |
| | | precursor | IPI00640599 |
| CORO1B; PTPRCAP Coronin-1B | IPI00007058 | PLXNB2 similar to Plexin-B2 precursor | IPI00398435, IPI00736693 |
| CORO1C Coronin-1C | IPI00008453 | PNN Protein | IPI00418458 |
| COTL1 Coactosin-like protein | IPI00017704 | PNPO Pyridoxine-5′-phosphate oxidase | IPI00018272 |
| COX5A Cytochrome c oxidase subunit 5A, mitochondrial | IPI00025086 | POFUT1 Isoform 1 of GDP-fucose protein O- | IPI00058192 |
| precursor | | fucosyltransferase 1 precursor | |
| COX6B1 Cytochrome c oxidase subunit VIb isoform 1 | IPI00216085, IPI00797738 | POSTN Isoform 1 of Periostin precursor | IPI00007960, IPI00218585, |
| | | | IPI00410241, IPI00641231, |
| | | | IPI00797227 |
| CPD Carboxypeptidase D precursor | IPI00027078 | PPA1 Inorganic pyrophosphatase | IPI00015018 |
| CPE Carboxypeptidase E precursor | IPI00031121 | PPA2 Isoform 1 of Inorganic pyrophosphatase 2, | IPI00301109, IPI00413014, |
| | | mitochondrial precursor | IPI00470503, IPI00654717 |
| CPNE1 59 kDa protein | IPI00640372 | PPIE Isoform B of Peptidyl-prolyl cis-trans isomerase E | IPI00220188 |
| CPNE1; RBM12 RNA-binding protein 12 | IPI00550308 | PPIF Peptidyl-prolyl cis-trans isomerase, mitochondrial | IPI00026519 |
| | | precursor | |
| CPNE3 Copine-3 | IPI00024403 | PPIL3 Isoform 1 of Peptidyl-prolyl cis-trans isomerase-like 3 | IPI00300952 |
| CPS1 Isoform 1 of Carbamoyl-phosphate synthase | IPI00011062 | PPM1A Isoform Alpha-1 of Protein phosphatase 2C isoform | IPI00020950, IPI00216196 |
| [ammonia], mitochondrial precursor | | alpha | |
| CPSF1 Cleavage and polyadenylation specificity factor | IPI00026219 | PPM1E Protein phosphatase 1E | IPI00103630 |
| subunit 1 | | | |
| CPSF2 Cleavage and polyadenylation specificity factor | IPI00419531 | PPM1G Protein phosphatase 2C isoform gamma | IPI00006167 |
| subunit 2 | | | |
| CPSF6 Isoform 1 of Cleavage and polyadenylation | IPI00012998, IPI00030654 | PPP1CA Serine/threonine-protein phosphatase PP1-alpha | IPI00550451, IPI00794133, |
| specificity factor 6 | | catalytic subunit | IPI00797955 |
| CPSF6 Isoform 2 of Cleavage and polyadenylation | IPI00030654 | PPP1CB Serine/threonine-protein phosphatase PP1-beta | IPI00218236 |
| specificity factor 6 | | catalytic subunit | |
| CPVL Probable serine carboxypeptidase CPVL precursor | IPI00301395 | PPP1R14B Similar to Protein phosphatase 1 regulatory | IPI00398922 |
| | | subunit 14B | |
| CRAMP1L; HN1L Isoform 1 of Hematological and | IPI00027397 | PPP2CA Serine/threonine-protein phosphatase 2A catalytic | IPI00008380, IPI00815784 |
| neurological expressed 1-like protein | | subunit alpha isoform | |
| CREG1 Protein CREG1 precursor | IPI00021997 | PPP2R1A alpha isoform of regulatory subunit A, protein | IPI00419307, IPI00554737 |
| | | phosphatase 2 | |
| CRH Corticoliberin precursor | IPI00027839 | PPP5C Serine/threonine-protein phosphatase 5 | IPI00019812, IPI00790133 |
| CRIM1 Cysteine-rich motor neuron 1 protein precursor | IPI00009294 | PPT1 Palmitoyl-protein thioesterase 1 | IPI00514424 |
| CRIP2 Cysteine-rich protein 2 | IPI00006034 | PPT1 Palmitoyl-protein thioesterase 1 precursor | IPI00002412 |
| CRK Isoform Crk-II of Proto-oncogene C-crk | IPI00004838 | PPT2 Isoform 1 of Lysosomal thioesterase PPT2 precursor | IPI00021421, IPI00453232, |
| | | | IPI00552460, IPI00647927, |
| | | | IPI00783249, IPI00789091 |
| CRK v-crk sarcoma virus CT10 oncogene homolog isoform b | IPI00305469 | PRCP prolylcarboxypeptidase isoform 2 preproprotein | IPI00399307 |
| CRKL Crk-like protein | IPI00004839 | PRDX4 Peroxiredoxin-4 | IPI00011937 |
| CRLF1 Cytokine receptor-like factor 1 precursor | IPI00289561 | PRDX5 peroxiredoxin 5 precursor, isoform b | IPI00375306 |
| CRTAP Cartilage-associated protein precursor | IPI00220959, IPI00748502, IPI00793277 | PRKCSH Glucosidase 2 subunit beta precursor | IPI00026154, IPI00792916 |
| CRYZ Quinone oxidoreductase | IPI00000792, IPI00641565, IPI00647366 | PRMT5 protein arginine methyltransferase 5 isoform b | IPI00064328, IPI00441473 |
| CS Citrate synthase, mitochondrial precursor | IPI00025366 | PROCR Endothelial protein C receptor precursor | IPI00009276 |
| CSDA Isoform 1 of DNA-binding protein A | IPI00031801, IPI00219148 | PROS1 Vitamin K-dependent protein S precursor | IPI00294004 |
| CSDE1 Hypothetical protein DKFZp779B0247 | IPI00398121, IPI00470891 | PROSC Proline synthetase co-transcribed bacterial | IPI00016346 |
| | | homolog protein | |
| CSF1 Isoform 1 of Macrophage colony-stimulating factor 1 | IPI00015881 | PRPF19 Pre-mRNA-processing factor 19 | IPI00004968 |
| precursor | | | |
| CSNK2A1 Casein kinase 2 alpha isoform | IPI00741317 | PRPF6 Pre-mRNA-processing factor 6 | IPI00305068 |
| CSRP1 Cysteine and glycine-rich protein 1 | IPI00442073 | PRPS1 Phosphoribosyl pyrophosphate synthetase 1 | IPI00552495 |
| CSTF1 Cleavage stimulation factor 50 kDa subunit | IPI00011528, IPI00644499 | PRPS2 Ribose-phosphate pyrophosphokinase II | IPI00219617, IPI00718888 |
| CSTF2 Isoform 1 of Cleavage stimulation factor 64 kDa | IPI00013256, IPI00607841, IPI00744127 | PRRC1 Hypothetical protein | IPI00217053, IPI00744319 |
| subunit | | | |
| CTAGE5 Isoform MEA6 of Cutaneous T-cell lymphoma- | IPI00006122, IPI00515009 | PRSS23 Serine protease 23 precursor | IPI00026941 |
| associated antigen 5 | | | |
| CTBS Di-N-acetylchitobiase precursor | IPI00007778 | PSAT1 Isoform 1 of Phosphoserine aminotransferase | IPI00001734, IPI00219478 |
| CTGF Isoform 1 of Connective tissue growth factor | IPI00020977 | PSIP1 Isoform 1 of PC4 and SFRS1-interacting protein | IPI00028122 |
| precursor | | | |
| CTHRC1 Isoform 1 of Collagen triple helix repeat-containing | IPI00060423 | PSMA2 Proteasome subunit alpha type 2 | IPI00219622 |
| protein 1 precursor | | | |
| CTNNA1 Isoform 1 of Catenin alpha-1 | IPI00215948 | PSMA3 Isoform 2 of Proteasome subunit alpha type 3 | IPI00171199, IPI00419249 |
| CTNNB1 Isoform 1 of Catenin beta-1 | IPI00017292, IPI00787027, IPI00787237 | PSMA4 26 kDa protein | IPI00795606 |
| CTNNB1 similar to Beta-catenin | IPI00787237 | PSMA4 Proteasome subunit alpha type 4 | IPI00299155, IPI00789638, |
| | | | IPI00790038, IPI00795606 |
| CTNND1 Isoform 1AB of Catenin delta-1 | IPI00182469, IPI00219725, IPI00219728, | PSMA4 PSMA4 protein | IPI00789638, IPI00790038 |
| | IPI00219730, IPI00219732, | | |
| | IPI00219734, IPI00219738, | | |
| | IPI00219739, IPI00219742, | | |
| | IPI00219870, IPI00219872 | | |
| CTPS CTP synthase 1 | IPI00290142 | PSMA5 Proteasome subunit alpha type 5 | IPI00291922 |
| CTPS2 CTP synthase 2 | IPI00645702 | PSMA7 Isoform 2 of Proteasome subunit alpha type 7 | IPI00218372 |
| CTSA 62 kDa protein | IPI00021794, IPI00640525, IPI00791457 | PSMB1 Proteasome subunit beta type 1 precursor | IPI00025019 |
| CTSA cathepsin A precursor | IPI00640525 | PSMB2 Proteasome subunit beta type 2 | IPI00028006 |
| CTSC Dipeptidyl-peptidase 1 precursor | IPI00022810 | PSMB3 Proteasome subunit beta type 3 | IPI00028004 |
| CTSL1 Cathepsin L precursor | IPI00012887 | PSMB4 Proteasome subunit beta type 4 precursor | IPI00555956 |
| CTSL2 Cathepsin L2 precursor | IPI00000013 | PSMB5 Hypothetical protein DKFZp686I0180 (Fragment) | IPI00375704, IPI00479306 |
| CTTN Src substrate cortactin | IPI00029601, IPI00062884 | PSMB7 Proteasome subunit beta type 7 precursor | IPI00003217 |
| CUL3 Isoform 1 of Cullin-3 | IPI00014312 | PSMC4 Isoform 1 of 26S protease regulatory subunit 6B | IPI00020042, IPI00216770 |
| CUTA Isoform A of Protein CutA precursor | IPI00034319, IPI00554556, IPI00554634 | PSMC5 26S protease regulatory subunit 8 | IPI00023919, IPI00745502 |
| CXCL1 Growth-regulated protein alpha precursor | IPI00013874 | PSMC6 26S protease regulatory subunit S10B | IPI00021926 |
| CXCL14 small inducible cytokine B14 precursor | IPI00396257 | PSMD1 Isoform 1 of 26S proteasome non-ATPase | IPI00299608, IPI00456695 |
| | | regulatory subunit 1 | |
| CXCL5 Small inducible cytokine B5 precursor | IPI00292936 | PSMD11 Proteasome 26S non-ATPase subunit 11 variant | IPI00105598 |
| | | (Fragment) | |
| CYB5B cytochrome b5 outer mitochondrial membrane | IPI00303954 | PSMD13 proteasome 26S non-ATPase subunit 13 isoform 2 | IPI00375380 |
| precursor | | | |
| CYCS Cytochrome c | IPI00465315 | PSMD2 26S proteasome non-ATPase regulatory subunit 2 | IPI00012268 |
| CYFIP1 145 kDa protein | IPI00644231, IPI00791837 | PSMD2 P67 | IPI00384420 |
| CYFIP1 Isoform 1 of Cytoplasmic FMR1-interacting protein 1 | IPI00644231, IPI00791837 | PSMD3 26S proteasome non-ATPase regulatory subunit 3 | IPI00011603 |
| CYR61 CYR61 protein | IPI00006273 | PSMD4 Isoform Rpn10A of 26S proteasome non-ATPase | IPI00022694 |
| | | regulatory subunit 4 | |
| CYR61 Protein CYR61 precursor | IPI00299219 | PSMD7 26S proteasome non-ATPase regulatory subunit 7 | IPI00019927 |
| D4ST1 Carbohydrate sulfotransferase D4ST1 | IPI00044326 | PSME2 Proteasome activator complex subunit 2 | IPI00384051, IPI00746205 |
| DAG1 Dystroglycan precursor | IPI00028911 | PSPC1 paraspeckle protein 1 | IPI00103525 |
| DARS Aspartyl-tRNA synthetase, cytoplasmic | IPI00216951 | PTBP1 Isoform 1 of Polypyrimidine tract-binding protein 1 | IPI00179964, IPI00183626, |
| | | | IPI00334175 |
| DAZAP1 Isoform 1 of DAZ-associated protein 1 | IPI00165230, IPI00335930 | PTGES3 15 kDa protein | IPI00790462 |
| DBN1 Drebrin | IPI00003406, IPI00295624 | PTGES3 19 kDa protein | IPI00789101 |
| DBN1 drebrin 1 isoform b | IPI00295624 | PTGES3 Prostaglandin E synthase 3 | IPI00015029, IPI00789101, |
| | | | IPI00789698 |
| DCI Isoform 1 of 3,2-trans-enoyl-CoA isomerase, | IPI00300567 | PTK7 PTK7 protein tyrosine kinase 7 isoform a variant | IPI00555762 |
| mitochondrial precursor | | (Fragment) | |
| DCTN2 dynactin 2 | IPI00220503, IPI00793544 | PTK7 Tyrosine-protein kinase-like 7 precursor | IPI00298292 |
| DDAH1 NG,NG-dimethylarginine dimethylaminohydrolase 1 | IPI00220342 | PTPN11 Isoform 2 of Tyrosine-protein phosphatase non- | IPI00298347, IPI00658023 |
| | | receptor type 11 | |
| DDB1 damage-specific DNA binding protein 1 | IPI00786914 | PTPN12 Tyrosine-protein phosphatase non-receptor type | IPI00289082 |
| | | 12 | |
| DDB1 DNA damage-binding protein 1 | IPI00293464, IPI00784120, IPI00786914 | PTPRD protein tyrosine phosphatase, receptor type, D | IPI00375547 |
| | | isoform 2 precursor | |
| DDC Aromatic-L-amino-acid decarboxylase | IPI00025394 | PTPRG Receptor-type tyrosine-protein phosphatase | IPI00011651 |
| | | gamma precursor | |
| DDR1 Isoform 1 of Epithelial discoidin domain-containing | IPI00001477, IPI00219996, IPI00657861 | PTPRS protein tyrosine phosphatase, receptor type, sigma | IPI00743517 |
| receptor 1 precursor | | isoform 2 precursor | |
| DDT D-dopachrome decarboxylase | IPI00293867 | PTPRZ1 protein tyrosine phosphatase, receptor-type, zeta1 | IPI00748312 |
| | | precursor | |
| DDX1 ATP-dependent RNA helicase DDX1 | IPI00293655 | PTRF Isoform 1 of Polymerase I and transcript release | IPI00176903, IPI00513773 |
| | | factor | |
| DDX17 Isoform 1 of Probable ATP-dependent RNA helicase | IPI00023785, IPI00651677 | PTX3 Pentraxin-related protein PTX3 precursor | IPI00029568 |
| DDX17 | | | |
| DDX17 Isoform 3 of Probable ATP-dependent RNA helicase | IPI00651653 | PVR Isoform Beta of Poliovirus receptor precursor | IPI00219425 |
| DDX17 | | | |
| DDX19A ATP-dependent RNA helicase DDX19A | IPI00008943, IPI00019918 | PVR Isoform Gamma of Poliovirus receptor precursor | IPI00219426 |
| DDX21 Isoform 1 of Nucleolar RNA helicase 2 | IPI00015953 | PVRL1 Isoform Delta of Poliovirus receptor-related protein 1 | IPI00003648 |
| | | precursor | |
| DDX39 ATP-dependent RNA helicase DDX39 | IPI00644431 | PVRL2 Isoform Alpha of Poliovirus receptor-related protein | IPI00215980 |
| | | 2 precursor | |
| DDX3X ATP-dependent RNA helicase DDX3X | IPI00215637 | PXDN peroxidasin homolog | IPI00016112, IPI00787655 |
| DDX42 Isoform 1 of ATP-dependent RNA helicase DDX42 | IPI00409671 | PXDN similar to peroxidasin | IPI00787655 |
| DDX42 Isoform 2 of ATP-dependent RNA helicase DDX42 | IPI00829889 | PYGB Glycogen phosphorylase, brain form | IPI00004358 |
| DDX46 Probable ATP-dependent RNA helicase DDX46 | IPI00329791 | PYGL Glycogen phosphorylase, liver form | IPI00470525, IPI00783313 |
| DDX5 Probable ATP-dependent RNA helicase DDX5 | IPI00017617 | QARS Glutaminyl-tRNA synthetase | IPI00026665 |
| DECR1 2,4-dienoyl-CoA reductase, mitochondrial precursor | IPI00003482 | QPCT Glutaminyl-peptide cyclotransferase precursor | IPI00003919 |
| DEK Protein DEK | IPI00020021 | QSCN6L1 78 kDa protein | IPI00376394, IPI00479085, |
| | | | IPI00783371 |
| DENR Density-regulated protein | IPI00306280 | RAB11B Ras-related protein Rab-11B | IPI00020436 |
| DFFA Isoform DFF45 of DNA fragmentation factor subunit | IPI00010882 | RAB14 Ras-related protein Rab-14 | IPI00291928 |
| alpha (Fragment) | | | |
| DHX15 DEAH (Asp-Glu-Ala-His) box polypeptide 15 | IPI00396435 | RAB2A 21 kDa protein | IPI00798089 |
| DHX9 ATP-dependent RNA helicase A | IPI00742905 | RAB7A Ras-related protein Rab-7 | IPI00016342 |
| DIDO1 Isoform 4 of Death-inducer obliterator 1 | IPI00619921 | RAD23A UV excision repair protein RAD23 homolog A | IPI00008219 |
| DKC1 H/ACA ribonucleoprotein complex subunit 4 | IPI00221394 | RAD23B UV excision repair protein RAD23 homolog B | IPI00008223 |
| DKFZp686D0972 hypothetical protein LOC345651 | IPI00003269 | RAE1 MRNA-associated protein Mrnp 41 | IPI00749517 |
| DKFZp686O24166 Hypothetical protein DKFZp686I21167 | IPI00398918 | RALY RNA binding protein (Fragment) | IPI00011268, IPI00216044 |
| DKK1 Dickkopf-related protein 1 precursor | IPI00016353 | RAN 24 kDa protein | IPI00793015 |
| DLD Dihydrolipoyl dehydrogenase, mitochondrial precursor | IPI00015911 | RAN 26 kDa protein | IPI00792352, IPI00793015 |
| DLG1 Isoform 1 of Disks large homolog 1 | IPI00030351, IPI00218729, IPI00552213, | RAN 27 kDa protein | IPI00796462 |
| | IPI00552376, IPI00552511, | | |
| | IPI00552682, IPI00553029 | | |
| DNAJA1 DnaJ homolog subfamily A member 1 | IPI00012535 | RAN GTP-binding nuclear protein Ran | IPI00643041, IPI00795671, |
| | | | IPI00796462 |
| DNAJA2 DnaJ homolog subfamily A member 2 | IPI00032406 | RANBP1 Ran-specific GTPase-activating protein | IPI00414127 |
| DNAJB1 DnaJ homolog subfamily B member 1 | IPI00015947 | RANBP2 E3 SUMO-protein ligase RanBP2 | IPI00221325 |
| DNAJB11 DnaJ homolog subfamily B member 11 precursor | IPI00008454 | RANBP3 Isoform 1 of Ran-binding protein 3 | IPI00026337, IPI00179121, |
| | | | IPI00456728, IPI00456729 |
| DNAJB6 Isoform A of DnaJ homolog subfamily B member 6 | IPI00024523 | RANBP5 127 kDa protein | IPI00329200, IPI00783829, |
| | | | IPI00793443 |
| DNAJC10 DnaJ homolog subfamily C member 10 | IPI00293260 | RANBP5 Importin beta-3 | IPI00783829, IPI00793443 |
| DNAJC3 Isoform 1 of DnaJ homolog subfamily C member 3 | IPI00006713 | RANGAP1 Ran GTPase-activating protein 1 | IPI00294879, IPI00411570 |
| DNAJC8 DnaJ homolog subfamily C member 8 | IPI00003438 | RAP1A Ras-related protein Rap-1A precursor | IPI00019345, IPI00640287 |
| DNASE2 Deoxyribonuclease-2-alpha precursor | IPI00010348 | RAP1B Ras-related protein Rap-1b precursor | IPI00015148 |
| DNER Delta and Notch-like epidermal growth factor-related | IPI00333140, IPI00783545 | RARS Isoform Complexed of Arginyl-tRNA synthetase, | IPI00004860, IPI00759723 |
| receptor precursor | | cytoplasmic | |
| DNM1L Isoform 1 of Dynamin-1-like protein | IPI00146935 | RBBP4 Histone-binding protein RBBP4 | IPI00328319, IPI00645329 |
| DNM1L Isoform 4 of Dynamin-1-like protein | IPI00146935, IPI00235412, IPI00473085, | RBBP7 Histone-binding protein RBBP7 | IPI00395865 |
| | IPI00555883 | | |
| DNM1L Isoform 5 of Dynamin-1-like protein | IPI00037283 | RBBP7 Retinoblastoma binding protein 7 | IPI00646512 |
| DNM2 Isoform 1 of Dynamin-2 | IPI00033022, IPI00181352, IPI00218889, | RBM12B RNA binding motif protein 12B | IPI00217626 |
| | IPI00477431, IPI00514550, | | |
| | IPI00743573, IPI00794575 | | |
| DNPEP Hypothetical protein DNPEP | IPI00015856, IPI00029820, IPI00658188, | RBM15 Isoform 1 of Putative RNA-binding protein 15 | IPI00102752, IPI00220716, |
| | IPI00658215 | | IPI00220717 |
| DOHH Deoxyhypusine hydroxylase | IPI00171856 | RBM22 Pre-mRNA-splicing factor RBM22 | IPI00019046, IPI00783867 |
| DPP3; BBS1 Isoform 1 of Dipeptidyl-peptidase 3 | IPI00020672 | RBM25 RNA binding motif protein 25 | IPI00004273 |
| DPYSL2 Dihydropyrimidinase-related protein 2 | IPI00257508 | RBM3 Putative RNA-binding protein 3 | IPI00024320, IPI00604407 |
| DPYSL3 DPYSL3 protein | IPI00029111 | RBM8A Isoform 1 of RNA-binding protein 8A | IPI00001757, IPI00216659 |
| DSC2 Isoform 2A of Desmocollin-2 precursor | IPI00025846, IPI00220146 | RBMX Heterogeneous nuclear ribonucleoprotein G | IPI00304692 |
| DSG2 desmoglein 2 preproprotein | IPI00028931 | RBMXL1; CCBL2 RNA binding motif protein, X-linked-like 1 | IPI00061178 |
| DSTN Destrin | IPI00473014, IPI00643237 | RBP1 Retinol-binding protein I, cellular | IPI00219718 |
| DSTN destrin isoform b | IPI00031045, IPI00473014, IPI00643237 | RCC1 regulator of chromosome condensation 1 isoform a | IPI00001661, IPI00747309, |
| | | | IPI00787306 |
| DUT 24 kDa protein | IPI00793322 | RCC2 Protein RCC2 | IPI00465044 |
| DUT dUTP pyrophosphatase isoform 1 precursor | IPI00749113 | RCN1 Reticulocalbin-1 precursor | IPI00015842 |
| DUT Isoform DUT-M of Deoxyuridine 5′-triphosphate | IPI00013679, IPI00749113, IPI00793322 | RECQL ATP-dependent DNA helicase Q1 | IPI00178431 |
| nucleotidohydrolase, mitochondrial precursor | | | |
| DYNC1H1 532 kDa protein | IPI00477531 | RELL1 RELL1 protein | IPI00216890 |
| DYNC1H1 Dynein heavy chain, cytosolic | IPI00456969, IPI00477531 | RFC1 Isoform 1 of Replication factor C subunit 1 | IPI00375358, IPI00375359 |
| DYNC1I2 Isoform 2E of Cytoplasmic dynein 1 intermediate | IPI00827859 | RFNG similar to Beta-1,3-N-acetylglucosaminyltransferase | IPI00788176 |
| chain 2 | | radical fringe | |
| DYNLL1 Dynein light chain 1, cytoplasmic | IPI00019329 | RHOA Transforming protein RhoA precursor | IPI00027500 |
| EBNA1BP2 EBNA1 binding protein 2 | IPI00745955 | RHOC Rho-related GTP-binding protein RhoC precursor | IPI00027434, IPI00647268 |
| ECH1 Delta(3,5)-Delta(2,4)-dienoyl-CoA isomerase, | IPI00011416 | RNASE4; ANG Angiogenin precursor | IPI00008554 |
| mitochondrial precursor | | | |
| ECHS1 Enoyl-CoA hydratase, mitochondrial precursor | IPI00024993 | RNASET2 Isoform 1 of Ribonuclease T2 precursor | IPI00414896 |
| ECM1 Extracellular matrix protein 1 precursor | IPI00003351, IPI00645849 | RNF40 Isoform 1 of E3 ubiquitin-protein ligase BRE1B | IPI00162563 |
| EDIL3 Isoform 1 of EGF-like repeat and discoidin 1-like | IPI00306046, IPI00399105 | RNH1 Ribonuclease/angiogenin inhibitor | IPI00783491 |
| domain-containing protein 3 precursor | | | |
| EEF1A1 Elongation factor 1-alpha 1 | IPI00396485 | RNPEP 68 kDa protein | IPI00647400 |
| EEF1A2 Elongation factor 1-alpha 2 | IPI00014424 | RNPEP Aminopeptidase B | IPI00642211 |
| EEF1B2 Elongation factor 1-beta | IPI00178440 | ROBO1 ROBO1 protein | IPI00829739 |
| EEF1D EEF1D protein | IPI00064086 | ROCK2 Rho-associated protein kinase 2 | IPI00307155 |
| EEF1D eukaryotic translation elongation factor 1 delta | IPI00642971 | RP6-166C19.3; RP6-166C19.5; RP6-166C19.4; RP6- | IPI00167090 |
| isoform 1 | | 166C19.1; CT47.8; RP6-166C19.6; RP6-166C19.10; RP6- | |
| | | 166C19.11; RP6-166C19.9; RP6-166C19.2; CT47.7 | |
| | | hypothetical protein LOC728036 | |
| EEF1E1 Eukaryotic translation elongation factor 1 epsilon-1 | IPI00003588 | RPA1 Replication protein A 70 kDa DNA-binding subunit | IPI00020127 |
| EFEMP1 Isoform 1 of EGF-containing fibulin-like | IPI00029658, IPI00220813, IPI00220814, | RPA2 Isoform 3 of Replication protein A 32 kDa subunit | IPI00646500 |
| extracellular matrix protein 1 precursor | IPI00220815 | | |
| EFHD2 EF-hand domain-containing protein 2 | IPI00060181 | RPA3 Replication protein A 14 kDa subunit | IPI00017373 |
| EFNA1 Isoform 1 of Ephrin-A1 precursor | IPI00025840 | RPL10A 60S ribosomal protein L10a | IPI00412579, IPI00827508 |
| EFNA5 Ephrin-A5 precursor | IPI00005517 | RPL11 Isoform 1 of 60S ribosomal protein L11 | IPI00376798, IPI00647168, |
| | | | IPI00647674, IPI00746438 |
| EFNB1 Ephrin-B1 precursor | IPI00024307 | RPL12 60S ribosomal protein L12 | IPI00024933 |
| EFTUD2 116 kDa U5 small nuclear ribonucleoprotein | IPI00003519 | RPL14 RPL14 protein | IPI00555744 |
| component | | | |
| EGFL9 Isoform 1 of EGF-like domain-containing protein 9 | IPI00028381 | RPL17 60S ribosomal protein L17 | IPI00413324, IPI00478208, |
| precursor | | | IPI00514874, IPI00644171 |
| EHD4 EH domain-containing protein 4 | IPI00005578 | RPL18 60S ribosomal protein L18 | IPI00215719 |
| EIF1 Eukaryotic translation initiation factor 1 | IPI00015077 | RPL22 60S ribosomal protein L22 | IPI00219153 |
| EIF2S1 Eukaryotic translation initiation factor 2 subunit 1 | IPI00219678 | RPL23A 60S ribosomal protein L23a | IPI00021266, IPI00789159, |
| | | | IPI00793523, IPI00794894 |
| EIF2S2 Eukaryotic translation initiation factor 2 subunit 2 | IPI00021728, IPI00793912 | RPL27 60S ribosomal protein L27 | IPI00219155, IPI00382885 |
| EIF2S3 Eukaryotic translation initiation factor 2 subunit 3 | IPI00297982 | RPL30 60S ribosomal protein L30 | IPI00219156 |
| EIF3S1 Eukaryotic translation initiation factor 3 subunit 1 | IPI00290461 | RPL38 60S ribosomal protein L38 | IPI00215790, IPI00792410 |
| EIF3S12 Eukaryotic translation initiation factor 3 subunit 12 | IPI00033143 | RPL4 60S ribosomal protein L4 | IPI00003918 |
| EIF3S2 Eukaryotic translation initiation factor 3 subunit 2 | IPI00012795 | RPL5 60S ribosomal protein L5 | IPI00000494, IPI00797983 |
| EIF3S3 Eukaryotic translation initiation factor 3 subunit 3 | IPI00647650 | RPL8 60S ribosomal protein L8 | IPI00012772, IPI00795138, |
| | | | IPI00797230 |
| EIF3S4 Eukaryotic translation initiation factor 3 subunit 4 | IPI00290460 | RPLP0 24 kDa protein | IPI00794884 |
| EIF3S5 Eukaryotic translation initiation factor 3 subunit 5 | IPI00654777 | RPLP0 60S acidic ribosomal protein P0 | IPI00008530, IPI00556485, |
| | | | IPI00791448 |
| EIF3S6 Eukaryotic translation initiation factor 3 subunit 6 | IPI00013068 | RPLP1; hCG_1641617 60S acidic ribosomal protein P1 | IPI00008527 |
| EIF3S6IP DJ1014D13.1 protein | IPI00465233 | RPLP2 60S acidic ribosomal protein P2 | IPI00008529 |
| EIF3S8 Eukaryotic translation initiation factor 3 subunit 8 | IPI00016910, IPI00646839 | RPS10 40S ribosomal protein S10 | IPI00008438 |
| EIF3S9 99 kDa protein | IPI00747447 | RPS11 40S ribosomal protein S11 | IPI00025091 |
| EIF4A2 Isoform 1 of Eukaryotic initiation factor 4A-II | IPI00328328, IPI00409717 | RPS12 40S ribosomal protein S12 | IPI00013917 |
| EIF4B Eukaryotic translation initiation factor 4B | IPI00012079, IPI00439415 | RPS13 40S ribosomal protein S13 | IPI00221089 |
| EIF4E Eukaryotic translation initiation factor 4E | IPI00027485 | RPS14 40S ribosomal protein S14 | IPI00026271 |
| EIF4E Similar to Eukaryotic translation initiation factor 4E | IPI00783643, IPI00788723 | RPS19 40S ribosomal protein S19 | IPI00215780 |
| EIF4G1 EIF4G1 variant protein (Fragment) | IPI00220365, IPI00384463, IPI00479262, | RPS2 40S ribosomal protein S2 | IPI00013485, IPI00479366, |
| | IPI00798400 | | IPI00744451, IPI00827598 |
| EIF4G1 Isoform E of Eukaryotic translation initiation factor 4 | IPI00386533 | RPS20 40S ribosomal protein S20 | IPI00012493, IPI00794659 |
| gamma 1 | | | |
| EIF4G2 Eukaryotic translation initiation factor 4 gamma 2 | IPI00015952 | RPS21 40S ribosomal protein S21 | IPI00017448, IPI00387084, |
| | | | IPI00797280 |
| EIF5 Eukaryotic translation initiation factor 5 | IPI00022648 | RPS27A; UBC; UBB 12 kDa protein | IPI00793330 |
| EIF5A Isoform 1 of Eukaryotic translation initiation factor 5A-1 | IPI00411704 | RPS27A; UBC; UBB ubiquitin and ribosomal protein S27a | IPI00179330, IPI00456429, |
| | | precursor | IPI00793330 |
| EIF5A Isoform 2 of Eukaryotic translation initiation factor 5A-1 | IPI00376005, IPI00411704 | RPS3 40S ribosomal protein S3 | IPI00011253 |
| EIF5B Eukaryotic translation initiation factor 5B | IPI00299254 | RPS3A 40S ribosomal protein S3a | IPI00419880, IPI00472119 |
| ELAVL1 ELAV-like protein 1 | IPI00301936 | RPS4X 40S ribosomal protein S4, X isoform | IPI00217030 |
| EML4 Echinoderm microtubule-associated protein-like 4 | IPI00001466 | RPS5 40S ribosomal protein S5 | IPI00008433 |
| ENAH Isoform 2 of Protein enabled homolog | IPI00374054, IPI00411623, IPI00646954 | RPS7 40S ribosomal protein S7 | IPI00013415 |
| ENO2 Gamma-enolase | IPI00216171 | RPS8 40S ribosomal protein S8 | IPI00216587 |
| ENOPH1 Isoform 1 of Enolase-phosphatase E1 | IPI00038378, IPI00795241 | RPSA; LOC388524 Ribosomal protein SA | IPI00413108, IPI00553164 |
| ENPP2 Isoform 1 of Ectonucleotide | IPI00156171, IPI00303210 | RRBP1 Isoform 1 of Ribosome-binding protein 1 | IPI00220967 |
| pyrophosphatase/phosphodiesterase family member 2 | | | |
| precursor | | | |
| ENSA Hypothetical protein DKFZp779B2258 | IPI00470835 | RRBP1 Isoform 3 of Ribosome-binding protein 1 | IPI00215743 |
| ENSA Isoform 2 of Alpha-endosulfine | IPI00220797, IPI00383552, IPI00410177, | RRM1 Ribonucleoside-diphosphate reductase large subunit | IPI00013871 |
| | IPI00410178, IPI00410179, | | |
| | IPI00410180, IPI00410181, | | |
| | IPI00470835, IPI00644717 | | |
| ENTPD6 Ectonucleoside triphosphate diphosphohydrolase 6 | IPI00478478 | RTBDN retbindin isoform 2 | IPI00027765, IPI00829898 |
| EPB41L3 Isoform A of Band 4.1-like protein 3 | IPI00032230 | RTN4 Isoform 1 of Reticulon-4 | IPI00021766 |
| EPHA2 ephrin receptor EphA2 | IPI00745296 | RUVBL1 Isoform 1 of RuvB-like 1 | IPI00021187 |
| EPHA4 Ephrin type-A receptor 4 precursor | IPI00008318 | RUVBL2 RuvB-like 2 | IPI00009104 |
| EPHA5 Isoform 1 of Ephrin type-A receptor 5 precursor | IPI00008290, IPI00215945 | S100A10 Protein S100-A10 | IPI00183695 |
| EPHX1 Epoxide hydrolase 1 | IPI00009896 | SAE2 SUMO-activating enzyme subunit 2 | IPI00023234 |
| EPRS glutamyl-prolyl tRNA synthetase | IPI00013452 | SAFB Scaffold attachment factor B | IPI00646058 |
| EPS15L1 Epidermal growth factor receptor substrate 15-like 1 | IPI00163849, IPI00645613, IPI00646339, | SAFB2 Scaffold attachment factor B2 | IPI00005648 |
| | IPI00794004, IPI00795804, | | |
| | IPI00797124 | | |
| ERH Enhancer of rudimentary homolog | IPI00029631 | SARS Seryl-tRNA synthetase | IPI00514587 |
| ERO1L ERO1-like protein alpha precursor | IPI00386755 | SART1 U4/U6.U5 tri-snRNP-associated protein 1 | IPI00021417 |
| ERP29 Endoplasmic reticulum protein ERp29 precursor | IPI00024911 | SCAMP3 Isoform 1 of Secretory carrier-associated | IPI00306382, IPI00453116, |
| | | membrane protein 3 | IPI00479205 |
| ESD S-formylglutathione hydrolase | IPI00411706 | SCARB1 Isoform 1 of Scavenger receptor class B member 1 | IPI00177968, IPI00291007, |
| | | | IPI00447020 |
| ESM1 Endothelial cell-specific molecule 1 precursor | IPI00015186 | SCG2 Secretogranin-2 precursor | IPI00009362 |
| EWSR1 CDNA FLJ31747 fis, clone NT2RI2007377, highly | IPI00009841, IPI00065554 | SCG3 Secretogranin-3 precursor | IPI00292071 |
| similar to RNA-BINDING PROTEIN EWS | | | |
| EXT1 Exostosin-1 | IPI00293128 | SCG5 Isoform 1 of Neuroendocrine protein 7B2 precursor | IPI00008944 |
| EXT2 Isoform 1 of Exostosin-2 | IPI00004047, IPI00414468 | SCPEP1 Isoform 1 of Retinoid-inducible serine | IPI00012426 |
| | | carboxypeptidase precursor | |
| EXTL2 EXTL2 protein (Fragment) | IPI00002732, IPI00790204 | SCUBE1 Signal peptide, CUB and EGF-like domain- | IPI00217435, IPI00747808 |
| | | containing protein 1 precursor | |
| F10 Coagulation factor X precursor | IPI00019576, IPI00797559 | SCYE1 37 kDa protein | IPI00793201 |
| F11R Isoform 1 of Putative thiosulfate sulfurtransferase KAT | IPI00607735, IPI00644969 | SCYE1 Multisynthetase complex auxiliary component p43 | IPI00006252, IPI00793201 |
| F11R Junctional adhesion molecule A precursor | IPI00001754, IPI00069985, IPI00294993 | SDF2 Stromel cell-derived factor 2 precursor | IPI00293167 |
| F5 Coagulation factor V | IPI00022937 | SDF2L1 Dihydropyrimidinase-like 2 | IPI00106642 |
| FAM10A4 Protein FAM10A4 | IPI00218038 | SDF4 45 kDa calcium-binding protein precursor | IPI00106646 |
| FAM129B Niban-like protein | IPI00456750, IPI00647286 | SDPR Serum deprivation-response protein | IPI00005809 |
| FAM20B Protein FAM20B precursor | IPI00006657 | SDSL Serine dehydratase-like | IPI00062419 |
| FAM3B 28 kDa protein | IPI00790820 | SEC23B Protein transport protein Sec23B | IPI00017376 |
| FAM50A Protein FAM50A | IPI00030098 | SECTM1 Secreted and transmembrane protein 1 precursor | IPI00170635 |
| FARSA Phenylalanyl-tRNA synthetase alpha chain | IPI00031820 | SELENBP1 selenium binding protein 1 | IPI00745729 |
| FARSB Phenylalanyl-tRNA synthetase beta chain | IPI00300074 | SEMA3A Semaphorin-3A precursor | IPI00031510 |
| FASN fatty acid synthase | IPI00645907 | SEMA3C Semaphorin-3C precursor | IPI00019209 |
| FAT Cadherin-related tumor suppressor homolog precursor | IPI00031411 | SEMA3D Semaphorin-3D precursor | IPI00028213, IPI00783087 |
| FBL rRNA 2′-O-methyltransferase fibrillarin | IPI00025039 | SEMA3E Semaphorin-3E precursor | IPI00004494 |
| FBLN2 fibulin 2 precursor, isoform a | IPI00465038 | SEMA3G Semaphorin-3G precursor | IPI00024570 |
| FBLN2 Fibulin-2 precursor | IPI00023824, IPI00465038 | SEMA4B semaphorin 4B precursor | IPI00419724 |
| FBN1 312 kDa protein | IPI00784458 | SEMA4C Semaphorin-4C precursor | IPI00073763 |
| FBN1 Fibrillin-1 precursor | IPI00328113, IPI00784458 | SEMA4D Semaphorin-4D precursor | IPI00023807 |
| FBN2 fibrillin 2 precursor | IPI00019439, IPI00784315 | SEMA7A Semaphorin-7A precursor | IPI00025257 |
| FDPS Farnesyl diphosphate synthase | IPI00101405, IPI00797614 | SEP15 15 kDa selenoprotein isoform 1 precursor | IPI00030877 |
| FEN1 Flap endonuclease 1 | IPI00026215 | SEPT7 Isoform 1 of Septin-7 | IPI00033025 |
| FER1L3 Isoform 1 of Myoferlin | IPI00021048, IPI00216269, IPI00645867, | SEPT9 Isoform 1 of Septin-9 | IPI00784614, IPI00784936 |
| | IPI00790914, IPI00827894 | | |
| FGF19 Fibroblast growth factor 19 precursor | IPI00032908 | SEPT9 Isoform 3 of Septin-9 | IPI00455033, IPI00784614, |
| | | | IPI00784808, IPI00784936 |
| FGFBP1 Fibroblast growth factor-binding protein 1 | IPI00021399 | SEPT9 Isoform 5 of Septin-9 | IPI00784808 |
| precursor | | | |
| FGFR1 Isoform 14 of Basic fibroblast growth factor receptor | IPI00328245 | SERBP1 Isoform 1 of Plasminogen activator inhibitor 1 | IPI00410693, IPI00412714, |
| 1 precursor | | RNA-binding protein | IPI00470497, IPI00470498 |
| FGFRL1 Fibroblast growth factor receptor-like 1 precursor | IPI00296561 | SERBP1 Isoform 4 of Plasminogen activator inhibitor 1 | IPI00412714, IPI00470498 |
| | | RNA-binding protein | |
| FH Isoform Mitochondrial of Fumarate hydratase, | IPI00296053, IPI00759715 | SERPINB2 Plasminogen activator inhibitor 2 precursor | IPI00007117 |
| mitochondrial precursor | | | |
| FHL1 Four and a half LIM domains 1 variant | IPI00014398, IPI00647207 | SERPINB9 Serpin B9 | IPI00032139 |
| FHL2 FHL2 isoform 5 | IPI00396967, IPI00743342 | SERPINE2 Glia-derived nexin precursor | IPI00009890 |
| FIP1L1 Isoform 1 of Pre-mRNA 3′-end-processing factor | IPI00395337, IPI00657863 | SERPINH1 Serpin H1 precursor | IPI00032140 |
| FIP1 | | | |
| FIP1L1 Isoform 3 of Pre-mRNA 3′-end-processing factor | IPI00008449, IPI00395337, IPI00657863, | SERPINI1 Neuroserpin precursor | IPI00016150 |
| FIP1 | IPI00658114 | | |
| FKBP10 FK506-binding protein 10 precursor | IPI00303300 | SET Isoform 1 of Protein SET | IPI00072377 |
| FKBP1A FKBP1A protein | IPI00413778 | SET Isoform 2 of Protein SET | IPI00301311 |
| FKBP3 FK506-binding protein 3 | IPI00024157 | SEZ6L2 Seizure 6-like protein 2 | IPI00419722 |
| FKBP4 FK506-binding protein 4 | IPI00219005 | SEZ6L2 Type I transmembrane receptor precursor | IPI00018276, IPI00419722, |
| | | | IPI00645828 |
| FLJ10292 Protein mago nashi homolog 2 | IPI00059292, IPI00219306 | SF1 Isoform 5 of Splicing factor 1 | IPI00386119 |
| FLJ11151 Hypothetical protein FLJ11151 | IPI00305010 | SF3A2 SF3A2 protein (Fragment) | IPI00017341 |
| FLJ12684 hypothetical protein LOC79584 | IPI00002191 | SF3A3 Splicing factor 3A subunit 3 | IPI00029764 |
| FLJ21908 CDNA: FLJ21908 fis, clone HEP03830 | IPI00002408 | SF3B1 Splicing factor 3B subunit 1 | IPI00026089 |
| FLJ37440 Hypothetical protein FLJ37440 | IPI00167710 | SF3B2 splicing factor 3B subunit 2 | IPI00221106 |
| FLNA filamin A, alpha | IPI00302592 | SF3B3 Isoform 1 of Splicing factor 3B subunit 3 | IPI00300371 |
| FLNA Filamin-A | IPI00333541 | SF3B4 Splicing factor 3B subunit 4 | IPI00017339 |
| FLNC Gamma filamin variant | IPI00783128 | SF3B5 Splicing factor 3B subunit 5 | IPI00010404 |
| FMR1 Isoform 1 of Fragile X mental retardation 1 protein | IPI00215720, IPI00215721, IPI00215723, | SFPQ Isoform Long of Splicing factor, proline- and | IPI00010740 |
| | IPI00215724, IPI00215725, | glutamine-rich | |
| | IPI00412343, IPI00645666, | | |
| | IPI00783298, IPI00795102 | | |
| FN1 Hypothetical protein DKFZp686K08164 | IPI00744362 | SFRP1 Secreted frizzled-related protein 1 precursor | IPI00749245 |
| FNBP1L Isoform 3 of Formin-binding protein 1-like | IPI00015580, IPI00028718, IPI00607808, | SFRS1 Isoform ASF-1 of Splicing factor, arginine/serine-rich 1 | IPI00215884, IPI00218591, |
| | IPI00646028, IPI00807364 | | IPI00218592 |
| FRAS1 Isoform 2 of Extracellular matrix protein FRAS1 | IPI00329327 | SFRS1 Isoform ASF-2 of Splicing factor, arginine/serine-rich 1 | IPI00218591 |
| precursor | | | |
| FREM2 Isoform 1 of FRAS1-related extracellular matrix | IPI00180707 | SFRS10 Isoform 1 of Arginine/serine-rich-splicing factor 10 | IPI00301503, IPI00472633, |
| protein 2 precursor | | | IPI00555647 |
| FST Isoform 1 of Follistatin precursor | IPI00021081, IPI00217070, IPI00217071 | SFRS2 Splicing factor, arginine/serine-rich 2 | IPI00005978, IPI00385786, |
| | | | IPI00796848 |
| FSTL1 Follistatin-related protein 1 precursor | IPI00029723 | SFRS3 Splicing factor, arginine/serine-rich 3 | IPI00010204 |
| FSTL3 Follistatin-related protein 3 precursor | IPI00025155 | SFRS7 Isoform 1 of Splicing factor, arginine/serine-rich 7 | IPI00003377 |
| FSTL4 Isoform 1 of Follistatin-related protein 4 precursor | IPI00477747 | SFRS9 Splicing factor, arginine/serine-rich 9 | IPI00012340, IPI00793205 |
| FSTL4 Isoform 2 of Follistatin-related protein 4 precursor | IPI00298956 | SGCE Isoform SGCE-1 of Epsilon-sarcoglycan precursor | IPI00414984, IPI00418183 |
| FSTL5 Follistatin-related protein 5 precursor | IPI00008087 | SGTA Small glutamine-rich tetratricopeptide repeat- | IPI00013949 |
| | | containing protein A | |
| FUBP1 Isoform 1 of Far upstream element-binding protein 1 | IPI00375441, IPI00644386, IPI00788671 | SHC1 SHC (Src homology 2 domain containing) | IPI00021326 |
| | | transforming protein 1 | |
| FUBP1 Isoform 2 of Far upstream element-binding protein 1 | IPI00788671 | SHH Sonic hedgehog protein precursor | IPI00017480 |
| FUCA1 fucosidase, alpha-L-1, tissue | IPI00745745 | SHMT2 Serine hydroxymethyltransferase, mitochondrial | IPI00002520, IPI00748411, |
| | | precursor | IPI00789370 |
| FUCA2 Plasma alpha-L-fucosidase precursor | IPI00012440 | SIAE Isoform 1 of Sialate O-acetylesterase precursor | IPI00010949 |
| FURIN Furin precursor | IPI00018387 | SIAHBP1 fuse-binding protein-interacting repressor isoform a | IPI00069750, IPI00100716, |
| | | | IPI00788826, IPI00797595 |
| FUS Fus-like protein (Fragment) | IPI00260715 | SIAHBP1 SIAHBP1 protein | IPI00788826 |
| FUSIP1 FUS interacting protein (Serine/arginine-rich) 1 | IPI00646643 | SIL1 Nucleotide exchange factor SIL1 precursor | IPI00296197 |
| FUSIP1 Isoform 2 of FUS-interacting serine-arginine-rich | IPI00412643, IPI00786930 | SKIV2L2 Superkiller viralicidic activity 2-like 2 | IPI00647217 |
| protein 1 | | | |
| FXR1 Isoform 1 of Fragile X mental retardation syndrome- | IPI00016249, IPI00554715 | SKP1A Isoform 1 of S-phase kinase-associated protein 1A | IPI00301364 |
| related protein 1 | | | |
| FXR1 isoform 2 of Fragile X mental retardation syndrome- | IPI00554715 | SLC1A5 Neutral amino acid transporter B | IPI00019472 |
| related protein 1 | | | |
| G3BP1 Ras GTPase-activating protein-binding protein 1 | IPI00012442 | SLC25A13 Mitochondrial aspartate-glutamate carrier protein | IPI00007084 |
| G3BP2 Isoform A of Ras GTPase-activating protein-binding | IPI00009057 | SLC2A1 Solute carrier family 2, facilitated glucose | IPI00220194 |
| protein 2 | | transporter member 1 | |
| G6PD glucose-6-phosphate dehydrogenase isoform a | IPI00760751 | SLC39A10 Solute carrier family 39 (Zinc transporter), | IPI00008085 |
| | | member 10 | |
| G6PD Isoform Long of Glucose-6-phosphate 1- | IPI00216008, IPI00289800, IPI00760751 | SLC3A2 solute carrier family 3 (activators of dibasic and | IPI00554702, IPI00604710 |
| dehydrogenase | | neutral amino acid transport), member 2 isoform d | |
| GAA 106 kDa protein | IPI00293088 | SLC44A1 Isoform 1 of Choline transporter-like protein 1 | IPI00221393 |
| GAA Lysosomal alpha-glucosidase precursor | IPI00783446 | SLIT3 Isoform 1 of Slit homolog 3 protein precursor | IPI00017640 |
| GAK Cyclin G-associated kinase | IPI00298949 | SLITRK6 Isoform 1 of SLIT and NTRK-like protein 6 | IPI00176398 |
| | | precursor | |
| GAL Galanin precursor | IPI00026637 | SMARCA5 SWI/SNF-related matrix-associated actin- | IPI00297211 |
| | | dependent regulator of chromatin subfamily A member 5 | |
| GALC galactosylceramidase isoform a precursor | IPI00008790 | SMARCC1 SWI/SNF related, matrix associated, actin | IPI00797830 |
| | | dependent regulator of chromatin, subfamily c, member 1 | |
| GALNAC4S-6ST Isoform 1 of N-acetylgalactosamine 4- | IPI00183321 | SMARCC1 SWI/SNF-related matrix-associated actin- | IPI00234252, IPI00797830 |
| sulfate 6-O-sulfotransferase | | dependent regulator of chromatin subfamily C member 1 | |
| GALNS N-acetylgalactosamine-6-sulfatase precursor | IPI00029605 | SMARCC2 Isoform 2 of SWI/SNF-related matrix-associated | IPI00150057, IPI00216047 |
| | | actin-dependent regulator of chromatin subfamily C member 2 | |
| GALNT10 Isoform 1 of Polypeptide N- | IPI00375205 | SMARCE1 Protein | IPI00794957 |
| acetylgalactosaminyltransferase 10 | | | |
| GALNT2 Polypeptide N-acetylgalactosaminyltransferase 2 | IPI00004669 | SMC1A Structural maintenance of chromosomes protein 1A | IPI00291939 |
| GALNT5 Polypeptide N-acetylgalactosaminyltransferase 5 | IPI00005401 | SMC3 Structural maintenance of chromosomes protein 3 | IPI00219420 |
| GALNT6 Polypeptide N-acetylgalactosaminyltransferase 6 | IPI00026991 | SMOC1 Isoform 1 of SPARC-related modular calcium- | IPI00301812, IPI00412898 |
| | | binding protein 1 precursor | |
| GALNT7 N-acetylgalactosaminyltransferase 7 | IPI00328391 | SMOC1 Isoform 2 of SPARC-related modular calcium- | IPI00412898 |
| | | binding protein 1 precursor | |
| GALNTL1 Isoform 1 of Putative polypeptide N- | IPI00166613 | SMOC2 Isoform 1 of SPARC-related modular calcium- | IPI00395336 |
| acetylgalactosaminyltransferase-like protein 1 | | binding protein 2 precursor | |
| GANAB 107 kDa protein | IPI00472068 | SMOC2 Isoform 2 of SPARC-related modular calcium- | IPI00301528, IPI00395336 |
| | | binding protein 2 precursor | |
| GANAB Isoform 2 of Neutral alpha-glucosidase AB | IPI00011454 | SMS Spermine synthase | IPI00005102 |
| precursor | | | |
| GARS Glycyl-tRNA synthetase | IPI00783097 | SND1 Staphylococcal nuclease domain-containing protein 1 | IPI00140420 |
| GART Isoform Long of Trifunctional purine biosynthetic | IPI00025273 | SNRP70 Isoform 1 of U1 small nuclear ribonucleoprotein 70 kDa | IPI00219483, IPI00290204 |
| protein adenosine-3 | | | |
| GAS6 Isoform 2 of Growth-arrest-specific protein 6 | IPI00032532, IPI00412410, IPI00412412 | SNRPA U1 small nuclear ribonucleoprotein A | IPI00012382 |
| precursor | | | |
| GATAD2B Transcriptional repressor p66 beta | IPI00103554 | SNRPB Isoform SM-B′ of Small nuclear ribonucleoprotein- | IPI00027285, IPI00329512, |
| | | associated proteins B and B′ | IPI00384173, IPI00395674, |
| | | | IPI00785142, IPI00792592 |
| GBA Isoform Long of Glucosylceramidase precursor | IPI00021807, IPI00759616 | SNRPB2 U2 small nuclear ribonucleoprotein B″ | IPI00029267 |
| GBE1 1,4-alpha-glucan branching enzyme | IPI00296635 | SNRPC U1 small nuclear ribonucleoprotein C | IPI00013396, IPI00641788 |
| GCG Glucagon precursor | IPI00306140 | SNRPD1 Small nuclear ribonucleoprotein Sm D1 | IPI00302850 |
| GCLC Glutamate--cysteine ligase catalytic subunit | IPI00215768 | SNRPD2 Small nuclear ribonucleoprotein Sm D2 | IPI00017963 |
| GCLM Glutamate--cysteine ligase regulatory subunit | IPI00010090 | SNRPD3 Small nuclear ribonucleoprotein Sm D3 | IPI00017964 |
| GCN1L1 GCN1-like protein 1 | IPI00001159 | SNRPE Small nuclear ribonucleoprotein E | IPI00029266, IPI00068430 |
| GCNT2 glucosaminyl (N-acetyl) transferase 2, I-branching | IPI00166086 | SNRPG Small nuclear ribonucleoprotein G | IPI00016572 |
| enzyme isoform A | | | |
| GDA Guanine deaminase | IPI00465184 | SNW1 SNW domain-containing protein 1 | IPI00013830 |
| GDF15 Growth/differentiation factor 15 precursor | IPI00306543 | SON Isoform J of SON protein | IPI00217930, IPI00218618, |
| | | | IPI00218619, IPI00401958 |
| GDI1 Rab GDP dissociation inhibitor alpha | IPI00010154 | SORD Sorbitol dehydrogenase | IPI00216057 |
| GFPT1 Isoform 1 of Glucosamine--fructose-6-phosphate | IPI00217952 | SORL1 Sortilin-related receptor precursor | IPI00022608 |
| aminotransferase [isomerizing] 1 | | | |
| GGH Gamma-glutamyl hydrolase precursor | IPI00023728 | SORT1 Sortilin precursor | IPI00217882 |
| GIPC1 PDZ domain-containing protein GIPC1 | IPI00024705 | SPINTI Isoform 2 of Kunitz-type protease inhibitor 1 | IPI00011643, IPI00376403 |
| | | precursor | |
| GJA10; MYCBP C-Myc-binding protein | IPI00554793 | SPOCK1 Testican-1 precursor | IPI00005292 |
| GLA Alpha-galactosidase A precursor | IPI00025869 | SPOCK2 Testican-2 precursor | IPI00006128 |
| GLB1 Beta-galactosidase precursor | IPI00441344, IPI00797646 | SPP1 Isoform A of Osteopontin precursor | IPI00021000, IPI00385896 |
| GLCE D-glucuronyl C5-epimerase | IPI00433284 | SPP1 secreted phosphoprotein 1 isoform b | IPI00306339 |
| GLG1 golgi apparatus protein 1 | IPI00414717, IPI00641153 | SPTAN1 Isoform 1 of Spectrin alpha chain, brain | IPI00478292, IPI00744706, |
| | | | IPI00745092 |
| GLO1 Lactoylglutathione lyase | IPI00220766 | SPTAN1 Isoform 2 of Spectrin alpha chain, brain | IPI00745092 |
| GLOD4 Uncharacterized protein C17orf25 | IPI00007102 | SPTAN1 Isoform 3 of Spectrin alpha chain, brain | IPI00744706 |
| GLRX Glutaredoxin-1 | IPI00219025 | SPTBN1 Isoform Long of Spectrin beta chain, brain 1 | IPI00005614 |
| GLT25D1 CDNA PSEC0241 fis, clone NT2RP3000234, | IPI00168262 | SQSTM1 Isoform 1 of Sequestosome-1 | IPI00783357, IPI00784104 |
| moderately similar to Homo sapiens cerebral cell adhesion | | | |
| molecule mRNA | | | |
| GMFB Glia maturation factor beta | IPI00412987, IPI00549557 | SR140 Isoform 1 of U2-associated protein SR140 | IPI00143753, IPI00829908 |
| GMPS GMP synthase | IPI00029079 | SRGN Secretory granule proteoglycan core protein | IPI00019372 |
| | | precursor | |
| GNB1 Guanine nucleotide-binding protein G(I)/G(S)/G(T) | IPI00026268 | SRP14 8 kDa protein | IPI00789296 |
| subunit beta 1 | | | |
| GNB2L1 Lung cancer oncogene 7 | IPI00641950 | SRP14 Signal recognition particle 14 kDa protein | IPI00293434 |
| GNPDA1 Glucosamine-6-phosphate isomerase | IPI00009305 | SRP54 Signal recognition particle 54 kDa protein | IPI00009822 |
| GNPDA2 Glucosamine-6-phosphate isomerase SB52 | IPI00550894, IPI00744859 | SRP9; hCG_1781062 Signal recognition particle 9 kDa | IPI00642816 |
| | | protein | |
| GNPTG N-acetylglucosamine-1-phosphotransferase subunit | IPI00000137 | SRPX Isoform 1 of Sushi repeat-containing protein SRPX | IPI00289795 |
| gamma precursor | | precursor | |
| GNS N-acetylglucosamine-6-sulfatase precursor | IPI00012102 | SRPX2 Sushi repeat-containing protein SRPX2 precursor | IPI00004446 |
| GOLPH2 Golgi phosphoprotein 2 | IPI00171411, IPI00759659, IPI00784293 | SRRM2 Isoform 1 of Serine/arginine repetitive matrix | IPI00782992 |
| | | protein 2 | |
| GOLPH2 Isoform 2 of Golgi phosphoprotein 2 | IPI00759659, IPI00784293 | SRXN1; SCRT2 Sulfiredoxin-1 | IPI00168554 |
| GOLPH4 golgi phosphoprotein 4 | IPI00004962 | SSB Lupus La protein | IPI00009032 |
| GORASP2 Isoform 1 of Golgi reassembly-stacking protein 2 | IPI00743931 | SSRP1 FACT complex subunit SSRP1 | IPI00005154 |
| GORASP2 Isoform 2 of Golgi reassembly-stacking protein 2 | IPI00031241, IPI00743931 | ST13 Hsc70-interacting protein | IPI00032826 |
| GOT1 Aspartate aminotransferase, cytoplasmic | IPI00219029 | ST14 Suppressor of tumorigenicity protein 14 | IPI00001922 |
| GPC4 Glypican-4 precursor | IPI00232571 | ST3GAL1 CMP-N-acetylneuraminate-beta-galactosamide- | IPI00009629 |
| | | alpha-2,3-sialyltransferase | |
| GPC6 Glypican-6 precursor | IPI00001755 | ST6GAL1 CMP-N-acetylneuraminate-beta-galactosamide- | IPI00013887 |
| | | alpha-2,6-sialyltransferase | |
| GPIAP1 GPI-anchored membrane protein 1 | IPI00030910, IPI00639921 | ST8SIA4 CMP-N-acetylneuraminate-poly-alpha-2,8- | IPI00020201 |
| | | sialyltransferase | |
| GPIAP1 membrane component chromosome 11 surface | IPI00150961, IPI00783872 | STAU1 Isoform Long of Double-stranded RNA-binding | IPI00000001, IPI00218609, |
| marker 1 isoform 1 | | protein Staufen homolog 1 | IPI00641873, IPI00643664 |
| GPR126 Developmentally regulated G-protein-coupled | IPI00651640 | STC1 Stanniocalcin-1 precursor | IPI00005564 |
| receptor alpha 2 | | | |
| GPR126 Developmentally regulated G-protein-coupled | IPI00217481, IPI00423340, IPI00423342, | STC2 Stanniocalcin-2 precursor | IPI00008780 |
| receptor beta 1 | IPI00651640, IPI00651770 | | |
| GPR37 Probable G-protein coupled receptor 37 precursor | IPI00006166 | STCH Stress 70 protein chaperone microsome-associated | IPI00299299 |
| | | 60 kDa protein precursor | |
| GPR56 G protein-coupled receptor 56 isoform b | IPI00397949, IPI00412420, IPI00783104 | STIP1 Stress-induced-phosphoprotein 1 | IPI00013894 |
| GREM2 Gremlin-2 precursor | IPI00328709 | STMN1 Stathmin 1 | IPI00744618 |
| GRHPR Glyoxylate reductase/hydroxypyruvate reductase | IPI00037448, IPI00550682 | STRAP Serine-threonine kinase receptor-associated protein | IPI00294536 |
| GRHPR GRHPR protein (Fragment) | IPI00550682 | STXBP2 Syntaxin-binding protein 2 | IPI00019971 |
| GRN Isoform 1 of Granulins precursor | IPI00296713 | SUB1 Activated RNA polymerase II transcriptional | IPI00221222 |
| | | coactivator p15 | |
| GRP Isoform 1 of Gastrin-releasing peptide precursor | IPI00011722, IPI00218672, IPI00647318 | SUMF2 Isoform 1 of Sulfatase-modifying factor 2 precursor | IPI00171412, IPI00334514, |
| | | | IPI00783919, IPI00784010 |
| GRP Isoform 2 of Gastrin-releasing peptide precursor | IPI00218671 | SUMF2 Isoform 3 of Sulfatase-modifying factor 2 precursor | IPI00334514 |
| GSPT1 G1 to S phase transition protein 1 homolog | IPI00218829 | SUPT16H FACT complex subunit SPT16 | IPI00026970 |
| GSR Isoform Mitochondrial of Glutathione reductase, | IPI00016862 | SUPT6H 199 kDa protein | IPI00456681, IPI00784161 |
| mitochondrial precursor | | | |
| GSS Glutathione synthetase | IPI00010706 | SVEP1 polydom | IPI00301288 |
| GSTA1 Glutathione S-transferase A1 | IPI00657682 | SWAP70 Switch-associated protein 70 | IPI00307200 |
| H1FX Histone H1x | IPI00021924 | SYNCRIP Isoform 1 of Heterogeneous nuclear | IPI00018140, IPI00402182, |
| | | ribonucleoprotein Q | IPI00402183, IPI00402184 |
| H2AFV Histone H2AV | IPI00018278, IPI00218448 | SYNE2 Isoform 1 of Nesprin-2 | IPI00239405, IPI00239406 |
| H2AFY2 Core histone macro-H2A.2 | IPI00220994 | TACC2 Transforming, acidic coiled-coil containing protein 2 | IPI00410127 |
| H3F3A; H3F3B Histone H3.3 | IPI00219038, IPI00413826 | TACSTD1 Tumor-associated calcium signal transducer 1 | IPI00296215 |
| | | precursor | |
| HAGH Hydroxyacylglutathione hydrolase | IPI00745553 | TAF15 Isoform Short of TATA-binding protein-associated | IPI00020194, IPI00294426 |
| | | factor 2N | |
| HARS Histidyl-tRNA synthetase, cytoplasmic | IPI00021808 | TARDBP TAR DNA-binding protein 43 | IPI00025815 |
| HBA1; HBA2 Hemoglobin subunit alpha | IPI00410714 | TARS Threonyl-tRNA synthetase, cytoplasmic | IPI00329633 |
| HBE1 Hemoglobin subunit epsilon | IPI00217471, IPI00220706, IPI00473011, | TBCB Tubulin-specific chaperone B | IPI00293126 |
| | IPI00654755, IPI00657660, | | |
| | IPI00657911, IPI00784636, | | |
| | IPI00796636, IPI00829896, | | |
| | IPI00830113 | | |
| hCG_2028557 similar to unactive progesterone receptor, 23 kD | IPI00176610, IPI00741006 | TCEB1 Transcription elongation factor B polypeptide 1 | IPI00300341, IPI00645048, |
| | | | IPI00791185, IPI00796346 |
| HDAC2 histone deacetylase 2 | IPI00289601 | TCEB2 Transcription elongation factor B polypeptide 2 | IPI00026670 |
| HDGF Hepatoma-derived growth factor | IPI00514330 | TCERG1 Transcription elongation regulator 1 | IPI00247871 |
| HDGFRP3 Hepatoma-derived growth factor-related protein 3 | IPI00007063 | TCN2 Transcobalamin-2 precursor | IPI00219465 |
| HDLBP Vigilin | IPI00022228 | TCP1 T-complex protein 1 subunit alpha | IPI00290566 |
| HEXA Beta-hexosaminidase alpha chain precursor | IPI00027851, IPI00829779 | TFG Protein TFG | IPI00294619, IPI00788849 |
| HEXB Beta-hexosaminidase beta chain precursor | IPI00012585 | TFPI Isoform Alpha of Tissue factor pathway inhibitor | IPI00021834 |
| | | precursor | |
| HGD Homogentisate 1,2-dioxygenase | IPI00303174 | TFPI2 Tissue factor pathway inhibitor 2 precursor | IPI00009198 |
| HINT1 Histidine triad nucleotide-binding protein 1 | IPI00239077 | TFRC Transferrin receptor protein 1 | IPI00022462 |
| HINT2 Histidine triad nucleotide-binding protein 2 | IPI00000335 | TGFB1 Transforming growth factor beta-1 precursor | IPI00000075 |
| HIP1R Huntingtin-interacting protein 1-related protein | IPI00024417, IPI00784470, IPI00788073, | TGFB2 Isoform A of Transforming growth factor beta-2 | IPI00235354 |
| | IPI00792558 | precursor | |
| HIP2 Isoform 1 of Ubiquitin-conjugating enzyme E2-25 kDa | IPI00021370, IPI00784038 | TGFBR3 transforming growth factor, beta receptor III | IPI00304865 |
| HIP2 Isoform 2 of Ubiquitin-conjugating enzyme E2-25 kDa | IPI00019894, IP100021370, IPI00784038 | TGM2 Isoform 1 of Protein-glutamine gamma- | IPI00294578 |
| | | glutamyltransferase 2 | |
| HIST1H1B Histone H1.5 | IPI00217468 | THBS3 Thrombospondin-3 precursor | IPI00329535 |
| HIST1H1C Histone H1.2 | IPI00217465 | THOC4 THO complex subunit 4 | IPI00328840 |
| HIST1H1D Histone H1.3 | IPI00217466 | THRAP3 Thyroid hormone receptor-associated protein 3 | IPI00104050 |
| HIST1H2AA Histone H2A type 1-A | IPI00045109, IPI00219037 | THSD4 thrombospondin, type I, domain containing 4 | IPI00794391 |
| HIST1H2AE; HIST1H2AB Histone H2A type 1-B | IPI00026272 | TIAL1 Nucleolysin TIAR | IPI00005615, IPI00183949, |
| | | | IPI00642600, IPI00644708 |
| HIST1H2AH Histone H2A type 1-H | IPI00081836, IPI00102165, IPI00216457, | TIMM8A Mitochondrial import inner membrane translocase | IPI00028376 |
| | IPI00220855, IPI00255316, | subunit Tim8 A | |
| | IPI00291764, IPI00339274, | | |
| | IPI00552873 | | |
| HIST1H2BD Histone H2B type 1-D | IPI00152906, IPI00303133, IPI00329665, | TIMP2 22 kDa protein | IPI00788747 |
| | IPI00719084, IPI00794461 | | |
| HIST1H2BO Histone H2B type 1-O | IPI00152785 | TIMP2 Metalloproteinase inhibitor 2 precursor | IPI00027166, IPI00787781, |
| | | | IPI00788747 |
| HIST1H3C; HIST1H3G; HIST1H3H; HIST1H3D; HIST1H3I; HIST1H3J; | IPI00465070 | TKT Transketolase | IPI00643920, IPI00788802 |
| HIST1H3A; HIST1H3F; HIST1H3E; HIST1H3B | | | |
| Histone H3.1 | | | |
| HIST2H2AA3; HIST2H2AA4 Histone H2A type 2-A | IPI00216457, IPI00339274 | TKT Transketolase variant (Fragment) | IPI00788802 |
| HIST2H2AC Histone H2A type 2-C | IPI00339274 | TLN1 271 kDa protein | IPI00298994, IPI00784273 |
| HIST2H2BC Histone H2B type 2-C | IPI00454695, IPI00746251 | TLN1 Talin-1 | IPI00784273 |
| HIST2H2BE Histone H2B type 2-E | IPI00003935, IPI00152785, IPI00166293, | TMEM132A transmembrane protein 132A isoform b | IPI00301865, IPI00383814 |
| | IPI00220403, IPI00515061 | | |
| HIST2H3A; HIST2H3C Histone H3.2 | IPI00171611, IPI00719351 | TMEM137; RBM14 Isoform 1 of RNA-binding protein 14 | IPI00013174 |
| HK1 Isoform 1 of Hexokinase-1 | IPI00018246 | TMEM4 Isoform 1 of MIR-interacting saposin-like protein | IPI00443909 |
| | | precursor | |
| HK2 Hexokinase-2 | IPI00102864 | TMOD3 Tropomodulin-3 | IPI00005087 |
| HLA-A HLA class I histocompatibility antigen, A-24 alpha | IPI00742968 | TMPO Isoform Beta of Lamina-associated polypeptide 2, | IPI00030131 |
| chain precursor | | isoforms beta/gamma | |
| HLA-A HLA class I histocompatibility antigen, A-68 alpha | IPI00472882 | TMPO Lamina-associated polypeptide 2 isoform alpha | IPI00216230 |
| chain precursor | | | |
| HLA-A HLA class I histocompatibility antigen, A-69 alpha | IPI00760554 | TNC Isoform 1 of Tenascin precursor | IPI00031008 |
| chain | | | |
| HLA-A HLA class I histocompatibility antigen, A-80 alpha | IPI00472736 | TNFRSF11B Tumor necrosis factor receptor superfamily | IPI00298362 |
| chain precursor | | member 11B precursor | |
| HLA-A Isoform 2 of HLA class I histocompatibility antigen, | IPI00472112, IPI00472448 | TNFRSF19L Tumor necrosis factor receptor superfamily | IPI00064377 |
| A-11 alpha chain precursor | | member 19L precursor | |
| HLA-C; HLA-B; MICA; LOC730410 HLA class I | IPI00471955, IPI00472284, IPI00472456, | TNFRSF1A Tumor necrosis factor receptor superfamily | IPI00018880, IPI00796532 |
| histocompatibility antigen, B-50 alpha chain precursor | IPI00646083, IPI00655604, | member 1A precursor | |
| | IPI00718924, IPI00744375, | | |
| | IPI00744689, IPI00746605, | | |
| | IPI00747198, IPI00816006 | | |
| HLA-C; HLA-B; MICA; LOC730410 HLA class I | IPI00472284 | TNPO1 Transportin 1 (Importin beta-)2 | IPI00024364 |
| histocompatibility antigen, B-56 alpha chain precursor | | | |
| HLA-C; HLA-B; MICA; LOC730410 HLA class I | IPI00472138, IPI00655604, IPI00747198 | TNPO2 Transportin 2 | IPI00419856 |
| histocompatibility antigen, B-58 alpha chain precursor | | | |
| HLA-C; HLA-B; MICA; LOC730410 HLA class I | IPI00471951, IPI00816057 | TOMM70A Mitochondrial precursor proteins import receptor | IPI00015602 |
| histocompatibility antigen, Cw-15 alpha chain precursor | | | |
| HLA-C; HLA-B; MICA; LOC730410 HLA class I | IPI00472605 | TOP1 DNA topoisomerase 1 | IPI00413611 |
| histocompatibility antigen, Cw-2 alpha chain precursor | | | |
| HLA-C; HLA-B; MICA; LOC730410 HLA class I | IPI00651697 | TOP2A 183 kDa protein | IPI00178667, IPI00218753, |
| histocompatibility antigen, Cw-3 alpha chain precursor | | | IPI00218754, IPI00414101, |
| | | | IPI00478232 |
| HLA-C; HLA-B; MICA; LOC730410 HLA class I | IPI00473131 | TOR1B Torsin B precursor | IPI00023137, IPI00477672 |
| histocompatibility antigen, Cw-6 alpha chain precursor | | | |
| HLA-C; HLA-B; MICA; LOC730410 Isoform 2 of HLA class I | IPI00472035, IPI00745649 | TOR3A Isoform 1 of Torsin-3A precursor | IPI00301631 |
| histocompatibility antigen, Cw-16 alpha chain precursor | | | |
| HLA-C; HLA-B; MICA; LOC730410 Major histocompatibility | IPI00789627 | TP53BP1 Isoform 1 of Tumor suppressor p53-binding | IPI00029778, IPI00657708, |
| complex, class I, C | | protein 1 | IPI00742743 |
| HLA-C; HLA-B; MICA; LOC730410 MHC class I antigen | IPI00795906 | TPD52 Tumor protein D52 | IPI00218323, IPI00619951, |
| heavy chain | | | IPI00619958, IPI00782968 |
| HLA-C; HLA-B; MICA; LOC730410 MHC class I chain-related | IPI00107380 | TPD52L1 Tumor protein D53 | IPI00383670, IPI00472076, |
| protein A | | | IPI00793361 |
| HLA-H HLA class I histocompatibility antigen, alpha chain H | IPI00004672 | TPD52L2 24 kDa protein | IPI00743469 |
| precursor | | | |
| HMBS Isoform 1 of Porphobilinogen deaminase | IPI00028160, IPI00219877 | TPD52L2 Isoform 1 of Tumor protein D54 | IPI00306825, IPI00399265, |
| | | | IPI00399267, IPI00743469 |
| HMGB1 High-mobility group box 1 variant (Fragment) | IPI00815806 | TPM1 Isoform 1 of Tropomyosin-1 alpha chain | IPI00014581, IPI00018853, |
| | | | IPI00216135, IPI00296039, |
| | | | IPI00604537, IPI00742825 |
| HMGB2 High mobility group protein B2 | IPI00219097 | TPM2 Isoform 2 of Tropomyosin beta chain | IPI00220709 |
| HMGB3 High mobility group protein B3 | IPI00217477, IPI00411540, IPI00640781, | TPM4 Isoform 1 of Tropomyosin alpha-4 chain | IPI00010779 |
| | IPI00643317 | | |
| HN1 Isoform 1 of Hematological and neurological expressed | IPI00007764, IPI00384857 | TPP1 Isoform 1 of Tripeptidyl-peptidase 1 precursor | IPI00298237, IPI00554538 |
| 1 protein | | | |
| HN1 Isoform 2 of Hematological and neurological expressed | IPI00384857 | TPP2 Tripeptidyl peptidase II | IPI00640197 |
| 1 protein | | | |
| HNRPA0 Heterogeneous nuclear ribonucleoprotein A0 | IPI00011913 | TPR Nucleoprotein TPR | IPI00022970, IPI00742682 |
| HNRPA3 Isoform 1 of Heterogeneous nuclear | IPI00419373 | TPT1 Tumor protein, translationally-controlled 1 | IPI00009943 |
| ribonucleoprotein A3 | | | |
| HNRPAB Isoform 2 of Heterogeneous nuclear | IPI00334587, IPI00334713, IPI00742926 | TPX2 Hepatocellular carcinoma-associated antigen 90 | IPI00102661 |
| ribonucleoprotein A/B | | | |
| HNRPAB Isoform 3 of Heterogeneous nuclear | IPI00334713 | TRIM28 Isoform 1 of Transcription intermediary factor 1- | IPI00438229, IPI00438230 |
| ribonucleoprotein A/B | | beta | |
| HNRPC Isoform C1 of Heterogeneous nuclear | IPI00216592 | TRIM72 Isoform 1 of Tripartite motif-containing protein 72 | IPI00301028 |
| ribonucleoproteins C1/C2 | | | |
| HNRPC Isoform C2 of Heterogeneous nuclear | IPI00477313 | TRIP11 Thyroid receptor-interacting protein 11 | IPI00003515 |
| ribonucleoproteins C1/C2 | | | |
| HNRPDL heterogeneous nuclear ribonucleoprotein D-like | IPI00011274, IPI00045498 | TSKU Tsukushi precursor | IPI00641368 |
| HNRPDL JKTBP1delta6 | IPI00045498 | TSN Translin | IPI00018768 |
| HNRPF Heterogeneous nuclear ribonucleoprotein F | IPI00003881 | TSPAN5 Tetraspanin-5 | IPI00030914 |
| HNRPH1 Heterogeneous nuclear ribonucleoprotein H | IPI00013881 | TTLL12 Tubulin--tyrosine ligase-like protein 12 | IPI00029048 |
| HNRPH1 HNRPH1 protein | IPI00479191 | TUBA1A Tubulin alpha-3 chain | IPI00180675 |
| HNRPH2 Heterogeneous nuclear ribonucleoprotein H′ | IPI00026230 | TUBA1B 46 kDa protein | IPI00792677 |
| HNRPH3 Isoform 1 of Heterogeneous nuclear | IPI00013877, IPI00216493 | TUBA1B Tubulin alpha-ubiquitous chain | IPI00387144, IPI00792677 |
| ribonucleoprotein H3 | | | |
| HNRPL heterogeneous nuclear ribonucleoprotein L isoform a | IPI00027834, IPI00796199 | TUBB Tubulin beta chain | IPI00011654 |
| HNRPM Isoform 1 of Heterogeneous nuclear | IPI00171903, IPI00383296 | TUBB4 Tubulin beta-4 chain | IPI00023598 |
| ribonucleoprotein M | | | |
| HNRPM Isoform 2 of Heterogeneous nuclear | IPI00383296 | TUBB6 46 kDa protein | IPI00641706 |
| ribonucleoprotein M | | | |
| HNRPR Heterogeneous nuclear ribonucleoprotein R | IPI00012074, IPI00644055 | TUBB6 TUBB6 protein | IPI00646779 |
| HNRPU heterogeneous nuclear ribonucleoprotein U isoform a | IPI00644079 | TWF1 twinfilin 1 | IPI00183508, IPI00385754 |
| HNRPU Isoform Short of Heterogeneous nuclear | IPI00479217 | TWF2 Twinfilin-2 | IPI00550917 |
| ribonucleoprotein U | | | |
| HNRPUL1 Isoform 1 of Heterogeneous nuclear | IPI00013070, IPI00167147 | TWSG1 Isoform 1 of Twisted gastrulation protein homolog 1 | IPI00410487, IPI00830092 |
| ribonucleoprotein U-like protein 1 | | precursor | |
| HNRPUL2 Heterogeneous nuclear ribonucleoprotein U-like | IPI00456887, IPI00827583 | TXNDC12 Thioredoxin domain-containing protein 12 | IPI00026328 |
| protein 2 | | precursor | |
| HP1BP3 HP1-BP74 | IPI00296291, IPI00642238, IPI00744429 | TXNDC4 Thioredoxin domain-containing protein 4 precursor | IPI00401264 |
| HRB Isoform 2 of Nucleoporin-like protein RIP | IPI00304693, IPI00607616, IPI00736669 | TXNDC5; NUTED thioredoxin domain containing 5 isoform 2 | IPI00395646 |
| HS6ST2 Isoform 1 of Heparan-sulfate 6-O-sulfotransferase 2 | IPI00157454, IPI00160316, IPI00395692 | TXNL1 Thioredoxin-like protein 1 | IPI00305692, IPI00642032 |
| HSPA4 Heat shock 70 kDa protein 4 | IPI00002966 | TXNL5 Thioredoxin-like protein 5 | IPI00646689 |
| HSPA4L Heat shock 70 kDa protein 4L | IPI00295485, IPI00828021 | U2AF1 Splicing factor U2AF 35 kDa subunit | IPI00005613, IPI00619914, |
| | | | IPI00619942 |
| HSPA9 Heat shock 70 kDa protein 9B variant (Fragment) | IPI00788958 | U2AF2 54 kDa protein | IPI00746657 |
| HSPA9 Stress-70 protein, mitochondrial precursor | IPI00007765 | U2AF2 Splicing factor U2AF 65 kDa subunit | IPI00031556, IPI00746657, |
| | | | IPI00830039 |
| HSPD1 60 kDa heat shock protein, mitochondrial precursor | IPI00784154 | UBA52 ubiquitin and ribosomal protein L40 precursor | IPI00456429, IPI00719280, |
| | | | IPI00743241, IPI00743650, |
| | | | IPI00744274, IPI00783060, |
| | | | IPI00784990, IPI00789107, |
| | | | IPI00789823, |
| | | | IPI00790633, IPI00792712, |
| | | | IPI00793330, IPI00793729, |
| | | | IPI00793810, IPI00794211, |
| | | | IPI00794925, IPI00795527, |
| | | | IPI00796007, |
| | | | IPI00796600, IPI00797400, |
| | | | IPI00797482, IPI |
| HSPD1 61 kDa protein | IPI00472102 | UBAP2L Isoform 3 of Ubiquitin-associated protein 2-like | IPI00181306, IPI00514856, |
| | | | IPI00646016 |
| HSPE1 10 kDa heat shock protein, mitochondrial | IPI00220362 | UBAP2L Ubiquitin associated protein 2-like | IPI00646016 |
| HSPH1 97 kDa protein | IPI00796127 | UBE1 Ubiquitin-activating enzyme E1 | IPI00645078 |
| HSPH1 Isoform Alpha of Heat-shock protein 105 kDa | IPI00514983 | UBE1C NEDD8-activating enzyme E1 catalytic subunit | IPI00328154, IPI00375533 |
| HSPH1 Isoform Beta of Heat-shock protein 105 kDa | IPI00218993, IPI00514983 | UBE2I SUMO-conjugating enzyme UBC9 | IPI00032957, IPI00450472 |
| HTATIP2 Isoform 2 of Oxidoreductase HTATIP2 | IPI00383665 | UBE2L3 Ubiquitin-conjugating enzyme E2 L3 | IPI00021347 |
| HTRA1 HTRA1 protein (Fragment) | IPI00643586 | UBE2N Ubiquitin-conjugating enzyme E2 N | IPI00003949 |
| HTRA1 Serine protease HTRA1 precursor | IPI00003176 | UBE2V2 16 kDa protein | IPI00797889 |
| HYAL1 Isoform 2 of Hyaluronidase-1 precursor | IPI00168847, IPI00178140, IPI00654773 | UBQLN1 Isoform 2 of Ubiquilin-1 | IPI00071180 |
| HYOU1 150 kDa oxygen-regulated protein precursor | IPI00000877 | UBQLN4 Ubiquilin-4 | IPI00024502 |
| IARS Isoleucyl-tRNA synthetase, cytoplasmic | IPI00644127 | UCHL5 Isoform 2 of Ubiquitin carboxyl-terminal hydrolase | IPI00219512, IPI00219513, |
| | | isozyme L5 | IPI00299313, IPI00549820, |
| | | | IPI00549849, IPI00642374 |
| ICAM1 Intercellular adhesion molecule 1 precursor | IPI00008494 | UFC1 Ufm1-conjugating enzyme 1 | IPI00294495 |
| ICAM5 Intercellular adhesion molecule 5 precursor | IPI00290456 | UFD1L ubiquitin fusion degradation 1-like isoform B | IPI00654779 |
| ICOSLG 52 kDa protein | IPI00790218 | UGCGL1 UDP-glucose ceramide glucosyltransferase-like 1 | IPI00024466, IPI00619903 |
| | | isoform 1 | |
| IDE Insulin-degrading enzyme | IPI00220373 | UGCGL1 UDP-glucose:glycoprotein glucosyltransferase 1 | IPI00619903 |
| | | precursor | |
| IDH2 Isocitrate dehydrogenase [NADP], mitochondrial | IPI00011107 | UGDH UDP-glucose 6-dehydrogenase | IPI00031420 |
| precursor | | | |
| IDI1 isopentenyl-diphosphate delta isomerase | IPI00220014 | UNQ2541 MSFL2541 | IPI00399139 |
| IGF1 Insulin-like growth factor IB precursor | IPI00433029 | UPF1 Isoform 1 of Regulator of nonsense transcripts 1 | IPI00034049, IPI00399170 |
| IGF2R Cation-independent mannose-6-phosphate receptor | IPI00289819 | UROD Uroporphyrinogen decarboxylase | IPI00301489 |
| precursor | | | |
| IGFBP3 insulin-like growth factor binding protein 3 isoform a | IPI00556155 | USP14 Ubiquitin carboxyl-terminal hydrolase 14 | IPI00219913 |
| precursor | | | |
| IGFBP3 Insulin-like growth factor-binding protein 3 | IPI00018305, IPI00556155 | USP15 Isoform 1 of Ubiquitin carboxyl-terminal hydrolase | IPI00000728 |
| precursor | | 15 | |
| IGFBP5 Insulin-like growth factor-binding protein 5 | IPI00029236 | USP5 Isoform Long of Ubiquitin carboxyl-terminal hydrolase 5 | IPI00024664, IPI00375145 |
| precursor | | | |
| IGFBPL1 Insulin-like growth factor binding protein-like 1 | IPI00291987 | USP5 Isoform Short of Ubiquitin carboxyl-terminal hydrolase 5 | IPI00375145 |
| IGSF8 Isoform 1 of Immunoglobulin superfamily member 8 | IPI00056478 | USP7 Ubiquitin carboxyl-terminal hydrolase 7 | IPI00003965, IPI00646721, |
| precursor | | | IPI00783739 |
| IL13RA1 Interleukin-13 receptor alpha-1 chain precursor | IPI00020354, IPI00647552 | USP7 Ubiquitin-specific protease 7 isoform | IPI00646721 |
| IL17C Interleukin-17C precursor | IPI00002316 | UTP14A Isoform 1 of U3 small nucleolar RNA-associated | IPI00107113 |
| | | protein 14 homolog A | |
| IL1R2 Interleukin-1 receptor type II precursor | IPI00021382 | UXS1 Isoform 2 of UDP-glucuronic acid decarboxylase 1 | IPI00410544, IPI00657807 |
| IL6 Interleukin-6 precursor | IPI00007793 | VAPA 14 kDa protein | IPI00642826 |
| IL6ST Isoform 1 of Interleukin-6 receptor subunit beta | IPI00297124 | VAPA Vesicle-associated membrane protein-associated | IPI00170692 |
| precursor | | protein A | |
| ILF2 Interleukin enhancer-binding factor 2 | IPI00005198 | VARS Valyl-tRNA synthetase | IPI00000873, IPI00829641 |
| ILF3 Isoform 1 of Interleukin enhancer-binding factor 3 | IPI00298788, IPI00418313 | VASN Vasorin precursor | IPI00395488, IPI00745461 |
| ILF3 Isoform 5 of Interleukin enhancer-binding factor 3 | IPI00219330 | VAT1 Synaptic vesicle membrane protein VAT-1 homolog | IPI00156689 |
| ILKAP Integrin-linked kinase-associated serine/threonine | IPI00006164 | VBP1 Von Hippel-Lindau binding protein 1 | IPI00334159 |
| phosphatase 2C | | | |
| IMMT Isoform 1 of Mitochondrial inner membrane protein | IPI00009960, IPI00554469 | VCAN Isoform V0 of Versican core protein precursor | IPI00009802, IPI00215628, |
| | | | IPI00215629, IPI00215631 |
| IMPA1 Inositol monophosphatase | IPI00020906 | VCL Isoform 2 of Vinculin | IPI00307162 |
| IMPDH2 Inosine-5′-monophosphate dehydrogenase 2 | IPI00291510 | VEGFC Vascular endothelial growth factor C precursor | IPI00028076 |
| INSM1 Insulinoma-associated protein 1 | IPI00005944 | VGF VGF nerve growth factor inducible precursor | IPI00069058 |
| IPO7 120 kDa protein | IPI00007402, IPI00784008 | VIL1 Villin-1 | IPI00218852 |
| IQGAP1 Ras GTPase-activating-like protein IQGAP1 | IPI00009342 | VISA Isoform 1 of Mitochondrial antiviral-signaling protein | IPI00020719 |
| ISG15 Interferon-induced 17 kDa protein precursor | IPI00375631 | VIT Hypothetical protein VIT (vitrin) | IPI00180759, IPI00784236, |
| | | | IPI00785025 |
| ISYNA1 55 kDa protein | IPI00478861, IPI00549569, IPI00640098, | VPS29 Isoform 1 of Vacuolar protein sorting-associated | IPI00170796, IPI00184284, |
| | IPI00644161, IPI00645069 | protein 29 | IPI00798102 |
| ITGB1 integrin beta 1 isoform 1A precursor | IPI00645194 | VPS35 Vacuolar protein sorting-associated protein 35 | IPI00018931 |
| ITGB4BP Eukaryotic translation initiation factor 6 | IPI00010105 | VSNL1 Visinin-like protein 1 | IPI00216313 |
| ITGBL1 Ten integrin EGF-like repeat domains protein | IPI00640865 | VWA1 von Willebrand factor A domain-related protein | IPI00396383 |
| precursor | | isoform 1 | |
| ITM2B Integral membrane protein 2B | IPI00031821 | VWA2 von Willebrand factor A domain-containing protein 2 | IPI00394834 |
| ITPA inosine triphosphatase isoform b | IPI00375446 | WARS tryptophanyl-tRNA synthetase isoform b | IPI00412737 |
| JAG1 Jagged-1 precursor | IPI00099650 | WARS Tryptophanyl-tRNA synthetase, cytoplasmic | IPI00295400, IPI00412737 |
| KARS Lysyl-tRNA synthetase | IPI00014238, IPI00307092 | WDR1 Isoform 1 of WD repeat protein 1 | IPI00746165 |
| KCTD12 BTB/POZ domain-containing protein KCTD12 | IPI00060715 | WDR1 Isoform 2 of WD repeat protein 1 | IPI00216256, IPI00746165 |
| KHDRBS1 Isoform 1 of KH domain-containing, RNA- | IPI00008575, IPI00082310, IPI00385834, | WDR61 WD repeat protein 61 | IPI00019269, IPI00789452 |
| binding, signal transduction-associated protein 1 | IPI00479209 | | |
| KHDRBS1 Isoform 3 of KH domain-containing, RNA- | IPI00082310 | WDR77 Methylosome protein 50 | IPI00012202 |
| binding, signal transduction-associated protein 1 | | | |
| KHSRP Far upstream element-binding protein 2 | IPI00298363 | WIF1 Wnt inhibitory factor 1 precursor | IPI00001863 |
| KHSRP KH-type splicing regulatory protein | IPI00479786 | WISP2 WNT1-inducible-signaling pathway protein 2 | IPI00022052 |
| | | precursor | |
| KIAA0310 hypothetical protein LOC9919 | IPI00641384 | WNT5A Protein Wnt-5a precursor | IPI00013178, IPI00795811 |
| KIAA0319L KIAA0319-like | IPI00472754, IPI00749513 | XPO1 Exportin-1 | IPI00298961, IPI00784388 |
| KIAA1598 Protein KIAA1598 | IPI00448751 | XRCC5 ATP-dependent DNA helicase 2 subunit 2 | IPI00220834 |
| KIAA1822L hypothetical protein LOC79802 | IPI00015504 | XRCC6 70 kDa protein | IPI00465430 |
| KIAA1967 Isoform 1 of Protein KIAA1967 | IPI00783537 | XTP3TPA CDNA: FLJ21190 fis, clone CAS12333 | IPI00012197 |
| KIF5B Kinesin heavy chain | IPI00012837 | XYLT1 Xylosyltransferase 1 | IPI00183487 |
| KIT Mast/stem cell growth factor receptor precursor | IPI00022296 | XYLT2 Isoform 1 of Xylosyltransferase 2 | IPI00432723 |
| KITLG Isoform 2 of Kit ligand precursor | IPI00220142 | YAP1 YAP1 protein | IPI00216919 |
| KLC1 Isoform K of Kinesin light chain 1 | IPI00394906 | YARS Tyrosyl-tRNA synthetase, cytoplasmic | IPI00007074 |
| KLK14 kallikrein 14 preproprotein | IPI00793215 | YBX1 35 kDa protein | IPI00031812, IPI00385699, |
| | | | IPI00450235 |
| KPNA2 Importin alpha-2 subunit | IPI00002214 | YBX1 Nuclease sensitive element binding protein-1 | IPI00450235 |
| KPNA4 Importin alpha-4 subunit | IPI00012578 | YBX1 Nuclease sensitive element-binding protein 1 | IPI00031812 |
| KPNB1 Importin beta-1 subunit | IPI00001639 | YES1 Proto-oncogene tyrosine-protein kinase Yes | IPI00013981, IPI00477734 |
| KREMEN1 Isoform 1 of Kremen protein 1 precursor | IPI00140177, IPI00218929, IPI00651704 | YKT6 Synaptobrevin homolog YKT6 | IPI00008569 |
| KRT16 Keratin, type I cytoskeletal 16 | IPI00217963 | ZFR 117 kDa protein | IPI00333858, IPI00748303 |
| KRT18 Keratin, type I cytoskeletal 18 | IPI00784347 | ZNF207 Isoform 1 of Zinc finger protein 207 | IPI00013457, IPI00219759, |
| | | | IPI00788670, IPI00792837 |
| | | ZNF207 Isoform 3 of Zinc finger protein 207 | IPI00219760 |

TABLE 9
Distribution of markers in serum of controls (0) and
patients with NSCLC (1)

| Marker | Control/Case | n* | AUC (95% CI) | P value |
|---|---|---|---|---|
| Osteoprotegerin | 0 | 25 | 0.81 | <0.0002 |
| | 1 | 25 | (0.68-0.94) | |
| Pentraxin 3 | 0 | 25 | 0.92 | <0.0001 |
| | 1 | 25 | (0.84-0.99) | |
| sTNF RI | 0 | 25 | 0.83 | <0.0001 |
| | 1 | 25 | (0.70-0.95) | |
| Follistatin | 0 | 25 | 0.94 | <0.0001 |
| | 1 | 25 | (0.88-1.00) | |
| ADAM-17 | 0 | 21 | 0.78 | <0.002 |
| | 1 | 21 | (0.63-0.94) | |

*Number of patients; AUC: area under curve; CI: confidence interval.

TABLE 10
Sequences
ADAM-17: a disintegrin and metalloprotease domain 17 preproprotein [Homo sapiens]
1. NCBI Reference Sequence: NP_003174.3
>gi|73747889|ref|NP_003174.3| a disintegrin and metalloprotease domain 17
preproprotein [Homo sapiens]
MRQSLLFLTSVVPFVLAPRPPDDPGFGPHQRLEKLDSLLSDYDILSLSNIQQHSVRKRDLQTSTHVETLL
TFSALKRHFKLYLTSSTERFSQNFKVVVVDGKNESEYTVKWQDFFTGHVVGEPDSRVLAHIRDDDVIIRI
NTDGAEYNIEPLWRFVNDTKDKRMLVYKSEDIKNVSRLQSPKVCGYLKVDNEELLPKGLVDREPPEELVH
RVKRRADPDPMKNTCKLLVVADHRFYRYMGRGEESTTTNYLIELIDRVDDIYRNTSWDNAGFKGYGIQIE
QIRILKSPQEVKPGEKHYNMAKSYPNEEKDAWDVKMLLEQFSFDIAEEASKVCLAHLFTYQDFDMGTLGL
AYVGSPRANSHGGVCPKAYYSPVGKKNIYLNSGLTSTKNYGKTILTKEADLVTTHELGHNFGAEHDPDGL
AECAPNEDQGGKYVMYPIAVSGDHENNKMFSNCSKQSIYKTIESKAQECFQERSNKVCGNSRVDEGEECD
PGIMYLNNDTCCNSDCTLKEGVQCSDRNSPCCKNCQFETAQKKCQEAINATCKGVSYCTGNSSECPPPGN
AEDDTVCLDLGKCKDGKCIPFCEREQQLESCACNETDNSCKVCCRDLSGRCVPYVDAEQKNLFLRKGKPC
TVGFCDMNGKCEKRVQDVIERFWDFIDQLSINTFGKFLADNIVGSVLVFSLIFWIPFSILVHCVDKKLDK
QYESLSLFHPSNVEMLSSMDSASVRIIKPFPAPQTPGRLQPAPVIPSAPAAPKLDHQRMDTIQEDPSTDS
HMDEDGFEKDPFPNSSTAAKSFEDLTDHPVTRSEKAASFKLQRQNRVDSKETEC
Osteoprotegerin precursor [Homo sapiens]
2. NCBI Reference Sequence: NP_002537.3
>gi|148743793|ref|NP_002537.3| osteoprotegerin precursor [Homo sapiens]
MNNLLCCALVFLDISIKWTTQETFPPKYLHYDEETSHQLLCDKCPPGTYLKQHCTAKWKTVCAPCPDHYY
TDSWHTSDECLYCSPVCKELQYVKQECNRTHNRVCECKEGRYLEIEFCLKHRSCPPGFGVVQAGTPERNT
VCKRCPDGFFSNETSSKAPCRKHTNCSVFGLLLTQKGNATHDNICSGNSESTQKCGIDVTLCEEAFFRFA
VPTKFTPNWLSVLVDNLPGTKVNAESVERIKRQHSSQEQTFQLLKLWKHQNKDQDIVKKIIQDIDLCENS
VQRHIGHANLTFEQLRSLMESLPGKKVGAEDIEKTIKACKPSDQILKLLSLWRIKNGDQDTLKGLMHALK
HSKTYHFPKTVTQSLKKTIRFLHSFTMYKLYQKLFLEMIGNQVQSVKISCL
Pentraxin 3 [Homo sapiens]
3. NCBI Reference Sequence: NP 002843.2
>gi|167900484|ref|NP_002843.2| pentraxin 3 [Homo sapiens]
MHLLAILFCALWSAVLAENSDDYDLMYVNLDNEIDNGLHPTEDPTPCACGQEHSEWDKLFIMLENSQMRE
RMLLQATDDVLRGELQRLREELGRLAESLARPCAPGAPAEARLTSALDELLQATRDAGRRLARMEGAEAQ
RPEEAGRALAAVLEELRQTRADLHAVQGWAARSWLPAGCETAILFPMRSKKIFGSVHPVRPMRLESFSAC
IWVKATDVLNKTILFSYGTKRNPYEIQLYLSYQSIVFVVGGEENKLVAEAMVSLGRWTHLCGTWNSEEGL
TSLWVNGELAATTVEMATGHIVPEGGILQIGQEKNGCCVGGGFDETLAFSGRLTGFNIWDSVLSNEEIRE
TGGAESCHIRGNIVGWGVTEIQPHGGAQYVS
Follistatin isoform FST344 precursor [Homo sapiens]
4. NCBI Reference Sequence: NP_037541.1
>gi|7242222|ref|NP_037541.1| follistatin isoform FST344 precursor [Homo sapiens]
MVRARHQPGGLCLLLLLLCQFMEDRSAQAGNCWLRQAKNGRCQVLYKTELSKEECCSTGRLSTSWTEEDV
NDNTLFKWMIFNGGAPNCIPCKETCENVDCGPGKKCRMNKKNKPRCVCAPDCSNITWKGPVCGLDGKTYR
NECALLKARCKEQPELEVQYQGRCKKTCRDVFCPGSSTCVVDQTNNAYCVTCNRICPEPASSEQYLCGND
GVTYSSACHLRKATCLLGRSIGLAYEGKCIKAKSCEDIQCTGGKKCLWDFKVGRGRCSLCDELCPDSKSD
EPVCASDNATYASECAMKEAACSSGVLLEVKHSGSCNSISEDTEEEEEDEDQDYSFPISSILEW
Tumor necrosis factor receptor 1 precursor [Homo sapiens]
5. NCBI Reference Sequence: NP_001056.1
>gi|4507575|ref|NP_001056.1| tumor necrosis factor receptor 1 precursor
[Homo sapiens]
MGLSTVPDLLLPLVLLELLVGIYPSGVIGLVPHLGDREKRDSVCPQGKYIHPQNNSICCTKCHKGTYLYN
DCPGPGQDTDCRECESGSFTASENHLRHCLSCSKCRKEMGQVEISSCTVDRDTVCGCRKNQYRHYWSENL
FQCFNCSLCLNGTVHLSCQEKQNTVCTCHAGFFLRENECVSCSNCKKSLECTKLCLPQIENVKGTEDSGT
TVLLPLVIFFGLCLLSLLFIGLMYRYQRWKSKLYSIVCGKSTPEKEGELEGTTTKPLAPNPSFSPTPGFT
PTLGFSPVPSSTFTSSSTYTPGDCPNFAAPRREVAPPYQGADPILATALASDPIPNPLQKWEDSAHKPQS
LDTDDPATLYAVVENVPPLRWKEFVRRLGLSDHEIDRLELQNGRCLREAQYSMLATWRRRTPRREATLEL
LGRVLRDMDLLGCLEDIEEALCGPAALPPAPSLLR

TABLE 11
The sensitivity of Pentraxin 3 versus high-risk controls and all controls
at various specificity cut-offs. The AUCs and confidence intervals
are compared to high-risk controls.

| | Sensitivity | | | |
|---|---|---|---|---|
| | Vs. High-Risk Controls | | Vs. All Controls | |
| Specificity | Estimate | 95% C.I. | Estimate | 95% C.I. |
| 0.99 | 0.054 | (0.000, 0.158) | 0.054 | (0.000, 0.133) |
| .095 | 0.192 | (0.094, 0.296) | 0.177 | (0.118, 0.281) |
| 0.90 | 0.374 | (0.212, 0.478) | 0.251 | (0.163, 0.399) |
| 0.80 | 0.478 | (0.394, 0.557) | 0.448 | (0.365, 0.542) |
| 0.70 | 0.611 | (0.493, 0.734) | 0.512 | (0.419, 0.611) |
| 0.60 | 0.719 | (0.621, 0.783) | 0.655 | (0.542, 0.734) |
| 0.50 | 0.744 | (0.670, 0.798) | 0.739 | (0.680, 0.808) |

TABLE 12
The AUC and confidence intervals for Pentraxin 3 in patients based on
histology of lung cancer.

| Lung Cancer Type | N | AUC | 95% CI |
|---|---|---|---|
| All types | 120 | 0.67 | 0.60-0.74 |
| NSCLC | 90 | 0.65 | 0.58-0.72 |
| SCLC | 13 | 0.67 | 0.47-0.83 |
| NSCLC | | | |
| Adenocarcinoma | 57 | 0.65 | 0.56-0.76 |
| Squamous | 30 | 0.63 | 0.52-0.75 |

### CITATIONS FOR REFERENCES REFERRED TO IN THE SPECIFICATION

### References

1 Jemal, A., Siegel, R., Ward, E., Murray, T., Xu, J. and Thun, M. J. (2007) Cancer statistics CA Cancer J. Clin. 57, 43-66
2 Bryborn, M., Adner, M. and Cardell, L. O. (2005) Psoriasin, one of several new proteins identified in nasal lavage fluid from allergic and non-allergic individuals using 2-dimensional gel electrophoresis and mass spectrometry Respir. Res. 6, 118
3 Casado, B., Pannell, L. K., ladarola, P. and Baraniuk, J. N. (2005) Identification of human nasal mucous proteins using proteomics Proteomics 5, 2949-59
4 Lindahl, M., Irander, K., Tagesson, C. and Stahlbom, B. (2004) Nasal lavage fluid and proteomics as means to identify the effects of the irritating epoxy chemical dimethylbenzylamine Biomarkers 9, 56-70
5 Sabounchi-Schutt, F., Astrom, J., Hellman, U., Eklund, A. and Grunewald, J. (2003) Changes in bronchoalveolar lavage fluid proteins in sarcoidosis: a proteomics approach Eur. Respir. J. 21, 414-20
6 Wu, J., Kobayashi, M., Sousa, E. A., Liu, W., Cai, J., Goldman, S. J., Dorner, A. J., Projan, S. J., Kavuru, M. S., Qiu, Y. and Thomassen, M. J. (2005) Differential proteomic analysis of bronchoalveolar lavage fluid in asthmatics following segmental antigen challenge Mol. Cell. Proteomics 4, 1251-64
7 Xie, H., Rhodus, N. L., Griffin, R. J., Carlis, J. V. and Griffin, T. J. (2005) A catalogue of human saliva proteins identified by free flow electrophoresis-based peptide separation and tandem mass spectrometry Mol. Cell. Proteomics 4, 1826-30
8 Hu, S., Xie, Y., Ramachandran, P., Ogorzalek Loo, R. R., Li, Y., Loo, J. A. and Wong, D. T. (2005) Large-scale identification of proteins in human salivary proteome by liquid chromatography/mass spectrometry and two-dimensional gel electrophoresis-mass spectrometry Proteomics 5, 1714-28
9 Nicholas, B., Skipp, P., Mould, R., Rennard, S., Davies, D. E., O'Connor, C. D. and Djukanovic, R. (2006) Shotgun proteomic analysis of human-induced sputum Proteomics 6, 4390-401
10 Casado, B., ladarola, P., Pannell, L. K., Luisetti, M., Corsico, A., Ansaldo, E., Ferrarotti, I., Boschetto, P. and Baraniuk, J. N. (2007) Protein expression in sputum of smokers and chronic obstructive pulmonary disease patients: a pilot study by CapLC-ESI-Q-TOF J. Proteome Res. 6, 4615-23
11 Tyan, Y. C., Wu, H. Y., Lai, W. W., Su, W. C. and Liao, P. C. (2005) Proteomic profiling of human pleural effusion using two-dimensional nano liquid chromatography tandem mass spectrometry J. Proteome Res. 4, 1274-86
12 Jacobs, J. M., Adkins, J. N., Qian, W. J., Liu, T., Shen, Y., Camp, D. G., 2nd and Smith, R. D. (2005) Utilizing human blood plasma for proteomic biomarker discovery J. Proteome Res. 4, 1073-85
13 Qian, W. J., Jacobs, J. M., Liu, T., Camp, D. G., 2nd and Smith, R. D. (2006) Advances and challenges in liquid chromatography-mass spectrometry-based proteomics profiling for clinical applications Mol. Cell. Proteomics 5, 1727-44
14 Kulasingam, V. and Diamandis, E. P. (2007) Proteomics analysis of conditioned media from three breast cancer cell lines: a mine for biomarkers and therapeutic targets Mol. Cell. Proteomics 6, 1997-2011
15 Sardana, G., Jung, K., Stephan, C. and Diamandis, E. P. (2008) Proteomic analysis of conditioned media from the PC3, LNCaP, and 22Rv1 prostate cancer cell lines: discovery and validation of candidate prostate cancer biomarkers J. Proteome Res. 7, 3329-38
16 Tachibana, I., Mori, M., Tanio, Y., Hosoe, S., Sakuma, T., Osaki, T., Ueno, K., Kumagai, T., Kijima, T. and Kishimoto, T. (1996) A 100-kDa protein tyrosine phosphorylation is concurrent with beta 1 integrin-mediated morphological differentiation in neuroblastoma and small cell lung cancer cells Exp. Cell. Res. 227, 230-9
17 Lou, X., Xiao, T., Zhao, K., Wang, H., Zheng, H., Lin, D., Lu, Y., Gao, Y., Cheng, S., Liu, S, and Xu, N. (2007) Cathepsin D is secreted from M-BE cells: its potential role as a biomarker of lung cancer J. Proteome Res. 6, 1083-92
18 Xiao, T., Ying, W., Li, L., Hu, Z., Ma, Y., Jiao, L., Ma, J., Cai, Y., Lin, D., Guo, S., Han, N., Di, X., Li, M., Zhang, D., Su, K., Yuan, J., Zheng, H., Gao, M., He, J., Shi, S., Li, W., Xu, N., Zhang, H., Liu, Y., Zhang, K., Gao, Y., Qian, X. and Cheng, S. (2005) An approach to studying lung cancer-related proteins in human blood Mol. Cell. Proteomics 4, 1480-6
Borgono, C. A., Michael, I. P., Shaw, J. L., Luo, L. Y., Ghosh, M. C., Soosaipillai, A., Grass, L., Katsaros, D. and Diamandis, E. P. (2007) Expression and functional characterization of the cancer-related serine protease, human tissue kallikrein 14 J. Biol. Chem. 282, 2405-22
20 Diamandis, E. P., Borgono, C. A., Scorilas, A., Harbeck, N., Dorn, J. and Schmitt, M. (2004) Human kallikrein 11: an indicator of favorable prognosis in ovarian cancer patients Clin. Biochem. 37, 823-9
21 Shaw, J. L. and Diamandis, E. P. (2007) Distribution of 15 human kallikreins in tissues and biological fluids Clin. Chem. 53, 1423-32
22 Kersey, P. J., Duarte, J., Williams, A., Karavidopoulou, Y., Birney, E. and Apweiler, R. (2004) The International Protein Index: an integrated database for proteomics experiments Proteomics 4, 1985-8
23 Keller, A., Nesvizhskii, A. I., Kolker, E. and Aebersold, R. (2002) Empirical statistical model to estimate the accuracy of peptide identifications made by MS/MS and database search Anal. Chem. 74, 5383-92
Nesvizhskii, A. I., Keller, A., Kolker, E. and Aebersold, R. (2003) A statistical model for identifying proteins by tandem mass spectrometry Anal. Chem. 75, 4646-58
25 Huang, L. J., Chen, S. X., Huang, Y., Luo, W. J., Jiang, H. H., Hu, Q. H., Zhang, P. F. and Yi, H. (2006) Proteomics-based identification of secreted protein dihydrodiol dehydrogenase as a novel serum markers of non-small cell lung cancer Lung Cancer 54, 87-94
26 Tian, T., Hao, J., Xu, A., Luo, C., Liu, C., Huang, L., Xiao, X. and He, D. (2007) Determination of metastasis-associated proteins in non-small cell lung cancer by comparative proteomic analysis Cancer Sci. 98, 1265-74
27 Salgia, R., Harpole, D., Herndon, J. E., 2nd, Pisick, E., Elias, A. and Skarin, A. T. (2001) Role of serum tumor markers CA 125 and CEA in non-small cell lung cancer Anticancer Res. 21, 1241-6
28 Shoji, F., Yoshino, I., Yano, T., Kometani, T., Ohba, T., Kouso, H., Takenaka, T., Miura, N., Okazaki, H. and Maehara, Y. (2007) Serum carcinoembryonic antigen level is associated with epidermal growth factor receptor mutations in recurrent lung adenocarcinomas Cancer 110, 2793-8
29 Nisman, B., Heching, N., Biran, H., Barak, V. and Peretz, T. (2006) The prognostic significance of circulating neuroendocrine markers chromogranin a, pro-gastrin-releasing peptide and neuron-specific enolase in patients with advanced non-small-cell lung cancer Tumour Biol. 27, 8-16
30 Totsch, M., Muller, L. C., Hittmair, A., Ofner, D., Gibbs, A. R. and Schmid, K. W. (1992) Immunohistochemical demonstration of chromogranins A and B in neuroendocrine tumors of the lung Hum. Pathol. 23, 312-6
31 Takada, M., Kusunoki, Y., Masuda, N., Matui, K., Yana, T., Ushijima, S., lida, K., Tamura, K., Komiya, T., Kawase, I., Kikui, N., Morino, H. and Fukuoka, M. (1996) Pro-gastrin-releasing peptide (31-98) as a tumour marker of small-cell lung cancer: comparative evaluation with neuron-specific enolase Br. J. Cancer 73, 1227-32
32 Bhattacharjee, A., Richards, W. G., Staunton, J., Li, C., Monti, S., Vasa, P Ladd, C., Beheshti, J., Bueno, R., Gillette, M., Loda, M., Weber, G., Mark, E. J., Lander, E. S., Wong, W., Johnson, B. E., Golub, T. R., Sugarbaker, D. J. and Meyerson, M. (2001) Classification of human lung carcinomas by mRNA expression profiling reveals distinct adenocarcinoma subclasses Proc. Natl. Acad. Sci. USA 98, 13790-5
33 Planque, C., Li, L., Zheng, Y., Soosaipillai, A., Reckamp, K., Chia, D., Diamandis, E. P. and Goodglick, L. (2008) A multiparametric serum kallikrein panel for diagnosis of non-small cell lung carcinoma Clin. Cancer Res. 14, 1355-62
34 Planque, C., Blechet, C., Ayadi-Kaddour, A., Heuze-Vourc'h, N., Dumont, P., Guyetant, S., Diamandis, E. P., El Mezni, F. and Courty, Y. (2008) Quantitative RT-PCR analysis and immunohistochemical localization of the kallikrein-related peptidases 13 and 14 in lung Biol. Chem. 389, 781-6
35 Lynch, D. F., Jr., Hassen, W., Clements, M. A., Schellhammer, P. F. and Wright, G. L., Jr. (1997) Serum levels of endothelial and neural cell adhesion molecules in prostate cancer Prostate 32, 214-20
Jaques, G., Auerbach, B., Pritsch, M., Wolf, M., Madry, N. and Havemann, K. (1993) Evaluation of serum neural cell adhesion molecule as a new tumor marker in small cell lung cancer Cancer 72, 418-25
37 Ledermann, J. A., Pasini, F., Olabiran, Y. and Pelosi, G. (1994) Detection of the neural cell adhesion molecule (NCAM) in serum of patients with small-cell lung cancer (SCLC) with “limited” or “extensive” disease, and bone-marrow infiltration Int. J. Cancer Suppl 8, 49-52
38 Magi, B., Bargagli, E., Bini, L. and Rottoli, P. (2006) Proteome analysis of bronchoalveolar lavage in lung diseases Proteomics 6, 6354-69
39 Issaq, H. J. (2001) The role of separation science in proteomics research Electrophoresis 22, 3629-38
40 Cho, C. K., Shan, S. J., Winsor, E. J. and Diamandis, E. P. (2007) Proteomics analysis of human amniotic fluid Mol. Cell. Proteomics 6, 1406-15
41 Shaw, J. L., Smith, C. R. and Diamandis, E. P. (2007) Proteomic analysis of human cervico-vaginal fluid J. Proteome Res. 6, 2859-65
42 Martin, D. B., Gifford, D. R., Wright, M. E., Keller, A., Yi, E., Goodlett, D. R., Aebersold, R. and Nelson, P. S. (2004) Quantitative proteomic analysis of proteins released by neoplastic prostate epithelium Cancer Res. 64, 347-55
43 Li, C., Hong, Y., Tan, Y. X., Zhou, H., Ai, J. H., Li, S. J., Zhang, L., Xia, Q. C., Wu, J. R., Wang, H. Y. and Zeng, R. (2004) Accurate qualitative and quantitative proteomic analysis of clinical hepatocellular carcinoma using laser capture microdissection coupled with isotope-coded affinity tag and two-dimensional liquid chromatography mass spectrometry Mol. Cell. Proteomics 3, 399-409
44 Yocum, A. K., Busch, C. M., Felix, C. A. and Blair, I. A. (2006) Proteomics-based strategy to identify biomarkers and pharmacological targets in leukemias with t(4;11) translocations J. Proteome Res. 5, 2743-53
45 Kapp, E. A., Schutz, F., Connolly, L. M., Chakel, J. A., Meza, J. E., Miller, C. A., Fenyo, D., Eng, J. K., Adkins, J. N., Omenn, G. S, and Simpson, R. J. (2005) An evaluation, comparison, and accurate benchmarking of several publicly available MS/MS search algorithms: sensitivity and specificity analysis Proteomics 5, 3475-90
46 Domon, B. and Aebersold, R. (2006) Challenges and opportunities in proteomics data analysis Mol. Cell. Proteomics 5, 1921-6
47 Kagohashi, K., Satoh, H., Kurishima, K., Kadono, K., Ishikawa, H., Ohtsuka, M. and Sekizawa, K. (2008) Squamous cell carcinoma antigen in lung cancer and nonmalignant respiratory diseases Lung 186, 323-6
48 Vassilakopoulos, T., Troupis, T., Sotiropoulou, C., Zacharatos, P., Katsaounou, P., Parthenis, D., Noussia, O., Troupis, G., Papiris, S., Kittas, C., Roussos, C., Zakynthinos, S, and Gorgoulis, V. (2001) Diagnostic and prognostic significance of squamous cell carcinoma antigen in non-small cell lung cancer Lung Cancer 32, 137-44
49 Schneider, J., Velcovsky, H. G., Morr, H., Katz, N., Neu, K. and Eigenbrodt, E. (2000) Comparison of the tumor markers tumor M2-PK, CEA, CYFRA 21-1, NSE and SCC in the diagnosis of lung cancer Anticancer Res. 20, 5053-8
50 Liotta, L. A., Ferrari, M. and Petricoin, E. (2003) Clinical proteomics: written in blood Nature 425, 905
51 Santiago-Josefat, B., Esselens, C., Bech-Serra, J. J. and Arribas, J. (2007) Post-transcriptional up-regulation of ADAM17 upon epidermal growth factor receptor activation and in breast tumors J. Biol. Chem. 282, 8325-31
52 McGowan, P. M., Ryan, B. M., Hill, A. D., McDermott, E., O'Higgins, N. and Duffy, M. J. (2007) ADAM-17 expression in breast cancer correlates with variables of tumor progression Clin. Cancer Res. 13, 2335-43
53 McGowan, P. M., McKiernan, E., Bolster, F., Ryan, B. M., Hill, A. D., McDermott, E. W., Evoy, D., O'Higgins, N., Crown, J. and Duffy, M. J. (2008) ADAM-17 predicts adverse outcome in patients with breast cancer Ann. Oncol. 19, 1075-81
Zhou, B. B., Peyton, M., He, B., Liu, C., Girard, L., Caudler, E., Lo, Y., Baribaud, F., Mikami, I., Reguart, N., Yang, G., Li, Y., Yao, W., Vaddi, K., Gazdar, A. F., Friedman, S. M., Jablons, D. M., Newton, R. C., Fridman, J. S., Minna, J. D. and Scherle, P. A. (2006) Targeting ADAM-mediated ligand cleavage to inhibit HER3 and EGFR pathways in non-small cell lung cancer Cancer Cell. 10, 39-50
55 Breviario, F., d'Aniello, E. M., Golay, J., Peri, G., Bottazzi, B., Bairoch, A., Saccone, S., Marzella, R., Predazzi, V., Rocchi, M. and et al. (1992) Interleukin-1-inducible genes in endothelial cells. Cloning of a new gene related to C-reactive protein and serum amyloid P component J. Biol. Chem. 267, 22190-7
56 He, X., Han, B. and Liu, M. (2007) Long pentraxin 3 in pulmonary infection and acute lung injury Am. J. Physiol. Lung Cell. Mol. Physiol. 292, L1039-49
57 Thomas, T. Z., Wang, H., Niclasen, P., O'Bryan, M. K., Evans, L. W., Groome, N. P., Pedersen, J. and Risbridger, G. P. (1997) Expression and localization of activin subunits and follistatins in tissues from men with high grade prostate cancer J. Clin. Endocrinol. Metab. 82, 3851-8
58 McPherson, S. J., Mellor, S. L., Wang, H., Evans, L. W., Groome, N. P. and Risbridger, G. P. (1999) Expression of activin A and follistatin core proteins by human prostate tumor cell lines Endocrinology 140, 5303-9
59 Nakagawa, H., Liyanarachchi, S., Davuluri, R. V., Auer, H., Martin, E. W., Jr., de la Chapelle, A. and Frankel, W. L. (2004) Role of cancer-associated stromal fibroblasts in metastatic colon cancer to the liver and their expression profiles Oncogene 23, 7366-77
60 Di Simone, N., Crowley, W. F., Jr., Wang, Q. F., Sluss, P. M. and Schneyer, A. L. (1996) Characterization of inhibin/activin subunit, follistatin, and activin type II receptors in human ovarian cancer cell lines: a potential role in autocrine growth regulation Endocrinology 137, 486-94
61 Ogino, H., Yano, S., Kakiuchi, S., Muguruma, H., Ikuta, K., Hanibuchi, M., Uehara, H., Tsuchida, K., Sugino, H. and Sone, S. (2008) Follistatin suppresses the production of experimental multiple-organ metastasis by small cell lung cancer cells in natural killer cell-depleted SCID mice Clin. Cancer Res. 14, 660-7
62 Tomita, Y., Yang, X., Ishida, Y., Nemoto-Sasaki, Y., Kondo, T., Oda, M., Watanabe, G., Chaldakov, G. N., Fujii, C. and Mukaida, N. (2004) Spontaneous regression of lung metastasis in the absence of tumor necrosis factor receptor p55 Int. J. Cancer 112, 927-33
63 Lipton, A., Ali, S. M., Leitzel, K., Chinchilli, V., Witters, L., Engle, L., Holloway, D., Bekker, P. and Dunstan, C. R. (2002) Serum osteoprotegerin levels in healthy controls and cancer patients Clin. Cancer Res. 8, 2306-10
64 Mizutani, Y., Matsubara, H., Yamamoto, K., Nan Li, Y., Mikami, K., Okihara, K., Kawauchi, A., Bonavida, B. and Miki, T. (2004) Prognostic significance of serum osteoprotegerin levels in patients with bladder carcinoma Cancer 101, 1794-802
65 Niklinski, J., Furman, M., Palynyczko, Z., Laudanski, J. and Bulatowicz, J. (1991) Carcinoembryonic antigen, neuron-specific enolase and creatine kinase-BB as tumor markers for carcinoma of the lung Neoplasma 38, 645-51
66 Niklinski, J., Furman, M., Laudanski, J., Palynyczko, Z. and Welk, M. (1991) Evaluation of carcinoembryonic antigen (CEA) and brain-type creatine kinase (CK-BB) in serum from patients with carcinoma of the lung Neoplasma 38, 129-35
67 Chen, Y., Zhang, H., Xu, A., Li, N., Liu, J., Liu, C., Lv, D., Wu, S., Huang, L., Yang, S., He, D. and Xiao, X. (2006) Elevation of serum l-lactate dehydrogenase B correlated with the clinical stage of lung cancer Lung Cancer 54, 95-102
68 Sun, T., Gao, Y., Tan, W., Ma, S., Zhang, X., Wang, Y., Zhang, Q., Guo, Y., Zhao, D., Zeng, C. and Lin, D. (2006) Haplotypes in matrix metalloproteinase gene cluster on chromosome 11q22 contribute to the risk of lung cancer development and progression Clin. Cancer Res. 12, 7009-17
69 Kim, J. H., Bogner, P. N., Baek, S. H., Ramnath, N., Liang, P., Kim, H. R., Andrews, C. and Park, Y. M. (2008) Up-regulation of peroxiredoxin 1 in lung cancer and its implication as a prognostic and therapeutic target Clin. Cancer Res. 14, 2326-33
70 Kuk C, Kulasingam V, Gunawardana C G, Smith C R, Batruch I, Diamandis E P. (2009) Mining the ovarian cancer ascites proteome for potential ovarian cancer biomarkers. Mol Cell Proteomics. 8, 661-9
71 Christopoulos, T K, Diamandis E P (1992) Enzymatically Amplified Time-Resolved Fluorescence Immunoassay with Terbium Chelates Anal Chem 64:342-46
72. Kirkpatrick D S, Gerber S A, Gygi S P (2005) The absolute quantification strategy: a general procedure for the quantification of proteins and post-translational modifications Methods 35: 265-73
73. Gerber S A, Rush J, Stemman O, Kirschner, M W, Gygi S P (2003) Absolute quantification of proteins and phosphoproteins from cell lysates by tandem MS Proc Natl Acad Sci 100:6940-45
74 Kulasingam V, Smith C R, Batruch I, Buckler A, Jeffery D A, Diamandis E P (2008) “Product ion monitoring” assay for prostate-specific antigen in serum using a linear ion-trap. J of Proteome Res 7: 640-647
75. Pepe M S, Etzioni R, Feng Z Potter J D, Thompson M L, Thornquist M, Winget M Yasui Y (2001) Phases of Biomarker Development for Early Detection of Cancer Natl Cancer Inst 93:1054-61
76. Pepe M S, Feng Z, Janes H, Bossuyt P M, Potter J D (2008) Pivotal Evaluation of the Accuracy of a Biomarker Used for Classification or Prediction: Standards for Study Design J Natl Cancer Inst 100:1432-38
77. Pepe, M S, Longton G, Janes, H (2009) Estimation and comparison of receiver operating characteristic curves. Stata Journal 9(1):1-16.
78. Janes, H, Longton G, Pepe, M S (2009) Accommodating covariates in receiver operating characteristic analysis. Stata Journal 9(1):17-39.

1. A method of screening for, diagnosing or detecting lung cancer in a subject, the method comprising:
a) determining a level of a biomarker or a plurality of biomarkers in a sample from the subject, wherein the biomarker(s) is/are selected from the biomarkers listed in Table 8, preferably Pentraxin 3, more preferably ADAM-17, Osteoprotegerin, Follistatin and/or sTNF RI; and
b) comparing the level of each biomarker in the sample with a control;
wherein an increased level of any one of the biomarkers compared to the control is indicative that the subject has lung cancer, and/or is in need of follow up lung cancer testing.

2. (canceled)

3. The method of claim 1 wherein the follow up testing is sputum analysis and/or imaging.

4. The method of claim 1 for prognosing lung cancer recurrence in a subject previously having lung cancer, the method comprising:
(a) determining the level of a biomarker or a plurality of biomarkers in a sample from the subject, optionally wherein the sample is obtained after treatment, optionally obtained after surgical resection, wherein the biomarker(s) is/are selected from the biomarkers listed in Table 8; and
(b) comparing the level of each biomarker in the sample with a positive control or a reference level associated with recurrence;
wherein the disease outcome associated with the positive control or reference level most similar to the level of each biomarker in the sample is the predicted prognosis.

5. (canceled)

6. The method of claim 1, wherein the lung cancer is a small cell lung cancer (SCLC) or a non-small cell lung cancer (NSCLC).

7. (canceled)

8. The method of claim 7, wherein the NSCLC is an adenocarcinoma, a squamous cell carcinoma or a large cell carcinoma.

9. The method of claim 1, wherein the biomarker(s) is/are selected from ADAM-17, Osteoprotegerin, Pentraxin 3, Follistatin, sTNF RI, and/or any combination thereof.

10-16. (canceled)

17. The method of claim 1, wherein the sample and/or control comprises a biological fluid, optionally blood, tumor biopsy, serum, plasma, sputum, pleural effusion, nasal lavage fluid, BAL fluid, saliva and/or tumor interstitial fluid.

18-32. (canceled)

33. The method of claim 1, wherein the biomarker is Osteoprotegerin and the level of Osteoprotegerin in the sample relative to the control is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 5.0, 7.5, 10, 15 or 20 fold.

34. The method of claim 1, wherein the biomarker is sTNF RI and the level of sTNF RI in the sample relative to the control is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 5.0, 6.0, 8.0 or 10 fold.

35. The method of claim 1, wherein the biomarker is Follistatin and the level of Follistatin in the sample relative to the control is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 5.0, 6.0, 8.0 or 10 fold.

36. The method of claim 1, wherein the biomarker is Pentraxin 3 and the level of Pentraxin 3 in the sample relative to the control is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.2, 5.4, 5.6, 5.8, 6.0, 6.5, 7.0, 7.5, 8.0, 8.5, 9.0, 9.5, 10, 15, 20 or 40 fold.

37. The method of claim 1, wherein the biomarker is ADAM-17 and the level of ADAM-17 in the sample relative to the control is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.2, 4.4, 4.6, 4.8, 5.0, 5.5, 6.0, 6.5, 7.0, 7.5, 8.0, 8.5, 9.0, 9.5, 10, 15, 20, 40, 60, 80 or 100 fold.

38. The method of claim 1, wherein the biomarker level determined is a polypeptide biomarker level.

39. The method according to claim 38, wherein the level of polypeptide biomarker determined is or comprises soluble polypeptide biomarker.

40. The method according to claim 38, wherein the level of polypeptide biomarker is determined by contacting the sample with a detection agent such as an antibody or antibody fragment wherein the detection agent forms a complex with the biomarker.

41-43. (canceled)

44. The method according to claim 38, wherein the level of at least one polypeptide biomarker is determined using immunohistochemistry or an immunoassay.

45-48. (canceled)

49. An immunoassay for detecting a biomarker comprising an antibody immobilized on a solid support, wherein the antibody binds a biomarker, the biomarker selected from ADAM-17, Osteoprotegerin, or a combination thereof for use in the method of claim 1.

50. (canceled)

51. A composition comprising at least two detection agents that bind a biomarker selected from the biomarkers listed in Table 8, preferably selected from ADAM-17, Osteoprotegerin, Pentraxin 3, Follistatin, or sTNF RI for use in the method of claim 1.

52. (canceled)

53. A kit for detecting a biomarker comprising:
(a) at least two agents, each of which binds a biomarker selected from the biomarkers listed in Table 8, preferably selected from ADAM-17, Osteoprotegerin, Pentraxin 3, Follistatin, or sTNF RI, or any combination thereof; and
(b) instructions for use, or a quantity of at least one purified standard, wherein the standard is selected from ADAM-17 polypeptide, Osteoprotegerin polypeptide, Pentraxin 3 polypeptide, Follistatin polypeptide or sTNF RI polypeptide
for use in the method of claim 1.

54. (canceled)

55. A method of monitoring response to treatment comprising:
a) determining a base-line level according to the method of claim 1a;
b) determining a level of a biomarker or a plurality of biomarkers in a post-treatment sample from the subject; and
c) comparing the level of each biomarker in the post-treatment sample with the base-line level;
wherein an increase in the biomarker level in the post-treatment sample compared to the baseline level is indicative the subject is not responding or is responding poorly to treatment, and a decrease in the biomarker level in the post treatment sample compared to the base-line level is indicative that the subject is responding to treatment.

56. A method of monitoring response to treatment according to claim 55, wherein the biomarker(s) is or comprises Pentraxin 3.

57. A method of monitoring disease progression comprising:
a) determining a base-line level according to the method of claim 1a;
b) determining a level of a biomarker or a plurality of biomarkers in a sample taken subsequent to the base-line sample from the subject; and
c) comparing the level of each biomarker in the sample with the base-line level;
wherein an increase in the biomarker level in the post-base-line sample compared to the base-line level is indicative the disease is progressing, and a decrease in the biomarker level in the post base-line sample compared to the base-line level is indicative that the disease is not progressing.

58. A method of monitoring disease progression according to claim 57, wherein the biomarker(s) is or comprises one or more of ADAM-17, Osteoprotegerin, Pentraxin 3, Follistatin, or sTNF RI, preferably Pentraxin 3.

59-61. (canceled)

62. The method of claim 4 for prognosing lung cancer recurrence in a subject previously having lung cancer, the method comprising:
(a) determining the level of Pentraxin 3 in a sample from the subject, optionally wherein the sample is obtained after treatment, optionally obtained after surgical resection; and
(b) comparing the level of Pentraxin 3 in the sample with a positive control or a reference level associated with recurrence;
wherein the disease outcome associated with the positive control or reference level most similar to the level of Pentraxin 3 in the sample is the predicted prognosis.

63. The method of claim 6, wherein the stage of said lung cancer is at stage I, stage II, stage III or stage IV.

64. (canceled)
